SEQUENCE LISTING (36 sequences)

SEQ ID NO: 1 - DNA, 6314 bp, Artificial Sequence
  Synthetic Construct
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcatcagccaacaagacgagcttctgggccggctccgttaactatggtccattgtactgcgtcaagttcgcacaagcgtgcttccacttctggcgcatcttttgcttcacagatcacgtgcacattagggccggcgtctatcgtccagtaggactgcaaattatcttgggctctccagcgttgaacggcttgcatgaccgctaaagtgcctggcaaccagtacattgttgaaggctgtgcggtcatcgctattacgtgcatagacatggcgtccgcctctgacgcccgtccgagccgttcaatatcacgctcaaggataccctgtcttacatctgctaaccgctgttcaattccttccagacgcacagaaaagtatggactagtggttgccacggagtggccgcttgtagatgcaacatgtttagcttccgtggagataacagcaacaatatcgacgagattccaatgttccggtggagcgatctgtgccgcataagagccagcatgggttccatcattgtaccactctacaaaaccagcagggatactgcgacaagcacttcccgaaccacttaagcgggtaaggcgagagagttctgcctcatctaactccagtctaaatgcactggcagcagcccgagtaagggcggcaaacgccgcagcggagctcgcgatacctgcatcagacgggaaattattacgactgcggacttccacgcgttcggttacaccagccagctggcgcaagcgctcaatctgctggataactctttcgaactggcgtcccttagcctgcacttcctcacctccagaaagtgccaaccacacggaatcgtcaactgcctctggaagacattgcacggttgtttcagtgaggcaaccatccaagttcatggaaatcgagccattggtaggaagggtcaactgactgtcgtgctggccccaatatttgatgaacgcaatgttggcacaagcgacagccgtcgctgcgtgagacagctgtttcattccgttccggacatacataccctggaagtataagttctctccaccggccccatggtgatgatggtggtgcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 2 - DNA, 6095 bp, Artificial Sequence
  Synthetic Construct
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcatcaatccaattttataatggttcccggcccattcagttggccactcaacgcagattggagctgttggggaccgcaaatccaaatttccaactgcggggcctgctggacaagctgccacatagcttctaccttattgcgcattcctcctgtgacgtccacgccatgagacccgccaagccgtgctataatggtagcgtagttggttctgttgatgagtggaataggctgggcatcggcatgttgccgagggtcggcatcatacacggcctgctctcccaacagaatgatctgcgtcggctgtaagggaccgaccagggcactaaaaatgcgctctgtactagcgatggtacaaccctgggccacatccagcagtacatcgccatatataactggaatcgtcccggctgctaaaagcgtcgccaacggctgagagccaatctgctgaatttcccccgcgttcgcgagactactggccatcggttgaataccaattgctggtaagtctgcgtctaagcaagctccgacaaccgcacgattcagccgggccatggcatccgccacacgagcaacgccccaccaactttgttcgttgataataccctgggcggtctggtaccgttctgcccagtaatggccgaatgagccacctccatgtcccaacagaattggctggttaggatgggcctggcgccatgcactcagatccgtcacgacctgtttaagtgtttggtcaactaaccgttcggccgttgtcttatctgtgagcatagaaccacccagcttgataaaaatcggcttgttcattccctggaagtacagattctctccgccagctccgtggtggtgatgatggtgcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 3 - DNA, 6317 bp, Artificial Sequence
  Synthetic Construct
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcatcaaatcagctgagcaccctgccccgcgcgagctttgaatattgattgtactcccggacattctttaagaagcgcctcgactttctcagcctcgctcgaaagggttaacacgtgcacatttggaccggcatccaccgttgaacagactggtatacccttcttgcgccaatgaattactttccataagatcacttcagtttccggtaaccaataattgagtggtggtttacttgttctcatgaccgcgtgcatgagattactatcctcctccacaacgctcgcgaagtgttcaaaatcacggtcaaggatcgctttccgacagatttctatgcgttcttctacacgttcctgccgtaaaaggtgaagatcggaagtgctcgccagagcatgaccgcctgtggagcctacagttttgtgttcggagttaaggacgcaaatcagatctacaagatcccaatgatccgccggtgctatactccatgcaaatgaatcctggtctgtcgagcccgcttgccattccacaaagccatccggaatgctacgacaggcagacccactacctctccgcgcgagacggctcagtgcttcttcatccagagaaaggccagcggctttagacgcggccaaagcaagggccgcaaacgcggaagctgaactagctatcccagctccagatgggaagctgttctccgactcgacttttgcgaagaaggaaatgcccgccagatcgcgaacgatttccaggaaatcgctaacgcgacgcagcgcgtcccactcaataggcttgccggacaacttaaactggtctgccgaaagggatggatcaaactgtaccgatgtttttgtttcgagccctgaaaggttcattgacaaggatccgttgcatggcagacgcaaatcattgtcgcgattaccccagtacttaatgaatgcgatattcgggtgagccagggccgacacttccagaaattctggcgatttcatagggatttcattattgttgatcacgcggtaatagtaatccatgccttgaaaatacagattctcgccgcctgcaccgtgatggtgatggtggtgcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 4 - DNA, 7750 bp, Artificial Sequence
  Synthetic Construct
atggccaagttgaccagtgccgttccggtgctcaccgcgcgcgacgtcgccggagcggtcgagttctggaccgaccggctcgggttctcccctagtaacggccgccagtgtgctggaattcaggcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagcctgaaaattaaccctcactaaagggcggccgcgaagttcctattctctagaaagtataggaacttcctcgagccctatagtgagtcgtattaaattcatataaaaaacatacagataaccatctgcggtgataaattatctctggcggtgttgacgtaaataccactggcggtgatactgagcacatcagcaggacgcactgaccaccatgaaggtgcaaaggaggtaaaaaaacatggtatcctgttctgcgccgggtaagatttacctgttcggtgaacacgccgtagtttatggcgaaactgcaattgcgtgtgcggtggaactgcgtacccgtgttcgcgcggaactcaatgactctatcactattcagagccagatcggccgcaccggtctggatttcgaaaagcacccttatgtgtctgcggtaattgagaaaatgcgcaaatctattcctattaacggtgttttcttgaccgtcgattccgacatcccggtgggctccggtctgggtagcagcgcagccgttactatcgcgtctattggtgcgctgaacgagctgttcggctttggcctcagcctgcaagaaatcgctaaactgggccacgaaatcgaaattaaagtacagggtgccgcgtccccaaccgatacgtatgtttctaccttcggcggcgtggttaccatcccggaacgtcgcaaactgaaaactccggactgcggcattgtgattggcgataccggcgttttctcctccaccaaagagttagtagctaacgtacgtcagctgcgcgaaagctacccggatttgatcgaaccgctgatgacctctattggcaaaatctctcgtatcggcgaacaactggttctgtctggcgactacgcatccatcggccgcctgatgaacgtcaaccagggtctcctggacgccctgggcgttaacatcttagaactgagccagctgatctattccgctcgtgcggcaggtgcgtttggcgctaaaatcacgggcgctggcggcggtggctgtatggttgcgctgaccgctccggaaaaatgcaaccaagtggcagaagcggtagcaggcgctggcggtaaagtgactatcactaaaccgaccgagcaaggtctgaaagtagattaagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggcccaagaaaaatgccccgcttacgcagggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaatgttggattttagtttttccagcaattcgttgttcggtgcaacgacgataaccggcatatcggcatcaatcagcgccagcggaccgtgtttcagttcacctgcagcgtaggcttcagcgtgaatgtaagagatctctttcagcttcaatgcgccttccagcgcgattgggtactgatcgccacggcccaggaacagcgcgtgatgtttgtcagagaaatcttctgccagagcttcaatgcgtttgtcctgagacagcatctgctcaatacggctcggcaacgcctgcagaccatgcacaatgtcatgttcaatggaggcatccagacctttcaggcgagacagcttcgccaccagcatcaacagcacagttaactgagtggtgaatgctttagtggatgccacgccgatttctgtacccgcgttggtcattagcgccagatggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccacc

SEQ ID NO: 5 - DNA, 7066 bp, Artificial Sequence
  Synthetic Construct
atggccaagttgaccagtgccgttccggtgctcaccgcgcgcgacgtcgccggagcggtcgagttctggaccgaccggctcgggttctcccctagtaacggccgccagtgtgctggaattcaggcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagcctgaaaattaaccctcactaaagggcggccgcgaagttcctattctctagaaagtataggaacttcctcgagccctatagtgagtcgtattaaattcatataaaaaacatacagataaccatctgcggtgataaattatctctggcggtgttgacgtaaataccactggcggtgatactgagcacatcagcaggacgcactgaccaccatgaaggtgcaaaggaggtaaaaaaacatggtatcctgttctgcgccgggtaagatttacctgttcggtgaacacgccgtagtttatggcgaaactgcaattgcgtgtgcggtggaactgcgtacccgtgttcgcgcggaactcaatgactctatcactattcagagccagatcggccgcaccggtctggatttcgaaaagcacccttatgtgtctgcggtaattgagaaaatgcgcaaatctattcctattaacggtgttttcttgaccgtcgattccgacatcccggtgggctccggtctgggtagcagcgcagccgttactatcgcgtctattggtgcgctgaacgagctgttcggctttggcctcagcctgcaagaaatcgctaaactgggccacgaaatcgaaattaaagtacagggtgccgcgtccccaaccgatacgtatgtttctaccttcggcggcgtggttaccatcccggaacgtcgcaaactgaaaactccggactgcggcattgtgattggcgataccggcgttttctcctccaccaaagagttagtagctaacgtacgtcagctgcgcgaaagctacccggatttgatcgaaccgctgatgacctctattggcaaaatctctcgtatcggcgaacaactggttctgtctggcgactacgcatccatcggccgcctgatgaacgtcaaccagggtctcctggacgccctgggcgttaacatcttagaactgagccagctgatctattccgctcgtgcggcaggtgcgtttggcgctaaaatcacgggcgctggcggcggtggctgtatggttgcgctgaccgctccggaaaaatgcaaccaagtggcagaagcggtagcaggcgctggcggtaaagtgactatcactaaaccgaccgagcaaggtctgaaagtagattaaccaggatagctctttgatcggaactgaacttcagtttagcaaaggagagtatcgatggattactattaccgcgtgatcaacaataatgaaatccctatgaaatcgccagaatttctggaagtgtcggccctggctcacccgaatatcgcattcattaagtactggggtaatcgcgacaatgatttgcgtctgccatgcaacggatccttgtcaatgaacctttcagggctcgaaacaaaaacatcggtacagtttgatccatccctttcggcagaccagtttaagttgtccggcaagcctattgagtgggacgcgctgcgtcgcgttagcgatttcctggaaatcgttcgcgatctggcgggcatttccttcttcgcaaaagtcgagtcggagaacagcttcccatctggagctgggatagctagttcagcttccgcgtttgcggcccttgctttggccgcgtctaaagccgctggcctttctctggatgaagaagcactgagccgtctcgcgcggagaggtagtgggtctgcctgtcgtagcattccggatggctttgtggaatggcaagcgggctcgacagaccaggattcatttgcatggagtatagcaccggcggatcattgggatcttgtagatctgatttgcgtccttaactccgaacacaaaactgtaggctccacaggcggtcatgctctggcgagcacttccgatcttcaccttttacggcaggaacgtgtagaagaacgcatagaaatctgtcggaaagcgatccttgaccgtgattttgaacacttcgcgagcgttgtggaggaggatagtaatctcatgcacgcggtcatgagaacaagtaaaccaccactcaattattggttaccggaaactgaagtgatcttatggaaagtaattcattggcgcaagaagggtataccagtctgttcaacggtggatgccggtccaaatgtgcacgtgttaaccctttcgagcgaggctgagaaagtcgaggcgcttcttaaagaatgtccgggagtacaatcaatattcaaagctcgcgcggggcagggtgctcagctgatttgatttgtagatgccacggaccatagcaatatactgcgagaagggagggttaacttatgaacaagccgatttttatcaagctgggtggttctatgctcacagataagacaacggccgaacggttagttgaccaaacacttaaacaggtcgtgacggatctgagtgcatggcgccaggcccatcctaaccagccaattctgttgggacatggaggtggctcattcggccattactgggcagaacggtaccagaccgcccagggtattatcaacgaacaaagttggtggggcgttgctcgtgtggcggatgccatggcccggctgaatcgtgcggttgtcggagcttgcttagacgcagacttaccagcaattggtattcaaccgatggccagtagtctcgcgaacgcgggggaaattcagcagattggctctcagccgttggcgacgcttttagcagccgggacgattccagttatatatggcgatgtactgctggatgtggcccagggttgtaccatcgctagtacagagcgcatttttagtgccctggtcggtcccttacagccgacgcagatcattctgttgggagagcaggccgtgtatgatgccgaccctcggcaacatgccgatgcccagcctattccactcatcaacagaaccaactacgctaccattatagcacggcttggcgggtctcatggcgtggacgtcacaggaggaatgcgcaataaggtagaagctatgtggcagcttgtccagcaggccccgcagttggaaatttggatttgcggtccccaacagctccaatctgcgttgagtggccaactgaatgggccgggaaccattataaaattggattgaaaatgactctgaattgctgccggctgaaaagcaggctctcggaggaggaaatatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggcccaagaaaaatgccccgcttacgcagggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaatgttggattttagtttttccagcaattcgttgttcggtgcaacgacgataaccggcatatcggcatcaatcagcgccagcggaccgtgtttcagttcacctgcagcgtaggcttcagcgtgaatgtaagagatctctttcagcttcaatgcgccttccagcgcgattgggtactgatcgccacggcccaggaacagcgcgtgatgtttgtcagagaaatcttctgccagagcttcaatgcgtttgtcctgagacagcatctgctcaatacggctcggcaacgcctgcagaccatgcacaatgtcatgttcaatggaggcatccagacctttcaggcgagacagcttcgccaccagcatcaacagcacagttaactgagtggtgaatgctttagtggatgccacgccgatttctgtacccgcgttggtcattagcgccagatggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccacc

SEQ ID NO: 6 - DNA, 7060 bp, Artificial Sequence
  Synthetic Construct
atggccaagttgaccagtgccgttccggtgctcaccgcgcgcgacgtcgccggagcggtcgagttctggaccgaccggctcgggttctcccctagtaacggccgccagtgtgctggaattcaggcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagcctgaaaattaaccctcactaaagggcggccgcgaagttcctattctctagaaagtataggaacttcctcgagccctatagtgagtcgtattaaattcatataaaaaacatacagataaccatctgcggtgataaattatctctggcggtgttgacgtaaataccactggcggtgatactgagcacatcagcaggacgcactgaccaccatgaaggtgcaaaggaggtaaaaaaacatggtatcctgttctgcgccgggtaagatttacctgttcggtgaacacgccgtagtttatggcgaaactgcaattgcgtgtgcggtggaactgcgtacccgtgttcgcgcggaactcaatgactctatcactattcagagccagatcggccgcaccggtctggatttcgaaaagcacccttatgtgtctgcggtaattgagaaaatgcgcaaatctattcctattaacggtgttttcttgaccgtcgattccgacatcccggtgggctccggtctgggtagcagcgcagccgttactatcgcgtctattggtgcgctgaacgagctgttcggctttggcctcagcctgcaagaaatcgctaaactgggccacgaaatcgaaattaaagtacagggtgccgcgtccccaaccgatacgtatgtttctaccttcggcggcgtggttaccatcccggaacgtcgcaaactgaaaactccggactgcggcattgtgattggcgataccggcgttttctcctccaccaaagagttagtagctaacgtacgtcagctgcgcgaaagctacccggatttgatcgaaccgctgatgacctctattggcaaaatctctcgtatcggcgaacaactggttctgtctggcgactacgcatccatcggccgcctgatgaacgtcaaccagggtctcctggacgccctgggcgttaacatcttagaactgagccagctgatctattccgctcgtgcggcaggtgcgtttggcgctaaaatcacgggcgctggcggcggtggctgtatggttgcgctgaccgctccggaaaaatgcaaccaagtggcagaagcggtagcaggcgctggcggtaaagtgactatcactaaaccgaccgagcaaggtctgaaagtagattaaccaggatagctctttgatcggaacaaacgaaaatcaaaggaggaaccaacaatgtatgtccggaacggaatgaaacagctgtctcacgcagcgacggctgtcgcttgtgccaacattgcgttcatcaaatattggggccagcacgacagtcagttgacccttcctaccaatggctcgatttccatgaacttggatggttgcctcactgaaacaaccgtgcaatgtcttccagaggcagttgacgattccgtgtggttggcactttctggaggtgaggaagtgcaggctaagggacgccagttcgaaagagttatccagcagattgagcgcttgcgccagctggctggtgtaaccgaacgcgtggaagtccgcagtcgtaataatttcccgtctgatgcaggtatcgcgagctccgctgcggcgtttgccgcccttactcgggctgctgccagtgcatttagactggagttagatgaggcagaactctctcgccttacccgcttaagtggttcgggaagtgcttgtcgcagtatccctgctggttttgtagagtggtacaatgatggaacccatgctggctcttatgcggcacagatcgctccaccggaacattggaatctcgtcgatattgttgctgttatctccacggaagctaaacatgttgcatctacaagcggccactccgtggcaaccactagtccatacttttctgtgcgtctggaaggaattgaacagcggttagcagatgtaagacagggtatccttgagcgtgatattgaacggctcggacgggcgtcagaggcggacgccatgtctatgcacgtaatagcgatgaccgcacagccttcaacaatgtactggttgccaggcactttagcggtcatgcaagccgttcaacgctggagagcccaagataatttgcagtcctactggacgatagacgccggccctaatgtgcacgtgatctgtgaagcaaaagatgcgccagaagtggaagcacgcttgtgcgaacttgacgcagtacaatggaccatagttaacggagccggcccagaagctcgtcttgttggctgatttgtagatgccacggaccatagcaatatactgcgagaagggagggttaacttatgaacaagccgatttttatcaagctgggtggttctatgctcacagataagacaacggccgaacggttagttgaccaaacacttaaacaggtcgtgacggatctgagtgcatggcgccaggcccatcctaaccagccaattctgttgggacatggaggtggctcattcggccattactgggcagaacggtaccagaccgcccagggtattatcaacgaacaaagttggtggggcgttgctcgtgtggcggatgccatggcccggctgaatcgtgcggttgtcggagcttgcttagacgcagacttaccagcaattggtattcaaccgatggccagtagtctcgcgaacgcgggggaaattcagcagattggctctcagccgttggcgacgcttttagcagccgggacgattccagttatatatggcgatgtactgctggatgtggcccagggttgtaccatcgctagtacagagcgcatttttagtgccctggtcggtcccttacagccgacgcagatcattctgttgggagagcaggccgtgtatgatgccgaccctcggcaacatgccgatgcccagcctattccactcatcaacagaaccaactacgctaccattatagcacggcttggcgggtctcatggcgtggacgtcacaggaggaatgcgcaataaggtagaagctatgtggcagcttgtccagcaggccccgcagttggaaatttggatttgcggtccccaacagctccaatctgcgttgagtggccaactgaatgggccgggaaccattataaaattggattgaaaatgactctgaattgctgccggctgaaaagcaggctctcggaggaggaaatatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggcccaagaaaaatgccccgcttacgcagggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaatgttggattttagtttttccagcaattcgttgttcggtgcaacgacgataaccggcatatcggcatcaatcagcgccagcggaccgtgtttcagttcacctgcagcgtaggcttcagcgtgaatgtaagagatctctttcagcttcaatgcgccttccagcgcgattgggtactgatcgccacggcccaggaacagcgcgtgatgtttgtcagagaaatcttctgccagagcttcaatgcgtttgtcctgagacagcatctgctcaatacggctcggcaacgcctgcagaccatgcacaatgtcatgttcaatggaggcatccagacctttcaggcgagacagcttcgccaccagcatcaacagcacagttaactgagtggtgaatgctttagtggatgccacgccgatttctgtacccgcgttggtcattagcgccagatggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccacc

SEQ ID NO: 7 - DNA, 2439 bp, Listeria grayi
atggttaaagacattgtaataattgatgccctccgtactcccatcggtaagtaccgcggtcagctctcaaagatgacggcggtggaattgggaaccgcagttacaaaggctctgttcgagaagaacgaccaggtcaaagaccatgtagaacaagtcatttttggcaacgttttacaggcagggaacggccagaatcccgcccgtcagatcgcccttaattctggcctgtccgcagagataccggcttcgactattaaccaggtgtgtggttctggcctgaaagcaataagcatggcgcgccaacagatcctactcggagaagcggaagtaatagtagcaggaggtatcgaatccatgacgaatgcgccgagtattacatattataataaagaagaagacaccctctcaaagcctgttcctacgatgaccttcgatggtctgaccgacgcgtttagcggaaagattatgggtttaacagccgaaaatgttgccgaacagtacggcgtatcacgtgaggcccaggacgcctttgcgtatggatcgcagatgaaagcagcaaaggcccaagaacagggcattttcgcagctgaaatactgcctcttgaaataggggacgaagttattactcaggacgagggggttcgtcaagagaccaccctcgaaaaattaagtctgcttcggaccatttttaaagaagatggtactgttacagcgggcaacgcctcaacgatcaatgatggcgcctcagccgtgatcattgcatcaaaggagtttgctgagacaaaccagattccctaccttgcgatcgtacatgatattacagagataggcattgatccatcaataatgggcattgctcccgtgagtgcgatcaataaactgatcgatcgtaaccaaattagcatggaagaaatcgatctctttgaaattaatgaggcatttgcagcatcctcggtggtagttcaaaaagagttaagcattcccgatgaaaagatcaatattggcggttccggtattgcactaggccatcctcttggcgccacaggagcgcgcattgtaaccaccctagcgcaccagttgaaacgtacacacggacgctatggtattgcctccctgtgcattggcggtggccttggcctagcaatattaatagaagtgcctcaggaagatcagccggttaaaaaattttatcaattggcccgtgaggaccgtctggctagacttcaggagcaagccgtgatcagcccagctacaaaacatgtactggcagaaatgacacttcctgaagatattgccgacaatctgatcgaaaatcaaatatctgaaatggaaatccctcttggtgtggctttgaatctgagggtcaatgataagagttataccatcccactagcaactgaggaaccgagtgtaatcgctgcctgtaataatggtgcaaaaatggcaaaccacctgggcggttttcagtcagaattaaaagatggtttcctgcgtgggcaaattgtacttatgaacgtcaaagaacccgcaactatcgagcatacgatcacggcagagaaagcggcaatttttcgtgccgcagcgcagtcacatccatcgattgtgaaacgaggtgggggtctaaaagagatagtagtgcgtacgttcgatgatgatccgacgttcctgtctattgatctgatagttgatactaaagacgcaatgggcgctaacatcattaacaccattctcgagggtgtagccggctttctgagggaaatccttaccgaagaaattctgttctctattttatctaattacgcaaccgaatcaattgtgaccgccagctgtcgcataccttacgaagcactgagtaaaaaaggtgatggtaaacgaatcgctgaaaaagtggctgctgcatctaaatttgcccagttagatccttatcgagctgcaacccacaacaaaggtattatgaatggtattgaggccgtcgttttggcctcaggaaatgacacacgggcggtcgcggcagccgcacatgcgtatgcttcacgcgatcagcactatcggggcttaagccagtggcaggttgcagaaggcgcgttacacggggagatcagtctaccacttgcactcggcagcgttggcggtgcaattgaggtcttgcctaaagcgaaggcggcattcgaaatcatggggatcacagaggcgaaggagctggcagaagtcacagctgcggtagggctggcgcaaaacctggcggcgttaagagcgcttgttagtgaaggaatacagcaaggtcacatgtcgctccaggctcgctctcttgcattatcggtaggtgctacaggcaaggaagttgaaatcctggccgaaaaattacagggctctcgtatgaatcaggcgaacgctcagaccatactcgcagagatcagatcgcaaaaagttgaattgtga

SEQ ID NO: 8 - DNA, 1158 bp, Enterococcus faecium
atgaccatgaacgttggaatcgataaaatgtcattctttgttccaccttactttgtggacatgactgatctggcagtagcacgggatgtcgatcccaataagtttctgattggtattggccaggaccagatggcagttaatccgaaaacgcaggatattgtgacatttgccacaaatgctgccaaaaacatactgtcagctgaggaccttgataaaattgatatggtcatagtcggcaccgagagtggaatcgatgaatccaaagcgagtgccgtagtgcttcacaggttgctcggtatccagaagtttgctcgctcctttgaaatcaaagaagcctgttatgggggtaccgcggctttacagttcgctgtaaaccacattaggaatcatcctgaatcaaaggttcttgtagttgcatcagatatcgcgaaatacggcctggcttctggaggtgaaccaacgcaaggtgcaggcgctgtggctatgctcgtctcaactgaccctaagatcattgctttcaacgacgatagcctcgcgcttacacaagatatctatgacttctggcgaccagttggacatgactatcctatggtcgacgggcctcttagtacagagacctacatccagtcatttcagaccgtatggcaggaatacacaaaacggtcgcagcatgcactggcagactttgctgcccttagctttcatatcccgtatactaaaatgggcaaaaaggcgctgcttgcaatccttgaaggcgaatcagaggaggctcagaaccgtatactagcaaaatatgaaaagagtatagcctactccagaaaggcgggtaacctgtataccggtagcctgtatctaggacttatttcacttctggaaaatgcagaagaccttaaagctggtgatttaataggcctcttttcttacggttccggtgctgttgcggagtttttctcaggaaggctggttgaggactatcaggaacagctacttaaaacaaaacatgccgaacagctggcccatagaaagcaactgacaatcgaggagtacgaaacgatgttctccgatcgcttggacgtggacaaagacgccgaatacgaagacacattagcttatagcatttcgtcagtccgaaacaccgtacgtgagtacaggagttga

SEQ ID NO: 9 - DNA, 2442 bp, Enterococcus gallinarum
atgaaagaagtggttatgattgatgcggctcgcacacccattgggaaatacagaggtagtcttagtccttttacagcggtggagctggggacactggtcacgaaagggctgctggataaaacaaagcttaagaaagacaagatagaccaagtgatattcggcaatgtgcttcaggcaggaaacggacaaaacgttgcaagacaaatagccctgaacagtggcttaccagttgacgtgccggcgatgactattaacgaagtttgcgggtccggaatgaaagcggtgattttagcccgccagttaatacagttaggggaggcagagttggtcattgcagggggtacggagtcaatgtcacaagcacccatgctgaaaccttaccagtcagagaccaacgaatacggagagccgatatcatcaatggttaatgacgggctgacggatgcgttttccaatgctcacatgggtcttactgccgaaaaggtggcgacccagttttcagtgtcgcgcgaggaacaagaccggtacgcattgtccagccaattgaaagcagcgcacgcggttgaagccggggtgttctcagaagagattattccggttaagattagcgacgaggatgtcttgagtgaagacgaggcagtaagaggcaacagcactttggaaaaactgggcaccttgcggacggtgttttctgaagagggcacggttaccgctggcaatgcttcaccgctgaatgacggcgctagtgtcgtgattcttgcatcaaaagaatacgcggaaaacaataatctgccttacctggcgacgataaaggaggttgcggaagttggtatcgatccttctatcatgggtattgccccaataaaggccattcaaaagttaacagatcggtcgggcatgaacctgtccacgattgatctgttcgaaattaatgaagcattcgcggcatctagcattgttgtttctcaagagctgcaattggacgaagaaaaagtgaatatctatggcggggcgatagctttaggccatccaatcggcgcaagcggagcccggatactgacaaccttagcatacggcctcctgcgtgagcaaaagcgttatggtattgcgtcattatgtatcggcggtggtcttggtctggccgtgctgttagaagctaatatggagcagacccacaaagacgttcagaagaaaaagttttaccagcttaccccctccgagcggagatcgcagcttatcgagaagaacgttctgactcaagaaacggcacttattttccaggagcagacgttgtccgaagaactgtccgatcacatgattgagaatcaggtctccgaagtggaaattccaatgggaattgcacaaaattttcagattaatggcaagaaaaaatggattcctatggcgactgaagaaccttcagtaatagcggcagcatcgaacggcgccaaaatctgcgggaacatttgcgcggaaacgcctcagcggcttatgcgcgggcagattgtcctgtctggcaaatcagaatatcaagccgtgataaatgccgtgaatcatcgcaaagaagaactgattctttgcgcaaacgagtcgtacccgagtattgttaaacgcgggggaggtgttcaggatatttctacgcgggagtttatgggttcttttcacgcgtatttatcaatcgactttctggtggacgtcaaggacgcaatgggggcaaacatgatcaactctattctcgaaagcgttgcaaataaactgcgtgaatggttcccggaagaggaaatactgttctccatcctgtcaaacttcgctacggagtccctggcatctgcatgttgcgagattccttttgaaagacttggtcgtaacaaagaaattggtgaacagatcgccaagaaaattcaacaggcaggggaatatgctaagcttgacccttaccgcgcggcaacccataacaaggggattatgaacggtatcgaagccgtcgttgccgcaacgggaaacgacacacgggctgtttccgcttctattcacgcatacgccgcccgtaatggcttgtaccaaggtttaacggattggcagatcaagggcgataaactggttggtaaattaacagtcccactggctgtggcgactgtcggtggcgcgtcgaacatattaccaaaagccaaagcttccctcgccatgctggatattgattccgcaaaagaactggcccaagtgatcgccgcggtaggtttagcacagaatctggcggcgttacgtgcattagtgacagaaggcattcagaaaggacacatgggcttgcaagcacgttctttagcgatttcgataggtgccatcggtgaggagatagagcaagtcgcgaaaaaactgcgtgaagctgaaaaaatgaatcagcaaacggcaatacagattttagaaaaaattcgcgagaaatga

SEQ ID NO: 10 - DNA, 1155 bp, Enterococcus casseliflavus
atgaaaatcggtattgaccgtctgtccttcttcatcccgaatttgtatttggacatgactgagctggcagaatcacgcggggatgatccagctaaatatcatattggaatcggacaagatcagatggcagtgaatcgcgcaaacgaggacatcataacactgggtgcaaacgctgcgagtaagatcgtgacagagaaagaccgcgagttgattgatatggtaatcgttggcacggaatcaggaattgaccactccaaagcaagcgccgtgattattcaccatctccttaaaattcagtcgttcgcccgttctttcgaggtaaaagaagcttgctatggcggaactgctgccctgcacatggcgaaggagtatgtcaaaaatcatccggagcgtaaggtcttggtaattgcgtcagacatcgcgcgttatggtttggccagcggaggagaagttactcaaggcgtgggggccgtagccatgatgattacacaaaacccccggattctttcgattgaagacgatagtgtttttctcacagaggatatctatgatttctggcggcctgattactccgagttccctgtagtggacgggcccctttcaaactcaacgtatatagagagttttcagaaagtttggaaccggcacaaggaattgtccggaagagggctggaagattatcaagctattgcttttcacataccctatacgaagatgggtaagaaagcgctccagagtgttttagaccaaaccgatgaagataaccaggagcgcttaatggctagatatgaggagtctattcgctatagccggagaattggtaacctgtacacaggcagcttgtaccttggtcttacaagcttgttggaaaactctaaaagtttacaaccgggagatcggatcggcctcttttcctatggcagtggtgcggtgtccgagttctttaccgggtatttagaagaaaattaccaagagtacctgttcgctcaaagccatcaagaaatgctggatagccggactcggattacggtcgatgaatacgagaccatcttttcagagactctgccagaacatggtgaatgcgccgaatatacgagcgacgtccccttttctataaccaagattgagaacgacattcgttattataaaatctga

SEQ ID NO: 11 - DNA, 2448 bp, Listeria grayi
atggaagaagtggtaattatagatgcacgtcggactccgattggtaaatatcacgggtcgttgaagaagttttcagcggtggcgctggggacggccgtggctaaagacatgttcgaacgcaaccagaaaatcaaagaggagatcgcgcaggtcataattggtaatgtcttgcaggcaggaaatggccagaaccccgcgcggcaagttgctcttcaatcagggttgtccgttgacattcccgcttctacaattaacgaggtttgtgggtctggtttgaaagctatcttgatgggcatggaacaaatccaactcggcaaagcgcaagtagtgctggcaggcggcattgaatcaatgacaaatgcgccaagcctgtcccactataacaaggcggaggatacgtatagtgtcccagtgtcgagcatgacactggatggtctgacagacgcattttctagtaaacctatgggattaacagcggaaaacgtcgcacagcgctacggtatctcccgtgaggcgcaagatcaattcgcatatcaatctcagatgaaagcagcaaaagcgcaggcagaaaacaaattcgctaaggaaattgtgccactggcgggtgaaactaaaaccatcacagctgacgaagggatcagatcccaaacaacgatggagaaactggcaagtctcaaacctgtttttaaaaccgatggcactgtaaccgcagggaatgctagcaccattaatgacggggccgcccttgtgctgcttgctagcaaaacttactgcgaaactaatgacataccgtaccttgcgacaatcaaagaaattgttgaagttggaatcgatccggagattatgggcatctctccgataaaagcgatacaaacattgttacaaaatcaaaaagttagcctcgaagatattggagtttttgaaataaatgaagcctttgccgcaagtagcatagtggttgaatctgagttgggattagatccggctaaagttaaccgttatgggggtggtatatccttaggtcatgcaattggggcaaccggcgctcgcctggccacttcactggtgtatcaaatgcaggagatacaagcacgttatggtattgcgagcctgtgcgttggtggtggacttggactggcaatgcttttagaacgtccaactattgagaaggctaaaccgacagacaaaaagttctatgaattgtcaccagctgaacggttgcaagagctggaaaatcaacagaaaatcagttctgaaactaaacagcagttatctcagatgatgcttgccgaggacactgcaaaccatttgatagaaaatcaaatatcagagattgaactcccaatgggcgtcgggatgaacctgaaggttgatgggaaagcctatgttgtgccaatggcgacggaagagccgtccgtcatcgcggccatgtctaatggtgccaaaatggccggcgaaattcacactcagtcgaaagaacggctgctcagaggtcagattgttttcagcgcgaagaatccgaatgaaatcgaacagagaatagctgagaaccaagctttgattttcgaacgtgccgaacagtcctatccttccattgtgaaaagagagggaggtctccgccgcattgcacttcgtcattttcctgccgattctcagcaggagtctgcggaccagtccacatttttatcagtggacctttttgtagatgtgaaagacgcgatgggggcaaatatcataaatgcaatacttgagggcgtcgcagccctgtttcgcgaatggttccccaatgaggaaattcttttttctattctctcgaacttggctacggagagcttagtcacggctgtttgtgaagtcccatttagtgcacttagcaagagaggtggtgcaacggtggcccagaaaattgtgcaggcgtcgctcttcgcaaagacagacccataccgcgcagtgacccacaacaaagggattatgaacggtgtagaggctgttatgcttgccacaggcaacgacacgcgcgcagtctcagccgcttgtcatggatacgcagcgcgcaccggtagctatcagggtctgactaactggacgattgagtcggatcgcctggtaggcgagataacactgccgctggccatcgctacagttggaggcgctaccaaagtgttgcccaaagctcaagcggcactggagattagtgatgttcactcttctcaagagcttgcagccttagcggcgtcagtaggtttagtacaaaatctcgcggccctgcgcgcactggtttccgaaggtatacaaaaagggcacatgtccatgcaagcccggtctctcgcaatcgcggtcggtgctgaaaaagccgagatcgagcaggtcgccgaaaagttgcggcagaacccgccaatgaatcagcagcaggcgctccgttttcttggcgagatccgcgaacaatga

SEQ ID NO: 12 - DNA, 1155 bp, Enterococcus faecium
atgaacgtcggcattgacaaaattaattttttcgttccaccgtattatctggatatggtcgacctggcccacgcacgcgaagtggacccgaacaaatttacaattggaattggacaggatcagatggctgtgagcaaaaagacgcacgatatcgtaacattcgcggctagtgccgcgaaggaaattttagaacctgaggacttgcaagctatagacatggttatagttggtaccgaatcgggcattgacgagagcaaagcatccgcggtcgttttacatcgtttgttgggcgtacaacctttcgctcgcagttttgaaattaaagaagcctgttacggggcaaccgcaggcattcagtttgccaagactcatatacaagcgaacccggagagcaaggtcctggtaattgcaagcgatatagctcggtatggtcttcggtcaggtggagagcccacacaaggcgcaggggcagttgctatgcttctcacggcaaatcccagaatcctgaccttcgaaaacgacaatctgatgttaacgcaggatatttatgacttctggagaccacttggtcacgcttaccctatggtagatggccacctttccaatcaagtctatattgacagttttaagaaggtctggcaagcacattgcgaacgcaatcaagcttctatatccgactatgccgcgattagttttcatattccgtatacaaaaatgggtaagaaagccctgctcgctgtttttgcagatgaagtggaaactgaacaggaacgcgttatggcacggtatgaagagtctatcgtatattcacgccggatcggcaacttgtatacgggatcattgtacctggggctgatatccttattggaaaacagttctcacctgtcggcgggcgaccggataggattgtttagttatgggagtggcgctgtcagcgaatttttctccggtcgtttagtggcaggctatgaaaatcaattgaacaaagaggcgcatacccagctcctggatcagcgtcagaagctttccatcgaagagtatgaggcgatttttacagattccttagaaattgatcaggatgcagcgttctcggatgacctgccatattccatccgcgagataaaaaacacgattcggtactataaggagagctga

SEQ ID NO: 13 - DNA, 2475 bp, Enterococcus gallinarum
atggaagaagttgtcatcattgacgcactgcgtactccaataggaaagtaccacggttcgctgaaagattacacagctgttgaactggggacagtagcagcaaaggcgttgctggcacgaaatcagcaagcaaaagaacacatagcgcaagttattattggcaacgtcctgcaagccggaagtgggcagaatccaggccgacaagtcagtttacagtcaggattgtcttctgatatccccgctagcacgatcaatgaagtgtgtggctcgggtatgaaagcgattctgatgggtatggagcaaattcagctgaacaaagcctctgtggtcttaacaggcggaattgaaagcatgaccaacgcgccgctgtttagttattacaacaaggctgaggatcaatattcggcgccggttagcacaatgatgcacgatggtctaacagatgctttcagttccaaaccaatgggcttaaccgcagagaccgtcgctgagagatatggaattacgcgtaaggaacaagatgaatttgcttatcactctcaaatgaaggcggccaaagcccaggcggcgaaaaagtttgatcaggaaattgtacccctgacggaaaaatccggaacggttctccaggacgaaggcatcagagccgcgacaacagtcgagaagctagctgagcttaaaacggtgttcaaaaaagacggaacagttacagcgggtaacgcctctacgataaatgatggcgctgctatggtattaatagcatcaaaatcttattgcgaagaacaccagattccttatctggccgttataaaggagatcgttgaggtgggttttgcccccgaaataatgggtatttcccccattaaggctatagacaccctgctgaaaaatcaagcactgaccatagaggatataggaatatttgagattaatgaagcctttgctgcgagttcgattgtggtagaacgcgagttgggcctggaccccaaaaaagttaatcgctatggcggtggtatatcactcggccacgcaattggggcgacgggagctcgcattgcgacgaccgttgcttatcagctgaaagatacccaggagcgctacggtatagcttccttatgcgttggtgggggtcttggattggcgatgcttctggaaaacccatcggccactgcctcacaaactaattttgatgaggaatctgcttccgaaaaaactgagaagaagaagttttatgcgctagctcctaacgaacgcttagcgtttttggaagcccaaggcgctattaccgctgctgaaaccctggtcttccaggagatgaccttaaacaaagagacagccaatcacttaatcgaaaaccaaatcagcgaagttgaaattcctttaggcgtgggcctgaacttacaggtgaatgggaaagcgtataatgttcctctggccacggaggaaccgtccgttatcgctgcgatgtcgaatggcgccaaaatggctggtcctattacaacaacaagtcaggagaggctgttacggggtcagattgtcttcatggacgtacaggacccagaagcaatattagcgaaagttgaatccgagcaagctaccattttcgcggtggcaaatgaaacatacccgtctatcgtgaaaagaggaggaggtctgcgtagagtcattggcaggaatttcagtccggccgaaagtgacttagccacggcgtatgtatcaattgacctgatggtagatgttaaggatgcaatgggtgctaatatcatcaatagtatcctagaaggtgttgcggaattgtttagaaaatggttcccagaagaagaaatcctgttctcaattctctccaatctcgcgacagaaagtctggtaacggcgacgtgctcagttccgtttgataaattgtccaaaactgggaatggtcgacaagtagctggtaaaatagtgcacgcggcggactttgctaagatagatccatacagagctgccacacacaataaaggtattatgaatggcgttgaagcgttaatcttagccaccggtaatgacacccgtgcggtgtcggctgcatgccacggttacgcggcacgcaatgggcgaatgcaagggcttacctcttggacgattatcgaagatcggctgataggctctatcacattacctttggctattgcgacagtggggggtgccacaaaaatcttgccaaaagcacaggccgccctggcgctaactggcgttgagacggcgtcggaactggccagcctggcggcgagtgtgggattagttcaaaatttggccgctttacgagcactagtgagcgagggcattcagcaagggcacatgagtatgcaagctagatccctggccattagcgtaggtgcgaaaggtactgaaatagagcaactagctgcgaagctgagggcagcgacgcaaatgaatcaggagcaggctcgtaaatttctgaccgaaataagaaattaa

SEQ ID NO: 14 - DNA, 1161 bp, Enterococcus casseliflavus
atgaacgttggaattgataaaatcaattttttcgttccgccctatttcattgatatggtggatctcgctcatgcaagagaagttgaccccaacaagttcactataggaataggccaagatcagatggcagtaaacaagaaaacgcaagatatcgtaacgttcgcgatgcacgccgcgaaggatattctgactaaggaagatttacaggccatagatatggtaatagtggggactgagtctgggatcgacgagagcaaggcaagtgctgtcgtattgcatcggcttttaggtattcagccttttgcgcgctcctttgaaattaaggaggcatgctatggggccactgccggccttcagtttgcaaaagctcatgtgcaggctaatccccagagcaaggtcctggtggtagcttccgatatagcacgctacggactggcatccggaggagaaccgactcaaggtgtaggtgctgtggcaatgttgatttccgctgatccagctatcttgcagttagaaaatgataatctcatgttgacccaagatatatacgatttttggcgcccggtcgggcatcaatatcctatggtagacggccatctgtctaatgccgtctatatagacagctttaaacaagtctggcaagcacattgcgagaaaaaccaacggactgctaaagattatgctgcattgtcgttccatattccgtacacgaaaatgggtaagaaagctctgttagcggtttttgcggaggaagatgagacagaacaaaagcggttaatggcacgttatgaagaatcaattgtatacagtcgtcggactggaaatctgtatactggctcactctatctgggcctgatttccttactggagaatagtagcagtttacaggcgaacgatcgcataggtctgtttagctatggttcaggggccgttgcggaatttttcagtggcctcttggtaccgggttacgagaaacaattagcgcaagctgcccatcaagctcttctggacgaccggcaaaaactgactatcgcagagtacgaagccatgtttaatgaaaccattgatattgatcaggaccagtcatttgaggatgacttactgtactccatcagagagatcaaaaacactattcgctactataacgaggagaatgaataa

SEQ ID NO: 15 - protein, 329 aa, Artificial Sequence
  Synthetic Construct
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 16 - protein, 334 aa, Herpetosiphon aurantiacus
MKQLSHAATAVACANIAFIKYWGQHDSQLTLPTNGSISMNLDGCLTETTVQCLPEAVDDSVWLALSGGEEVQAKGRQFERVIQQIERLRQLAGVTERVEVRSRNNFPSDAGIASSAAAFAALTRAAASAFRLELDEAELSRLTRLSGSGSACRSIPAGFVEWYNDGTHAGSYAAQIAPPEHWNLVDIVAVISTEAKHVASTSGHSVATTSPYFSVRLEGIEQRLADVRQGILERDIERLGRASEADAMSMHVIAMTAQPSTMYWLPGTLAVMQAVQRWRAQDNLQSYWTIDAGPNVHVICEAKDAPEVEARLCELDAVQWTIVNGAGPEARLVG

SEQ ID NO: 17 - protein, 326 aa, Anaerolinea thermophila
MGQATAIAHPNIAFIKYWGNRDAVLRIPENGSISMNLAELTVKTTVIFEKHSREDTLILNGALADEPALKRVSHFLDRVREFAGISWHAHVISENNFPTGAGIASSAAAFAALALAATSAIGLHLSERDLSRLARKGSGSACRSIPGGFVEWIPGETDEDSYAVSIAPPEHWALTDCIAILSTQHKPIGSTQGHALASTSPLQPARVADTPRRLEIVRRAILERDFLSLAEMIEHDSNLMHAVMMTSTPPLFYWEPVSLVIMKSVREWRESGLPCAYTLDAGPNVHVICPSEYAEEVIFRLTSIPGVQTVLKASAGDSAKLIEQSL

SEQ ID NO: 18 - protein, 341 aa, Unknown
  Isolated from a metagenomic library constructed from soil samples
MDYYYRVINNNEIPMKSPEFLEVSALAHPNIAFIKYWGNRDNDLRLPCNGSLSMNLSGLETKTSVQFDPSLSADQFKLSGKPIEWDALRRVSDFLEIVRDLAGISFFAKVESENSFPSGAGIASSASAFAALALAASKAAGLSLDEEALSRLARRGSGSACRSIPDGFVEWQAGSTDQDSFAWSIAPADHWDLVDLICVLNSEHKTVGSTGGHALASTSDLHLLRQERVEERIEICRKAILDRDFEHFASVVEEDSNLMHAVMRTSKPPLNYWLPETEVILWKVIHWRKKGIPVCSTVDAGPNVHVLTLSSEAEKVEALLKECPGVQSIFKARAGQGAQLI

SEQ ID NO: 19 - protein, 267 aa, Herpetosiphon aurantiacus
MNKPIFIKLGGSMLTDKTTAERLVDQTLKQVVTDLSAWRQAHPNQPILLGHGGGSFGHYWAERYQTAQGIINEQSWWGVARVADAMARLNRAVVGACLDADLPAIGIQPMASSLANAGEIQQIGSQPLATLLAAGTIPVIYGDVLLDVAQGCTIASTERIFSALVGPLQPTQIILLGEQAVYDADPRQHADAQPIPLINRTNYATIIARLGGSHGVDVTGGMRNKVEAMWQLVQQAPQLEIWICGPQQLQSALSGQLNGPGTIIKLD

SEQ ID NO: 20 - protein, 260 aa, Methanocaldococcus jannaschii
MLTILKLGGSILSDKNVPYSIKWDNLERIAMEIKNALDYYKNQNKEIKLILVHGGGAFGHPVAKKYLKIEDGKKIFINMEKGFWEIQRAMRRFNNIIIDTLQSYDIPAVSIQPSSFVVFGDKLIFDTSAIKEMLKRNLVPVIHGDIVIDDKNGYRIISGDDIVPYLANELKADLILYATDVDGVLIDNKPIKRIDKNNIYKILNYLSGSNSIDVTGGMKYKIDMIRKNKCRGFVFNGNKANNIYKALLGEVEGTEIDFSE

SEQ ID NO: 21 - protein, 271 aa, Methanobrevibacter ruminantium
MIILKIGGSILTEKDSAEPKVDYANLNRIAEEIRQSLYSDEMSNDLIDGLVIVHGAGSFGHPPAKKYRIGEPFDMEDYLSKKIGFSEVQNEVKKLNSIICQSLIEHGIPAVAIPPSAFITSHNKRIYDCNLELIKTYIGEGFVPVLFGDVVLDDEVKIAVISGDQILQYIAKFLKSDRIVLGTDVDGVYTKNPKTHDDAVHIDKVSSIEDIKFLESTTNVDVTGGMVGKVKELLDLAEYGISSEIIDANEKGAISKALQGMEVRGTKISKE

SEQ ID NO: 22 - protein, 266 aa, Methanobacterium thermoautotrophicum
MIILKLGGSVITRKDSEEPAIDRDNLERIASEIGNASPSSLMIVHGAGSFGHPFAGEYRIGSEIENEEDLRRRRFGFALTQNWVKKLNSHVCDALLAEGIPAVSMQPSAFIRAHAGRISHADISLIRSYLEEGMVPVVYGDVVLDSDRRLKFSVISGDQLINHFSLRLMPERVILGTDVDGVYTRNPKKHPDARLLDVIGSLDDLESLDGTLNTDVTGGMVGKIRELLLLAEKGVESEIINAAVPGNIERALLGEEVRGTRITGKH

SEQ ID NO: 23 - protein, 270 aa, Anaerolinea thermophila
MSMDSNLTFLKLGGSLITEKDKPRTPRAKIIQQIAWEIREALREIPNLRLIIGHGSGSFGHATAKKYRTREGVYTLEDWYGFVHVWYDARALNQLVIDALFSAGLPVIAFPPSAITFREGKKVQIATQLIQIAIEKGLIPVVQGDVIFDLDQGGTILSTEEVFAELSFHLRPQRILLAGVEEGVWADFPLRHSLVTEISEDTIKSENIQISGSIATDVTGGMAEKVKSMLDLCQRVPGLEVWIFNGLKKGNVLNALRGFPMGTKILSRNS

SEQ ID NO: 24 - protein, 796 aa, Mycoplasma hominis
MISKIYDDKKYLEKMDKWFRAANYLGVCQMYLRDNPLLKKPLTSNDIKLYPIGHWGTVPGQNFIYTHLNRVIKKYDLNMFYIEGPGHGGQVMISNSYLDGSYSEIYPEISQDEAGLAKMFKRFSFPGGTASHAAPETPGSIHEGGELGYSISHGTGAILDNPDVICAAVVGDGEAETGPLATSWFSNAFINPVNDGAILPILHLNGGKISNPTLLSRKPKEEIKKYFEGLGWNPIFVEWSEDKSNLDMHELMAKSLDKAIESIKEIQAEARKKPAEEATRPTWPMIVLRTPKGWTGPKQWNNEAIEGSFRAHQVPIPVSAFKMEKIADLEKWLKSYKPEELFDENGTIIKEIRDLAPEGLKRMAVNPITNGGIDSKPLKLQDWKKYALKIDYPGEIKAQDMAEMAKFAADIMKDNPSSFRVFGPDETKSNRMFALFNVTNRQWLEPVSKKYDEWISPAGRIIDSQLSEHQCEGFLEGYVLTGRHGFFASYEAFLRVVDSMLTQHMKWIKKASELSWRKTYPSLNIIATSNAFQQDHNGYTHQDPGLLGHLADKRPEIIREYLPADTNSLLAVMNKALTERNVINLIVASKQPREQFFTVEDAEELLEKGYKVVPWASNISENEEPDIVFASSGVEPNIESLAAISLINQEYPHLKIRYVYVLDLLKLRSRKIDPRGISDEEFDKVFTKNKPIIFAFHGFEGLLRDIFFTRSNHNLIAHGYRENGDITTSFDIRQLSEMDRYHIAKDAAEAVYGKDAKAFMNKLDQKLEYHRNYIDEYGYDMPEVVEWKWKNINKEN

SEQ ID NO: 25 - DNA, 26 bp, Artificial Sequence
  Synthetic Construct
tcggttacggttgagtaataaatgga

SEQ ID NO: 26 - DNA, 30 bp, Artificial Sequence
  Synthetic Construct
aaagtagccgaagatgacggtttgtcacat

SEQ ID NO: 27 - DNA, 20 bp, Artificial Sequence
  Synthetic Construct
tggccgtcgttttacaacgt

SEQ ID NO: 28 - DNA, 22 bp, Artificial Sequence
  Synthetic Construct
ttcaggctgtcagccgttaagt

SEQ ID NO: 29 - DNA, 73 bp, Artificial Sequence
  Synthetic Construct
aaatgactctgaattgctgccggctgaaaagcaggctctcggaggaggaaatatgactgccgacaacaatagt

SEQ ID NO: 30 - DNA, 52 bp, Artificial Sequence
  Synthetic Construct
gttccgatcaaagagctatcctggttaatctactttcagaccttgctcggtc

SEQ ID NO: 31 - DNA, 69 bp, Artificial Sequence
  Synthetic Construct
ccaggatagctctttgatcggaacaaacgaaaatcaaaggaggaaccaacaatgtatgtccggaacgga

SEQ ID NO: 32 - DNA, 42 bp, Artificial Sequence
  Synthetic Construct
gctatggtccgtggcatctacaaatcagccaacaagacgagc

SEQ ID NO: 33 - DNA, 71 bp, Artificial Sequence
  Synthetic Construct
tttgtagatgccacggaccatagcaatatactgcgagaagggagggttaacttatgaacaagccgattttt

SEQ ID NO: 34 - DNA, 53 bp, Artificial Sequence
  Synthetic Construct
gccggcagcaattcagagtcattttcaatccaattttataatggttcccggcc

SEQ ID NO: 35 - DNA, 74 bp, Artificial Sequence
  Synthetic Construct
ccaggatagctctttgatcggaactgaacttcagtttagcaaaggagagtatcgatggattactattaccgcgt

SEQ ID NO: 36 - DNA, 46 bp, Artificial Sequence
  Synthetic Construct
gctatggtccgtggcatctacaaatcaaatcagctgagcaccctgc

(19) United States
(12) Patent Application Publication
(10) Pub. No.: US 2016/0002672 A1
(43) Pub. Date: Jan. 7, 2016

(54) PRODUCTION OF ISOPRENE, ISOPRENOID, AND ISOPRENOID PRECURSORS USING AN ALTERNATIVE LOWER MEVALONATE PATHWAY

(71) Applicants: DANISCO US INC., Palo Alto, CA (US); THE GOODYEAR TIRE & RUBBER COMPANY, Akron, OH (US)

(72) Inventors: Zachary Q. BECK, Palo Alto, CA (US); Jörg MAMPEL, Bensheim (DE); Guido MEURER, Seeheim-Jugenheim (DE); Michael C. MILLER, San Francisco, CA (US); Karl J. SANFORD, Cupertino, CA (US); Dmitrii V. VAVILINE, Palo Alto, CA (US); Walter WEYLER, San Francisco, CA (US); Gregory M. WHITED, Belmont, CA (US)

(21) Appl. No.: 14/654,424

(22) PCT Filed: Dec. 20, 2013
(86) PCT No.: PCT/US2013/077245

§ 371 Date: Jun. 19, 2015

Related U.S. Application Data
(60) Provisional application No. 61/865,978, filed on Aug. 14, 2013.
(60) Provisional application No. 61/745,530, filed on Dec. 21, 2012.

(51) Int. Cl. C12P 5/00; C07F 9/09; C12N 9/12; C12P 9/00; C12N 9/88; C07C 11/18

(52) U.S. Cl. CPC C12P 5/007; C12N 9/88; C07C 11/18; C12N 9/1229; C12P 9/00; C07F 9/09; C12Y 401/01; C12Y 207/04026

(57) ABSTRACT

The invention provides for compositions and methods for the production of isoprene, isoprenoid precursor, and/or isoprenoids in cells via the expression (e.g., heterologous expression) of phosphomevalonate decarboxylases and/or isopentenyl kinases.

### CROSS-REFERENCE TO RELATED APPLICATIONS

This application claims priority to U.S. Provisional Patent Application No. 61/745,530, filed Dec. 21, 2012; and U.S. Provisional Patent Application No. 61/865,978, filed Aug. 14, 2013; the content of each of which is incorporated herein by reference in its entirety.

### INCORPORATION BY REFERENCE

The content of the following submission on ASCII text file is incorporated herein by reference in its entirety: a computer readable form (CRF) of the Sequence Listing (file name: 643842004740SEQLIST.txt, date recorded: Dec. 20, 2013, size: 99,996 bytes).

### FIELD OF THE INVENTION

This present invention relates to recombinant cells comprising a phosphomevalonate decarboxylase, an isopentenyl kinase, and one or more mevalonate (MVA) pathway polypeptides capable of producing isoprenoid precursors, isoprene and isoprenoids and compositions that include these cultured cells, as well as methods for producing and using the same.

### BACKGROUND OF THE INVENTION

Isoprene (2-methyl-1,3-butadiene) is the critical starting material for a variety of synthetic polymers, most notably synthetic rubbers. Isoprene can be obtained by fractionating petroleum; however, the purification of this material is expensive and time-consuming. Petroleum cracking of the C5 stream of hydrocarbons produces only about 15% isoprene. About 800,000 tons per year of cis-polyisoprene are produced from the polymerization of isoprene; most of this polyisoprene is used in the tire and rubber industry. Isoprene is also copolymerized for use as a synthetic elastomer in other products such as footwear, mechanical products, medical products, sporting goods, and latex. Isoprene can also be naturally produced by a variety of microbial, plant, and animal species. In particular, two pathways have been identified for the natural biosynthesis of isoprene: the mevalonate (MVA) pathway and the non-mevalonate (DXP) pathway.

Over 29,000 isoprenoid compounds have been identified and new isoprenoids are being discovered each year. Isoprenoids can be isolated from natural products, such as microorganisms and species of plants that use isoprenoid precursor molecules as a basic building block to form the relatively complex structures of isoprenoids. Isoprenoids are vital to most living organisms and cells, providing a means to maintain cellular membrane fluidity and electron transport. In nature, isoprenoids function in roles as diverse as natural pesticides in plants to contributing to the scents associated with cinnamon, cloves, and ginger. Moreover, the pharmaceutical and chemical communities use isoprenoids as pharmaceuticals, nutraceuticals, flavoring agents, and agricultural pest control agents. Given their importance in biological systems and usefulness in a broad range of applications, isoprenoids have been the focus of much attention by scientists.

Conventional means for obtaining isoprenoids include extraction from biological materials (e.g., plants, microbes, and animals) and partial or total organic synthesis in the laboratory. Such means, however, have generally proven to be unsatisfactory. In particular for isoprenoids, given the often times complex nature of their molecular structure, organic synthesis is impractical given that several steps are usually required to obtain the desired product. Additionally, these chemical synthesis steps can involve the use of toxic solvents as can extraction of isoprenoids from biological materials. Moreover, these extraction and purification methods usually result in a relatively low yield of the desired isoprenoid, as biological materials typically contain only minute amounts of these molecules. Unfortunately, the difficulty involved in obtaining relatively large amounts of isoprenoids has limited their practical use.

Recent developments in the production of isoprene, isoprenoid precursor molecules, and isoprenoids disclose methods for the production of these compounds at various rates, titers, and purities. See, for example, International Patent Application Publication No. WO 2009/076676 A2. However, alternative pathways to improve production and yields that are sufficient to meet the demands of a robust commercial process are still needed.

Provided herein are cultured recombinant cells, compositions of these cells and methods of using these cells to increase production of molecules derived from mevalonate, such as isoprenoid precursors, isoprene and/or isoprenoids via an alternative lower MVA pathway.

Throughout this specification, various patents, patent applications and other types of publications (e.g., journal articles) are referenced. The disclosure of all patents, patent applications, and publications cited herein are hereby incorporated by reference in their entirety for all purposes.

### BRIEF SUMMARY OF INVENTION

The invention provided herein discloses, inter alia, compositions of matter comprising recombinant cells comprising a phosphomevalonate decarboxylase and methods of making and using these recombinant cells for the production of isoprene, isoprenoid precursors, and isoprenoids. In some aspects, the recombinant cells further comprise an isopentenyl kinase for the production of isoprene, isoprenoid precursors, and isoprenoids.

Accordingly, in one aspect, the invention provides recombinant cells capable of producing isoprene, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide, wherein culturing of said recombinant cells provides for the production of isoprene. In one embodiment, the nucleic acid encoding a polypeptide having phosphomevalonate decarboxylase activity catalyzes the conversion of mevalonate 5-phosphate to isopentenyl phosphate. In another embodiment, the nucleic acid encoding a polypeptide having phosphomevalonate decarboxylase activity catalyzes the conversion of mevalonate 5-pyrophosphate to isopentenyl phosphate and/or isopentenyl pyrophosphate. In yet another embodiment, the nucleic acid encoding a polypeptide having phosphomevalonate decarboxylase activity is from an archaea. In a further embodiment, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In one embodiment, the nucleic acid encoding a polypeptide having phosphomevalonate decarboxylase activity is from a microorganism selected from the group consisting of: Herpetosiphon aurantiacus, S378Pa3-2, and Anaerolinea thermophila. In another embodiment, the nucleic acid encoding a polypeptide having isopentenyl kinase activity is from an archaea. In a further embodiment, the archaea is selected from the group consisting of desulforococcales, sulfolobales, thermoproteales, cenarchaeales, nitro sopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In an embodiment, the nucleic acid sequence encoding a polypeptide having phosphomevalonate decarboxylase activity comprises at least 85% sequence identity to a nucleic acid sequence encoding a phosphomevalonate decarboxylase comprising the amino acid sequence selected from the group consisting of SEQ ID NOs:16-18. In another embodiment, the nucleic acid sequence encoding a polypeptide having phosphomevalonate decarboxylase activity encodes a polypeptide having an amino acid sequence with at least 85% sequence identity to an amino acid sequence selected from the group consisting of SEQ ID NOs:16-18. In an embodiment, the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila. In a further embodiment, the microorganism is Herpetosiphon aurantiacus or Methanococcus jannaschii. In one embodiment, the nucleic acid sequence encoding a polypeptide having isopentenyl kinase activity comprises at least 85% sequence identity to a nucleic acid sequence encoding an isopentenyl kinase comprising the amino acid sequence selected from the group consisting of SEQ ID NOs:19-23. In another embodiment, the nucleic acid sequence encoding a polypeptide having isopentenyl kinase activity encodes a polypeptide having an amino acid sequence with at least 85% sequence identity to an amino acid sequence selected from the group consisting of SEQ ID NOs:19-23. In another embodiment, the isoprene synthase polypeptide is a plant isoprene synthase polypeptide. In a further embodiment, the plant isoprene synthase polypeptide is a polypeptide or variant thereof from Pueraria or Populus. In another further embodiment, the plant isoprene synthase polypeptide is a polypeptide or variant thereof from Pueraria montana or Pueraria lobata, Populus tremuloides, Populus alba, Populus nigra, Populus trichocarpa, or a hybrid Populus alba×Populus tremula. In an embodiment, one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate. In another embodiment, one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate. In any of the embodiments herein, the recombinant cells further comprise one or more nucleic acids encoding an isopentenyl-diphosphate delta-isomerase (IDI) polypeptide. In a further embodiment, the recombinant cells comprise an attenuated enzyme that converts mevalonate 5-pyrophosphate to isopentenyl pyrophosphate. In another further embodiment, recombinant cells comprise an attenuated enzyme that converts mevalonate 5-phosphate to mevalonate 5-pyrophosphate. In any of the embodiments herein, the recombinant cells further comprise one or more nucleic acids encoding one or more 1-deoxy-D-xylulose 5-phosphate (DXP) pathway polypeptides. In any of the embodiments herein, the recombinant cells comprise one or more attenuated enzymes of the 1-deoxy-D-xylulose 5-phosphate (DXP) pathway. In any of the embodiments herein, the recombinant cells further comprise a heterologous nucleic acid encoding a polypeptide having phosphoketolase activity. In any of the embodiments herein, the nucleic acid encoding a polypeptide having phosphomevalonate decarboxylase activity is a heterologous nucleic acid. In any of the embodiments herein, the nucleic acid encoding a polypeptide having phosphomevalonate decarboxylase activity is an endogenous nucleic acid. In any embodiments herein, the nucleic acid encoding a polypeptide having isopentenyl kinase activity is a heterologous nucleic acid. In any of the embodiments herein, the nucleic acid encoding a polypeptide having isopentenyl kinase activity is an endogenous nucleic acid. In any of the embodiments herein, at least one of the nucleic acids encoding a polypeptide of (i)-(iv) is placed under an inducible promoter or a constitutive promoter. In any of the embodiments herein, at least one of the nucleic acids encoding a polypeptide of (i)-(iv) is cloned into one or more multicopy plasmids. In any of the embodiments herein, at least one of the nucleic acids encoding a polypeptide of (i)-(iv) is integrated into a chromosome of the cells. In any of the embodiments herein, the recombinant cells are gram-positive bacterial cells, gram-negative bacterial cells, fungal cells, filamentous fungal cells, plant cells, algal cells or yeast cells. In further embodiments, the bacterial cells are selected from the group consisting of E. coli, L. acidophilus, Corynebacterium sp., P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells. In other further embodiments, the yeast cells are selected from the group consisting of Saccharomyces sp., Schizosaccharomyces sp., Pichia sp., or Candida sp. In an embodiment, the recombinant cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisieae and Yarrowia lipolytica.

In another aspect, the invention provides recombinant cells capable of producing isoprenoid precursors, wherein the cells comprise (i) a nucleic acid encoding a polypeptide having phosphomevalonate decarboxylase activity, (ii) a nucleic acid encoding a polypeptide having isopentenyl kinase activity, and (iii) one or more nucleic acids encoding one or more polypeptides of the MVA pathway, wherein culturing of said recombinant cells provides for the production of isoprenoid precursors. In one embodiment, the nucleic acid encoding a polypeptide having phosphomevalonate decarboxylase activity catalyzes the conversion of mevalonate 5-phosphate to isopentenyl phosphate. In another embodiment, the nucleic acid encoding a polypeptide having phosphomevalonate decarboxylase activity catalyzes the conversion of mevalonate 5-pyrophosphate to isopentenyl phosphate and/or isopentenyl pyrophosphate. In yet another embodiment, the nucleic acid encoding a polypeptide having phosphomevalonate decarboxylase activity is from an archaea. In a further embodiment, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In an embodiment, the nucleic acid encoding a polypeptide having phosphomevalonate decarboxylase activity is from a microorganism selected from the group consisting of: Herpetosiphon aurantiacus, S378Pa3-2, and Anaerolinea thermophila. In one embodiment, the nucleic acid sequence encoding a polypeptide having phosphomevalonate decarboxylase activity comprises at least 85% sequence identity to a nucleic acid sequence encoding a phosphomevalonate decarboxylase comprising the amino acid sequence selected from the group consisting of SEQ ID NOs:16-18. In another embodiment, the nucleic acid sequence encoding a polypeptide having phosphomevalonate decarboxylase activity encodes a polypeptide having an amino acid sequence with at least 85% sequence identity to an amino acid sequence selected from the group consisting of SEQ ID NOs:16-18. In another embodiment, the nucleic acid encoding a polypeptide having isopentenyl kinase activity is from an archaea. In a further embodiment, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In one embodiment, the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila. In a further embodiment, the microorganism is Herpetosiphon aurantiacus or Methanococcus jannaschii. In one embodiment, the nucleic acid sequence encoding a polypeptide having isopentenyl kinase activity comprises at least 85% sequence identity to a nucleic acid sequence encoding an isopentenyl kinase comprising the amino acid sequence selected from the group consisting of SEQ ID NOs:19-23. In another embodiment, the nucleic acid sequence encoding a polypeptide having isopentenyl kinase activity encodes a polypeptide having an amino acid sequence with at least 85% sequence identity to an amino acid sequence selected from the group consisting of SEQ ID NOs:19-23. In an embodiment, one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate. In another embodiment, one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate. In yet another embodiment, the recombinant cells comprise an attenuated enzyme that converts mevalonate 5-pyrophosphate to isopentenyl pyrophosphate. In an embodiment, the recombinant cells comprise an attenuated enzyme that converts mevalonate 5-phosphate to mevalonate 5-pyrophosphate. In any of the embodiments herein, the recombinant cells further comprise one or more nucleic acids encoding one or more 1-deoxy-D-xylulose 5-phosphate (DXP) pathway polypeptides. In any of the embodiments herein, the recombinant cells comprise one or more attenuated enzymes of the 1-deoxy-D-xylulose 5-phosphate (DXP) pathway. In any of the embodiments herein, the recombinant cells further comprise a heterologous nucleic acid encoding a polypeptide having phosphoketolase activity. In any of the embodiments herein, the nucleic acid encoding a polypeptide having phosphomevalonate decarboxylase activity is a heterologous nucleic acid. In any of the embodiments herein, the nucleic acid encoding a polypeptide having phosphomevalonate decarboxylase activity is an endogenous nucleic acid. In any of the embodiments herein, the nucleic acid encoding a polypeptide having isopentenyl kinase activity is a heterologous nucleic acid. In any of the embodiments herein, the nucleic acid encoding a polypeptide having isopentenyl kinase activity is an endogenous nucleic acid. In any of the embodiments herein, at least one of the nucleic acids encoding a polypeptide of (i)-(iv) is placed under an inducible promoter or a constitutive promoter. In any of the embodiments herein, at least one of the nucleic acids encoding a polypeptide of (i)-(iv) is cloned into one or more multicopy plasmids. In any of the embodiments herein, at least one of the nucleic acids encoding a polypeptide of (i)-(iv) is integrated into a chromosome of the cells. In any of the embodiments herein, the recombinant cells are gram-positive bacterial cells, gram-negative bacterial cells, fungal cells, filamentous fungal cells, plant cells, algal cells or yeast cells. In further embodiments, the bacterial cells are selected from the group consisting of E. coli, L. acidophilus, Corynebacterium sp., P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells. In yet further embodiments, the yeast cells are selected from the group consisting of Saccharomyces sp., Schizosaccharomyces sp., Pichia sp., or Candida sp. In an embodiment, the recombinant cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisieae and Yarrowia lipolytica.

In yet another aspect, the invention provides recombinant cells capable of producing of isoprenoids, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an polyprenyl pyrophosphate synthase polypeptide, wherein culturing of said recombinant cells in a suitable media provides for the production of isoprenoids. In an embodiment, the nucleic acid encoding a polypeptide having phosphomevalonate decarboxylase activity catalyzes the conversion of mevalonate 5-phosphate to isopentenyl phosphate. In another embodiment, the nucleic acid encoding a polypeptide having phosphomevalonate decarboxylase activity catalyzes the conversion of mevalonate 5-pyrophosphate to isopentenyl phosphate and/or isopentenyl pyrophosphate. In yet another embodiment, the nucleic acid encoding a polypeptide having phosphomevalonate decarboxylase activity is from an archaea. In a further embodiment, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In an embodiment, the nucleic acid encoding a polypeptide having phosphomevalonate decarboxylase activity is from a microorganism selected from the group consisting of: Herpetosiphon aurantiacus, S378Pa3-2, and Anaerolinea thermophila. In one embodiment, the nucleic acid sequence encoding a polypeptide having phosphomevalonate decarboxylase activity comprises at least 85% sequence identity to a nucleic acid sequence encoding a phosphomevalonate decarboxylase comprising the amino acid sequence selected from the group consisting of SEQ ID NOs:16-18. In another embodiment, the nucleic acid sequence encoding a polypeptide having phosphomevalonate decarboxylase activity encodes a polypeptide having an amino acid sequence with at least 85% sequence identity to an amino acid sequence selected from the group consisting of SEQ ID NOs:16-18. In another embodiment, nucleic acid encoding a polypeptide having isopentenyl kinase activity is from an archaea. In a further embodiment, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In an embodiment, the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila. In a further embodiment, the microorganism is Herpetosiphon aurantiacus or Methanococcus jannaschii. In one embodiment, the nucleic acid sequence encoding a polypeptide having isopentenyl kinase activity comprises at least 85% sequence identity to a nucleic acid sequence encoding an isopentenyl kinase comprising the amino acid sequence selected from the group consisting of SEQ ID NOs:19-23. In another embodiment, the nucleic acid sequence encoding a polypeptide having isopentenyl kinase activity encodes a polypeptide having an amino acid sequence with at least 85% sequence identity to an amino acid sequence selected from the group consisting of SEQ ID NOs:19-23. In an embodiment, the isoprenoid is selected from group consisting of monoterpenes, diterpenes, triterpenes, tetraterpenes, sesquiterpene, and polyterpene. In another embodiment, the isoprenoid is a sesquiterpene. In yet another embodiment, the isoprenoid is selected from the group consisting of abietadiene, amorphadiene, carene, α-famesene, β-farnesene, farnesol, geraniol, geranylgeraniol, linalool, limonene, myrcene, nerolidol, ocimene, patchoulol, β-pinene, sabinene, γ-terpinene, terpindene and valencene. In still another embodiment, one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate. In an embodiment, one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate. In an embodiment, the recombinant cells comprise an attenuated enzyme that converts mevalonate 5-pyrophosphate to isopentenyl pyrophosphate. In an another embodiment, the recombinant cells comprise an attenuated enzyme that converts mevalonate 5-phosphate to mevalonate 5-pyrophosphate. In any of the embodiments herein, the recombinant cells further comprise one or more nucleic acids encoding one or more 1-deoxy-D-xylulose 5-phosphate (DXP) pathway polypeptides. In any of the embodiments herein, the recombinant cells comprise one or more attenuated enzymes of the 1-deoxy-D-xylulose 5-phosphate (DXP) pathway. In any of the embodiments herein, the recombinant cells further comprise a heterologous nucleic acid encoding a polypeptide having phosphoketolase activity. In any of the embodiments herein, the nucleic acid encoding a polypeptide having phosphomevalonate decarboxylase activity is a heterologous nucleic acid. In any of the embodiments herein, the nucleic acid encoding a polypeptide having phosphomevalonate decarboxylase activity is an endogenous nucleic acid. In any of the embodiments herein, wherein the nucleic acid encoding a polypeptide having isopentenyl kinase activity is a heterologous nucleic acid. In any of the embodiments herein, wherein the nucleic acid encoding a polypeptide having isopentenyl kinase activity is an endogenous nucleic acid. In any of the embodiments herein, at least one of the nucleic acids encoding a polypeptide of (i)-(iv) is placed under an inducible promoter or a constitutive promoter. In any of the embodiments herein, at least one of the nucleic acids encoding a polypeptide of (i)-(iv) is cloned into one or more multicopy plasmids. In any of the embodiments herein, at least one of the nucleic acids encoding a polypeptide of (i)-(iv) is integrated into a chromosome of the cells. In any of the embodiments herein, the recombinant cells are gram-positive bacterial cells, gram-negative bacterial cells, fungal cells, filamentous fungal cells, plant cells, algal cells or yeast cells. In further embodiments, the bacterial cells are selected from the group consisting of E. coli, L. acidophilus, Corynebacterium sp., P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells. In other further embodiments, the yeast cells are selected from the group consisting of Saccharomyces sp., Schizosaccharomyces sp., Pichia sp., or Candida sp. In an embodiment, the recombinant cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisieae and Yarrowia lipolytica.

In one aspect, the invention herein also provides for a method of producing isoprene comprising: (a) culturing any of the recombinant cells disclosed herein under conditions suitable for producing isoprene and (b) producing the isoprene. In a further embodiment, the method further comprises (c) recovering the isoprene.

In another aspect, the invention herein also provides for a method of producing an isoprenoid precursor comprising: (a) culturing any of the recombinant cells disclosed herein under conditions suitable for producing an isoprenoid precursor and (b) producing an isoprenoid precursor. In a further embodiment, the method further comprises (c) recovering the isoprenoid precursor.

In yet another aspect, the invention provides for a method of producing an isoprenoid comprising: (a) culturing any of the recombinant cells disclosed herein under conditions suitable for producing an isoprenoid and (b) producing an isoprenoid. In a further embodiment, the method further comprises (c) recovering the isoprenoid.

In another aspect, the invention herein provides for a composition comprising isoprene produced by a recombinant cell described herein. In some embodiments, the composition comprising isoprene produced by a recombinant cell described herein can be produced by any method contemplated herein.

In still another aspect, the invention herein also provides for a composition comprising an isoprenoid precursor produced by a recombinant cell described herein. In some embodiments, the composition comprising an isoprenoid precursor produced by a recombinant cell described herein can be produced by any method contemplated herein.

In another aspect, the invention herein also provides for a composition comprising an isoprenoid produced by a recombinant cell described herein. In some embodiments, the composition comprising an isoprenoid produced by a recombinant cell described herein can be produced by any method contemplated herein.

In another aspect, the invention herein provides for an isolated nucleic acid encoding a polypeptide having phosphomevalonate decarboxylase activity, wherein said polypeptide comprises at least 85% sequence identity to the amino acid sequence of SEQ ID NO:18. In another aspect, the invention herein provides for an isolated polypeptide having phosphomevalonate decarboxylase activity, wherein said polypeptide comprises at least 85% sequence identity to the amino acid sequence of SEQ ID NO:18.

In one aspect, also provided herein is an isolated cell comprising a nucleic acid encoding a polypeptide having phosphomevalonate decarboxylase activity, wherein said polypeptide comprises at least 85% sequence identity to the amino acid sequence of SEQ ID NO:18. In some embodiments, the nucleic acid is a heterologous nucleic acid. In some embodiments, the nucleic acid is an endogenous nucleic acid. In some aspects, provided herein is a recombinant cell comprising a nucleic acid encoding a polypeptide having phosphomevalonate decarboxylase activity, wherein said polypeptide comprises at least 85% sequence identity to the amino acid sequence of SEQ ID NO:18. In some embodiments, the nucleic acid is a heterologous nucleic acid. In some embodiments, the nucleic acid is an endogenous nucleic acid.

In some aspects, the invention herein provides a cell extract comprising a polypeptide having phosphomevalonate decarboxylase activity, wherein said polypeptide comprises at least 85% sequence identity to the amino acid sequence of SEQ ID NO:18.

### BRIEF DESCRIPTION OF THE DRAWINGS

FIG. 1 shows the upper and classical lower MVA pathway and the DXP pathways for production of isoprene, isoprenoid precursors, and isoprenoids (based on F. Bouvier et al., Progress in Lipid Res. 44: 357-429, 2005). The following description includes alternative names for each polypeptide in the pathways and a reference that discloses an assay for measuring the activity of the indicated polypeptide. Mevalonate Pathway: AACT; Acetyl-CoA acetyltransferase, MvaE, EC 2.3.1.9. Assay: J. Bacteriol., 184: 2116-2122, 2002; HMGS; Hydroxymethylglutaryl-CoA synthase, MvaS, EC 2.3.3.10. Assay: J. Bacteriol., 184: 4065-4070, 2002; HMGR; 3-Hydroxy-3-methylglutaryl-CoA reductase, MvaE, EC 1.1.1.34. Assay: J. Bacteriol., 184: 2116-2122, 2002; MVK; Mevalonate kinase, ERG12, EC 2.7.1.36. Assay: Curr Genet 19:9-14, 1991. PMK; Phosphomevalonate kinase, ERGS, EC 2.7.4.2, Assay: Mol Cell Biol., 11:620-631, 1991; DPMDC; Diphosphomevalonate decarboxylase, MVD1, EC 4.1.1.33. Assay: Biochemistry, 33:13355-13362, 1994; IDI; Isopentenyl-diphosphate delta-isomerase, IDI1, EC 5.3.3.2. Assay: J. Biol. Chem. 264:19169-19175, 1989. DXP Pathway: DXS; 1-Deoxyxylulose-5-phosphate synthase, dxs, EC 2.2.1.7. Assay: PNAS, 94:12857-62, 1997; DXR; 1-Deoxy-D-xylulose 5-phosphate reductoisomerase, dxr, EC 2.2.1.7. Assay: Eur. J. Biochem. 269:4446-4457, 2002; MCT; 4-Diphosphocytidyl-2C-methyl-D-erythritol synthase, IspD, EC 2.7.7.60. Assay: PNAS, 97: 6451-6456, 2000; CMK; 4-Diphosphocytidyl-2-C-methyl-D-erythritol kinase, IspE, EC 2.7.1.148. Assay: PNAS, 97:1062-1067, 2000; MCS; 2C-Methyl-D-erythritol 2,4-cyclodiphosphate synthase, IspF, EC 4.6.1.12. Assay: PNAS, 96:11758-11763, 1999; HDS; 1-Hydroxy-2-methyl-2-(E)-butenyl 4-diphosphate synthase, ispG, EC 1.17.4.3. Assay: J. Org. Chem., 70:9168-9174, 2005; HDR; 1-Hydroxy-2-methyl-2-(E)-butenyl 4-diphosphate reductase, IspH, EC 1.17.1.2. Assay: JACS, 126:12847-12855, 2004.

FIG. 2 is a schematic of the alternative lower MVA pathway shown in parallel with the DXP pathway for production of isoprene, isoprenoid precursors, and isoprenoids. Mevalonate kinase (MVK); phosphomevalonate decarboxylase (PMevDC); isopentenyl phosphate kinase (IPK); isopentenyl diphosphate isomerase (IDI). The alternative lower MVA pathway is present, for example, in some archaeal organisms, such as Methanosarcina mazei.

FIG. 3 is a plasmid map of pMCM2200.

FIG. 4 is a plasmid map of pMCM2201.

FIG. 5 is a plasmid map of pMCM2212.

FIG. 6 is a plasmid map of pMCM2244.

FIG. 7 is a plasmid map of pMCM2246.

FIG. 8 is a plasmid map of pMCM2248.

FIG. 9 is an SDS-PAGE gel stained with SafeStain. Lane: 1) 10 μL of Marker, 2) Herpetosiphon aurantiacus ATCC 23779 phosphomevalonate decarboxylase with His-tag, 3) Herpetosiphon aurantiacus ATCC 23779 phosphomevalonate decarboxylase without His-tag, 4) Herpetosiphon aurantiacus ATCC 23779 isopentenyl phosphate kinase with His-tag, 5) Herpetosiphon aurantiacus ATCC 23779 isopentenyl phosphate kinase without His-tag, 6) S378Pa3-2 phosphomevalonate decarboxylase with His-tag, 7) S378Pa3-2 phosphomevalonate decarboxylase without His-tag.

FIG. 10 is a series of graphs showing the growth of strains MCM2257, MCM2258, MCM2259, MCM2260, MCM2261, and MCM2262 in four different media formulations after IPTG induction over the course four hours.

FIG. 11 is a series of graphs showing isoprene production by strains MCM2257, MCM2258, MCM2259, MCM2260, MCM2261, and MCM2262 in four different media formulations after IPTG induction over the course four hours.

### DETAILED DESCRIPTION

Mevalonate is an intermediate of the mevalonate-dependent pathway that converts acetyl-CoA to isopentenyl pyrophosphate (IPP) and dimethylallyl diphosphate (DMAPP). The conversion of acetyl-CoA to mevalonate can be catalyzed by the thiolase, HMG-CoA synthase and the HMG-CoA reductase activities of the upper MVA pathway. The classical lower MVA pathway utilizes mevalonate as substrate for generating IPP and DMAPP as the terminal products of the MVA pathway. The DXP pathway also produces IPP and DMAPP. Both IPP and DMAPP are precursors to isoprene as well as to isoprenoids. Although the MVA pathway is typically found in animals, plants, and in many bacteria, the full MVA pathway has not been identified in archaea even though a distinguishing characteristic of archaeal organisms is that isoprenoids make up a major component of their membrane lipids. Putative isopentenyl phosphate kinases (IPKs) have been identified and characterized from archaea, suggesting the possible utilization of a modified mevalonate pathway for the production of isoprenoids in archaea. However, a phosphomevalonate decarboxylase that catalyzes the conversion of mevalonate 5-phosphate to isopentenyl phosphate has not been previously described.

The invention provided herein discloses, inter alia, compositions and methods for the production of isoprenoid precursor molecules, isoprene and/or isoprenoids in recombinant cells that have been engineered to express a phosphomevalonate decarboxylase polypeptide and/or an isopentenyl kinase polypeptide. The phosphomevalonate decarboxylase of this invention can use mevalonate 5-phosphate and/or mevalonate 5-pyrophosphate as a substrate. In certain embodiments, the invention provides for compositions and methods for the production of isoprenoid precursor molecules, isoprene and/or isoprenoids in recombinant cells that have been engineered to express a phosphomevalonate decarboxylase polypeptide capable of catalyzing the conversion of mevalonate 5-phosphate to isopentenyl phosphate. In other embodiments, the invention provides for compositions and methods for the production of isoprenoid precursor molecules, isoprene and/or isoprenoids in recombinant cells that have been engineered to express a phosphomevalonate decarboxylase polypeptide capable of catalyzing the conversion of mevalonate 5-pyrophosphate to isopentenyl phosphate and/or isopentenyl pyrophosphate.

### GENERAL TECHNIQUES

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Singleton et al., Dictionary of Microbiology and Molecular Biology 2nd ed., J. Wiley & Sons (New York, N. Y. 1994), and March, Advanced Organic Chemistry Reactions, Mechanisms and Structure 4th ed., John Wiley & Sons (New York, N. Y. 1992), provide one skilled in the art with a general guide to many of the terms used in the present application.

### DEFINITIONS

As used herein, the term “polypeptides” includes polypeptides, proteins, peptides, fragments of polypeptides, and fusion polypeptides.

As used herein, an “isolated polypeptide” is not part of a library of polypeptides, such as a library of 2, 5, 10, 20, 50 or more different polypeptides and is separated from at least one component with which it occurs in nature. An isolated polypeptide can be obtained, for example, by expression of a recombinant nucleic acid encoding the polypeptide.

By “heterologous polypeptide” is meant a polypeptide encoded by a nucleic acid sequence derived from a different organism, species, or strain than the host cell. In some embodiments, a heterologous polypeptide is not identical to a wild-type polypeptide that is found in the same host cell in nature.

As used herein, a “nucleic acid” refers to two or more deoxyribonucleotides and/or ribonucleotides covalently joined together in either single or double-stranded form.

By “recombinant nucleic acid” is meant a nucleic acid of interest that is free of one or more nucleic acids (e.g., genes) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA, a genomic DNA fragment, or a cDNA fragment produced by PCR or restriction endonuclease digestion) independent of other sequences.

By “heterologous nucleic acid” is meant a nucleic acid sequence derived from a different organism, species or strain than the host cell. In some embodiments, the heterologous nucleic acid is not identical to a wild-type nucleic acid that is found in the same host cell in nature. For example, a nucleic acid encoded by the phosphomevalonate decarboxylase gene from Herpetosiphon aurantiacus and/or S378Pa3-2 and used to transform an E. coli is a heterologous nucleic acid.

As used herein, the terms “phosphomevalonate decarboxylase,” “phosphomevalonate decarboxylase enzyme,” “phosphomevalonate decarboxylase polypeptide,” and “PMevDC” are used interchangeably and refer to a polypeptide that converts mevalonate 5-phosphate to isopentenyl phosphate and/or converts mevalonate 5-pyrophosphate to isopentenyl phosphate and/or isopentenyl pyrophosphate. In some embodiments, the phosphomevalonate decarboxylase polypeptide catalyzes the conversion of mevalonate 5-phosphate to isopentenyl phosphate. In other embodiments, the phosphomevalonate decarboxylase polypeptide catalyzes the conversion of mevalonate 5-pyrophosphate to isopentenyl phosphate. In other embodiments, the phosphomevalonate decarboxylase polypeptide catalyzes the conversion of mevalonate 5-pyrophosphate to isopentenyl pyrophosphate. In some embodiments, the phosphomevalonate decarboxylase polypeptide catalyzes the conversion of mevalonate 5-pyrophosphate to isopentenyl phosphate and isopentenyl pyrophosphate.

As used herein, the terms “isopentenyl kinase,” “isopentenyl kinase enzyme,” “isopentenyl kinase polypeptide,” “isopentenyl phosphate kinase,” and “IPK” are used interchangeably and refer to a polypeptide that converts isopentenyl phosphate to isopentenyl pyrophosphate. In some embodiments, the isopentenyl kinase polypeptide catalyzes the conversion of isopentenyl phosphate to isopentenyl pyrophosphate.

The term “isoprene” refers to 2-methyl-1,3-butadiene (CAS#78-79-5). It can be the direct and final volatile C5 hydrocarbon product from the elimination of pyrophosphate from 3,3-dimethylallyl diphosphate (DMAPP). It may not involve the linking or polymerization of IPP molecules to DMAPP molecules. The term “isoprene” is not generally intended to be limited to its method of production unless indicated otherwise herein.

As used herein, the term “isoprenoid” refers to a large and diverse class of naturally-occurring class of organic compounds composed of two or more units of hydrocarbons, with each unit consisting of five carbon atoms arranged in a specific pattern. As used herein, “isoprene” is expressly excluded from the definition of “isoprenoid.”

As used herein, “isoprenoid precursor” refers to any molecule that is used by organisms in the biosynthesis of terpenoids or isoprenoids. Non-limiting examples of isoprenoid precursor molecules include, e.g., isopentenyl pyrophosphate (IPP) and dimethylallyl diphosphate (DMAPP).

As used herein, the term “mass yield” refers to the mass of the product produced by the recombinant cells divided by the mass of the glucose consumed by the recombinant cells expressed as a percentage.

By “specific productivity,” it is meant the mass of the product produced by the recombinant cell divided by the product of the time for production, the cell density, and the volume of the culture.

By “titer,” it is meant the mass of the product produced by the recombinant cells divided by the volume of the culture.

As used herein, the term “cell productivity index (CPI)” refers to the mass of the product produced by the recombinant cells divided by the mass of the recombinant cells produced in the culture.

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Reference to “about” a value or parameter herein also includes (and describes) embodiments that are directed to that value or parameter per se.

It is understood that all aspects and embodiments of the invention described herein include “comprising,” “consisting,” and “consisting essentially of” aspects and embodiments. It is to be understood that methods or compositions “consisting essentially of” the recited elements include only the specified steps or materials and those that do not materially affect the basic and novel characteristics of those methods and compositions.

It is to be understood that this invention is not limited to the particular methodology, protocols, and reagents described, as these may vary, depending upon the context they are used by those of skill in the art.

### Phosphomevalonate Decarboxylases

The mevalonate-dependent biosynthetic pathway (MVA pathway) is a key metabolic pathway present in all higher eukaryotes and certain bacteria. In addition to being important for the production of molecules used in processes as diverse as protein prenylation, cell membrane maintenance, protein anchoring, and N-glycosylation, the mevalonate pathway provides a major source of the isoprenoid precursor molecules DMAPP and IPP, which serve as the basis for the biosynthesis of terpenes, terpenoids, isoprenoids, and isoprene.

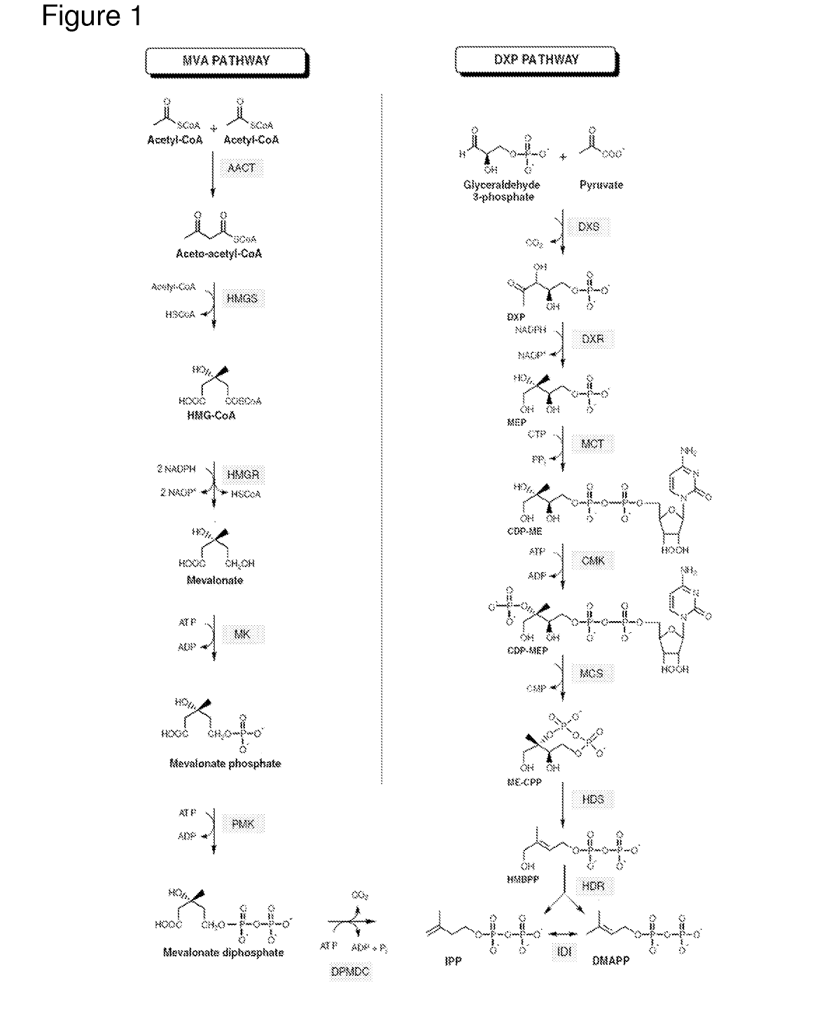

The complete MVA pathway can be subdivided into two groups: an upper and lower pathway (FIG. 1). In the upper portion of the MVA pathway, acetyl Co-A produced during cellular metabolism is converted to mevalonate via the actions of polypeptides having either: (a) (i) thiolase activity or (ii) acetoacetyl-CoA synthase activity, (b) HMG-CoA reductase, and (c) HMG-CoA synthase enzymatic activity. First, acetyl Co-A is converted to acetoacetyl CoA via the action of a thiolase or an acetoacetyl-CoA synthase (which utilizes acetyl-CoA and malonyl-CoA). Next, acetoacetyl-CoA is converted to 3-hydroxy-3-methylglutaryl-CoA (HMG-CoA) by the enzymatic action of HMG-CoA synthase. This Co-A derivative is reduced to mevalonate by HMG-CoA reductase, which is a rate-limiting step of the mevalonate pathway of isoprenoid production. In the classical lower MVA pathway, mevalonate is then converted into mevalonate-5-phosphate (PM) via the action of mevalonate kinase (MVK) which is subsequently transformed into 5-diphosphomevalonate (DPM) by the enzymatic activity of phosphomevalonate kinase (PMK). Finally, IPP is formed from 5-diphosphomevalonate by the activity of the enzyme mevalonate-5-pyrophosphate decarboxylase (MVD), also known as diphosphomevalonate decarboxylase (DPMDC). The terms “classical lower mevalonate pathway” or “classical lower MVA pathway” refer to the series of reactions in cells catalyzed by the enzymes mevalonate kinase (MVK), phosphomevalonate kinase (PMK), and diphosphomevalonate decarboxylase (MVD).

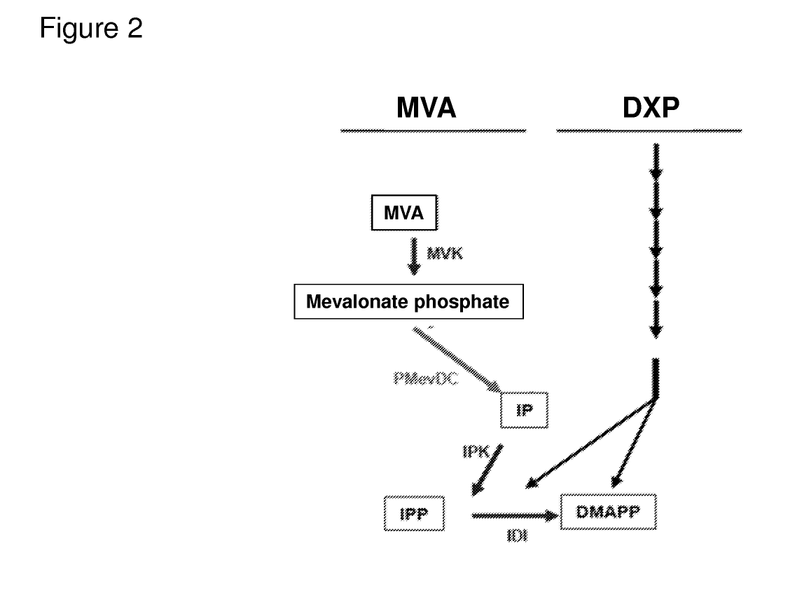

As provided herein, an alternative lower MVA pathway (e.g, mevalonate monophosphate pathway) has been identified wherein the mevalonate is converted into mevalonate-5-phosphate (PM) via the action of mevalonate kinase (MVK) which is subsequently transformed into isopentenyl phosphate by the enzymatic activity of a phosphomevalonate decarboxylase (PMevDC) and wherein the isopentenyl phosphate is converted to IPP by the enzymatic activity of isopentenyl kinase (IPK) (FIG. 2). The terms “alternative lower mevalonate pathway” or “alternative lower MVA pathway” refer to the series of reactions in cells catalyzed by the enzymes mevalonate kinase (MVK), phosphomevalonate decarboxylase (PMevDC), and isopentenyl kinase (IPK).

Thus, in certain embodiments, the recombinant cells of the present invention are recombinant cells having the ability to produce isoprenoid precursors, isoprene or isoprenoids via the mevalonate monophosphate pathway wherein the recombinant cells comprise: (i) a nucleic acid encoding a phosphomevalonate decarboxylase capable of synthesizing isopentenyl phosphate from mevalonate 5-phosphate, (ii) a nucleic acid encoding an isopentenyl kinase capable of synthesizing isopentenyl pyrophosphate from isopentenyl phosphate, (iii) one or more nucleic acid encoding one or more MVA polypeptides, and (iv) one or more heterologous nucleic acid involved in isoprenoid precursor, or isoprene or isoprenoid biosynthesis that enables the synthesis of isoprenoid precursors, isoprene or isoprenoids from acetoacetyl-CoA in the host cell. In other embodiments, recombinant cells of the present invention are recombinant cells having the ability to produce isoprenoid precursors, isoprene or isoprenoids wherein the recombinant cells comprise: (i) a nucleic acid encoding a phosphomevalonate decarboxylase capable of synthesizing isopentenyl phosphate from mevalonate 5-pyrophosphate, (ii) a nucleic acid encoding an isopentenyl kinase capable of synthesizing isopentenyl pyrophosphate from isopentenyl phosphate, (iii) one or more nucleic acid encoding one or more MVA polypeptides, and (iv) one or more heterologous nucleic acid involved in isoprenoid precursor, or isoprene or isoprenoid biosynthesis that enables the synthesis of isoprenoid precursors, isoprene or isoprenoids from acetoacetyl-CoA in the host cell. In another embodiments, recombinant cells of the present invention are recombinant cells having the ability to produce isoprenoid precursors, isoprene or isoprenoids wherein the recombinant cells comprise: (i) a nucleic acid encoding a phosphomevalonate decarboxylase capable of synthesizing isopentenyl pyrophosphate from mevalonate 5-pyrophosphate, (ii) a nucleic acid encoding an isopentenyl kinase capable of synthesizing isopentenyl pyrophosphate from isopentenyl phosphate, (iii) one or more nucleic acid encoding one or more MVA polypeptides, and (iv) one or more heterologous nucleic acid involved in isoprenoid precursor, or isoprene or isoprenoid biosynthesis that enables the synthesis of isoprenoid precursors, isoprene or isoprenoids from acetoacetyl-CoA in the host cell.

### Exemplary Phosphomevalonate Decarboxylase Nucleic Acids and Polypeptides

Phosphomevalonate decarboxylase enzymes catalyze the conversion of mevalonate 5-phosphate to isopentenyl phosphate. In certain embodiments, the phosphomevalonate decarboxylase is capable of catalyzing the conversion of mevalonate 5-pyrophosphate to isopentenyl phosphate. In other embodiments, the phosphomevalonate decarboxylase is capable of catalyzing the conversion of mevalonate 5-pyrophosphate to isopentenyl pyrophosphate. Thus, without being bound by theory, the expression of a phosphomevalonate decarboxylase as set forth herein can result in an increase in the amount of isopentenyl phosphate and/or isopentenyl pyrophosphate produced from a carbon source (e.g., a carbohydrate). Isopentenyl phosphate can be converted to isopentenyl pyrophosphate which can be used to produce isoprene or can be used as an isoprenoid precursor to produce isoprenoids. Thus the amount of these compounds produced from a carbon source may be increased. Alternatively, production of isopentenyl phosphate and isopentenyl pyrophosphate can be increased without the increase being reflected in higher intracellular concentration. In certain embodiments, intracellular isopentenyl phosphate and isopentenyl pyrophosphate concentrations will remain unchanged or even decrease, even though the phosphomevalonate decarboxylase reaction is taking place.

Exemplary phosphomevalonate decarboxylase nucleic acids include nucleic acids that encode a polypeptide, fragment of a polypeptide, peptide, or fusion polypeptide that has at least one activity of a phosphomevalonate decarboxylase polypeptide. Exemplary phosphomevalonate decarboxylase polypeptides and nucleic acids include naturally-occurring polypeptides and nucleic acids from any of the source organisms described herein as well as mutant polypeptides and nucleic acids derived from any of the source organisms described herein (See Example 2). Additionally, Table 1 provides a non-limiting list of species with nucleic acids that may encode exemplary phosphomevalonate decarboxylases which may be utilized within embodiments of the invention.

TABLE 1
Species that may express a candidate phosphomevalonate decarboxylase.

| Classification | Species | Reference |
|---|---|---|
| Desulfurococcales | Aeropyrum prenix | Matsumi et al.(2011) Res. Microbiol., v. |
| | Desulfurococcus kamchatkensis | 162, pp. 2929-2936. |
| | Hyperthmus butylicus | Grochowski et al. (2006) J. Bacteriol., V. |
| | Ignicoccus hospitalis | 188 (9), pp. 3192-3198. |
| | Staphylothermus marinus | |
| Sulfolobales | Metallosphaera sedula | Matsumi et al.(2011) Res. Microbiol., v. |
| | Sulfolobus acidocaldarius | 162, pp. 2929-2936. |
| | Sulfolobus islandicus | Grochowski et al. (2006) J. Bacteriol., V. |
| | Sulfolobus solfataricus | 188 (9), pp. 3192-3198. |
| | Sulfolobus tokodaii | |
| Thermoproteales | Caldivirga maquilingensis | Matsumi et al.(2011) Res. Microbiol., v. |
| | Pyrobaculum aerophilum | 162, pp. 2929-2936. |
| | Pyrobaculum arsenaticum | |
| | Pyrobaculum calidifontis | |
| | Pyrobaculum islandicum | |
| | Thermofilum pendens | |
| | Themoproteus neutrophilus | |
| Cenarchaeales | Cenarchaeum symbiosum | Matsumi et al.(2011) Res. Microbiol., v. |
| | | 162, pp. 2929-2936. |
| Nitrosopumilales | Nitrosopumilus maritimus | Matsumi et al.(2011) Res. Microbiol., v. |
| | | 162, pp. 2929-2936. |
| Archeaoglobales | Archaeoglobus fulgidus | Matsumi et al.(2011) Res. Microbiol., v. |
| | Archaeoglobus profundus | 162, pp. 2929-2936. |
| Halobacteriales | Halorhabdus utahensis | Matsumi et al.(2011) Res. Microbiol., v. |
| | | 162, pp. 2929-2936. |
| Methanococcales | Methanocaldococcus fervens | Matsumi et al.(2011) Res. Microbiol., v. |
| | Methanocaldococcus jannaschii | 162, pp. 2929-2936. |
| | Methanocaldococcus vulcanius | Grochowski et al. (2006) J. Bacteriol., V. |
| | Methanococcus aeolicus | 188 (9), pp. 3192-3198. |
| | Methanococcus maripaludis | |
| | Methanococcus vannielii | |
| Methanocellales | Methanocella paludicola | Matsumi et al.(2011) Res. Microbiol., v. |
| | Methanocella sp. RC-1 | 162, pp. 2929-2936. |
| Methanosarcinales | Methanococcoides burtonii | Matsumi et al.(2011) Res. Microbiol., v. |
| | Methanosaeta thermophile | 162, pp. 2929-2936. |
| | Methanosarcina acetivorans | Grochowski et al. (2006) J. Bacteriol., V. |
| | Methanosarcina barkeri | 188 (9), pp. 3192-3198. |
| | Methanosarcina mazei | |
| Methanobacteriales | Methanobrevibactor ruminantium | Matsumi et al.(2011) Res. Microbiol., v. |
| | Methanobrevibacter smithii | 162, pp. 2929-2936. |
| | Methanothermobacter | Grochowski et al. (2006) J. Bacteriol., V. |
| | thermautotrophicus | 188 (9), pp. 3192-3198. |
| | Methanosphaera stadtmanae | |
| Methanomicrobiales | Methanocorpusculum labreanum | Matsumi et al.(2011) Res. Microbiol., v. |
| | Methanoculleus marisnigri | 162, pp. 2929-2936. |
| | Candidatus Methanoregula boonei | |
| | Methanosphaerula palustris | |
| | Methanospirillum hungatei | |
| Methanopyrales | Methanopyrus kandleri | Matsumi et al.(2011) Res. Microbiol., v. |
| | | 162, pp. 2929-2936. |
| Thermococcales | Pyrococcus abyssi | Matsumi et al.(2011) Res. Microbiol., v. |
| | Pyrococcus furiosus | 162, pp. 2929-2936. |
| | Pyrococcus horikoshii | Grochowski et al. (2006) J. Bacteriol., V. |
| | Thermococcus gammatolerans | 188 (9), pp. 3192-3198. |
| | Thermococcus kodakaranesis | |
| | Thermococcus onnurineus | |
| | Thermococcus sibiricus | |
| Thermoplasmatales | Picrophilus torridus | Matsumi et al.(2011) Res. Microbiol., v. |
| | Thermoplasma acidophilum | 162, pp. 2929-2936. |
| | Thermoplasma volcanium | |
| Korarchaeota | Candidatus Korarchaeum cryptofilum | Matsumi et al.(2011) Res. Microbiol., v. |
| | | 162, pp. 2929-2936. |
| Nanoarchaeota | Nanosrchaeum equitans | Matsumi et al.(2011) Res. Microbiol., v. |
| | | 162, pp. 2929-2936. |

Other phosphomevalonate decarboxylases that can be used include members of Chloroflexi such as Herpetosiphonales (e.g., Herpetosiphon aurantiacus ATCC 23779) and Anaerolineae (e.g., Anaerolinea thermophila). Provided herein is also a phosphomevalonate decarboxylase isolated from a metagenomic library prepared from soil termed S378Pa3-2. Unless explicitly disclosed herein S378Pa3-2 is used interchangeably to describe the monophosphate decarboxylase and the microorganism the monophosphate decarboxylase is from.

The novel organism termed S378Pa3-2 expresses a polypeptide with phosphomevalonate decarboxylase activity wherein the polypeptide comprises the amino acid sequence of SEQ ID NO:18. It is contemplated herein that this organism and cell extracts from this organism has use in the methods and compositions disclosed herein. In some embodiments, provided herein is an isolated cell (e.g., a S378Pa3-2 cell) comprising a nucleic acid that can express a polypeptide having phosphomevalonate decarboxylase activity (e.g., a polypeptide with at least 85% sequence identity to the amino acid sequence of SEQ ID NO:18). In some embodiments, provided herein is a cell extract comprising a nucleic acid encoding a polypeptide with phosphomevalonate decarboxylase activity, wherein the cell extract is from an isolated cell (e.g., a S378Pa3-2 cell) comprising the nucleic acid encoding the polypeptide with phosphomevalonate decarboxylase activity (e.g., a polypeptide with at least 85% sequence identity to the amino acid sequence of SEQ ID NO:18). In some embodiments, provided herein is a cell extract comprising a polypeptide with phosphomevalonate decarboxylase activity, wherein the cell extract is from an isolated cell (e.g., a S378Pa3-2 cell) comprising the nucleic acid encoding the polypeptide with phosphomevalonate decarboxylase activity (e.g., a polypeptide with at least 85% sequence identity to the amino acid sequence of SEQ ID NO:18). In some aspects, provided herein is an isolated nucleic acid encoding a polypeptide with phosphomevalonate decarboxylase activity wherein the polypeptide comprises the amino acid sequence of SEQ ID NO:18. In some embodiments, the isolated nucleic acid sequence encoding a polypeptide having phosphomevalonate decarboxylase activity comprises at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a nucleic acid sequence encoding a phosphomevalonate decarboxylase comprising an amino acid sequence of SEQ ID NO:18. In some embodiments, the isolated nucleic acid sequence encoding a polypeptide having phosphomevalonate decarboxylase activity encodes a polypeptide having an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO:18. In some embodiments, the isolated nucleic acid encoding the polypeptide comprising the amino acid sequence of SEQ ID NO:18 (or polypeptide variant thereof) is complementary DNA (cDNA). The isolated nucleic acid encoding the polypeptide comprising the amino acid sequence of SEQ ID NO:18 (or polypeptide variant thereof) can be placed in a suitable vector (such as a vector described herein) for optimized expression of one or more copies of the nucleic acid. For example, the isolated nucleic acid encoding the polypeptide comprising the amino acid sequence of SEQ ID NO:18 (or polypeptide variant thereof) can be placed under an inducible promoter or a constitutive promoter. As another example, the isolated nucleic acid encoding the polypeptide comprising the amino acid sequence of SEQ ID NO:18 (or polypeptide variant thereof) can be cloned into one or more multicopy plasmids or integrated into a chromosome in a host cell. The host cell can be any host cell described herein such as a gram-positive bacterial cell, gram-negative bacterial cell, fungal cell, filamentous fungal cell, plant cell, algal cell, archaeal cell, or yeast cell. Accordingly, provided herein are recombinant cells comprising a nucleic acid encoding a polypeptide with phosphomevalonate decarboxylase activity wherein the polypeptide comprises the amino acid sequence of SEQ ID NO:18 or polypeptide variant thereof. For example, the recombinant cell can comprise a nucleic acid encoding a polypeptide comprising the amino acid of SEQ ID NO:18 and/or can comprise a nucleic acid encoding a polypeptide having an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO:18. Also provided herein is an isolated polypeptide comprising the amino acid of SEQ ID NO:18 or variant thereof. For example, the isolated polypeptide can comprise the amino acid of SEQ ID NO:18 or can comprise an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of SEQ ID NO:18. Also provided herein is a polypeptide comprising the amino acid sequence of SEQ ID NO:18, wherein the polypeptide further comprises a linker (e.g., affinity tag, a label, etc) or other sequence that aids in the synthesis, purification, or identification of the polypeptide, to enhance binding of the polypeptide to a solid support, or to increase solubility of the polypeptide. Exemplary linkers include, but are not limited to, a poly-histidine tag (e.g., 6×His-tag), maltose binding protein tag, glutathione S-transferase tag, FLAG epitope, MYC epitope, etc. Also contemplated herein are methods of culturing a cell (e.g., a S378Pa3-2 cell) encoding a nucleic acid that can express a polypeptide having phosphomevalonate decarboxylase activity. In some embodiments, provided herein are methods of culturing a cell (e.g., a S378Pa3-2 cell) encoding a nucleic acid that can express a polypeptide having phosphomevalonate decarboxylase activity under conditions suitable for expressing the polypeptide having phosphomevalonate decarboxylase activity.

In some aspects of the invention, provided herein is a phosphomevalonate decarboxylase isolated from a microorganism. In some aspects, a phosphomevalonate decarboxylase isolated from the group consisting of a gram positive bacterium, a gram negative bacterium, an aerobic bacterium, an anaerobic bacterium, a thermophilic bacterium, a psychrophilic bacterium, a halophilic bacterium or a cyanobacterium. In some aspects, a phosphomevalonate decarboxylase isolated from an archaea. In other aspects, a phosphomevalonate decarboxylase isolated from a soil metagenomic library. In some aspects, the phosphomevalonate decarboxylase is isolated from Herpetosiphon aurantiacus, Anaerolinea thermophila, or S378Pa3-2.

Provided herein are nucleic acids encoding a polypeptide with phosphomevalonate decarboxylase activity. In some aspects, the nucleic acid sequence encoding a polypeptide with phosphomevalonate decarboxylase activity comprises a nucleic acid sequence isolated from an archaea. In further aspects, the nucleic acid sequence encoding a polypeptide with phosphomevalonate decarboxylase activity comprises a nucleic acid sequence isolated from an archaea selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, methanomicrobiales, methanopyrales, thermococcales, thermoplasmatales, korarchaeota, and nanoarchaeota. In other aspects, the nucleic acid sequence encoding a polypeptide with phosphomevalonate decarboxylase activity comprises a nucleic acid sequence isolated from Herpetosiphon aurantiacus, Anaerolinea thermophila, or S378Pa3-2. In other aspects, the nucleic acid sequence encoding a polypeptide with phosphomevalonate decarboxylase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the nucleic acid sequence encoding a phosphomevalonate decarboxylase isolated from Herpetosiphon aurantiacus, Anaerolinea thermophila, or S378Pa3-2. In other aspects, the nucleic acid sequence encoding a polypeptide having phosphomevalonate decarboxylase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a nucleic acid sequence encoding a phosphomevalonate decarboxylase comprising an amino acid sequence selected from the group consisting of SEQ ID NOs:16-18. In other aspects, the nucleic acid sequence encoding a polypeptide having phosphomevalonate decarboxylase activity encodes a polypeptide having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs:16-18.

Also provided herein are polypeptides with phosphomevalonate decarboxylase activity. In some aspects, the polypeptide with phosphomevalonate decarboxylase activity is from an archaea. In further aspects, the polypeptide with phosphomevalonate decarboxylase activity is from an archaea selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, methanomicrobiales, methanopyrales, thermococcales, thermoplasmatales, korarchaeota, and nanoarchaeota. In other aspects, the polypeptide with phosphomevalonate decarboxylase activity is from Herpetosiphon aurantiacus, Anaerolinea thermophila, or S378Pa3-2. In some aspects, the polypeptide with phosphomevalonate decarboxylase activity comprises the amino acid sequence selected from the group consisting of SEQ ID NOs:16-18. Variants of any of the phosphomevalonate decarboxylases disclosed herein are also contemplated. In some aspects, a polypeptide with phosphomevalonate decarboxylase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of a phosphomevalonate decarboxylase isolated from Herpetosiphon aurantiacus, Anaerolinea thermophila, or S378Pa3-2. In some aspects, a polypeptide with phosphomevalonate decarboxylase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs:16-18.

Standard methods can be used to determine whether a polypeptide has phosphomevalonate decarboxylase activity by measuring the ability of the polypeptide to convert mevalonate 5-phosphate to isopentenyl phosphate. Another method for determining whether a polypeptide has phosphomevalonate decarboxylase activity is by measuring the ability of the polypeptide to convert mevalonate 5-pyrophosphate to isopentenyl phosphate or isopentenyl pyrophosphate. For example, conversion of the substrate to the product of the reaction can be detected by liquid chromatography-mass spectrometry (LC/MS). In another exemplary assay, a strain engineered to have a silenced DXP pathway and an inactivated classical lower MVA pathway can be used to identify a polypeptide with phosphomevalonate decarboxylase activity. In this assay, PMK and MVD of the lower classical MVA pathway are inactivated and replaced with a gene-cassette encoding a polypeptide with isopentenyl kinase activity (e.g., M. jannaschii IPK) without affecting the expression of MVK and IDI. The engineered strain is subsequently transformed with a nucleic acid encoding a candidate polypeptide with possible monophosphate decarboxylase activity and grown in media supplemented with IP. Growth of the engineered strain in the supplemented media indicates that the IP is converted to IPP and DMAPP, and confirms the candidate polypeptide has monophosphate decarboxylase activity. Any polypeptide identified as having phosphomevalonate decarboxylase activity as described herein is suitable for use in the present invention.

Phosphomevalonate decarboxylases can also be selected on the basis of biochemical characteristics including, but not limited to, protein expression, protein solubility, and activity. Phosphomevalonate decarboxylases can also be selected on the basis of other characteristics, including, but not limited to, diversity amongst different types of organisms (e.g., bacteria or archaea), close relatives to a desired species (e.g., Herpetosiphon aurantiacus), and thermotolerance.

As provided herein, phosphomevalonate decarboxylases allow production of isoprenoid precursors (e.g., IPP), isoprene, and/or isoprenoids. Provided herein is a recombinant host comprising a phosphomevalonate decarboxylase wherein the cells display at least one property of interest to for production of isoprenoid precursors (e.g., IPP), isoprene, and/or isoprenoids. In some embodiments, the recombinant host further comprises an isopentenyl kinase. In some aspects, said at least one property of interest is selected from, but not limited to, the group consisting of specific productivity, yield, titer and cellular performance index.

In certain embodiments, suitable phosphomevalonate decarboxylases for use herein include soluble phosphomevalonate decarboxylases. Techniques for measuring protein solubility are well known in the art and include those disclosed herein in the Examples. In some embodiments, a phosphomevalonate decarboxylase for use herein includes those with a solubility of at least 20% of total cellular phosphomevalonate decarboxylase protein. In some embodiments, phosphomevalonate decarboxylase protein solubility is between about any of 5% to about 100%, between about 10% to about 100%, between about 15% to about 100%, between about 20% to about 100%, between about 25% to about 100%, between about 30% to about 100%, between about 35% to about 100%, between about 40% to about 100%, between about 45% to about 100%, between about 50% to about 100%, between about 55% to about 100%, between about 60% to about 100%, between about 65% to about 100%, between about 70% to about 100%, between about 75% to about 100%, between about 80% to about 100%, between about 85% to about 100%, or between about 90% to about 100% of total cellular phosphomevalonate decarboxylase protein. In some embodiments, phosphomevalonate decarboxylase protein solubility is between about 5% to about 100% of total cellular phosphomevalonate decarboxylase protein. In some embodiments, phosphomevalonate decarboxylase protein solubility is between 5% and 100% of total cellular phosphomevalonate decarboxylase protein. In some embodiments, phosphomevalonate decarboxylase protein solubility is less than about any of 100, 90, 80, 70, 60, 50, 40, 30, 20, or 10 but no less than about 5% of total cellular phosphomevalonate decarboxylase protein. In some embodiments, solubility is greater than about any of 5, 10, 15, 20, 25, 30, 35, 40, 45, 50, 55, 60, 65, 70, 75, 80, 85, 90, or 95% of total cellular phosphomevalonate decarboxylase protein.

A phosphomevalonate decarboxylase with a desired kinetic characteristic increases the production of isoprene. Kinetic characteristics include, but are not limited to, specific activity, K_{cat}, Kᵢ, and Kₘ. In some aspects, the k_{cat} is at least about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In some aspects, the phosphomevalonate decarboxylase catalyzes the decarboxylation of phosphomevalonate with a k_{cat} of at least about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In other aspects, the phosphomevalonate decarboxylase catalyzes the decarboxylation of diphosphomevalonate with a k_{cat} of at least about 0.001, 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In some aspects, the Kₘ is at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, or 70. In some aspects, the phosphomevalonate decarboxylase catalyzes the decarboxylation of phosphomevalonate with a k_{M} of at least about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, or 70. In other aspects, the phosphomevalonate decarboxylase catalyzes the decarboxylation of diphosphomevalonate with a k_{cat} of at least about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, or 51.

Properties of interest include, but are not limited to, increased intracellular activity, specific productivity, yield, and cellular performance index as compared to a recombinant cell that does not comprise the phosphomevalonate decarboxylase polypeptide. In some embodiments, specific productivity increase at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6 7, 8, 9, 10 times or more. In one embodiment, isoprene specific productivity is about 15 mg/L/OD/hr. In some embodiments, isoprene yield increase of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5 times or more. In other embodiments, cell performance index increase at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5 times or more. In other embodiments, intracellular activity increase at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10 times or more.

### Recombinant Cells Expressing an Isopentenyl Kinase Polypeptide, a Phosphomevalonate Decarboxylase Polypeptide, and One or More Polypeptides of the MVA Pathway.

As provided herein, an alternative lower MVA pathway (e.g, mevalonate monophosphate pathway) has been identified wherein a phosphomevalonate decarboxylase (PMevDC) converts mevalonate 5-phosphate and/or mevalonate 5-pyrophosphate into isopentenyl phosphate. For production of isoprene, isoprenoid precursors, and/or isoprenoids, an isopentenyl kinase (IPK) catalyzes the conversion of isopentenyl phosphate to isopentenyl pyrophosphate (IPP) by an isopentenyl kinase (IPK). Therefore, use of a phosphomevalonate decarboxylase and an isopentenyl kinase from the alternative lower MVA pathway can bypass the enzymatic steps mediated by PMK and MVD of the classical lower MVA pathway. Each enzymatic step mediated by PMK and MVD of the classical lower MVA pathway utilizes an ATP. In the alternative lower MVA pathway, the enzymatic step mediated by IPK utilizes an ATP. Without being bound by theory, it is possible that the enzymatic step mediated by PMevDC in the alternative lower MVA pathway does not result in the utilization of ATP, thereby resulting in a reduction of the total amount of ATP consumed during the production of isopentenyl pyrophosphate (IPP) from mevalonate 5-phosphate via the alternative lower MVA pathway as compared to the classical lower MVA pathway.

Thus, in certain embodiments, the recombinant cells of the present invention are recombinant cells having the ability to produce isoprenoid precursors, isoprene or isoprenoids via the alternative lower MVA pathway wherein the recombinant cells comprise: (i) a nucleic acid encoding a phosphomevalonate decarboxylase capable of synthesizing isopentenyl phosphate from mevalonate 5-phosphate, (ii) a nucleic acid encoding an isopentenyl kinase capable of synthesizing isopentenyl pyrophosphate from isopentenyl phosphate, (iii) one or more nucleic acid encoding one or more MVA polypeptides, and (iv) one or more heterologous nucleic acid involved in isoprenoid precursor, or isoprene or isoprenoid biosynthesis that enables the synthesis of isoprenoid precursors, isoprene or isoprenoids from acetoacetyl-CoA in the host cell. In other embodiments, recombinant cells of the present invention are recombinant cells having the ability to produce isoprenoid precursors, isoprene or isoprenoids wherein the recombinant cells comprise: (i) a nucleic acid encoding a phosphomevalonate decarboxylase capable of synthesizing isopentenyl phosphate from mevalonate 5-pyrophosphate, (ii) a nucleic acid encoding an isopentenyl kinase capable of synthesizing isopentenyl pyrophosphate from isopentenyl phosphate, (iii) one or more nucleic acid encoding one or more MVA polypeptides, and (iv) one or more heterologous nucleic acid involved in isoprenoid precursor, or isoprene or isoprenoid biosynthesis that enables the synthesis of isoprenoid precursors, isoprene or isoprenoids from acetoacetyl-CoA in the host cell. In another embodiments, recombinant cells of the present invention are recombinant cells having the ability to produce isoprenoid precursors, isoprene or isoprenoids wherein the recombinant cells comprise: (i) a nucleic acid encoding a phosphomevalonate decarboxylase capable of synthesizing isopentenyl pyrophosphate from mevalonate 5-pyrophosphate, (ii) a nucleic acid encoding an isopentenyl kinase capable of synthesizing isopentenyl pyrophosphate from isopentenyl phosphate, (iii) one or more nucleic acid encoding one or more MVA polypeptides, and (iv) one or more heterologous nucleic acid involved in isoprenoid precursor, or isoprene or isoprenoid biosynthesis that enables the synthesis of isoprenoid precursors, isoprene or isoprenoids from acetoacetyl-CoA in the host cell. In some of the embodiments herein, the total amount of ATP utilized by the alternative lower MVA pathway for the production of isoprenoid precursors, isoprene or isoprenoids is reduced as compared to the total amount of ATP utilized by the classical lower MVA pathway for the production of isoprenoid precursors, isoprene, or isoprenoids. In some embodiments, the total amount of ATP utilized by the alternative lower MVA pathway for the production of isopentenyl pyrophosphate (IPP) from mevalonate 5-phosphate is reduced by a net of 1 ATP as compared to the total amount of ATP utilized by the classical lower MVA pathway for the production of isopentenyl pyrophosphate (IPP) from mevalonate 5-phosphate.

It is contemplated that any phosphomevalonate decarboxylase disclosed herein can be used in the present invention. Thus, in certain aspects, any of the nucleic acids encoding a phosphomevalonate decarboxylase contemplated herein or any of the polypeptides with phosphomevalonate decarboxylase activity contemplated herein can be expressed in recombinant cells in any of the ways described herein. The nucleic acid encoding a phosphomevalonate decarboxylase can be expressed in a recombinant cell on a multicopy plasmid. The plasmid can be a high copy plasmid, a low copy plasmid, or a medium copy plasmid. Alternatively, the nucleic acid encoding a phosphomevalonate decarboxylase can be integrated into the host cell's chromosome. For both heterologous expression of a nucleic acid encoding a phosphomevalonate decarboxylase on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the nucleic acid encoding a phosphomevalonate decarboxylase. In some embodiments, the nucleic acid encoding a polypeptide having phosphomevalonate decarboxylase activity is a heterologous nucleic acid. In some embodiments, the nucleic acid encoding a polypeptide having phosphomevalonate decarboxylase activity is an endogenous nucleic acid.

### Upper MVA Pathway Nucleic Acids and Polypeptides

The upper portion of the MVA pathway uses acetyl Co-A produced during cellular metabolism as the initial substrate for conversion to mevalonate via the actions of polypeptides having either: (a) (i) thiolase activity or (ii) acetoacetyl-CoA activity, (b) HMG-CoA reductase, and (c) HMG-CoA synthase enzymatic activity. First, acetyl Co-A is converted to acetoacetyl CoA via the action of a thiolase or an acetoacetyl-CoA synthase (which utilizes acetyl-CoA and malonyl-CoA). Next, acetoacetyl-CoA is converted to 3-hydroxy-3-methylglutaryl-CoA (HMG-CoA) by the enzymatic action of HMG-CoA synthase. This Co-A derivative is reduced to mevalonate by HMG-CoA reductase, which is a rate-limiting step of the mevalonate pathway of isoprenoid production.

Non-limiting examples of upper MVA pathway polypeptides include acetyl-CoA acetyltransferase (AA-CoA thiolase) polypeptides, acetoacetyl-CoA synthase polypeptides, 3-hydroxy-3-methylglutaryl-CoA synthase (HMG-CoA synthase) polypeptides, 3-hydroxy-3-methylglutaryl-CoA reductase (HMG-CoA reductase) polypeptides. Upper MVA pathway polypeptides can include polypeptides, fragments of polypeptides, peptides, and fusions polypeptides that have at least one activity of an upper MVA pathway polypeptide. Exemplary upper MVA pathway nucleic acids include nucleic acids that encode a polypeptide, fragment of a polypeptide, peptide, or fusion polypeptide that has at least one activity of an upper MVA pathway polypeptide. Exemplary MVA pathway polypeptides and nucleic acids include naturally-occurring polypeptides and nucleic acids from any of the source organisms described herein. Thus, it is contemplated herein that any gene encoding an upper MVA pathway polypeptide can be used in the present invention.

In certain embodiments, various options of mvaE and mvaS genes from L. grayi, E. faecium, E. gallinarum, E. casseliflavus and/or E. faecalis alone or in combination with one or more other mvaE and mvaS genes encoding proteins from the upper MVA pathway are contemplated within the scope of the invention. In other embodiments, an acetoacetyl-CoA synthase gene is contemplated within the scope of the present invention in combination with one or more other genes encoding: (i) 3-hydroxy-3-methylglutaryl-CoA synthase (HMG-CoA synthase) polypeptides and 3-hydroxy-3-methylglutaryl-CoA reductase (HMG-CoA reductase) polypeptides. Thus, in certain aspects, any of the combinations of genes contemplated herein can be expressed in recombinant cells in any of the ways described herein.

Additional non-limiting examples of upper MVA pathway polypeptides which can be used herein are described in International Patent Application Publication No. WO2009/076676; WO2010/003007 and WO2010/148150.

In certain embodiments, various options of mvaE and mvaS genes from L. grayi, E. faecium, E. gallinarum, E. casseliflavus and/or E. faecalis alone or in combination with one or more other mvaE and mvaS genes encoding proteins from the upper MVA pathway are contemplated within the scope of the invention. In L. grayi, E. faecium, E. gallinarum, E. casseliflavus, and E. faecalis, the mvaE gene encodes a polypeptide that possesses both thiolase and HMG-CoA reductase activities. In fact, the mvaE gene product represented the first bifunctional enzyme of IPP biosynthesis found in eubacteria and the first example of HMG-CoA reductase fused to another protein in nature (Hedl, et al., J Bacteriol. 2002 April; 184(8): 2116-2122). The mvaS gene, on the other hand, encodes a polypeptide having an HMG-CoA synthase activity.

Accordingly, recombinant cells (e.g., E. coli) can be engineered to express one or more mvaE and mvaS genes from L. grayi, E. faecium, E. gallinarum, E. casseliflavus and/or E. faecalis, to produce mevalonate. The one or more mvaE and mvaS genes can be expressed on a multicopy plasmid. The plasmid can be a high copy plasmid, a low copy plasmid, or a medium copy plasmid. Alternatively, the one or more mvaE and mvaS genes can be integrated into the host cell's chromosome. For both heterologous expression of the one or more mvaE and mvaS genes on a plasmid or as an integrated part of the host cell's chromosome, expression of the genes can be driven by either an inducible promoter or a constitutively expressing promoter. The promoter can be a strong driver of expression, it can be a weak driver of expression, or it can be a medium driver of expression of the one or more mvaE and mvaS genes.

Exemplary mvaE Polypeptides and Nucleic Acids

The mvaE gene encodes a polypeptide that possesses both thiolase and HMG-CoA reductase activities. The thiolase activity of the polypeptide encoded by the mvaE gene converts acetyl Co-A to acetoacetyl CoA whereas the HMG-CoA reductase enzymatic activity of the polypeptide converts 3-hydroxy-3-methylglutaryl-CoA to mevalonate. Exemplary mvaE polypeptides and nucleic acids include naturally-occurring polypeptides and nucleic acids from any of the source organisms described herein as well as mutant polypeptides and nucleic acids derived from any of the source organisms described herein that have at least one activity of a mvaE polypeptide.

Mutant mvaE polypeptides include those in which one or more amino acid residues have undergone an amino acid substitution while retaining mvaE polypeptide activity (i.e., the ability to convert acetyl Co-A to acetoacetyl CoA as well as the ability to convert 3-hydroxy-3-methylglutaryl-CoA to mevalonate). The amino acid substitutions can be conservative or non-conservative and such substituted amino acid residues can or can not be one encoded by the genetic code. The standard twenty amino acid “alphabet” has been divided into chemical families based on similarity of their side chains. Those families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A “conservative amino acid substitution” is one in which the amino acid residue is replaced with an amino acid residue having a chemically similar side chain (i.e., replacing an amino acid having a basic side chain with another amino acid having a basic side chain). A “non-conservative amino acid substitution” is one in which the amino acid residue is replaced with an amino acid residue having a chemically different side chain (i.e., replacing an amino acid having a basic side chain with another amino acid having an aromatic side chain).

Amino acid substitutions in the mvaE polypeptide can be introduced to improve the functionality of the molecule. For example, amino acid substitutions that increase the binding affinity of the mvaE polypeptide for its substrate, or that improve its ability to convert acetyl Co-A to acetoacetyl CoA and/or the ability to convert 3-hydroxy-3-methylglutaryl-CoA to mevalonate can be introduced into the mvaE polypeptide. In some aspects, the mutant mvaE polypeptides contain one or more conservative amino acid substitutions.

In one aspect, mvaE proteins that are not degraded or less prone to degradation can be used for the production of mevalonate, isoprenoid precursors, isoprene, and/or isoprenoids. Examples of gene products of mvaEs that are not degraded or less prone to degradation which can be used include, but are not limited to, those from the organisms E. faecium, E. gallinarum, E. casseliflavus, E. faecalis, and L. grayi. One of skill in the art can express mvaE protein in E. coli BL21 (DE3) and look for absence of fragments by any standard molecular biology techniques. For example, absence of fragments can be identified on Safestain stained SDS-PAGE gels following His-tag mediated purification or when expressed in mevalonate, isoprene, isoprenoid precursor, or isoprenoid producing E. coli BL21 using the methods of detection described herein.

Standard methods, such as those described in Hedl et al., (J Bacteriol. 2002, April; 184(8): 2116-2122) can be used to determine whether a polypeptide has mvaE activity, by measuring acetoacetyl-CoA thiolase as well as HMG-CoA reductase activity. In an exemplary assay, acetoacetyl-CoA thiolase activity is measured by spectrophotometer to monitor the change in absorbance at 302 nm that accompanies the formation or thiolysis of acetoacetyl-CoA. Standard assay conditions for each reaction to determine synthesis of acetoacetyl-CoA, are 1 mM acetyl-CoA, 10 mM MgCl₂, 50 mM Tris, pH 10.5 and the reaction is initiated by addition of enzyme. Assays can employ a final volume of 200 μl. For the assay, 1 enzyme unit (eu) represents the synthesis or thiolysis in 1 min of 1 μmol of acetoacetyl-CoA. In another exemplary assay, of HMG-CoA reductase activity can be monitored by spectrophotometer by the appearance or disappearance of NADP(H) at 340 nm. Standard assay conditions for each reaction measured to show reductive deacylation of HMG-CoA to mevalonate are 0.4 mM NADPH, 1.0 mM (R,S)-HMG-CoA, 100 mM KCl, and 100 mM KₓPO₄, pH 6.5. Assays employ a final volume of 200 μl. Reactions are initiated by adding the enzyme. For the assay, 1 eu represents the turnover, in 1 min, of 1 μmol of NADP(H). This corresponds to the turnover of 0.5 μmol of HMG-CoA or mevalonate.

Alternatively, production of mevalonate in recombinant cells can be measured by, without limitation, gas chromatography (see U.S. Patent Application Publication No.: US 2005/0287655 A1) or HPLC (See U.S. Patent Application Publication No.: 2011/0159557 A1). As an exemplary assay, cultures can be inoculated in shake tubes containing LB broth supplemented with one or more antibiotics and incubated for 14 h at 34° C. at 250 rpm. Next, cultures can be diluted into well plates containing TM3 media supplemented with 1% Glucose, 0.1% yeast extract, and 200 μM IPTG to final OD of 0.2. The plate are then sealed with a Breath Easier membrane (Diversified Biotech) and incubated at 34° C. in a shaker/incubator at 600 rpm for 24 hours. 1 mL of each culture is then centrifuged at 3,000×g for 5 min. Supernatant is then added to 20% sulfuric acid and incubated on ice for 5 min. The mixture is then centrifuged for 5 min at 3000×g and the supernatant was collected for HPLC analysis. The concentration of mevalonate in samples is determined by comparison to a standard curve of mevalonate (Sigma). The glucose concentration can additionally be measured by performing a glucose oxidase assay according to any method known in the art. Using HPLC, levels of mevalonate can be quantified by comparing the refractive index response of each sample versus a calibration curve generated by running various mevalonate containing solutions of known concentration.

Exemplary mvaE nucleic acids include nucleic acids that encode a polypeptide, fragment of a polypeptide, peptide, or fusion polypeptide that has at least one activity of a mvaE polypeptide. Exemplary mvaE polypeptides and nucleic acids include naturally-occurring polypeptides and nucleic acids from any of the source organisms described herein as well as mutant polypeptides and nucleic acids derived from any of the source organisms described herein. Exemplary mvaE nucleic acids include, for example, mvaE nucleic acids isolated from Listeria grayi_DSM 20601, Enterococcus faecium, Enterococcus gallinarum EG2, Enterococcus faecalis, and/or Enterococcus casseliflavus. The mvaE nucleic acid encoded by the Listeria grayi_DSM 20601 mvaE gene can have at least about 99%, 98%, 97%, 96%, 95%, 95%, 93%, 92%, 91%, 90%, 89%, 88%, 87%, 86%, or 85% sequence identity to SEQ ID NO:7. The mvaE nucleic acid encoded by the Enterococcus faecium mvaE gene can have at least about 99%, 98%, 97%, 96%, 95%, 95%, 93%, 92%, 91%, 90%, 89%, 88%, 87%, 86%, or 85% sequence identity to SEQ ID NO:8. The mvaE nucleic acid encoded by the Enterococcus gallinarum EG2 mvaE gene can have at least about 99%, 98%, 97%, 96%, 95%, 95%, 93%, 92%, 91%, 90%, 89%, 88%, 87%, 86%, or 85% sequence identity to SEQ ID NO:9. The mvaE nucleic acid encoded by the Enterococcus casseliflavus mvaE gene can have at least about 99%, 98%, 97%, 96%, 95%, 95%, 93%, 92%, 91%, 90%, 89%, 88%, 87%, 86%, or 85% sequence identity to SEQ ID NO:10. The mvaE nucleic acid encoded by the Enterococcus faecalis mvaE gene can have at least about 99%, 98%, 97%, 96%, 95%, 95%, 93%, 92%, 91%, 90%, 89%, 88%, 87%, 86%, or 85% sequence identity to the mvaE gene previously disclosed in E. coli to produce mevalonate (see US 2005/0287655 A1; Tabata, K. and Hashimoto, S.-I. Biotechnology Letters 26: 1487-1491, 2004).

Sequence of Listeria grayi DSM 20601 mvaE
(SEQ ID NO: 7)
atggttaaagacattgtaataattgatgccctccgtactcccatcgg
taagtaccgcggtcagctctcaaagatgacggcggtggaattgggaa
ccgcagttacaaaggctctgttcgagaagaacgaccaggtcaaagac
catgtagaacaagtcatttttggcaacgttttacaggcagggaacgg
ccagaatcccgcccgtcagatcgcccttaattctggcctgtccgcag
agataccggcttcgactattaaccaggtgtgtggttctggcctgaaa
gcaataagcatggcgcgccaacagatcctactcggagaagcggaagt
aatagtagcaggaggtatcgaatccatgacgaatgcgccgagtatta
catattataataaagaagaagacaccctctcaaagcctgttcctacg
atgaccttcgatggtctgaccgacgcgtttagcggaaagattatggg
tttaacagccgaaaatgttgccgaacagtacggcgtatcacgtgagg
cccaggacgcctttgcgtatggatcgcagatgaaagcagcaaaggcc
caagaacagggcattttcgcagctgaaatactgcctcttgaaatagg
ggacgaagttattactcaggacgagggggttcgtcaagagaccaccc
tcgaaaaattaagtctgcttcggaccatttttaaagaagatggtact
gttacagcgggcaacgcctcaacgatcaatgatggcgcctcagccgt
gatcattgcatcaaaggagtttgctgagacaaaccagattccctacc
ttgcgatcgtacatgatattacagagataggcattgatccatcaata
atgggcattgctcccgtgagtgcgatcaataaactgatcgatcgtaa
ccaaattagcatggaagaaatcgatctctttgaaattaatgaggcat
ttgcagcatcctcggtggtagttcaaaaagagttaagcattcccgat
gaaaagatcaatattggcggttccggtattgcactaggccatcctct
tggcgccacaggagcgcgcattgtaaccaccctagcgcaccagttga
aacgtacacacggacgctatggtattgcctccctgtgcattggcggt
ggccttggcctagcaatattaatagaagtgcctcaggaagatcagcc
ggttaaaaaattttatcaattggcccgtgaggaccgtctggctagac
ttcaggagcaagccgtgatcagcccagctacaaaacatgtactggca
gaaatgacacttcctgaagatattgccgacaatctgatcgaaaatca
aatatctgaaatggaaatccctcttggtgtggctttgaatctgaggg
tcaatgataagagttataccatcccactagcaactgaggaaccgagt
gtaatcgctgcctgtaataatggtgcaaaaatggcaaaccacctggg
cggttttcagtcagaattaaaagatggtttcctgcgtgggcaaattg
tacttatgaacgtcaaagaacccgcaactatcgagcatacgatcacg
gcagagaaagcggcaatttttcgtgccgcagcgcagtcacatccatc
gattgtgaaacgaggtgggggtctaaaagagatagtagtgcgtacgt
tcgatgatgatccgacgttcctgtctattgatctgatagttgatact
aaagacgcaatgggcgctaacatcattaacaccattctcgagggtgt
agccggctttctgagggaaatccttaccgaagaaattctgttctcta
ttttatctaattacgcaaccgaatcaattgtgaccgccagctgtcgc
ataccttacgaagcactgagtaaaaaaggtgatggtaaacgaatcgc
tgaaaaagtggctgctgcatctaaatttgcccagttagatccttatc
gagctgcaacccacaacaaaggtattatgaatggtattgaggccgtc
gttttggcctcaggaaatgacacacgggcggtcgcggcagccgcaca
tgcgtatgcttcacgcgatcagcactatcggggcttaagccagtggc
aggttgcagaaggcgcgttacacggggagatcagtctaccacttgca
ctcggcagcgttggcggtgcaattgaggtcttgcctaaagcgaaggc
ggcattcgaaatcatggggatcacagaggcgaaggagctggcagaag
tcacagctgcggtagggctggcgcaaaacctggcggcgttaagagcg
cttgttagtgaaggaatacagcaaggtcacatgtcgctccaggctcg
ctctcttgcattatcggtaggtgctacaggcaaggaagttgaaatcc
tggccgaaaaattacagggctctcgtatgaatcaggcgaacgctcag
accatactcgcagagatcagatcgcaaaaagttgaattgtga
Sequence of Enterococcus faecium mvaE
(SEQ ID NO: 8)
atgaccatgaacgttggaatcgataaaatgtcattctttgttccacc
ttactttgtggacatgactgatctggcagtagcacgggatgtcgatc
ccaataagtttctgattggtattggccaggaccagatggcagttaat
ccgaaaacgcaggatattgtgacatttgccacaaatgctgccaaaaa
catactgtcagctgaggaccttgataaaattgatatggtcatagtcg
gcaccgagagtggaatcgatgaatccaaagcgagtgccgtagtgctt
cacaggttgctcggtatccagaagtttgctcgctcctttgaaatcaa
agaagcctgttatgggggtaccgcggctttacagttcgctgtaaacc
acattaggaatcatcctgaatcaaaggttcttgtagttgcatcagat
atcgcgaaatacggcctggcttctggaggtgaaccaacgcaaggtgc
aggcgctgtggctatgctcgtctcaactgaccctaagatcattgctt
tcaacgacgatagcctcgcgcttacacaagatatctatgacttctgg
cgaccagttggacatgactatcctatggtcgacgggcctcttagtac
agagacctacatccagtcatttcagaccgtatggcaggaatacacaa
aacggtcgcagcatgcactggcagactttgctgcccttagctttcat
atcccgtatactaaaatgggcaaaaaggcgctgcttgcaatccttga
aggcgaatcagaggaggctcagaaccgtatactagcaaaatatgaaa
agagtatagcctactccagaaaggcgggtaacctgtataccggtagc
ctgtatctaggacttatttcacttctggaaaatgcagaagaccttaa
agctggtgatttaataggcctcttttcttacggttccggtgctgttg
cggagtttttctcaggaaggctggttgaggactatcaggaacagcta
cttaaaacaaaacatgccgaacagctggcccatagaaagcaactgac
aatcgaggagtacgaaacgatgttctccgatcgcttggacgtggaca
aagacgccgaatacgaagacacattagcttatagcatttcgtcagtc
cgaaacaccgtacgtgagtacaggagttga
Sequence of Enterococcus gallinarum EG2 mvaE
(SEQ ID NO: 9)
atgaaagaagtggttatgattgatgcggctcgcacacccattgggaa
atacagaggtagtcttagtccttttacagcggtggagctggggacac
tggtcacgaaagggctgctggataaaacaaagcttaagaaagacaag
atagaccaagtgatattcggcaatgtgcttcaggcaggaaacggaca
aaacgttgcaagacaaatagccctgaacagtggcttaccagttgacg
tgccggcgatgactattaacgaagtttgcgggtccggaatgaaagcg
gtgattttagcccgccagttaatacagttaggggaggcagagttggt
cattgcagggggtacggagtcaatgtcacaagcacccatgctgaaac
cttaccagtcagagaccaacgaatacggagagccgatatcatcaatg
gttaatgacgggctgacggatgcgttttccaatgctcacatgggtct
tactgccgaaaaggtggcgacccagttttcagtgtcgcgcgaggaac
aagaccggtacgcattgtccagccaattgaaagcagcgcacgcggtt
gaagccggggtgttctcagaagagattattccggttaagattagcga
cgaggatgtcttgagtgaagacgaggcagtaagaggcaacagcactt
tggaaaaactgggcaccttgcggacggtgttttctgaagagggcacg
gttaccgctggcaatgcttcaccgctgaatgacggcgctagtgtcgt
gattcttgcatcaaaagaatacgcggaaaacaataatctgccttacc
tggcgacgataaaggaggttgcggaagttggtatcgatccttctatc
atgggtattgccccaataaaggccattcaaaagttaacagatcggtc
gggcatgaacctgtccacgattgatctgttcgaaattaatgaagcat
tcgcggcatctagcattgttgtttctcaagagctgcaattggacgaa
gaaaaagtgaatatctatggcggggcgatagctttaggccatccaat
cggcgcaagcggagcccggatactgacaaccttagcatacggcctcc
tgcgtgagcaaaagcgttatggtattgcgtcattatgtatcggcggt
ggtcttggtctggccgtgctgttagaagctaatatggagcagaccca
caaagacgttcagaagaaaaagttttaccagcttaccccctccgagc
ggagatcgcagcttatcgagaagaacgttctgactcaagaaacggca
cttattttccaggagcagacgttgtccgaagaactgtccgatcacat
gattgagaatcaggtctccgaagtggaaattccaatgggaattgcac
aaaattttcagattaatggcaagaaaaaatggattcctatggcgact
gaagaaccttcagtaatagcggcagcatcgaacggcgccaaaatctg
cgggaacatttgcgcggaaacgcctcagcggcttatgcgcgggcaga
ttgtcctgtctggcaaatcagaatatcaagccgtgataaatgccgtg
aatcatcgcaaagaagaactgattctttgcgcaaacgagtcgtaccc
gagtattgttaaacgcgggggaggtgttcaggatatttctacgcggg
agtttatgggttcttttcacgcgtatttatcaatcgactttctggtg
gacgtcaaggacgcaatgggggcaaacatgatcaactctattctcga
aagcgttgcaaataaactgcgtgaatggttcccggaagaggaaatac
tgttctccatcctgtcaaacttcgctacggagtccctggcatctgca
tgttgcgagattccttttgaaagacttggtcgtaacaaagaaattgg
tgaacagatcgccaagaaaattcaacaggcaggggaatatgctaagc
ttgacccttaccgcgcggcaacccataacaaggggattatgaacggt
atcgaagccgtcgttgccgcaacgggaaacgacacacgggctgtttc
cgcttctattcacgcatacgccgcccgtaatggcttgtaccaaggtt
taacggattggcagatcaagggcgataaactggttggtaaattaaca
gtcccactggctgtggcgactgtcggtggcgcgtcgaacatattacc
aaaagccaaagcttccctcgccatgctggatattgattccgcaaaag
aactggcccaagtgatcgccgcggtaggtttagcacagaatctggcg
gcgttacgtgcattagtgacagaaggcattcagaaaggacacatggg
cttgcaagcacgttctttagcgatttcgataggtgccatcggtgagg
agatagagcaagtcgcgaaaaaactgcgtgaagctgaaaaaatgaat
cagcaaacggcaatacagattttagaaaaaattcgcgagaaatga
Sequence of Enterococcus casseliflavus mvaE
(SEQ ID NO: 10)
atgaaaatcggtattgaccgtctgtccttcttcatcccgaatttgta
tttggacatgactgagctggcagaatcacgcggggatgatccagcta
aatatcatattggaatcggacaagatcagatggcagtgaatcgcgca
aacgaggacatcataacactgggtgcaaacgctgcgagtaagatcgt
gacagagaaagaccgcgagttgattgatatggtaatcgttggcacgg
aatcaggaattgaccactccaaagcaagcgccgtgattattcaccat
ctccttaaaattcagtcgttcgcccgttctttcgaggtaaaagaagc
ttgctatggcggaactgctgccctgcacatggcgaaggagtatgtca
aaaatcatccggagcgtaaggtcttggtaattgcgtcagacatcgcg
cgttatggtttggccagcggaggagaagttactcaaggcgtgggggc
cgtagccatgatgattacacaaaacccccggattctttcgattgaag
acgatagtgtttttctcacagaggatatctatgatttctggcggcct
gattactccgagttccctgtagtggacgggcccctttcaaactcaac
gtatatagagagttttcagaaagtttggaaccggcacaaggaattgt
ccggaagagggctggaagattatcaagctattgcttttcacataccc
tatacgaagatgggtaagaaagcgctccagagtgttttagaccaaac
cgatgaagataaccaggagcgcttaatggctagatatgaggagtcta
ttcgctatagccggagaattggtaacctgtacacaggcagcttgtac
cttggtcttacaagcttgttggaaaactctaaaagtttacaaccggg
agatcggatcggcctcttttcctatggcagtggtgcggtgtccgagt
tctttaccgggtatttagaagaaaattaccaagagtacctgttcgct
caaagccatcaagaaatgctggatagccggactcggattacggtcga
tgaatacgagaccatcttttcagagactctgccagaacatggtgaat
gcgccgaatatacgagcgacgtccccttttctataaccaagattgag
aacgacattcgttattataaaatctga

The mvaE nucleic acid can be expressed in a recombinant cell on a multicopy plasmid. The plasmid can be a high copy plasmid, a low copy plasmid, or a medium copy plasmid. Alternatively, the mvaE nucleic acid can be integrated into the host cell's chromosome. For both heterologous expression of an mvaE nucleic acid on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the mvaE nucleic acid.

Exemplary mvaS Polypeptides and Nucleic Acids

The mvaS gene encodes a polypeptide that possesses HMG-CoA synthase activity. This polypeptide can convert acetoacetyl CoA to 3-hydroxy-3-methylglutaryl-CoA (HMG-CoA). Exemplary mvaS polypeptides and nucleic acids include naturally-occurring polypeptides and nucleic acids from any of the source organisms described herein as well as mutant polypeptides and nucleic acids derived from any of the source organisms described herein that have at least one activity of a mvaS polypeptide.

Mutant mvaS polypeptides include those in which one or more amino acid residues have undergone an amino acid substitution while retaining mvaS polypeptide activity (i.e., the ability to convert acetoacetyl CoA to 3-hydroxy-3-methylglutaryl-CoA). Amino acid substitutions in the mvaS polypeptide can be introduced to improve the functionality of the molecule. For example, amino acid substitutions that increase the binding affinity of the mvaS polypeptide for its substrate, or that improve its ability to convert acetoacetyl CoA to 3-hydroxy-3-methylglutaryl-CoA can be introduced into the mvaS polypeptide. In some aspects, the mutant mvaS polypeptides contain one or more conservative amino acid substitutions.

Standard methods, such as those described in Quant et al. (Biochem J., 1989, 262:159-164), can be used to determine whether a polypeptide has mvaS activity, by measuring HMG-CoA synthase activity. In an exemplary assay, HMG-CoA synthase activity can be assayed by spectrophotometrically measuring the disappearance of the enol form of acetoacetyl-CoA by monitoring the change of absorbance at 303 nm. A standard 1 ml assay system containing 50 mm-Tris/HCl, pH 8.0, 10 mM-MgCl2 and 0.2 mM-dithiothreitol at 30° C.; 5 mM-acetyl phosphate, 10, M-acetoacetyl-CoA and 5 μl samples of extracts can be added, followed by simultaneous addition of acetyl-CoA (100 μM) and 10 units of PTA. HMG-CoA synthase activity is then measured as the difference in the rate before and after acetyl-CoA addition. The absorption coefficient of acetoacetyl-CoA under the conditions used (pH 8.0, 10 mM-MgCl₂), is 12.2×10³ M⁻¹ cm⁻¹. By definition, 1 unit of enzyme activity causes 1 μmol of acetoacetyl-CoA to be transformed per minute.

Alternatively, production of mevalonate in recombinant cells can be measured by, without limitation, gas chromatography (see U.S. Patent Application Publication No.: US 2005/0287655 A1) or HPLC (See U.S. Patent Application Publication No.: 2011/0159557 A1). As an exemplary assay, cultures can be inoculated in shake tubes containing LB broth supplemented with one or more antibiotics and incubated for 14 h at 34° C. at 250 rpm. Next, cultures can be diluted into well plates containing TM3 media supplemented with 1% Glucose, 0.1% yeast extract, and 200 μM IPTG to final OD of 0.2. The plate are then sealed with a Breath Easier membrane (Diversified Biotech) and incubated at 34° C. in a shaker/incubator at 600 rpm for 24 hours. 1 mL of each culture is then centrifuged at 3,000×g for 5 min. Supernatant is then added to 20% sulfuric acid and incubated on ice for 5 min. The mixture is then centrifuged for 5 min at 3000×g and the supernatant was collected for HPLC analysis. The concentration of mevalonate in samples is determined by comparison to a standard curve of mevalonate (Sigma). The glucose concentration can additionally be measured by performing a glucose oxidase assay according to any method known in the art. Using HPLC, levels of mevalonate can be quantified by comparing the refractive index response of each sample versus a calibration curve generated by running various mevonate containing solutions of known concentration.

Exemplary mvaS nucleic acids include nucleic acids that encode a polypeptide, fragment of a polypeptide, peptide, or fusion polypeptide that has at least one activity of a mvaS polypeptide. Exemplary mvaS polypeptides and nucleic acids include naturally-occurring polypeptides and nucleic acids from any of the source organisms described herein as well as mutant polypeptides and nucleic acids derived from any of the source organisms described herein. Exemplary mvaS nucleic acids include, for example, mvaS nucleic acids isolated from Listeria grayi DSM 20601, Enterococcus faecium, Enterococcus gallinarum EG2, Enterococcus faecalis, and/or Enterococcus casseliflavus. The mvaS nucleic acid encoded by the Listeria grayi_DSM 20601 mvaS gene can have at least about 99%, 98%, 97%, 96%, 95%, 95%, 93%, 92%, 91%, 90%, 89%, 88%, 87%, 86%, or 85% sequence identity to SEQ ID NO:11. The mvaS nucleic acid encoded by the Enterococcus faecium mvaS gene can have at least about 99%, 98%, 97%, 96%, 95%, 95%, 93%, 92%, 91%, 90%, 89%, 88%, 87%, 86%, or 85% sequence identity to SEQ ID NO:12. The mvaS nucleic acid encoded by the Enterococcus gallinarum EG2 mvaS gene can have at least about 99%, 98%, 97%, 96%, 95%, 95%, 93%, 92%, 91%, 90%, 89%, 88%, 87%, 86%, or 85% sequence identity to SEQ ID NO:13. The mvaS nucleic acid encoded by the Enterococcus casseliflavus mvaS gene can have at least about 99%, 98%, 97%, 96%, 95%, 95%, 93%, 92%, 91%, 90%, 89%, 88%, 87%, 86%, or 85% sequence identity to SEQ ID NO:14. The mvaS nucleic acid encoded by the Enterococcus faecalis mvaS gene can have at least about 99%, 98%, 97%, 96%, 95%, 95%, 93%, 92%, 91%, 90%, 89%, 88%, 87%, 86%, or 85% sequence identity to the mvaE gene previously disclosed in E. coli to produce mevalonate (see US 2005/0287655 A1; Tabata, K. and Hashimoto, S.-I. Biotechnology Letters 26: 1487-1491, 2004).

Sequence of Listeria grayi DSM 20601 mvaS
(SEQ ID NO: 11)
atggaagaagtggtaattatagatgcacgtcggactccgattggtaa
atatcacgggtcgttgaagaagttttcagcggtggcgctggggacgg
ccgtggctaaagacatgttcgaacgcaaccagaaaatcaaagaggag
atcgcgcaggtcataattggtaatgtcttgcaggcaggaaatggcca
gaaccccgcgcggcaagttgctcttcaatcagggttgtccgttgaca
ttcccgcttctacaattaacgaggtttgtgggtctggtttgaaagct
atcttgatgggcatggaacaaatccaactcggcaaagcgcaagtagt
gctggcaggcggcattgaatcaatgacaaatgcgccaagcctgtccc
actataacaaggcggaggatacgtatagtgtcccagtgtcgagcatg
acactggatggtctgacagacgcattttctagtaaacctatgggatt
aacagcggaaaacgtcgcacagcgctacggtatctcccgtgaggcgc
aagatcaattcgcatatcaatctcagatgaaagcagcaaaagcgcag
gcagaaaacaaattcgctaaggaaattgtgccactggcgggtgaaac
taaaaccatcacagctgacgaagggatcagatcccaaacaacgatgg
agaaactggcaagtctcaaacctgtttttaaaaccgatggcactgta
accgcagggaatgctagcaccattaatgacggggccgcccttgtgct
gcttgctagcaaaacttactgcgaaactaatgacataccgtaccttg
cgacaatcaaagaaattgttgaagttggaatcgatccggagattatg
ggcatctctccgataaaagcgatacaaacattgttacaaaatcaaaa
agttagcctcgaagatattggagtttttgaaataaatgaagcctttg
ccgcaagtagcatagtggttgaatctgagttgggattagatccggct
aaagttaaccgttatgggggtggtatatccttaggtcatgcaattgg
ggcaaccggcgctcgcctggccacttcactggtgtatcaaatgcagg
agatacaagcacgttatggtattgcgagcctgtgcgttggtggtgga
cttggactggcaatgcttttagaacgtccaactattgagaaggctaa
accgacagacaaaaagttctatgaattgtcaccagctgaacggttgc
aagagctggaaaatcaacagaaaatcagttctgaaactaaacagcag
ttatctcagatgatgcttgccgaggacactgcaaaccatttgataga
aaatcaaatatcagagattgaactcccaatgggcgtcgggatgaacc
tgaaggttgatgggaaagcctatgttgtgccaatggcgacggaagag
ccgtccgtcatcgcggccatgtctaatggtgccaaaatggccggcga
aattcacactcagtcgaaagaacggctgctcagaggtcagattgttt
tcagcgcgaagaatccgaatgaaatcgaacagagaatagctgagaac
caagctttgattttcgaacgtgccgaacagtcctatccttccattgt
gaaaagagagggaggtctccgccgcattgcacttcgtcattttcctg
ccgattctcagcaggagtctgcggaccagtccacatttttatcagtg
gacctttttgtagatgtgaaagacgcgatgggggcaaatatcataaa
tgcaatacttgagggcgtcgcagccctgtttcgcgaatggttcccca
atgaggaaattcttttttctattctctcgaacttggctacggagagc
ttagtcacggctgtttgtgaagtcccatttagtgcacttagcaagag
aggtggtgcaacggtggcccagaaaattgtgcaggcgtcgctcttcg
caaagacagacccataccgcgcagtgacccacaacaaagggattatg
aacggtgtagaggctgttatgcttgccacaggcaacgacacgcgcgc
agtctcagccgcttgtcatggatacgcagcgcgcaccggtagctatc
agggtctgactaactggacgattgagtcggatcgcctggtaggcgag
ataacactgccgctggccatcgctacagttggaggcgctaccaaagt
gttgcccaaagctcaagcggcactggagattagtgatgttcactctt
ctcaagagcttgcagccttagcggcgtcagtaggtttagtacaaaat
ctcgcggccctgcgcgcactggtttccgaaggtatacaaaaagggca
catgtccatgcaagcccggtctctcgcaatcgcggtcggtgctgaaa
aagccgagatcgagcaggtcgccgaaaagttgcggcagaacccgcca
atgaatcagcagcaggcgctccgttttcttggcgagatccgcgaaca
atga
Sequence of Enterococcus faecium mvaS
(SEQ ID NO: 12)
atgaacgtcggcattgacaaaattaattttttcgttccaccgtatta
tctggatatggtcgacctggcccacgcacgcgaagtggacccgaaca
aatttacaattggaattggacaggatcagatggctgtgagcaaaaag
acgcacgatatcgtaacattcgcggctagtgccgcgaaggaaatttt
agaacctgaggacttgcaagctatagacatggttatagttggtaccg
aatcgggcattgacgagagcaaagcatccgcggtcgttttacatcgt
ttgttgggcgtacaacctttcgctcgcagttttgaaattaaagaagc
ctgttacggggcaaccgcaggcattcagtttgccaagactcatatac
aagcgaacccggagagcaaggtcctggtaattgcaagcgatatagct
cggtatggtcttcggtcaggtggagagcccacacaaggcgcaggggc
agttgctatgcttctcacggcaaatcccagaatcctgaccttcgaaa
acgacaatctgatgttaacgcaggatatttatgacttctggagacca
cttggtcacgcttaccctatggtagatggccacctttccaatcaagt
ctatattgacagttttaagaaggtctggcaagcacattgcgaacgca
atcaagcttctatatccgactatgccgcgattagttttcatattccg
tatacaaaaatgggtaagaaagccctgctcgctgtttttgcagatga
agtggaaactgaacaggaacgcgttatggcacggtatgaagagtcta
tcgtatattcacgccggatcggcaacttgtatacgggatcattgtac
ctggggctgatatccttattggaaaacagttctcacctgtcggcggg
cgaccggataggattgtttagttatgggagtggcgctgtcagcgaat
ttttctccggtcgtttagtggcaggctatgaaaatcaattgaacaaa
gaggcgcatacccagctcctggatcagcgtcagaagctttccatcga
agagtatgaggcgatttttacagattccttagaaattgatcaggatg
cagcgttctcggatgacctgccatattccatccgcgagataaaaaac
acgattcggtactataaggagagctga
Sequence of Enterococcus gallinarum EG2 mvaS
(SEQ ID NO: 13)
atggaagaagttgtcatcattgacgcactgcgtactccaataggaaa
gtaccacggttcgctgaaagattacacagctgttgaactggggacag
tagcagcaaaggcgttgctggcacgaaatcagcaagcaaaagaacac
atagcgcaagttattattggcaacgtcctgcaagccggaagtgggca
gaatccaggccgacaagtcagtttacagtcaggattgtcttctgata
tccccgctagcacgatcaatgaagtgtgtggctcgggtatgaaagcg
attctgatgggtatggagcaaattcagctgaacaaagcctctgtggt
cttaacaggcggaattgaaagcatgaccaacgcgccgctgtttagtt
attacaacaaggctgaggatcaatattcggcgccggttagcacaatg
atgcacgatggtctaacagatgctttcagttccaaaccaatgggctt
aaccgcagagaccgtcgctgagagatatggaattacgcgtaaggaac
aagatgaatttgcttatcactctcaaatgaaggcggccaaagcccag
gcggcgaaaaagtttgatcaggaaattgtacccctgacggaaaaatc
cggaacggttctccaggacgaaggcatcagagccgcgacaacagtcg
agaagctagctgagcttaaaacggtgttcaaaaaagacggaacagtt
acagcgggtaacgcctctacgataaatgatggcgctgctatggtatt
aatagcatcaaaatcttattgcgaagaacaccagattccttatctgg
ccgttataaaggagatcgttgaggtgggttttgcccccgaaataatg
ggtatttcccccattaaggctatagacaccctgctgaaaaatcaagc
actgaccatagaggatataggaatatttgagattaatgaagcctttg
ctgcgagttcgattgtggtagaacgcgagttgggcctggaccccaaa
aaagttaatcgctatggcggtggtatatcactcggccacgcaattgg
ggcgacgggagctcgcattgcgacgaccgttgcttatcagctgaaag
atacccaggagcgctacggtatagcttccttatgcgttggtgggggt
cttggattggcgatgcttctggaaaacccatcggccactgcctcaca
aactaattttgatgaggaatctgcttccgaaaaaactgagaagaaga
agttttatgcgctagctcctaacgaacgcttagcgtttttggaagcc
caaggcgctattaccgctgctgaaaccctggtcttccaggagatgac
cttaaacaaagagacagccaatcacttaatcgaaaaccaaatcagcg
aagttgaaattcctttaggcgtgggcctgaacttacaggtgaatggg
aaagcgtataatgttcctctggccacggaggaaccgtccgttatcgc
tgcgatgtcgaatggcgccaaaatggctggtcctattacaacaacaa
gtcaggagaggctgttacggggtcagattgtcttcatggacgtacag
gacccagaagcaatattagcgaaagttgaatccgagcaagctaccat
tttcgcggtggcaaatgaaacatacccgtctatcgtgaaaagaggag
gaggtctgcgtagagtcattggcaggaatttcagtccggccgaaagt
gacttagccacggcgtatgtatcaattgacctgatggtagatgttaa
ggatgcaatgggtgctaatatcatcaatagtatcctagaaggtgttg
cggaattgtttagaaaatggttcccagaagaagaaatcctgttctca
attctctccaatctcgcgacagaaagtctggtaacggcgacgtgctc
agttccgtttgataaattgtccaaaactgggaatggtcgacaagtag
ctggtaaaatagtgcacgcggcggactttgctaagatagatccatac
agagctgccacacacaataaaggtattatgaatggcgttgaagcgtt
aatcttagccaccggtaatgacacccgtgcggtgtcggctgcatgcc
acggttacgcggcacgcaatgggcgaatgcaagggcttacctcttgg
acgattatcgaagatcggctgataggctctatcacattacctttggc
tattgcgacagtggggggtgccacaaaaatcttgccaaaagcacagg
ccgccctggcgctaactggcgttgagacggcgtcggaactggccagc
ctggcggcgagtgtgggattagttcaaaatttggccgctttacgagc
actagtgagcgagggcattcagcaagggcacatgagtatgcaagcta
gatccctggccattagcgtaggtgcgaaaggtactgaaatagagcaa
ctagctgcgaagctgagggcagcgacgcaaatgaatcaggagcaggc
tcgtaaatttctgaccgaaataagaaattaa
Sequence of Enterococcus casseliflavus mvaS
(SEQ ID NO: 14)
atgaacgttggaattgataaaatcaattttttcgttccgccctattt
cattgatatggtggatctcgctcatgcaagagaagttgaccccaaca
agttcactataggaataggccaagatcagatggcagtaaacaagaaa
acgcaagatatcgtaacgttcgcgatgcacgccgcgaaggatattct
gactaaggaagatttacaggccatagatatggtaatagtggggactg
agtctgggatcgacgagagcaaggcaagtgctgtcgtattgcatcgg
cttttaggtattcagccttttgcgcgctcctttgaaattaaggaggc
atgctatggggccactgccggccttcagtttgcaaaagctcatgtgc
aggctaatccccagagcaaggtcctggtggtagcttccgatatagca
cgctacggactggcatccggaggagaaccgactcaaggtgtaggtgc
tgtggcaatgttgatttccgctgatccagctatcttgcagttagaaa
atgataatctcatgttgacccaagatatatacgatttttggcgcccg
gtcgggcatcaatatcctatggtagacggccatctgtctaatgccgt
ctatatagacagctttaaacaagtctggcaagcacattgcgagaaaa
accaacggactgctaaagattatgctgcattgtcgttccatattccg
tacacgaaaatgggtaagaaagctctgttagcggtttttgcggagga
agatgagacagaacaaaagcggttaatggcacgttatgaagaatcaa
ttgtatacagtcgtcggactggaaatctgtatactggctcactctat
ctgggcctgatttccttactggagaatagtagcagtttacaggcgaa
cgatcgcataggtctgtttagctatggttcaggggccgttgcggaat
ttttcagtggcctcttggtaccgggttacgagaaacaattagcgcaa
gctgcccatcaagctcttctggacgaccggcaaaaactgactatcgc
agagtacgaagccatgtttaatgaaaccattgatattgatcaggacc
agtcatttgaggatgacttactgtactccatcagagagatcaaaaac
actattcgctactataacgaggagaatgaataa

The mvaS nucleic acid can be expressed in a recombinant cell on a multicopy plasmid. The plasmid can be a high copy plasmid, a low copy plasmid, or a medium copy plasmid. Alternatively, the mvaS nucleic acid can be integrated into the host cell's chromosome. For both heterologous expression of an mvaS nucleic acid on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the mvaS nucleic acid.

Acetoacetyl-CoA Synthase Nucleic Acids and Polypeptides

The acetoacetyl-CoA synthase gene (aka nphT7) is a gene encoding an enzyme having the activity of synthesizing acetoacetyl-CoA from malonyl-CoA and acetyl-CoA and having minimal activity (e.g., no activity) of synthesizing acetoacetyl-CoA from two acetyl-CoA molecules. See, e.g., Okamura et al., PNAS Vol 107, No. 25, pp. 11265-11270 (2010), the contents of which are expressly incorporated herein for teaching about nphT7. An acetoacetyl-CoA synthase gene from an actinomycete of the genus Streptomyces CL190 strain was described in JP Patent Publication (Kokai) No. 2008-61506 A and US2010/0285549.

In any of the aspects or embodiments described herein, an enzyme that has the ability to synthesize acetoacetyl-CoA from malonyl-CoA and acetyl-CoA can be used. Non-limiting examples of such an enzyme are described herein. In certain embodiments described herein, an acetoacetyl-CoA synthase gene derived from an actinomycete of the genus Streptomyces having the activity of synthesizing acetoacetyl-CoA from malonyl-CoA and acetyl-CoA can be used. An example of such an acetoacetyl-CoA synthase gene is the gene encoding a protein having the amino acid sequence of SEQ ID NO: 15. Such a protein having the amino acid sequence of SEQ ID NO: 15 corresponds to an acetoacetyl-CoA synthase having activity of synthesizing acetoacetyl-CoA from malonyl-CoA and acetyl-CoA and having no activity of synthesizing acetoacetyl-CoA from two acetyl-CoA molecules.

Sequence of acetoacetyl-CoA synthase
(SEQ ID NO: 15)
MTDVRFRIIGTGAYVPERIVSNDEVGAPAGVDDDWITRKTGIRQRRW
AADDQATSDLATAAGRAALKAAGITPEQLTVIAVATSTPDRPQPPTA
AYVQHHLGATGTAAFDVNAVCSGTVFALSSVAGTLVYRGGYALVIGA
DLYSRILNPADRKTVVLFGDGAGAMVLGPTSTGTGPIVRRVALHTFG
GLTDLIRVPAGGSRQPLDTDGLDAGLQYFAMDGREVRRFVTEHLPQL
IKGFLHEAGVDAADISHFVPHQANGVMLDEVFGELHLPRATMHRTVE
TYGNTGAASIPITMDAAVRAGSFRPGELVLLAGFGGGMAASFALIEW.

The acetoacetyl-CoA synthase activity of a polypeptide can be evaluated as described below. Specifically, a gene encoding a polypeptide to be evaluated is first introduced into a host cell such that the gene can be expressed therein, followed by purification of the protein by a technique such as chromatography. Malonyl-CoA and acetyl-CoA are added as substrates to a buffer containing the obtained protein to be evaluated, followed by, for example, incubation at a desired temperature (e.g., 10° C. to 60° C.). After the completion of reaction, the amount of substrate lost and/or the amount of product (acetoacetyl-CoA) produced are determined. Thus, it is possible to evaluate whether or not the protein being tested has the function of synthesizing acetoacetyl-CoA from malonyl-CoA and acetyl-CoA and to evaluate the degree of synthesis. In such case, it is possible to examine whether or not the protein has the activity of synthesizing acetoacetyl-CoA from two acetyl-CoA molecules by adding acetyl-CoA alone as a substrate to a buffer containing the obtained protein to be evaluated and determining the amount of substrate lost and/or the amount of product produced in a similar manner.

### Classical and Alternative Lower MVA Pathway Nucleic Acids and Polypeptides

As provided herein, the classical lower mevalonate biosynthetic pathway comprises mevalonate kinase (MVK), phosphomevalonate kinase (PMK), and diphosphomevalonte decarboxylase (MVD). Also as provided herein, the alternative lower MVA pathway utilizes the classical lower MVK polypeptide and therefore comprises mevalonate kinase (MVK), phosphomevalonate decarboxylase (PMevDc), and isopentenyl kinase (IPK). In some aspects, the classical lower MVA pathway can further comprise isopentenyl diphosphate isomerase (IDI). In some aspects, the alternative lower MVA pathway can further comprise isopentenyl diphosphate isomerase (IDI). The MVK polypeptide used in both the alternative lower MVA pathway and the classical lower MVA pathway can be from the genus Methanosarcina and, more specifically, from Methanosarcina mazei. In some embodiments, the MVK polypeptide can be from M. burtonii. Additional examples of lower MVA pathway polypeptides can be found in U.S. Patent Application Publication 2010/0086978 the contents of which are expressly incorporated herein by reference in their entirety with respect to MVK polypeptides and MVK polypeptide variants.

In a preferred embodiment, cells provided herein comprise one or more upper MVA pathway polypeptides and one or more alternative lower MVA pathway polypeptides. Polypeptides of the alternative lower MVA pathway can be any enzyme (a) that phosphorylates mevalonate to mevalonate 5-phosphate; (b) that converts mevalonate 5-phosphate to isopentenyl phosphate; (c) that converts mevalonate 5-pyrophosphate to isopentenyl phosphate; (d) that converts mevalonate 5-pyrophosphate to isopentenyl pyrophosphate; and (e) that converts isopentenyl phosphate to isopentenyl pyrophosphate. In a preferred embodiment, polypeptides of the alternative lower MVA pathway can be any enzyme (a) that phosphorylates mevalonate to mevalonate 5-phosphate; (b) that converts mevalonate 5-phosphate to isopentenyl phosphate; and (c) that converts isopentenyl phosphate to isopentenyl pyrophosphate. More particularly, the enzyme that phosphorylates mevalonate to mevalonate 5-phosphate can be from the group consisting of M. mazei mevalonate kinase, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, Streptomyces CL190 mevalonate kinase polypeptide, and M. Burtonii mevalonate kinase polypeptide. In another aspect, the enzyme that phosphorylates mevalonate to mevalonate 5-phosphate is M. mazei mevalonate kinase. In some aspects, the enzyme that converts mevalonate 5-phosphate to isopentenyl phosphate can be from the group consisting of Herpetosiphon aurantiacus phosphomevalonate decarboxylase polypeptide, Anaerolinea thermophila phosphomevalonate decarboxylase polypeptide, and S378Pa3-2 phosphomevalonate decarboxylase polypeptide. In another aspect, the enzyme that converts isopentenyl phosphate to isopentenyl pyrophosphate can be from the group consisting of Herpetosiphon aurantiacus isopentenyl kinase polypeptide, Methanocaldococcus jannaschii isopentenyl kinase polypeptide, and Methanobrevibacter ruminantium isopentenyl kinase polypeptide.

Any of the cells described herein can comprise MVK nucleic acid(s) (e.g., endogenous or heterologous nucleic acid(s) encoding MVK polypeptide). In some aspects, the MVK nucleic acid(s) is from the group consisting of M. mazei, Lactobacillus, Lactobacillus sakei, yeast, Saccharomyces cerevisiae, Streptococcus, Streptococcus pneumoniae, Streptomyces, Streptomyces CL190, and M. Burtonii. Any of the cells described herein can comprise PMevDC nucleic acid(s) (e.g., endogenous or heterologous nucleic acid(s) encoding PMevDC polypeptide). In some aspects, the PMevDC nucleic acids(s) can be from an archaea. In some aspects, the PMevDC nucleic acid(s) can be from the genus Herpetosiphon. In some aspects, the PMevDC nucleic acid(s) is from the group consisting of Herpetosiphon aurantiacus, Anaerolinea thermophila, or S378Pa3-2. Any of the cells described herein can comprise IPK nucleic acid(s) (e.g., endogenous or heterologous nucleic acid(s) encoding IPK polypeptide). In some aspects, the IPK nucleic acid(s) can be from an archaea. In some aspects, the IPK nucleic acid(s) can be from the genus selected from the group consisting of Methanocaldococcus, Methanobrevibacter, and Herpetosiphon. In some aspects, the IPK nucleic acid(s) is from Herpetosiphon aurantiacus, Methanocaldococcus jannaschii, or Methanobrevibacter ruminantium.

In one aspect, any one of the cells described herein can comprise nucleic acid(s) encoding a PMK polypeptide. The nucleic acid encoding a PMK can be a heterologous nucleic acid or an endogenous nucleic acid. In another aspect, any one of the cells described herein can comprise nucleic acid(s) encoding an MVD polypeptide. The nucleic acid encoding an MVD can be a heterologous nucleic acid or an endogenous nucleic acid. In some cases, attenuating the activity of the endogenous PMK gene and/or the endogenous MVD gene in cells with MVK, PMevDC, and IPK gene expression results in more carbon flux into the alternative lower MVA pathway in comparison to cells that do not have attenuated endogenous PMK gene and/or endogenous MVD gene expression. In some aspects, the activity of PMK and/or MVD is modulated by attenuating the activity of an endogenous PMK gene and/or an endogenous MVD gene. In some aspects, endogenous PMK and/or endogenous MVD gene expression is attenuated by deletion of the endogenous PMK gene and/or the endogenous MVD gene. In some aspects, endogenous PMK and/or endogenous MVD gene expression is attenuated by mutation of the endogenous PMK gene and/or the endogenous MVD gene. In some aspects of any of the aspects provided herein, the cells produce decreased amounts of mevalonate 5-pyrophosphate in comparison to microorganisms that do not have attenuated endogenous PMK gene and/or endogenous MVD gene expression. In some aspects of any of the aspects provided herein, attenuating the activity of the endogenous PMK gene and/or endogenous MVD gene results in more carbon flux into the alternative lower MVA pathway in comparison to microorganisms that do not have attenuated endogenous PMK gene and/or endogenous MVD gene expression. In other aspects, any of the cells herein comprise a heterologous nucleic acid encoding a PMK polypeptide and/or MVD polypeptide. In some cases, attenuating the activity of the heterologous PMK gene and/or the heterologous MVD gene in cells with MVK, PMevDC, and IPK gene expression results in more carbon flux into the alternative lower MVA pathway in comparison to cells that do not have attenuated heterologous PMK gene and/or heterologous MVD gene expression. In some aspects, the activity of PMK and/or MVD is modulated by attenuating the activity of a heterologous PMK gene and/or a heterologous MVD gene. In some aspects, heterologous PMK and/or heterologous MVD gene expression is attenuated by deletion of the heterologous PMK gene and/or the heterologous MVD gene. In some aspects, heterologous PMK and/or heterologous MVD gene expression is attenuated by mutation of the heterologous PMK gene and/or the heterologous MVD gene. In some aspects, any of the cells herein do not comprise a heterologous nucleic acid encoding a PMK polypeptide and/or MVD polypeptide.

In some aspects, the lower MVA pathway polypeptide (e.g., classical and alternative) is a heterologous polypeptide. In some aspects, the cells comprise more than one copy of a heterologous nucleic acid encoding a lower MVA pathway polypeptide (e.g., classical and alternative). In some aspects, the heterologous nucleic acid encoding a lower MVA pathway polypeptide (e.g., classical and alternative) is operably linked to a constitutive promoter. In some aspects, the heterologous nucleic acid encoding a lower MVA pathway polypeptide (e.g., classical and alternative) is operably linked to an inducible promoter. In some aspects, the heterologous nucleic acid encoding a lower MVA pathway polypeptide (e.g., classical and alternative) is operably linked to a strong promoter. In some aspects, the heterologous nucleic acid encoding a lower MVA pathway polypeptide (e.g., classical and alternative) is operably linked to a weak promoter. The heterologous nucleic acids encoding a lower MVA pathway polypeptide (e.g., classical and alternative) can be integrated into a genome of the cells or can be stably expressed in the cells. The heterologous nucleic acids encoding a lower MVA pathway polypeptide (e.g., classical and alternative) can additionally be on a vector.

In some aspects of the invention, the cells described in any of the compositions or methods described herein further comprise one or more nucleic acids encoding a lower mevalonate (MVA) pathway polypeptide(s) (e.g., classical and alternative). In some aspects, the lower MVA pathway polypeptide (e.g., classical and alternative) is an endogenous polypeptide. In some aspects, the endogenous nucleic acid encoding a lower MVA pathway polypeptide (e.g., classical and alternative) is operably linked to a constitutive promoter. In some aspects, the endogenous nucleic acid encoding a lower MVA pathway polypeptide (e.g., classical and alternative) is operably linked to an inducible promoter. In some aspects, the endogenous nucleic acid encoding a lower MVA pathway polypeptide (e.g., classical and alternative) is operably linked to a strong promoter. In a particular aspect, the cells are engineered to over-express the endogenous lower MVA pathway polypeptide (e.g., classical and alternative) relative to wild-type cells. In some aspects, the endogenous nucleic acid encoding a lower MVA pathway polypeptide (e.g., classical and alternative) is operably linked to a weak promoter.

Any one of the promoters described herein (e.g., promoters described herein and identified in the Examples of the present disclosure including inducible promoters and constitutive promoters) can be used to drive expression of any of the MVA polypeptides described herein.

Lower MVA pathway polypeptides (e.g., classical and alternative) include polypeptides, fragments of polypeptides, peptides, and fusions polypeptides that have at least one activity of a lower MVA pathway polypeptide. Exemplary lower MVA pathway nucleic acids include nucleic acids that encode a polypeptide, fragment of a polypeptide, peptide, or fusion polypeptide that has at least one activity of a lower MVA pathway polypeptide. Exemplary lower MVA pathway polypeptides and nucleic acids include naturally-occurring polypeptides and nucleic acids from any of the source organisms described herein. In addition, variants of lower MVA pathway polypeptides that confer the result of better isoprene production can also be used as well.

Any one of the cells described herein can comprise IDI nucleic acid(s) (e.g., endogenous or heterologous nucleic acid(s) encoding IDI). Isopentenyl diphosphate isomerase polypeptides (isopentenyl-diphosphate delta-isomerase or IDI) catalyzes the interconversion of isopentenyl diphosphate (IPP) and dimethylallyl diphosphate (DMAPP) (e.g., converting IPP into DMAPP and/or converting DMAPP into IPP). Exemplary IDI polypeptides include polypeptides, fragments of polypeptides, peptides, and fusions polypeptides that have at least one activity of an IDI polypeptide. Standard methods (such as those described herein) can be used to determine whether a polypeptide has IDI polypeptide activity by measuring the ability of the polypeptide to interconvert IPP and DMAPP in vitro, in a cell extract, or in vivo. Exemplary IDI nucleic acids include nucleic acids that encode a polypeptide, fragment of a polypeptide, peptide, or fusion polypeptide that has at least one activity of an IDI polypeptide. Exemplary IDI polypeptides and nucleic acids include naturally-occurring polypeptides and nucleic acids from any of the source organisms described herein as well as mutant polypeptides and nucleic acids derived from any of the source organisms described herein.

### Isopentenyl Kinase Polypeptides and Nucleic Acids

Isopentenyl kinase enzymes catalyze the conversion of isopentenyl phosphate to isopentenyl pyrophosphate. Thus, without being bound by theory, the expression of an isopentenyl kinase as set forth herein can result in an increase in the amount of isopentenyl pyrophosphate produced from a carbon source (e.g., a carbohydrate). Isopentenyl pyrophosphate can be used to produce isoprene or can be used as an isoprenoid precursor to produce isoprenoids. Thus the amount of isopentenyl pyrophosphate produced from a carbon source may be increased. Alternatively, production of isopentenyl pyrophosphate can be increased without the increase being reflected in a higher intracellular concentration. In certain embodiments, intracellular isopentenyl pyrophosphate concentrations will remain unchanged or even decrease, even though the isopentenyl kinase reaction is taking place.

Exemplary isopentenyl kinase nucleic acids include nucleic acids that encode a polypeptide, fragment of a polypeptide, peptide, or fusion polypeptide that has at least one activity of an isopentenyl kinase polypeptide. Exemplary isopentenyl kinase polypeptides and nucleic acids include naturally-occurring polypeptides and nucleic acids from any of the source organisms described herein as well as mutant polypeptides and nucleic acids derived from any of the source organisms described herein (See Example 1). In addition, Table 2 provides a non-limiting list of species with nucleic acids that encode or may encode exemplary isopentenyl kinase which may be utilized within embodiments of the invention.

TABLE 2
Species that express or may express an isopentenyl kinase.

| Classification | Species | Reference |
|---|---|---|
| Desulfurococcales | Aeropyrum prenix | Matsumi et al.(2011) Res. Microbiol., v. |
| | Desulfurococcus kamchatkensis | 162, pp. 2929-2936. |
| | Hyperthmus butylicus | Grochowski et al. (2006) J. Bacteriol., V. |
| | Ignicoccus hospitalis | 188 (9), pp. 3192-3198. |
| | Staphylothermus marinus | |
| Sulfolobales | Metallosphaera sedula | Matsumi et al.(2011) Res. Microbiol., v. |
| | Sulfolobus islandicus | 162, pp. 2929-2936. |
| | Sulfolobus solfataricus | Grochowski et al. (2006) J. Bacteriol., V. |
| | | 188 (9), pp. 3192-3198. |
| Thermoproteales | Caldivirga maquilingensis | Matsumi et al.(2011) Res. Microbiol., v. |
| | Pyrobaculum aerophilum | 162, pp. 2929-2936. |
| | Pyrobaculum arsenaticum | |
| | Pyrobaculum calidifontis | |
| | Pyrobaculum islandicum | |
| | Thermofilum pendens | |
| | Themoproteus neutrophilus | |
| Cenarchaeales | Cenarchaeum symbiosum | Matsumi et al.(2011) Res. Microbiol., v. |
| | | 162, pp. 2929-2936. |
| Nitrosopumilales | Nitrosopumilus maritimus | Matsumi et al.(2011) Res. Microbiol., v. |
| | | 162, pp. 2929-2936. |
| Archeaoglobales | Archaeoglobus fulgidus | Matsumi et al.(2011) Res. Microbiol., v. |
| | Archaeoglobus profundus | 162, pp. 2929-2936. |
| | | Grochowski et al. (2006) J. Bacteriol., V. |
| | | 188 (9), pp. 3192-3198. |
| Halobacteriales | Haloarcula marismortui | Matsumi et al.(2011) Res. Microbiol., v. |
| | Halobacterium salinarum | 162, pp. 2929-2936. |
| | Halobacterium sp. NRC-1 | |
| | Halomicrobium mukohataei | |
| | Haloquadratum walsbyi | |
| | Halorhabdus utahensis | |
| | Halorubrum lacusprofundi | |
| | Haloterrigena turkmenica | |
| | Notronomonas pharaonis | |
| Methanococcales | Methanocaldococcus fervens | Matsumi et al.(2011) Res. Microbiol., v. |
| | Methanocaldococcus jannaschii | 162, pp. 2929-2936. |
| | Methanocaldococcus vulcanius | Grochowski et al. (2006) J. Bacteriol., V. |
| | Methanococcus aeolicus | 188 (9), pp. 3192-3198. |
| | Methanococcus maripaludis | |
| | Methanococcus vannielii | |
| Methanocellales | Methanocella paludicola | Matsumi et al.(2011) Res. Microbiol., v. |
| | Methanocella sp. RC-1 | 162, pp. 2929-2936. |
| Methanosarcinales | Methanococcoides burtonii | Matsumi et al.(2011) Res. Microbiol., v. |
| | Methanosaeta thermophile | 162, pp. 2929-2936. |
| | Methanosarcina acetivorans | Grochowski et al. (2006) J. Bacteriol., V. |
| | Methanosarcina barkeri | 188 (9), pp. 3192-3198. |
| | Methanosarcina mazei | |
| Methanobacteriales | Methanobrevibactor ruminantium | Matsumi et al.(2011) Res. Microbiol., v. |
| | Methanobrevibacter smithii | 162, pp. 2929-2936. |
| | Methanothermobacter | Chen et al. (2010), Biochemistry., v. 49, |
| | thermautotrophicus | pp. 207-217. |
| | Methanosphaera stadtmanae | Grochowski et al. (2006) J. Bacteriol., V. |
| | | 188 (9), pp. 3192-3198. |
| Methanomicrobiales | Methanocorpusculum labreanum | Matsumi et al.(2011) Res. Microbiol., v. |
| | Methanoculleus marisnigri | 162, pp. 2929-2936. |
| | Candidatus Methanoregula boonei | |
| | Methanosphaerula palustris | |
| | Methanospirillum hungatei | |
| Methanopyrales | Methanopyrus kandleri | Matsumi et al.(2011) Res. Microbiol., v. |
| | | 162, pp. 2929-2936. |
| Thermococcales | Pyrococcus abyssi | Matsumi et al.(2011) Res. Microbiol., v. |
| | Pyrococcus furiosus | 162, pp. 2929-2936. |
| | Pyrococcus horikoshii | Grochowski et al. (2006) J. Bacteriol., V. |
| | Thermococcus gammatolerans | 188 (9), pp. 3192-3198. |
| | Thermococcus kodakaranesis | |
| | Thermococcus onnurineus | |
| | Thermococcus sibiricus | |
| Thermoplasmatales | Picrophilus torridus | Matsumi et al.(2011) Res. Microbiol., v. |
| | Thermoplasma acidophilum | 162, pp. 2929-2936. |
| | Thermoplasma volcanium | Chen et al. (2010), Biochemistry., v. 49, |
| | | pp. 207-217. |
| Korarchaeota | Candidatus Korarchaeum cryptofilum | Matsumi et al.(2011) Res. Microbiol., v. |
| | | 162, pp. 2929-2936. |

Other isopentenyl kinases that can be used include members of Chloroflexi such as Herpetosiphonales (e.g., Herpetosiphon aurantiacus ATCC 23779).

Provided herein is an isopentenyl kinase isolated from a microorganism. In some aspects, an isopentenyl kinase isolated from the group consisting of a gram positive bacterium, a gram negative bacterium, an aerobic bacterium, an anaerobic bacterium, a thermophilic bacterium, a psychrophilic bacterium, a halophilic bacterium or a cyanobacterium. In some aspects, an isopentenyl kinase isolated from an archaea. In some aspects, the isopentenyl kinase is isolated from Herpetosiphon aurantiacus, Methanocaldococcus jannaschii, or Methanobrevibacter ruminantium. Provided herein are nucleic acids encoding a polypeptide with isopentenyl kinase activity. In some aspects, the nucleic acid sequence encoding a polypeptide with isopentenyl kinase activity comprises a nucleic acid sequence isolated from an archaea. In further aspects, the nucleic acid sequence encoding a polypeptide with isopentenyl kinase activity comprises a nucleic acid sequence isolated from an archaea selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, methanomicrobiales, methanopyrales, thermococcales, thermoplasmatales, korarchaeota, and nanoarchaeota. In other aspects, the nucleic acid sequence encoding a polypeptide with isopentenyl kinase activity comprises a nucleic acid sequence isolated from Herpetosiphon aurantiacus, Methanocaldococcus jannaschii, or Methanobrevibacter ruminantium. In other aspects, the nucleic acid sequence encoding a polypeptide with isopentenyl kinase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the nucleic acid sequence encoding an isopentenyl kinase isolated from Herpetosiphon aurantiacus, Methanocaldococcus jannaschii, or Methanobrevibacter ruminantium. In other aspects, the nucleic acid sequence encoding a polypeptide having isopentenyl kinase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a nucleic acid sequence encoding an isopentenyl kinase comprising an amino acid sequence selected from the group consisting of SEQ ID NOs:19-23. In other aspects, the nucleic acid sequence encoding a polypeptide having isopentenyl kinase activity encodes a polypeptide having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence selected from the group consisting of SEQ ID NOs:19-23.

Also provided herein are polypeptides with isopentenyl kinase activity. In some aspects, the polypeptide with isopentenyl kinase activity is from an archaea. In further aspects, the polypeptide with isopentenyl kinase activity is from an archaea selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, methanomicrobiales, methanopyrales, thermococcales, thermoplasmatales, korarchaeota, and nanoarchaeota. In other aspects, the polypeptide with isopentenyl kinase activity is from Herpetosiphon aurantiacus, Methanocaldococcus jannaschii, or Methanobrevibacter ruminantium. In some aspects, the polypeptide with isopentenyl kinase activity comprises the amino acid sequence selected from the group consisting of SEQ ID NOs:19-23. Variants of any of the isopentenyl kinases disclosed herein are also contemplated. In some aspects, a polypeptide with isopentenyl kinase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to the amino acid sequence of a isopentenyl kinase isolated from Herpetosiphon aurantiacus, Methanocaldococcus jannaschii, or Methanobrevibacter ruminantium. In some aspects, a polypeptide with isopentenyl kinase activity comprises at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to an amino acid sequence selected from the group consisting of SEQ ID NOs:19-23.

Standard methods can be used to determine whether a polypeptide has isopentenyl kinase activity by measuring the ability of the polypeptide to convert isopentenyl phosphate to isopentenyl pyrophosphate. For example, conversion of the substrate to the product of the reaction can be detected by LC/MS. In another exemplary assay, a strain engineered to express the classical lower MVA pathway is transformed with a plasmid expressing a candidate isopentenyl kinase and grown in media supplemented with IP. Growth of the engineered strain in the supplemented media indicates that the IP is converted to IPP and DMAPP, and confirms the candidate polypeptide has isopentenyl kinase activity. Any polypeptide identified as having isopentenyl kinase activity as described herein is suitable for use in the present invention.

Biochemical characteristics of exemplary isopentenyl kinases include, but are not limited to, protein expression, protein solubility, and activity. Isopentenyl kinases can also be selected on the basis of other characteristics, including, but not limited to, diversity amongst different types of organisms (e.g., bacteria, archaea), close relatives to a desired species (e.g., Herpetosiphon aurantiacus, Methanocaldococcus jannaschii, etc.), and thermotolerance.

Provided herein is a recombinant host comprising phosphomevalonate decarboxylases and isopentenyl kinases wherein the cells display at least one property of interest to improve production of isoprenoid precursors (e.g., IPP), isoprene, and/or isoprenoids. In some aspects, said at least one property of interest is selected from, but not limited to, the group consisting of specific productivity, yield, titer and cellular performance index.

In certain embodiments, suitable isopentenyl kinases for use herein include soluble isopentenyl kinases. Techniques for measuring protein solubility are well known in the art and include those disclosed herein in the Examples. In some embodiments, isopentenyl kinases for use herein include those with a solubility of at least 20% of total cellular isopentenyl kinase protein. In some embodiments, isopentenyl kinase protein solubility is between about any of 5% to about 100%, between about 10% to about 100%, between about 15% to about 100%, between about 20% to about 100%, between about 25% to about 100%, between about 30% to about 100%, between about 35% to about 100%, between about 40% to about 100%, between about 45% to about 100%, between about 50% to about 100%, between about 55% to about 100%, between about 60% to about 100%, between about 65% to about 100%, between about 70% to about 100%, between about 75% to about 100%, between about 80% to about 100%, between about 85% to about 100%, or between about 90% to about 100% of total cellular isopentenyl kinase protein. In some embodiments, isopentenyl kinase protein solubility is between about 5% to about 100% of total cellular isopentenyl kinase protein. In some embodiments, isopentenyl kinase protein solubility is between 5% and 100% of total cellular isopentenyl kinase protein. In some embodiments, isopentenyl kinase protein solubility is less than about any of 100, 90, 80, 70, 60, 50, 40, 30, 20, or 10 but no less than about 5% of total cellular isopentenyl kinase protein. In some embodiments, solubility is greater than about any of 5, 10, 15, 20, 25, 30, 35, 40, 45, 50, 55, 60, 65, 70, 75, 80, 85, 90, or 95% of total cellular isopentenyl kinase protein.

An isopentenyl kinase with a desired kinetic characteristic increases the production of isoprene. Kinetic characteristics include, but are not limited to, specific activity, K_{cat}, Kᵢ, and Kₘ. In some aspects, the k_{cat} is at least about 0.001, 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6, 6.5, 7, 7.5, 8, 8.5, 9, 9.5, 10, 10.5, 11, 11.5, 12, 12.5, 13, 13.5, 14, 14.5, 15, 16, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30. In some aspects, the isopentenyl kinase catalyzes the conversion of isopentenyl phosphate to isopentenyl pyrophosphate with a k_{cat} of at least about 0.001, 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6, 6.5, 7, 7.5, 8, 8.5, 9, 9.5, 10, 10.5, 11, 11.5, 12, 12.5, 13, 13.5, 14, 14.5, 15, 16, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30. In some embodiments, the isopentenyl kinase catalyzes the conversion of isopentenyl phosphate to isopentenyl pyrophosphate with a k_{cat} of at least about 27.5. In other embodiments, the isopentenyl kinase catalyzes the conversion of isopentenyl phosphate to isopentenyl pyrophosphate with a k_{cat} of at least about 8.0. In yet other embodiments, the isopentenyl kinase catalyzes the conversion of isopentenyl phosphate to isopentenyl pyrophosphate with a k_{cat} of at least about 0.03.

In some aspects, the Kₘ is at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 75, 80, 85, 90, 95, 100, 105, 110, 115, 120, 125, 130, 135, 140, 145, 150, 175, 200, 225, 250, or 275. In some aspects, the isopentenyl kinase catalyzes the conversion of isopentenyl phosphate to isopentenyl pyrophosphate with a k_{M} of at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 75, 80, 85, 90, 95, 100, 105, 110, 115, 120, 125, 130, 135, 140, 145, 150, 175, 200, 225, 250, or 275. In some embodiments, the isopentenyl kinase catalyzes the conversion of isopentenyl phosphate to isopentenyl pyrophosphate with a k_{M} of at least about 12.7. In other embodiments, the isopentenyl kinase catalyzes the conversion of isopentenyl phosphate to isopentenyl pyrophosphate with a k_{M} of at least about 4.4. In yet other embodiments, the isopentenyl kinase catalyzes the conversion of isopentenyl phosphate to isopentenyl pyrophosphate with a k_{M} of at least about 256.

Properties of interest include, but are not limited to, increased intracellular activity, specific productivity, yield, and cellular performance index as compared to a recombinant cell that does not comprise the isopentenyl kinase polypeptide. In some embodiments, specific productivity increase at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6 7, 8, 9, 10 times or more. In one embodiment, isoprene specific productivity is about 15 mg/L/OD/hr. In some embodiments, isoprene yield increase of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5 times or more. In other embodiments, cell performance index increase at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5 times or more. In other embodiments, intracellular activity increase at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10 times or more.

It is contemplated that any isopentenyl kinase disclosed herein can be used in the present invention. Thus, in certain aspects, any of the nucleic acids encoding an isopentenyl kinase contemplated herein or any of the polypeptides with isopentenyl kinase activity contemplated herein can be expressed in recombinant cells in any of the ways described herein. The nucleic acid encoding an isopentenyl kinase can be expressed in a recombinant cell on a multicopy plasmid. The plasmid can be a high copy plasmid, a low copy plasmid, or a medium copy plasmid. Alternatively, the nucleic acid encoding an isopentenyl kinase can be integrated into the host cell's chromosome. For both heterologous expression of a nucleic acid encoding an isopentenyl kinase on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the nucleic acid encoding an isopentenyl kinase. In some embodiments, the nucleic acid encoding a polypeptide having isopentenyl kinase activity is a heterologous nucleic acid. In some embodiments, the nucleic acid encoding a polypeptide having isopentenyl kinase activity is an endogenous nucleic acid.

### Recombinant Cells Capable of Utilizing the Alternative Mevalonate Monophosphate Pathway

The recombinant cells (e.g., recombinant bacterial cells) described herein can produce isopentenyl pyrophosphate from mevalonate via the alternative lower MVA pathway. In some aspects, recombinant cells produce isopentenyl pyrophosphate from mevalonate via the alternative lower MVA pathway at an amount and/or concentration greater than that of the same cells without any manipulation to the various enzymatic pathways described herein. Thus, the recombinant cells described herein are useful in the production of isopentenyl pyrophosphate via the alternative lower MVA pathway.

Accordingly, in certain aspects, the invention provides recombinant cells capable of isopentenyl pyrophosphate production, wherein the cells comprise (i) a nucleic acid encoding a polypeptide having phosphomevalonate decarboxylase activity, (ii) a nucleic acid encoding a polypeptide having isopentenyl kinase activity, and (iii) one or more nucleic acids encoding one or more polypeptides of the MVA pathway, wherein the cells produce increased amounts of isopentenyl pyrophosphate compared to cells that do not comprise a nucleic acid encoding a polypeptide having phosphomevalonate decarboxylase activity and/or a nucleic acid encoding a polypeptide having isopentenyl kinase activity.

In certain aspects, the recombinant cells described herein comprise a nucleic acid encoding a phosphomevalonate decarboxylase from Herpetosiphon aurantiacus, Anaerolinea thermophila, or S378Pa3-2. In certain aspects, the recombinant cells described herein comprise one or more copies of a heterologous nucleic acid encoding a phosphomevalonate decarboxylase isolated from Herpetosiphon aurantiacus, Anaerolinea thermophila, or S378Pa3-2. In some aspects, the recombinant cells described herein comprise one or more copies of a heterologous nucleic acid encoding a phosphomevalonate decarboxylase comprising an amino acid sequence selected from the group consisting of SEQ ID NOs:16-18. In another aspect, the recombinant cells described herein comprise one or more copies of an endogenous nucleic acid encoding a phosphomevalonate decarboxylase from Herpetosiphon aurantiacus, Anaerolinea thermophila, or S378Pa3-2. In certain aspects, the recombinant cells described herein comprise a nucleic acid encoding an isopentenyl kinase from Herpetosiphon aurantiacus, Methanocaldococcus jannaschii, or Methanobrevibacter ruminantium. In certain aspects, the recombinant cells described herein comprise one or more copies of a heterologous nucleic acid encoding an isopentenyl kinase isolated from Herpetosiphon aurantiacus, Methanocaldococcus jannaschii, or Methanobrevibacter ruminantium. In some aspects, the recombinant cells described herein comprise one or more copies of a heterologous nucleic acid encoding an isopentenyl kinase comprising an amino acid sequence selected from the group consisting of SEQ ID NOs:19-23. In another aspect, the recombinant cells described herein comprise one or more copies of an endogenous nucleic acid encoding an isopentenyl kinase from Herpetosiphon aurantiacus, Methanocaldococcus jannaschii, or Methanobrevibacter ruminantium. In certain aspects, the recombinant cells described herein comprise one or more copies of a heterologous nucleic acid encoding an MVK isolated from M. mazei, Lactobacillus, Lactobacillus sakei, yeast, Saccharomyces cerevisiae, Streptococcus, Streptococcus pneumoniae, Streptomyces, Streptomyces CL190, or M. burtonii.

In one embodiment, the recombinant cells further comprise one or more copies of a heterologous nucleic acid encoding mvaE and mvaS polypeptides from L. grayi, E. faecium, E. gallinarum, E. casseliflavus, and/or E. faecalis. In another embodiment, the recombinant cells further comprise a nucleic acid encoding an acetoacetyl-CoA synthase and one or more nucleic acids encoding one or more polypeptides of the upper MVA pathway. In any of the embodiments herein, the recombinant cells comprise one or more polypeptides of the upper MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate.

In any of the embodiments herein, the recombinant cells further comprise one or more polypeptides of the classical lower MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate. In any of the embodiments herein, the recombinant cells comprise an attenuated enzyme that converts mevalonate 5-phosphate to mevalonate 5-pyrophosphate (e.g., PMK) and/or an attenuated enzyme that converts mevalonate 5-pyrophosphate to isopentenyl pyrophosphate (e.g., MVD).

Phosphoketolase Nucleic Acids and Polypeptides

Phosphoketolase enzymes catalyze the conversion of xylulose 5-phosphate to glyceraldehyde 3-phosphate and acetyl phosphate and/or the conversion of fructose 6-phosphate to erythrose 4-phosphate and acetyl phosphate. In certain embodiments, the phosphoketolase polypeptide catalyzes the conversion of sedoheptulose-7-phosphate to a product (e.g., ribose-5-phosphate) and acetyl phosphate. Thus, without being bound by theory, the expression of phosphoketolase as set forth herein can result in an increase in the amount of acetyl phosphate produced from a carbon (e.g., a carbohydrate) source. This acetyl phosphate can be converted into acetyl-CoA which can then be utilized by the enzymatic activities of the MVA pathway to produce mevalonate, isoprenoid precursor molecules, isoprene and/or isoprenoids or can be used to produce acetyl-CoA-derived metabolites. Thus the amount of these compounds produced from a carbon source may be increased. Alternatively, production of Acetyl-P and AcCoA can be increased without the increase being reflected in higher intracellular concentration. In certain embodiments, intracellular acetyl-P or acetyl-CoA concentrations will remain unchanged or even decrease, even though the phosphoketolase reaction is taking place.

As used herein, the term “acetyl-CoA-derived metabolite” can refer to a metabolite resulting from the catalytic conversion of acetyl-CoA to said metabolite. The conversion can be a one step reaction or a multi-step reaction. For example, acetone is an acetyl-CoA derived metabolite that is produced from acetyl-CoA by a three step reaction (e.g., a multi-step reaction): 1) the condensation of two molecules of acetyl-CoA into acetoacetyl-CoA by acetyl-CoA acetyltransferase; 2) conversion of acetoacetyl-CoA into acetoacetate by a reaction with acetic acid or butyric acid resulting in the production of acetyl-CoA or butyryl-CoA; and 3) conversion of acetoacetate into acetone by a decarboxylation step catalyzed by acetoacetate decarboxylase. Acetone can be subsequently converted to isopropanol, isobutene and/or propene which are also expressly contemplated herein to be acetyl-CoA-derived metabolites. In some embodiments, the acetyl CoA-derived metabolite is selected from the group consisting of polyketides, polyhydroxybutyrate, fatty alcohols, and fatty acids. In some embodiments, the acetyl CoA-derived metabolite is selected from the group consisting of glutamic acid, glutamine, aspartate, asparagine, proline, arginine, methionine, threonine, cysteine, succinate, lysine, leucine, and isoleucine. In some embodiments, the acetyl CoA-derived metabolite is selected from the group consisting of acetone, isopropanol, isobutene, and propene. Thus the amount of these compounds (e.g., acetyl-CoA, acetyl-CoA-derived metabolite, acetyl-P, E4P, etc.) produced from a carbohydrate substrate may be increased.

Accordingly, in certain embodiments, the recombinant cells described herein in any of the methods described herein further comprise one or more nucleic acids encoding a phosphosphoketolase polypeptide or a polypeptide having phosphoketolase activity. In some aspects, the phosphoketolase polypeptide is an endogenous polypeptide. In some aspects, the endogenous nucleic acid encoding a phosphoketolase polypeptide is operably linked to a constitutive promoter. In some aspects, the endogenous nucleic acid encoding a phosphoketolase polypeptide is operably linked to an inducible promoter. In some aspects, the endogenous nucleic acid encoding a phosphoketolase polypeptide is operably linked to a strong promoter. In some aspects, more than one endogenous nucleic acid encoding a phosphoketolase polypeptide is used (e.g, 2, 3, 4, or more copies of an endogenous nucleic acid encoding a phosphoketolase polypeptide). In a particular aspect, the cells are engineered to overexpress the endogenous phosphoketolase polypeptide relative to wild-type cells. In some aspects, the endogenous nucleic acid encoding a phosphoketolase polypeptide is operably linked to a weak promoter.

Exemplary phosphoketolase nucleic acids include nucleic acids that encode a polypeptide, fragment of a polypeptide, peptide, or fusion polypeptide that has at least one activity of a phosphoketolase polypeptide. Exemplary phosphoketolase polypeptides and nucleic acids include naturally-occurring polypeptides and nucleic acids from any of the source organisms described herein as well as mutant polypeptides and nucleic acids derived from any of the source organisms described herein. In some aspects, a nucleic acid encoding a phosphoketolase is from Clostridium acetobutylicum, Lactobacillus reuteri, Lactobacillus plantarum, Lactobacillus paraplantarum, Bifidobacterium longum, Bifidobacterium animalis, Bifidobacterium breve, Enterococcus gallinarum, Gardnerella vaginalis, Ferrimonas balearica, Mucilaginibacter paludis, Nostoc punctiforme, Nostoc punctiforme PCC 73102, Pantoea, Pedobactor saltans, Rahnella aquatilis, Rhodopseudomonas palustris, Streptomyces griseus, Streptomyces avermitilis, Nocardiopsis dassonvillei, and/or Thermobifida fusca. In other aspects, a nucleic acid encoding a phosphoketolase is from Mycobacterium gilvum, Shewanella baltica, Lactobacillus rhamnosus, Lactobacillus crispatus, Bifidobacterium longum, Leuconostoc citreum, Bradyrhizobium sp., Enterococcus faecium, Brucella microti, Lactobacillus salivarius, Streptococcus agalactiae, Rhodococcus imtechensis, Burkholderia xenovorans, Mycobacterium intracellulare, Nitrosomonas sp., Schizosaccharomyces pombe, Leuconostoc mesenteroides, Streptomyces sp., Lactobacillus buchneri, Streptomyces ghanaensis, Cyanothece sp., and/or Neosartorya fischeri. In other aspects, a nucleic acid encoding a phosphoketolase is from Enterococcus faecium, Listeria grayi, Enterococcus gallinarum, Enterococcus saccharolyticus, Enterococcus casseliflavus, Mycoplasma alligatoris, Carnobacterium sp., Melissococcus plutonius, Tetragenococcus halophilus, and/or Mycoplasma arthritidis. In yet other aspects, a nucleic acid encoding a phosphoketolase is from Streptococcus agalactiae, Mycoplasma agalactiae, Streptococcus gordonii, Kingella oralis, Mycoplasma fermentans, Granulicatella adiacens, Mycoplasma hominis, Mycoplasma crocodyli, Mycobacterium bovis, Neisseria sp., Streptococcus sp., Eremococcus coleocola, Granulicatella elegans, Streptococcus parasanguinis, Aerococcus urinae, Kingella kingae, Streptococcus australis, Streptococcus criceti, and/or Mycoplasma columbinum. An example of a nucleic acid encoding a phosphoketolase polypeptide is a nucleic acid sequence encoding a polypeptide having the amino acid sequence of SEQ ID NO:24. Additional examples of phosphoketolase enzymes which can be used herein are described in U.S. Pat. No. 7,785,858, International Patent Application Publication No. WO 2011/159853, and U.S. Patent Application Publication No.: 2013/0089906, which are all incorporated by reference herein.

Amino acid sequence for a phosphoketolase
polypeptide from Mycoplasma hominis ATCC 23114
(SEQ ID NO: 24)
MISKIYDDKKYLEKMDKWFRAANYLGVCQMYLRDNPLLKKPLTSNDI
KLYPIGHWGTVPGQNFIYTHLNRVIKKYDLNMFYIEGPGHGGQVMIS
NSYLDGSYSEIYPEISQDEAGLAKMFKRFSFPGGTASHAAPETPGSI
HEGGELGYSISHGTGAILDNPDVICAAVVGDGEAETGPLATSWFSNA
FINPVNDGAILPILHLNGGKISNPTLLSRKPKEEIKKYFEGLGWNPI
FVEWSEDKSNLDMHELMAKSLDKAIESIKEIQAEARKKPAEEATRPT
WPMIVLRTPKGWTGPKQWNNEAIEGSFRAHQVPIPVSAFKMEKIADL
EKWLKSYKPEELFDENGTIIKEIRDLAPEGLKRMAVNPITNGGIDSK
PLKLQDWKKYALKIDYPGEIKAQDMAEMAKFAADIMKDNPSSFRVFG
PDETKSNRMFALFNVTNRQWLEPVSKKYDEWISPAGRIIDSQLSEHQ
CEGFLEGYVLTGRHGFFASYEAFLRVVDSMLTQHMKWIKKASELSWR
KTYPSLNIIATSNAFQQDHNGYTHQDPGLLGHLADKRPEIIREYLPA
DTNSLLAVMNKALTERNVINLIVASKQPREQFFTVEDAEELLEKGYK
VVPWASNISENEEPDIVFASSGVEPNIESLAAISLINQEYPHLKIRY
VYVLDLLKLRSRKIDPRGISDEEFDKVFTKNKPIIFAFHGFEGLLRD
IFFTRSNHNLIAHGYRENGDITTSFDIRQLSEMDRYHIAKDAAEAVY
GKDAKAFMNKLDQKLEYHRNYIDEYGYDMPEVVEWKWKNINKEN

Biochemical characteristics of exemplary phosphoketolases include, but are not limited to, protein expression, protein solubility, and activity. Phosphoketolases can also be selected on the basis of other characteristics, including, but not limited to, diversity amongst different types of organisms (e.g., gram positive bacteria, cyanobacteria, actinomyces), facultative low temperature aerobe, close relatives to a desired species (e.g., E. coli), and thermotolerance. In some instances, phosphoketolases from certain organisms can be selected if the organisms lack a phosphofructokinase gene in its genome. In some aspects, phosphoketolases can be selected based on an assay and/or method described in U.S. Patent Application Publication No.: 2013/0089906. For example, a method is provided herein for determining the presence of in vivo phosphoketolase activity of a polypeptide, wherein the method comprises (a) culturing a recombinant cell comprising a heterologous nucleic acid sequence encoding said polypeptide wherein the recombinant cell is defective in transketolase activity (tktAB) under culture conditions with glucose or xylose as a carbon source; (b) assessing cell growth of the recombinant cell and (c) determining the presence of in vivo phosphoketolase activity of said polypeptide based upon the amount of observed cell growth.

Standard methods can be used to determine whether a polypeptide has phosphoketolase peptide activity by measuring the ability of the peptide to convert D-fructose 6-phosphate or D-xylulose 5-phosphate into acetyl-P. Acetyl-P can then be converted into ferryl acetyl hydroxamate, which can be detected spectrophotometrically (Meile et al., 2001, J. Bact. 183:2929-2936). Any polypeptide identified as having phosphoketolase peptide activity as described herein is suitable for use in the present invention.

In any of the embodiments herein, the recombinant cells can be further engineered to increase the activity of one or more of the following genes selected from the group consisting of ribose-5-phosphate isomerase (rpiA and/or rpiB), D-ribulose-5-phosphate 3-epimerase (rpe), transketolase (tktA and/or tktB), transaldolase B (tal B), phosphate acetyltransferase (pta and/or eutD). In another embodiment, the recombinant cells can be further engineered to decrease the activity of one or more genes of the following genes including glucose-6-phosphate dehydrogenase (zwf), 6-phosphofructokinase-1 (pfkA and/or pfkB), fructose bisphosphate aldolase (fba, fbaA, fbaB, and/or fbaC), glyceraldehyde-3-phosphate dehydrogenase (gapA and/or gapB), acetate kinase (ackA), citrate synthase (gltA), EI (ptsI), EIICB^{Glc} (ptsG), EIIA^{Glc} (crr), and/or HPr (ptsH).

In some aspects, in any of the embodiments above and/or herein, culturing of the recombinant cell in a suitable media increases one or more of an intracellular amount of erythrose 4-phosphate, an intracellular amount of glyceraldehyde 3-phosphate, or yield of acetyl phosphate. In other aspects, in any of the embodiments above and/or herein, the polypeptide having phosphoketolase activity is capable of synthesizing glyceraldehyde 3-phosphate and acetyl phosphate from xylulose 5-phosphate. In other aspects, in any of the embodiments above and/or herein, the polypeptide having phosphoketolase activity is capable of synthesizing erythrose 4-phosphate and acetyl phosphate from fructose 6-phosphate.

### Recombinant Cells Capable of Producing Isoprene

Isoprene (2-methyl-1,3-butadiene) is an important organic compound used in a wide array of applications. For instance, isoprene is employed as an intermediate or a starting material in the synthesis of numerous chemical compositions and polymers, including in the production of synthetic rubber. Isoprene is also an important biological material that is synthesized naturally by many plants and animals.

Isoprene is produced from DMAPP by the enzymatic action of isoprene synthase. Therefore, without being bound to theory, it is thought that increasing the cellular production of isopentenyl pyrophosphate from mevalonate via the alternative lower MVA pathway in recombinant cells by any of the compositions and methods described above will likewise result in the production of higher amounts of isoprene. Increasing the molar yield of isopentenyl pyrophosphate production from glucose translates into higher molar yields of isoprene and/or isoprenoids produced from glucose when combined with appropriate enzymatic activity levels of mevalonate kinase, phosphomevalonate decarboxylase, isopentenyl kinase, isopentenyl diphosphate isomerase (e.g., the alternative lower MVA pathway) and other appropriate enzymes for isoprene and isoprenoid production.

As described herein, the present invention provides recombinant cells capable of producing of isoprene, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide, wherein culturing the cells in a suitable media provides for the production of isoprene. In a further embodiment, the recombinant cells further comprise one or more nucleic acids encoding an isopentenyl diphosphate isomerase (IDI) polypeptide. In certain embodiments, the present invention provides recombinant cells capable of isoprene production, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide, wherein the cells produce increased amounts of isoprene compared to isoprene-producing cells that do not comprise a nucleic acid encoding a polypeptide having phosphomevalonate decarboxylase activity and/or a nucleic acid encoding a polypeptide having isopentenyl kinase activity. In a further embodiment, the recombinant cells further comprise one or more nucleic acids encoding an isopentenyl diphosphate isomerase (IDI) polypeptide. In some of the embodiments, provided herein are recombinant cells capable of producing isoprene, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide, wherein the total amount of ATP utilized by the cells during production of isoprene is reduced as compared to isoprene-producing cells that do not comprise a nucleic acid encoding a polypeptide having phosphomevalonate decarboxylase activity and/or a nucleic acid encoding a polypeptide having isopentenyl kinase activity. In some embodiments, the total amount of ATP utilized by the cells during production of isoprene is reduced by at least 1 ATP net, 2 ATP net, 3ATP net, 4 ATP net or 5 ATP net. In some embodiments, the total amount of ATP utilized by the cells during production of isoprene is reduced by 1 ATP net.

Production of isoprene can also be made by using any of the recombinant host cells described herein further comprising one or more of the enzymatic pathways manipulations wherein enzyme activity is modulated to increase carbon flow towards mevalonate production. The recombinant cells described herein that have various enzymatic pathways manipulated for increased carbon flow to mevalonate production can be used to produce isoprene. In one embodiment, the recombinant cells further comprise a nucleic acid encoding a phosphoketolase. In another embodiment, the recombinant cells can be further engineered to increase the activity of one or more of the following genes selected from the group consisting of ribose-5-phosphate isomerase (rpiA and/or rpiB), D-ribulose-5-phosphate 3-epimerase (rpe), transketolase (tktA and/or tktB), transaldolase B (tal B), phosphate acetyltransferase (pta and/or eutD). In another embodiment, these recombinant cells can be further engineered to decrease the activity of one or more genes of the following genes including glucose-6-phosphate dehydrogenase (zwf), 6-phosphofructokinase-1 (pfkA and/or pfkB), fructose bisphosphate aldolase (fba, fbaA, fbaB, and/or fbaC), glyceraldehyde-3-phosphate dehydrogenase (gapA and/or gapB), acetate kinase (ackA), citrate synthase (gltA), EI (ptsI), EIICB^{Glc} (ptsG), EIIA^{Glc} (crr), and/or HPr (ptsH).

### Isoprene Synthase Nucleic Acids and Polypeptides

In some aspects of the invention, the cells described in any of the compositions or methods described herein (including host cells that have been modified as described herein) further comprise one or more nucleic acids encoding an isoprene synthase polypeptide or a polypeptide having isoprene synthase activity. In some aspects, the isoprene synthase polypeptide is an endogenous polypeptide. In some aspects, the endogenous nucleic acid encoding an isoprene synthase polypeptide is operably linked to a constitutive promoter. In some aspects, the endogenous nucleic acid encoding an isoprene synthase polypeptide is operably linked to an inducible promoter. In some aspects, the endogenous nucleic acid encoding an isoprene synthase polypeptide is operably linked to a strong promoter. In a particular aspect, the cells are engineered to overexpress the endogenous isoprene synthase pathway polypeptide relative to wild-type cells. In some aspects, the endogenous nucleic acid encoding an isoprene synthase polypeptide is operably linked to a weak promoter.

In some aspects, the isoprene synthase polypeptide is a heterologous polypeptide. In some aspects, the cells comprise more than one copy of a heterologous nucleic acid encoding an isoprene synthase polypeptide. In some aspects, the heterologous nucleic acid encoding an isoprene synthase polypeptide is operably linked to a constitutive promoter. In some aspects, the heterologous nucleic acid encoding an isoprene synthase polypeptide is operably linked to an inducible promoter. In some aspects, the heterologous nucleic acid encoding an isoprene synthase polypeptide is operably linked to a strong promoter. In some aspects, the heterologous nucleic acid encoding an isoprene synthase polypeptide is operably linked to a weak promoter. In some aspects, the isoprene synthase polypeptide is a polypeptide or variant thereof from Pueraria or Populus or a hybrid such as Populus alba×Populus tremula. In some aspects, the isoprene synthase polypeptide is a polypeptide or variant thereof from Pueraria montana or Pueraria lobata, Populus tremuloides, Populus alba, Populus nigra, and Populus trichocarpa. In some aspects, the isoprene synthase polypeptide is from Eucalyptus.

The nucleic acids encoding an isoprene synthase polypeptide(s) can be integrated into a genome of the host cells or can be stably expressed in the cells. The nucleic acids encoding an isoprene synthase polypeptide(s) can additionally be on a vector.

Exemplary isoprene synthase nucleic acids include nucleic acids that encode a polypeptide, fragment of a polypeptide, peptide, or fusion polypeptide that has at least one activity of an isoprene synthase polypeptide. Isoprene synthase polypeptides convert dimethylallyl diphosphate (DMAPP) into isoprene. Exemplary isoprene synthase polypeptides include polypeptides, fragments of polypeptides, peptides, and fusions polypeptides that have at least one activity of an isoprene synthase polypeptide. Exemplary isoprene synthase polypeptides and nucleic acids include naturally-occurring polypeptides and nucleic acids from any of the source organisms described herein. In addition, variants of isoprene synthase can possess improved activity such as improved enzymatic activity. In some aspects, an isoprene synthase variant has other improved properties, such as improved stability (e.g., thermo-stability), and/or improved solubility.

Standard methods can be used to determine whether a polypeptide has isoprene synthase polypeptide activity by measuring the ability of the polypeptide to convert DMAPP into isoprene in vitro, in a cell extract, or in vivo. Isoprene synthase polypeptide activity in the cell extract can be measured, for example, as described in Silver et al., J. Biol. Chem. 270:13010-13016, 1995. In one exemplary assay, DMAPP (Sigma) can be evaporated to dryness under a stream of nitrogen and rehydrated to a concentration of 100 mM in 100 mM potassium phosphate buffer pH 8.2 and stored at −20° C. To perform the assay, a solution of 5 μL of 1M MgCl₂, 1 mM (250 μg/ml) DMAPP, 65 μL of Plant Extract Buffer (PEB) (50 mM Tris-HCl, pH 8.0, 20 mM MgCl₂, 5% glycerol, and 2 mM DTT) can be added to 25 μL of cell extract in a 20 ml Headspace vial with a metal screw cap and teflon coated silicon septum (Agilent Technologies) and cultured at 37° C. for 15 minutes with shaking. The reaction can be quenched by adding 200 μL of 250 mM EDTA and quantified by GC/MS.

In some aspects, the isoprene synthase polypeptide is a plant isoprene synthase polypeptide or a variant thereof. In some aspects, the isoprene synthase polypeptide is an isoprene synthase from Pueraria or a variant thereof. In some aspects, the isoprene synthase polypeptide is an isoprene synthase from Populus or a variant thereof. In some aspects, the isoprene synthase polypeptide is a poplar isoprene synthase polypeptide or a variant thereof. In some aspects, the isoprene synthase polypeptide is a kudzu isoprene synthase polypeptide or a variant thereof. In some aspects, the isoprene synthase polypeptide is a willow isoprene synthase polypeptide or a variant thereof. In some aspects, the isoprene synthase polypeptide is a eucalyptus isoprene synthase polypeptide or a variant thereof. In some aspects, the isoprene synthase polypeptide is a polypeptide from Pueraria or Populus or a hybrid, Populus alba×Populus tremula, or a variant thereof. In some aspects, the isoprene synthase polypeptide is from Robinia, Salix, or Melaleuca or variants thereof.

In some embodiments, the plant isoprene synthase is from the family Fabaceae, the family Salicaceae, or the family Fagaceae. In some aspects, the isoprene synthase polypeptide or nucleic acid is a polypeptide or nucleic acid from Pueraria montana (kudzu) (Sharkey et al., Plant Physiology 137: 700-712, 2005), Pueraria lobata, poplar (such as Populus alba, Populus nigra, Populus trichocarpa, or Populus alba×tremula (CAC35696) (Miller et al., Planta 213: 483-487, 2001), aspen (such as Populus tremuloides) (Silver et al., JBC 270(22): 13010-1316, 1995), English Oak (Quercus robur) (Zimmer et al., WO 98/02550), or a variant thereof. In some aspects, the isoprene synthase polypeptide is an isoprene synthase from Pueraria montana, Pueraria lobata, Populus tremuloides, Populus alba, Populus nigra, or Populus trichocarpa or a variant thereof. In some aspects, the isoprene synthase polypeptide is an isoprene synthase from Populus alba or a variant thereof. In some aspects, the isoprene synthase is Populus balsamifera (Genbank JN173037), Populus deltoides (Genbank JN173039), Populus fremontii (Genbank JN173040), Populus granididenta (Genbank JN173038), Salix (Genbank JN173043), Robinia pseudoacacia (Genbank JN173041), Wisteria (Genbank JN173042), Eucalyptus globulus (Genbank AB266390) or Melaleuca alterniflora (Genbank AY279379) or variant thereof. In some aspects, the nucleic acid encoding the isoprene synthase (e.g., isoprene synthase from Populus alba or a variant thereof) is codon optimized.

In some aspects, the isoprene synthase nucleic acid or polypeptide is a naturally-occurring polypeptide or nucleic acid (e.g., naturally-occurring polypeptide or nucleic acid from Populus). In some aspects, the isoprene synthase nucleic acid or polypeptide is not a wild-type or naturally-occurring polypeptide or nucleic acid. In some aspects, the isoprene synthase nucleic acid or polypeptide is a variant of a wild-type or naturally-occurring polypeptide or nucleic acid (e.g., a variant of a wild-type or naturally-occurring polypeptide or nucleic acid from Populus).

In some aspects, the isoprene synthase polypeptide is a variant. In some aspects, the isoprene synthase polypeptide is a variant of a wild-type or naturally occurring isoprene synthase. In some aspects, the variant has improved activity such as improved catalytic activity compared to the wild-type or naturally occurring isoprene synthase. The increase in activity (e.g., catalytic activity) can be at least about any of 10%, 20%, 30%, 40%, 50%, 60%, 70%, 80%, 90%, or 95%. In some aspects, the increase in activity such as catalytic activity is at least about any of 1 fold, 2 folds, 5 folds, 10 folds, 20 folds, 30 folds, 40 folds, 50 folds, 75 folds, or 100 folds. In some aspects, the increase in activity such as catalytic activity is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the variant has improved solubility compared to the wild-type or naturally occurring isoprene synthase. The increase in solubility can be at least about any of 10%, 20%, 30%, 40%, 50%, 60%, 70%, 80%, 90%, or 95%. The increase in solubility can be at least about any of 1 fold, 2 folds, 5 folds, 10 folds, 20 folds, 30 folds, 40 folds, 50 folds, 75 folds, or 100 folds. In some aspects, the increase in solubility is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the isoprene synthase polypeptide is a variant of naturally occurring isoprene synthase and has improved stability (such as thermo-stability) compared to the naturally occurring isoprene synthase.

In some aspects, the variant has at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200% of the activity of a wild-type or naturally occurring isoprene synthase. The variant can share sequence similarity with a wild-type or naturally occurring isoprene synthase. In some aspects, a variant of a wild-type or naturally occurring isoprene synthase can have at least about any of 40%, 50%, 60%, 70%, 75%, 80%, 90%, 91%, 92%, 93%, 94%, 95%, 96%, 97%, 98%, 99%, 99.5%, or 99.9% amino acid sequence identity as that of the wild-type or naturally occurring isoprene synthase. In some aspects, a variant of a wild-type or naturally occurring isoprene synthase has any of about 70% to about 99.9%, about 75% to about 99%, about 80% to about 98%, about 85% to about 97%, or about 90% to about 95% amino acid sequence identity as that of the wild-type or naturally occurring isoprene synthase.

In some aspects, the variant comprises a mutation in the wild-type or naturally occurring isoprene synthase. In some aspects, the variant has at least one amino acid substitution, at least one amino acid insertion, and/or at least one amino acid deletion. In some aspects, the variant has at least one amino acid substitution. In some aspects, the number of differing amino acid residues between the variant and wild-type or naturally occurring isoprene synthase can be one or more, e.g. 1, 2, 3, 4, 5, 10, 15, 20, 30, 40, 50, or more amino acid residues. Naturally occurring isoprene synthases can include any isoprene synthases from plants, for example, kudzu isoprene synthases, poplar isoprene synthases, English oak isoprene synthases, willow isoprene synthases, and eucalyptus isoprene synthases. In some aspects, the variant is a variant of isoprene synthase from Populus alba. In some aspects, the variant of isoprene synthase from Populus alba has at least one amino acid substitution, at least one amino acid insertion, and/or at least one amino acid deletion. In some aspects, the variant is a truncated Populus alba isoprene synthase. In some aspects, the nucleic acid encoding variant (e.g., variant of isoprene synthase from Populus alba) is codon optimized (for example, codon optimized based on host cells where the heterologous isoprene synthase is expressed).

Suitable isoprene synthases include, but are not limited to, those identified by Genbank Accession Nos. AY341431, AY316691, AB198180, AJ294819.1, EU693027.1, EF638224.1, AM410988.1, EF147555.1, AY279379, AJ457070, and AY182241. Types of isoprene synthases which can be used in any one of the compositions or methods including methods of making microorganisms encoding isoprene synthase described herein are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/124146, WO2010/078457, and WO2010/148256, U.S. Patent Application Publication No.: 2010/0086978, U.S. patent application Ser. No. 13/283,564, and Sharkey et al., “Isoprene Synthase Genes Form A Monophyletic Clade Of Acyclic Terpene Synthases In The Tps-B Terpene Synthase Family”, Evolution (2012) (available on line at DOI: 10.1111/evo.12013), the contents of which are expressly incorporated herein by reference in their entirety with respect to the isoprene synthases and isoprene synthase variants.

Any one of the promoters described herein (e.g., promoters described herein and identified in the Examples of the present disclosure including inducible promoters and constitutive promoters) can be used to drive expression of any of the isoprene synthases described herein.

### Isoprene Biosynthetic Pathway

Isoprene can be produced from two different alcohols, 3-methyl-2-buten-1-ol and 2-methyl-3-buten-2-ol. For example, in a two-step isoprene biosynthetic pathway, dimethylallyl diphosphate is converted to 2-methyl-3-buten-2-ol by an enzyme such as a synthase (e.g., a 2-methyl-3-buten-2-ol synthase), followed by conversion of 2-methyl-3-buten-2-ol to isoprene by a 2-methyl-3-buten-2-ol dehydratase. As another example, in a three-step isoprene biosynthetic pathway, dimethylallyl diphosphate is converted to 3-methyl-2-buten-1-ol by either a phosphatase or a synthase (e.g., a geraniol synthase or farnesol synthase) capable of converting dimethylallyl diphosphate to 3-methyl-2-buten-1-ol, 3-methyl-2-buten-1-ol is converted to 2-methyl-3-buten-2-ol by a 2-methyl-3-buten-2-ol isomerase, and 2-methyl-3-buten-2-ol is converted to isoprene by a 2-methyl-3-buten-2-ol dehydratase. See for example, U.S. Patent Application Publication No.: US 20130309742 A1 and U.S. Patent Application Publication No.: US 20130309741 A1.

In some aspects of the invention, the cells described in any of the compositions or methods described herein (including host cells that have been modified as described herein) further comprise one or more nucleic acids encoding a polypeptide of an isoprene biosynthetic pathway selected from the group consisting of 2-methyl-3-buten-2-ol dehydratase, 2-methyl-3-butene-2-ol isomerase, and 3-methyl-2-buten-1-ol synthase. In some aspects, the polypeptide of an isoprene biosynthetic pathway is an endogenous polypeptide. In some aspects, the endogenous nucleic acid encoding a polypeptide of an isoprene biosynthetic pathway is operably linked to a constitutive promoter. In some aspects, the endogenous nucleic acid encoding a polypeptide of an isoprene biosynthetic pathway is operably linked to an inducible promoter. In some aspects, the endogenous nucleic acid encoding a polypeptide of an isoprene biosynthetic pathway is operably linked to a strong promoter. In a particular aspect, the cells are engineered to overexpress the endogenous polypeptide of an isoprene biosynthetic pathway relative to wild-type cells. In some aspects, the endogenous nucleic acid encoding a polypeptide of an isoprene biosynthetic pathway is operably linked to a weak promoter.

In some aspects, the polypeptide of an isoprene biosynthetic pathway is a heterologous polypeptide. In some aspects, the cells comprise more than one copy of a heterologous nucleic acid encoding a polypeptide of an isoprene biosynthetic pathway. In some aspects, the heterologous nucleic acid encoding a polypeptide of an isoprene biosynthetic pathway is operably linked to a constitutive promoter. In some aspects, the heterologous nucleic acid encoding a polypeptide of an isoprene biosynthetic pathway is operably linked to an inducible promoter. In some aspects, the heterologous nucleic acid encoding a polypeptide of an isoprene biosynthetic pathway is operably linked to a strong promoter. In some aspects, the heterologous nucleic acid encoding a polypeptide of an isoprene biosynthetic pathway is operably linked to a weak promoter.

The nucleic acids encoding a polypeptide(s) of an isoprene biosynthetic pathway can be integrated into a genome of the host cells or can be stably expressed in the cells. The nucleic acids encoding a polypeptide(s) of an isoprene biosynthetic pathway can additionally be on a vector.

Exemplary nucleic acids encoding a polypeptide(s) of an isoprene biosynthetic pathway include nucleic acids that encode a polypeptide, fragment of a polypeptide, peptide, or fusion polypeptide that has at least one activity of a polypeptide of an isoprene biosynthetic pathway such as a 2-methyl-3-buten-2-ol dehydratase polypeptide, 2-methyl-3-butene-2-ol isomerase polypeptide, and 3-methyl-2-buten-1-ol synthase polypeptide. Exemplary polypeptide(s) of an isoprene biosynthetic pathway and nucleic acids encoding polypeptide(s) of an isoprene biosynthetic pathway include naturally-occurring polypeptides and nucleic acids from any of the source organisms described herein. In addition, variants of polypeptide(s) of an isoprene biosynthetic pathway (e.g., a 2-methyl-3-buten-2-ol dehydratase polypeptide, 2-methyl-3-butene-2-ol isomerase polypeptide, and 3-methyl-2-buten-1-ol synthase polypeptide) can possess improved activity such as improved enzymatic activity.

In some aspects, a polypeptide of an isoprene biosynthetic pathway is a phosphatase. Exemplary phosphatases include a phosphatase from Bacillus subtilis or Escherichia coli. In some embodiments, the phosphatase is a 3-methyl-2-buten-1-ol synthase polypeptide or variant thereof. In some aspects, a polypeptide of an isoprene biosynthetic pathway is a terpene synthase (e.g., a geraniol synthase, farnesol synthase, linalool synthase or nerolidol synthase). Exemplary terpene synthases include a terpene synthase from Ocimum basilicum, Perilla citriodora, Perilla frutescans, Cinnamomom tenuipile, Zea mays or Oryza sativa. Additional exemplary terpene synthases include a terpene synthase from Clarkia breweri, Arabidopsis thaliana, Perilla setoyensis, Perilla frutescans, Actinidia arguta, Actinidia polygama, Artemesia annua, Ocimum basilicum, Mentha aquatica, Solanum lycopersicum, Medicago trunculata, Populus trichocarpa, Fragaria vesca, or Fragraria ananassa. In some embodiments, the terpene synthase is a 3-methyl-2-buten-1-ol synthase polypeptide or variant thereof. For example, a terpene synthase described herein can catalyze the conversion of dimethylallyl diphosphate to 3-methyl-2-buten-1-ol (e.g., a 3-methyl-2-buten-1-ol synthase). In some aspects, a terpene synthase described herein can catalyze the conversion of dimethylallyl diphosphate to 2-methyl-3-buten-2-ol (e.g., a 2-methyl-3-buten-2-ol synthase). In some aspects, a polypeptide of an isoprene biosynthetic pathway is a 2-methyl-3-buten-2-ol dehydratase polypeptide (e.g., a 2-methyl-3-buten-2-ol dehydratase polypeptide from Aquincola tertiaricarbonis) or variant thereof. In some aspects, the 2-methyl-3-buten-2-ol dehydratase polypeptide is a linalool dehydratase-isomerase polypeptide (e.g., a linalool dehydratase-isomerase polypeptide from Castellaniella defragrans Genbank accession number FR669447) or variant thereof. In some aspects, a polypeptide of an isoprene biosynthetic pathway is a 2-methyl-3-buten-2-ol isomerase polypeptide or variant thereof. In some aspects, the 2-methyl-3-butene-2-ol isomerase polypeptide is a linalool dehydratase-isomerase polypeptide (e.g., a linalool dehydratase-isomerase polypeptide from Castellaniella defragrans Genbank accession number FR669447) or variant thereof.

Standard methods can be used to determine whether a polypeptide has the desired isoprene biosynthetic pathway enzymatic activity (e.g., a 2-methyl-3-buten-2-ol dehydratase activity, 2-methyl-3-butene-2-ol isomerase activity, and 3-methyl-2-buten-1-ol activity) by measuring the ability of the polypeptide to convert DMAPP into isoprene in vitro, in a cell extract, or in vivo. See for example, U.S. Patent Application Publication No.: US 20130309742 A1 and U.S. Patent Application Publication No.: US 20130309741 A1.

In some aspects, the polypeptide(s) of an isoprene biosynthetic pathway (e.g., a 2-methyl-3-buten-2-ol dehydratase polypeptide, 2-methyl-3-butene-2-ol isomerase polypeptide, and 3-methyl-2-buten-1-ol synthase polypeptide) is a variant. In some aspects, polypeptide(s) of an isoprene biosynthetic pathway (e.g., a 2-methyl-3-buten-2-ol dehydratase polypeptide, 2-methyl-3-butene-2-ol isomerase polypeptide, and 3-methyl-2-buten-1-ol synthase polypeptide) is a variant of a wild-type or naturally occurring polypeptide(s) of an isoprene biosynthetic pathway. In some aspects, the variant has improved activity such as improved catalytic activity compared to the wild-type or naturally occurring polypeptide(s) of an isoprene biosynthetic pathway. The increase in activity (e.g., catalytic activity) can be at least about any of 10%, 20%, 30%, 40%, 50%, 60%, 70%, 80%, 90%, or 95%. In some aspects, the increase in activity such as catalytic activity is at least about any of 1 fold, 2 folds, 5 folds, 10 folds, 20 folds, 30 folds, 40 folds, 50 folds, 75 folds, or 100 folds. In some aspects, the increase in activity such as catalytic activity is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the variant has improved solubility compared to the wild-type or naturally occurring polypeptide(s) of an isoprene biosynthetic pathway. The increase in solubility can be at least about any of 10%, 20%, 30%, 40%, 50%, 60%, 70%, 80%, 90%, or 95%. The increase in solubility can be at least about any of 1 fold, 2 folds, 5 folds, 10 folds, 20 folds, 30 folds, 40 folds, 50 folds, 75 folds, or 100 folds. In some aspects, the increase in solubility is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the polypeptide(s) of an isoprene biosynthetic pathway is a variant of naturally occurring polypeptide(s) of an isoprene biosynthetic pathway and has improved stability (such as thermo-stability) compared to the naturally occurring polypeptide(s) of an isoprene biosynthetic pathway.

In some aspects, the variant has at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200% of the activity of a wild-type or naturally occurring polypeptide(s) of an isoprene biosynthetic pathway (e.g., a 2-methyl-3-buten-2-ol dehydratase polypeptide, 2-methyl-3-butene-2-ol isomerase polypeptide, and 3-methyl-2-buten-1-ol synthase polypeptide). The variant can share sequence similarity with a wild-type or naturally occurring polypeptide(s) of an isoprene biosynthetic pathway. In some aspects, a variant of a wild-type or naturally occurring polypeptide(s) of an isoprene biosynthetic pathway can have at least about any of 40%, 50%, 60%, 70%, 75%, 80%, 90%, 91%, 92%, 93%, 94%, 95%, 96%, 97%, 98%, 99%, 99.5%, or 99.9% amino acid sequence identity as that of the wild-type or naturally occurring polypeptide(s) of an isoprene biosynthetic pathway (e.g., a 2-methyl-3-buten-2-ol dehydratase polypeptide, 2-methyl-3-butene-2-ol isomerase polypeptide, and 3-methyl-2-buten-1-ol synthase polypeptide). In some aspects, a variant of a wild-type or naturally occurring polypeptide(s) of an isoprene biosynthetic pathway has any of about 70% to about 99.9%, about 75% to about 99%, about 80% to about 98%, about 85% to about 97%, or about 90% to about 95% amino acid sequence identity as that of the wild-type or naturally occurring polypeptide(s) of an isoprene biosynthetic pathway (e.g., a 2-methyl-3-buten-2-ol dehydratase polypeptide, 2-methyl-3-butene-2-ol isomerase polypeptide, and 3-methyl-2-buten-1-ol synthase polypeptide).

In some aspects, the variant comprises a mutation in the wild-type or naturally occurring polypeptide(s) of an isoprene biosynthetic pathway (e.g., a 2-methyl-3-buten-2-ol dehydratase polypeptide, 2-methyl-3-butene-2-ol isomerase polypeptide, and 3-methyl-2-buten-1-ol synthase polypeptide). In some aspects, the variant has at least one amino acid substitution, at least one amino acid insertion, and/or at least one amino acid deletion. In some aspects, the variant has at least one amino acid substitution. In some aspects, the number of differing amino acid residues between the variant and wild-type or naturally occurring polypeptide(s) of an isoprene biosynthetic pathway can be one or more, e.g. 1, 2, 3, 4, 5, 10, 15, 20, 30, 40, 50, or more amino acid residues. In some aspects, the nucleic acid encoding the variant (e.g., a 2-methyl-3-buten-2-ol dehydratase polypeptide, 2-methyl-3-butene-2-ol isomerase polypeptide, and 3-methyl-2-buten-1-ol synthase polypeptide) is codon optimized (for example, codon optimized based on host cells where the heterologous polypeptide(s) of an isoprene biosynthetic pathway is expressed).

Any one of the promoters described herein (e.g., promoters described herein and identified in the Examples of the present disclosure including inducible promoters and constitutive promoters) can be used to drive expression of any of the polypeptides of an isoprene biosynthetic pathway described herein.

### DXP Pathway Nucleic Acids and Polypeptides

In some aspects of the invention, the cells described in any of the compositions or methods described herein (including host cells that have been modified as described herein) further comprise one or more heterologous nucleic acids encoding a DXS polypeptide or other DXP pathway polypeptides. In some aspects, the cells further comprise a chromosomal copy of an endogenous nucleic acid encoding a DXS polypeptide or other DXP pathway polypeptides. In some aspects, the E. coli cells further comprise one or more nucleic acids encoding an IDI polypeptide and a DXS polypeptide or other DXP pathway polypeptides. In some aspects, one nucleic acid encodes the isoprene synthase polypeptide, IDI polypeptide, and DXS polypeptide or other DXP pathway polypeptides. In some aspects, one plasmid encodes the isoprene synthase polypeptide, IDI polypeptide, and DXS polypeptide or other DXP pathway polypeptides. In some aspects, multiple plasmids encode the isoprene synthase polypeptide, IDI polypeptide, and DXS polypeptide or other DXP pathway polypeptides.

Exemplary DXS polypeptides include polypeptides, fragments of polypeptides, peptides, and fusions polypeptides that have at least one activity of a DXS polypeptide. Standard methods (such as those described herein) can be used to determine whether a polypeptide has DXS polypeptide activity by measuring the ability of the polypeptide to convert pyruvate and D-glyceraldehyde 3-phosphate into 1-deoxy-D-xylulose-5-phosphate in vitro, in a cell extract, or in vivo. Exemplary DXS polypeptides and nucleic acids and methods of measuring DXS activity are described in more detail in International Publication Nos. WO 2009/076676, WO 2010/003007, WO 2009/132220, and U.S. Patent Publ. Nos. US 2009/0203102, 2010/0003716 and 2010/0048964.

Exemplary DXP pathways polypeptides include, but are not limited to any of the following polypeptides: DXS polypeptides, DXR polypeptides, MCT polypeptides, CMK polypeptides, MCS polypeptides, HDS polypeptides, HDR polypeptides, and polypeptides (e.g., fusion polypeptides) having an activity of one, two, or more of the DXP pathway polypeptides. In particular, DXP pathway polypeptides include polypeptides, fragments of polypeptides, peptides, and fusions polypeptides that have at least one activity of a DXP pathway polypeptide. Exemplary DXP pathway nucleic acids include nucleic acids that encode a polypeptide, fragment of a polypeptide, peptide, or fusion polypeptide that has at least one activity of a DXP pathway polypeptide. Exemplary DXP pathway polypeptides and nucleic acids include naturally-occurring polypeptides and nucleic acids from any of the source organisms described herein as well as mutant polypeptides and nucleic acids derived from any of the source organisms described herein. Exemplary DXP pathway polypeptides and nucleic acids and methods of measuring DXP pathway polypeptide activity are described in more detail in International Publication No. WO 2010/148150

Exemplary DXS polypeptides include polypeptides, fragments of polypeptides, peptides, and fusions polypeptides that have at least one activity of a DXS polypeptide. Standard methods (such as those described herein) can be used to determine whether a polypeptide has DXS polypeptide activity by measuring the ability of the polypeptide to convert pyruvate and D-glyceraldehyde 3-phosphate into 1-deoxy-D-xylulose-5-phosphate in vitro, in a cell extract, or in vivo. Exemplary DXS polypeptides and nucleic acids and methods of measuring DXS activity are described in more detail in International Publication No. WO 2009/076676, WO 2010/003007, WO 2009/132220, and U.S. Patent Publ. Nos. US 2009/0203102, 2010/0003716, and 2010/0048964.

In particular, DXS polypeptides convert pyruvate and D-glyceraldehyde 3-phosphate into 1-deoxy-D-xylulose 5-phosphate (DXP). Standard methods can be used to determine whether a polypeptide has DXS polypeptide activity by measuring the ability of the polypeptide to convert pyruvate and D-glyceraldehyde 3-phosphate in vitro, in a cell extract, or in vivo.

DXR polypeptides convert 1-deoxy-D-xylulose 5-phosphate (DXP) into 2-C-methyl-D-erythritol 4-phosphate (MEP). Standard methods can be used to determine whether a polypeptide has DXR polypeptides activity by measuring the ability of the polypeptide to convert DXP in vitro, in a cell extract, or in vivo.

MCT polypeptides convert 2-C-methyl-D-erythritol 4-phosphate (MEP) into 4-(cytidine 5′-diphospho)-2-methyl-D-erythritol (CDP-ME). Standard methods can be used to determine whether a polypeptide has MCT polypeptides activity by measuring the ability of the polypeptide to convert MEP in vitro, in a cell extract, or in vivo.

CMK polypeptides convert 4-(cytidine 5′-diphospho)-2-C-methyl-D-erythritol (CDP-ME) into 2-phospho-4-(cytidine 5′-diphospho)-2-C-methyl-D-erythritol (CDP-MEP). Standard methods can be used to determine whether a polypeptide has CMK polypeptides activity by measuring the ability of the polypeptide to convert CDP-ME in vitro, in a cell extract, or in vivo.

MCS polypeptides convert 2-phospho-4-(cytidine 5′-diphospho)-2-C-methyl-D-erythritol (CDP-MEP) into 2-C-methyl-D-erythritol 2, 4-cyclodiphosphate (ME-CPP or cMEPP). Standard methods can be used to determine whether a polypeptide has MCS polypeptides activity by measuring the ability of the polypeptide to convert CDP-MEP in vitro, in a cell extract, or in vivo.

HDS polypeptides convert 2-C-methyl-D-erythritol 2, 4-cyclodiphosphate into (E)-4-hydroxy-3-methylbut-2-en-1-yl diphosphate (HMBPP or HDMAPP). Standard methods can be used to determine whether a polypeptide has HDS polypeptides activity by measuring the ability of the polypeptide to convert ME-CPP in vitro, in a cell extract, or in vivo.

HDR polypeptides convert (E)-4-hydroxy-3-methylbut-2-en-1-yl diphosphate into isopentenyl diphosphate (IPP) and dimethylallyl diphosphate (DMAPP). Standard methods can be used to determine whether a polypeptide has HDR polypeptides activity by measuring the ability of the polypeptide to convert HMBPP in vitro, in a cell extract, or in vivo.

### Source Organisms for Isoprene Synthase, IDI, and DXP Pathway Polypeptides

Isoprene synthase, IDI, and/or DXP pathway nucleic acids (and their encoded polypeptides) can be obtained from any organism that naturally contains isoprene synthase, IDI, and/or DXP pathway nucleic acids. Isoprene is formed naturally by a variety of organisms, such as bacteria, yeast, plants, and animals. Some organisms contain the MVA pathway for producing isoprene. Isoprene synthase nucleic acids can be obtained, e.g., from any organism that contains an isoprene synthase. MVA pathway nucleic acids can be obtained, e.g., from any organism that contains the MVA pathway. IDI and DXP pathway nucleic acids can be obtained, e.g., from any organism that contains the IDI and DXP pathway.

The nucleic acid sequence of the isoprene synthase, DXP pathway, and/or IDI nucleic acids can be isolated from a bacterium, fungus, plant, algae, or cyanobacterium. Exemplary source organisms include, for example, yeasts, such as species of Saccharomyces (e.g., S. cerevisiae), bacteria, such as species of Escherichia (e.g., E. coli), or species of Methanosarcina (e.g., Methanosarcina mazei), plants, such as kudzu or poplar (e.g., Populus alba or Populus alba×tremula CAC35696) or aspen (e.g., Populus tremuloides). Exemplary sources for isoprene synthases, and/or IDI polypeptides which can be used are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/078457, and WO2010/148256.

In some aspects, the source organism is a yeast, such as Saccharomyces sp., Schizosaccharomyces sp., Pichia sp., or Candida sp.

In some aspects, the source organism is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, strains of Escherichia such as E. coli, strains of Enterobacter, strains of Streptococcus, or strains of Archaea such as Methanosarcina mazei.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some aspects, the source organism is a gram-positive bacterium. Non-limiting examples include strains of Streptomyces (e.g., S. lividans, S. coelicolor, or S. griseus) and Bacillus. In some aspects, the source organism is a gram-negative bacterium, such as E. coli or Pseudomonas sp.

In some aspects, the source organism is a plant, such as a plant from the family Fabaceae, such as the Faboideae subfamily. In some aspects, the source organism is kudzu, poplar (such as Populus alba×tremula CAC35696), aspen (such as Populus tremuloides), or Quercus robur.

In some aspects, the source organism is an algae, such as a green algae, red algae, glaucophytes, chlorarachniophytes, euglenids, chromista, or dinoflagellates.

In some aspects, the source organism is a cyanobacteria, such as cyanobacteria classified into any of the following groups based on morphology: Chroococcales, Pleurocapsales, Oscillatoriales, Nostocales, or Stigonematales.

### Recombinant Cells Capable of Production of Isoprene Via the Alternative Lower MVA Pathway

Accordingly, the recombinant cells described herein (including host cells that have been modified as described herein) have the ability to produce isoprene concentration greater than that of the same cells lacking (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide when cultured under the same conditions. The cells can further comprise one or more heterologous nucleic acids encoding an IDI polypeptide. In some aspects, the cells can further comprise one or more heterologous nucleic acids encoding a phosphoketolase.

In some aspects, the nucleic acid encoding a polypeptide having phosphomevalonate decarboxylase activity, the nucleic acid encoding a polypeptide having isopentenyl kinase activity, the one or more nucleic acids encoding one or more polypeptides of the MVA pathway, and the nucleic acid encoding an isoprene synthase polypeptide are heterologous nucleic acids that are integrated into the host cell's chromosomal nucleotide sequence. In other aspects, the one or more heterologous nucleic acids are integrated into plasmid. In still other aspects, at least one of the one or more heterologous nucleic acids is integrated into the cell's chromosomal nucleotide sequence while at least one of the one or more heterologous nucleic acid sequences is integrated into a plasmid. The recombinant cells can produce at least 5% greater amounts of isoprene compared to isoprene-producing cells that do not comprise the phosphomevalonate decarboxylase and/or isopentenyl kinase polypeptide. Alternatively, the recombinant cells can produce greater than about 1%, 2%, 3%, 4%, 5%, 6%, 7%, 8%, 9%, 10%, 11%, 12%, 13%, 14%, or 15% of isoprene, inclusive, as well as any numerical value in between these numbers.

In one aspect of the invention, provided herein are recombinant cells comprising (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, (iv) a heterologous nucleic acid encoding an isoprene synthase polypeptide, and (v) one or more heterologous nucleic acids encoding a DXP pathway polypeptide(s). The cells can further comprise one or more heterologous nucleic acids encoding an IDI polypeptide. In some aspects, the cells can further comprise one or more heterologous nucleic acids encoding a phosphoketolase. Any of the one or more heterologous nucleic acids can be operably linked to constitutive promoters, can be operably linked to inducible promoters, or can be operably linked to a combination of inducible and constitutive promoters. The one or more heterologous nucleic acids can additionally be operably linked to strong promoters, weak promoters, and/or medium promoters. One or more of the heterologous nucleic acids encoding phosphomevalonate decarboxylase, isopentenyl kinase, a mevalonate (MVA) pathway polypeptide(s), a DXP pathway polypeptide(s), and an isoprene synthase polypeptide can be integrated into a genome of the host cells or can be stably expressed in the cells. The one or more heterologous nucleic acids can additionally be on a vector.

The production of isoprene by the cells according to any of the compositions or methods described herein can be enhanced (e.g., enhanced by the expression of one or more heterologous nucleic acids encoding a phosphomevalonate decarboxylase polypeptide, an isopentenyl kinase polypeptide, an isoprene synthase polypeptide, MVA pathway polypeptide(s), and/or a DXP pathway polypeptide(s)). As used herein, “enhanced” isoprene production refers to an increased cell productivity index (CPI) for isoprene, an increased titer of isoprene, an increased mass yield of isoprene, and/or an increased specific productivity of isoprene by the cells described by any of the compositions and methods described herein compared to cells which do not have one or more nucleic acids encoding a phosphomevalonate decarboxylase polypeptide and/or an isopentenyl kinase polypeptide. In certain embodiments described herein, the host cells have been further engineered increased carbon flux to MVA production.

The production of isoprene by the recombinant cells described herein can be enhanced by about 5% to about 1,000,000 folds. In certain aspects, the production of isoprene can be enhanced by about 10% to about 1,000,000 folds (e.g., about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of isoprene by cells that do not express one or more nucleic acids encoding a phosphomevalonate decarboxylase polypeptide and/or an isopentenyl kinase polypeptide. In certain embodiments described herein, the host cells have been further modified and/or engineered for increased carbon flux to MVA production thereby providing enhanced production of isoprene as compared to the production of isoprene by cells that do not express one or more nucleic acids encoding a phosphomevalonate decarboxylase polypeptide and/or an isopentenyl kinase polypeptide and which have not been modified and/or engineered for increased carbon flux to mevalonate production.

In other aspects, the production of isoprene by the recombinant cells described herein can also be enhanced by at least about any of 5%, 10%, 20%, 30%, 40%, 50%, 60%, 70%, 80%, 90%, 1 fold, 2 folds, 5 folds, 10 folds, 20 folds, 50 folds, 100 folds, 200 folds, 500 folds, 1000 folds, 2000 folds, 5000 folds, 10,000 folds, 20,000 folds, 50,000 folds, 100,000 folds, 200,000 folds, 500,000 folds, or 1,000,000 folds as compared to the production of isoprene by cells that do not express one or more nucleic acids encoding a phosphomevalonate decarboxylase polypeptide and/or an isopentenyl kinase polypeptide. In certain embodiments described herein, the host cells have been further modified and/or engineered for increased carbon flux to MVA production thereby providing enhanced production of isoprene as compared to the production of isoprene by cells that do not express one or more nucleic acids encoding a phosphomevalonate decarboxylase polypeptide and/or an isopentenyl kinase polypeptide and which have not been modified and/or engineered for increased carbon flux to mevalonate production.

### Methods of Using the Recombinant Cells to Produce Isoprene Via the Alternative Lower MVA Pathway

Also provided herein are methods for producing isoprene comprising culturing any of the recombinant cells described herein. In one aspect, isoprene can be produced by culturing recombinant cells comprising (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide. In another aspect, isoprene can be produced by culturing recombinant cells comprising modulation in any of the enzymatic pathways described herein and (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide. In certain aspects, the recombinant cells described herein comprise one or more copies of an endogenous nucleic acid encoding a phosphomevalonate decarboxylase from Herpetosiphon aurantiacus, Anaerolinea thermophila, or S378Pa3-2. In certain aspects, the recombinant cells described herein comprise a nucleic acid encoding an isopentenyl kinase from Herpetosiphon aurantiacus, Methanocaldococcus jannaschii, or Methanobrevibacter ruminantium.

Thus, provided herein are methods of producing isoprene comprising culturing cells comprising a nucleic acid encoding a polypeptide having phosphomevalonate decarboxylase activity and a nucleic acid encoding a polypeptide having isopentenyl kinase activity (a) in a suitable condition for producing isoprene and (b) producing isoprene. The cells can further comprise one or more nucleic acid molecules encoding the MVA pathway polypeptide(s) described above (e.g., the upper MVA pathway and MVK) and any of the isoprene synthase polypeptide(s) described above (e.g. Pueraria isoprene synthase). In some aspects, the recombinant cells can be one of any of the cells described herein. Any of the isoprene synthases or variants thereof described herein, any of the host cell strains described herein, any of the promoters described herein, and/or any of the vectors described herein can also be used to produce isoprene using any of the energy sources (e.g. glucose or any other six carbon sugar) described herein can be used in the methods described herein. In some aspects, the method of producing isoprene further comprises a step of recovering the isoprene. In certain aspects, the recombinant cells described herein comprise one or more copies of an endogenous nucleic acid encoding a phosphomevalonate decarboxylase from Herpetosiphon aurantiacus, Anaerolinea thermophila, or S378Pa3-2. In certain aspects, the recombinant cells described herein comprise a nucleic acid encoding an isopentenyl kinase from Herpetosiphon aurantiacus, Methanocaldococcus jannaschii, or Methanobrevibacter ruminantium.

In certain aspects, provided herein are methods of making isoprene comprising culturing recombinant cells comprising one or more heterologous nucleic acids encoding a phosphomevalonate decarboxylase polypeptide from Herpetosiphon aurantiacus, Anaerolinea thermophila, or S378Pa3-2 and an isopentenyl kinase polypeptide from Herpetosiphon aurantiacus, Methanocaldococcus jannaschii, or Methanobrevibacter ruminantium (a) in a suitable condition for producing isoprene and (b) producing isoprene. The cells can further comprise one or more nucleic acid molecules encoding the upper MVA pathway polypeptide(s) described above, any MVK polypeptide(s) described above, and any of the isoprene synthase polypeptide(s) described above. In some aspects, the recombinant cells can be any of the cells described herein.

The recombinant cells described herein that have various enzymatic pathways manipulated for increased carbon flow to mevalonate production can be used to produce isoprene. In some embodiments, the recombinant cells can further comprise one or more nucleic acids encoding a phosphoketolase polypeptide. In some aspects, the recombinant cells can be further engineered to increase the activity of one or more of the following genes selected from the group consisting of ribose-5-phosphate isomerase (rpiA and/or rpiB), D-ribulose-5-phosphate 3-epimerase (rpe), transketolase (tktA and/or tktB), transaldolase B (tal B), phosphate acetyltransferase (pta and/or eutD). In another embodiment, these recombinant cells can be further engineered to decrease the activity of one or more genes of the following genes including glucose-6-phosphate dehydrogenase (zwf), 6-phosphofructokinase-1 (pfkA and/or pfkB), fructose bisphosphate aldolase (fba, fbaA, fbaB, and/or fbaC), glyceraldehyde-3-phosphate dehydrogenase (gapA and/or gapB), acetate kinase (ackA), citrate synthase (OA), EI (ptsI), EIICB^{Glc} (ptsG), EIIA^{Glc} (crr), and/or HPr (ptsH).

In some aspects, the recombinant cells are cultured in a culture medium under conditions permitting the production of isoprene by the recombinant cells. In some embodiments, the isoprene amount is measured at the peak absolute productivity time point. In some embodiments, the peak absolute productivity for the cells is about any of the isoprene amounts disclosed herein. By “peak absolute productivity” is meant the maximum absolute amount of isoprene in the off-gas during the culturing of cells for a particular period of time (e.g., the culturing of cells during a particular fermentation run). By “peak absolute productivity time point” is meant the time point during a fermentation run when the absolute amount of isoprene in the off-gas is at a maximum during the culturing of cells for a particular period of time (e.g., the culturing of cells during a particular fermentation run).

In some embodiments, the isoprene amount is measured at the peak specific productivity time point. In some embodiments, the peak specific productivity for the cells is about any of the isoprene amounts per cell disclosed herein. By “peak specific productivity” is meant the maximum amount of isoprene produced per cell during the culturing of cells for a particular period of time (e.g., the culturing of cells during a particular fermentation run). By “peak specific productivity time point” is meant the time point during the culturing of cells for a particular period of time (e.g., the culturing of cells during a particular fermentation run) when the amount of isoprene produced per cell is at a maximum. The peak specific productivity is determined by dividing the total productivity by the amount of cells, as determined by optical density at 600 nm (OD₆₀₀).

In some embodiments, the isoprene amount is measured at the peak specific volumetric productivity time point. In some embodiments, the peak specific volumetric productivity for the cells is about any of the isoprene amounts per volume per time disclosed herein. By “peak volumetric productivity” is meant the maximum amount of isoprene produced per volume of broth (including the volume of the cells and the cell medium) during the culturing of cells for a particular period of time (e.g., the culturing of cells during a particular fermentation run). By “peak specific volumetric productivity time point” is meant the time point during the culturing of cells for a particular period of time (e.g., the culturing of cells during a particular fermentation run) when the amount of isoprene produced per volume of broth is at a maximum. The peak specific volumetric productivity is determined by dividing the total productivity by the volume of broth and amount of time.

In some embodiments, the isoprene amount is measured at the peak concentration time point. In some embodiments, the peak concentration for the cells is about any of the isoprene amounts disclosed herein. By “peak concentration” is meant the maximum amount of isoprene produced during the culturing of cells for a particular period of time (e.g., the culturing of cells during a particular fermentation run). By “peak concentration time point” is meant the time point during the culturing of cells for a particular period of time (e.g., the culturing of cells during a particular fermentation run) when the amount of isoprene produced per cell is at a maximum.

In some embodiments, the average specific volumetric productivity for the cells is about any of the isoprene amounts per volume per time disclosed herein. By “average volumetric productivity” is meant the average amount of isoprene produced per volume of broth (including the volume of the cells and the cell medium) during the culturing of cells for a particular period of time (e.g., the culturing of cells during a particular fermentation run). The average volumetric productivity is determined by dividing the total productivity by the volume of broth and amount of time.

In some embodiments, the cumulative, total amount of isoprene is measured. In some embodiments, the cumulative total productivity for the cells is about any of the isoprene amounts disclosed herein. By “cumulative total productivity” is meant the cumulative, total amount of isoprene produced during the culturing of cells for a particular period of time (e.g., the culturing of cells during a particular fermentation run).

In some aspects, any of the recombinant cells described herein (for examples the cells in culture) produce isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, or more nmole of isoprene/gram of cells for the wet weight of the cells/hour (nmole/g_{wcm}/hr). In some aspects, the amount of isoprene is between about 2 to about 5,000 nmole/g_{wcm}/hr, such as between about 2 to about 100 nmole/g_{wcm}/hr, about 100 to about 500 nmole/g_{wcm}/hr, about 150 to about 500 nmole/g_{wcm}/hr, about 500 to about 1,000 nmole/g_{wcm}/hr, about 1,000 to about 2,000 nmole/g_{wcm}/hr, or about 2,000 to about 5,000 nmole/g_{wcm}/hr. In some aspects, the amount of isoprene is between about 20 to about 5,000 nmole/g_{wcm}/hr, about 100 to about 5,000 nmole/g_{wcm}/hr, about 200 to about 2,000 nmole/g_{wcm}/hr, about 200 to about 1,000 nmole/g_{wcm}/hr, about 300 to about 1,000 nmole/g_{wcm}/hr, or about 400 to about 1,000 nmole/g_{wcm}/hr.

The amount of isoprene in units of nmole/g_{wcm}/hr can be measured as disclosed in U.S. Pat. No. 5,849,970, which is hereby incorporated by reference in its entirety, particularly with respect to the measurement of isoprene production. For example, two mL of headspace (e.g., headspace from a culture such as 2 mL of culture cultured in sealed vials at 32° C. with shaking at 200 rpm for approximately 3 hours) are analyzed for isoprene using a standard gas chromatography system, such as a system operated isothermally (85° C.) with an n-octane/porasil C column (Alltech Associates, Inc., Deerfield, Ill.) and coupled to a RGD2 mercuric oxide reduction gas detector (Trace Analytical, Menlo Park, Calif.) (see, for example, Greenberg et al, Atmos. Environ. 27A: 2689-2692, 1993; Silver et al., Plant Physiol. 97:1588-1591, 1991, which are each hereby incorporated by reference in their entireties, particularly with respect to the measurement of isoprene production). The gas chromatography area units are converted to nmol isoprene via a standard isoprene concentration calibration curve. In some embodiments, the value for the grams of cells for the wet weight of the cells is calculated by obtaining the A₆₀₀ value for a sample of the cell culture, and then converting the A₆₀₀ value to grams of cells based on a calibration curve of wet weights for cell cultures with a known A₆₀₀ value. In some embodiments, the grams of the cells is estimated by assuming that one liter of broth (including cell medium and cells) with an A₆₀₀ value of 1 has a wet cell weight of 1 gram. The value is also divided by the number of hours the culture has been incubating for, such as three hours.

In some aspects, the recombinant cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 100,000, or more ng of isoprene/gram of cells for the wet weight of the cells/hr (ng/g_{wcm}/h). In some aspects, the amount of isoprene is between about 2 to about 5,000 ng/g_{wcm}/h, such as between about 2 to about 100 ng/g_{wcm}/h, about 100 to about 500 ng/g_{wcm}/h, about 500 to about 1,000 ng/g_{wcm}/h, about 1,000 to about 2,000 ng/g_{wcm}/h, or about 2,000 to about 5,000 ng/g_{wcm}/h. In some aspects, the amount of isoprene is between about 20 to about 5,000 ng/g_{wcm}/h, about 100 to about 5,000 ng/g_{wcm}/h, about 200 to about 2,000 ng/g_{wcm}/h, about 200 to about 1,000 ng/g_{wcm}/h, about 300 to about 1,000 ng/g_{wcm}/h, or about 400 to about 1,000 ng/g_{wcm}/h. The amount of isoprene in ng/g_{wcm}/h can be calculated by multiplying the value for isoprene production in the units of nmole/g_{wcm}/hr discussed above by 68.1 (as described in Equation 5 below).

In some aspects, the recombinant cells in culture produce a cumulative titer (total amount) of isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 50,000, 100,000, or more mg of isoprene/L of broth (mg/L_{broth}, wherein the volume of broth includes the volume of the cells and the cell medium). In some aspects, the amount of isoprene is between about 2 to about 5,000 mg/L_{broth}, such as between about 2 to about 100 mg/L_{broth}, about 100 to about 500 mg/L_{broth}, about 500 to about 1,000 mg/L_{broth}, about 1,000 to about 2,000 mg/L_{broth}, or about 2,000 to about 5,000 mg/L_{broth}. In some aspects, the amount of isoprene is between about 20 to about 5,000 mg/L_{broth}, about 100 to about 5,000 mg/L_{broth}, about 200 to about 2,000 mg/L_{broth}, about 200 to about 1,000 mg/L_{broth}, about 300 to about 1,000 mg/L_{broth}, or about 400 to about 1,000 mg/L_{broth}.

The specific productivity of isoprene in mg of isoprene/L of headspace from shake flask or similar cultures can be measured by taking a 1 ml sample from the cell culture at an OD₆₀₀ value of approximately 1.0, putting it in a 20 mL vial, incubating for 30 minutes, and then measuring the amount of isoprene in the headspace. If the OD₆₀₀ value is not 1.0, then the measurement can be normalized to an OD₆₀₀ value of 1.0 by dividing by the OD₆₀₀ value. The value of mg isoprene/L headspace can be converted to mg/L_{broth}/hr/OD₆₀₀ of culture broth by multiplying by a factor of 38. The value in units of mg/L_{broth}/hr/OD₆₀₀ can be multiplied by the number of hours and the OD₆₀₀ value to obtain the cumulative titer in units of mg of isoprene/L of broth.

In some embodiments, the cells in culture have an average volumetric productivity of isoprene at greater than or about 0.1, 1.0, 10, 25, 50, 100, 150, 200, 250, 300, 400, 500, 600, 700, 800, 900, 1,000, 1100, 1200, 1300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, or more mg of isoprene/L of broth/hr (mg/L_{broth}/hr, wherein the volume of broth includes the volume of the cells and the cell medium). In some embodiments, the average volumetric productivity of isoprene is between about 0.1 to about 3,500 mg/L_{broth}/hr, such as between about 0.1 to about 100 mg/L_{broth}/hr, about 100 to about 500 mg/L_{broth}/hr, about 500 to about 1,000 mg/L_{broth}/hr, about 1,000 to about 1,500 mg/L_{broth}/hr, about 1,500 to about 2,000 mg/L_{broth}/hr, about 2,000 to about 2,500 mg/L_{broth}/hr, about 2,500 to about 3,000 mg/L_{broth}/hr, or about 3,000 to about 3,500 mg/L_{broth}/hr. In some embodiments, the average volumetric productivity of isoprene is between about 10 to about 3,500 mg/L_{broth}/hr, about 100 to about 3,500 mg/L_{broth}/hr, about 200 to about 1,000 mg/L_{broth}/hr, about 200 to about 1,500 mg/L_{broth}/hr, about 1,000 to about 3,000 mg/L_{broth}/hr, or about 1,500 to about 3,000 mg/L_{broth}/hr.

In some embodiments, the cells in culture have a peak volumetric productivity of isoprene at greater than or about 0.5, 1.0, 10, 25, 50, 100, 150, 200, 250, 300, 400, 500, 600, 700, 800, 900, 1,000, 1100, 1200, 1300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,750, 4,000, 4,250, 4,500, 4,750, 5,000, 5,250, 5,500, 5,750, 6,000, 6,250, 6,500, 6,750, 7,000, 7,250, 7,500, 7,750, 8,000, 8,250, 8,500, 8,750, 9,000, 9,250, 9,500, 9,750, 10,000, 12,500, 15,000, or more mg of isoprene/L of broth/hr (mg/L_{broth}/hr, wherein the volume of broth includes the volume of the cells and the cell medium). In some embodiments, the peak volumetric productivity of isoprene is between about 0.5 to about 15,000 mg/L_{broth}/hr, such as between about 0.5 to about 10 mg/L_{broth}/hr, about 1.0 to about 100 mg/L_{broth}/hr, about 100 to about 500 mg/L_{broth}/hr, about 500 to about 1,000 mg/L_{broth}/hr, about 1,000 to about 1,500 mg/L_{broth}/hr, about 1,500 to about 2,000 mg/L_{broth}/hr, about 2,000 to about 2,500 mg/L_{broth}/hr, about 2,500 to about 3,000 mg/L_{broth}/hr, about 3,000 to about 3,500 mg/L_{broth}/hr, about 3,500 to about 5,000 mg/L_{broth}/hr, about 5,000 to about 7,500 mg/L_{broth}/hr, about 7,500 to about 10,000 mg/L_{broth}/hr, about 10,000 to about 12,500 mg/L_{broth}/h, or about 12,500 to about 15,000 mg/L_{broth}/hr. In some embodiments, the peak volumetric productivity of isoprene is between about 10 to about 15,000 mg/L_{broth}/hr, about 100 to about 2,500 mg/L_{broth}/hr, about 1,000 to about 5,000 mg/L_{broth}/hr, about 2,500 to about 7,500 mg/L_{broth}/hr, about 5,000 to about 10,000 mg/L_{broth}/hr, about 7,500 to about 12,500 mg/L_{broth}/hr, or about 10,000 to about 15,000 mg/L_{broth}/hr.

The instantaneous isoprene production rate in mg/L_{broth}/hr in a fermentor can be measured by taking a sample of the fermentor off-gas, analyzing it for the amount of isoprene (in units such as mg of isoprene per L_{gas}) and multiplying this value by the rate at which off-gas is passed though each liter of broth (e.g., at 1 vvm (volume of air/volume of broth/minute) this is 60 L_{gas} per hour). Thus, an off-gas level of 1 mg/L_{gas} corresponds to an instantaneous production rate of 60 mg/L_{broth}/hr at air flow of 1 vvm. If desired, the value in the units mg/L_{broth}/hr can be divided by the OD₆₀₀ value to obtain the specific rate in units of mg/L_{broth}/hr/OD. The average value of mg isoprene/L_{gas} can be converted to the total product productivity (grams of isoprene per liter of fermentation broth, mg/L_{broth}) by multiplying this average off-gas isoprene concentration by the total amount of off-gas sparged per liter of fermentation broth during the fermentation. Thus, an average off-gas isoprene concentration of 0.5 mg/L_{broth}/hr over 10 hours at 1 vvm corresponds to a total product concentration of 300 mg isoprene/L_{broth}.

In some embodiments, the cells in culture convert greater than or about 0.0015, 0.002, 0.005, 0.01, 0.02, 0.05, 0.1, 0.12, 0.14, 0.16, 0.2, 0.3, 0.4, 0.5, 0.6, 0.7, 0.8, 0.9, 1.0, 1.2, 1.4, 1.6, 2.0, 2.2, 2.4, 2.6, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 23.2, 23.4, 23.6, 23.8, 24.0, 25.0, 30.0, 31.0, 32.0, 33.0, 35.0, 37.5, 40.0, 45.0, 47.5, 50.0, 55.0, 60.0, 65.0, 70.0, 75.0, 80.0, 85.0, or 90.0 molar % of the carbon in the cell culture medium into isoprene. In some embodiments, the percent conversion of carbon into isoprene is between about 0.002 to about 90.0 molar %, such as about 0.002 to about 0.005%, about 0.005 to about 0.01%, about 0.01 to about 0.05%, about 0.05 to about 0.15%, 0.15 to about 0.2%, about 0.2 to about 0.3%, about 0.3 to about 0.5%, about 0.5 to about 0.8%, about 0.8 to about 1.0%, about 1.0 to about 1.6%, about 1.6 to about 3.0%, about 3.0 to about 5.0%, about 5.0 to about 8.0%, about 8.0 to about 10.0%, about 10.0 to about 15.0%, about 15.0 to about 20.0%, about 20.0 to about 25.0%, about 25.0% to 30.0%, about 30.0% to 35.0%, about 35.0% to 40.0%, about 45.0% to 50.0%, about 50.0% to 55.0%, about 55.0% to 60.0%, about 60.0% to 65.0%, about 65.0% to 70.0%, about 75.0% to 80.0%, about 80.0% to 85.0%, or about 85.0% to 90.0%. In some embodiments, the percent conversion of carbon into isoprene is between about 0.002 to about 0.4 molar %, 0.002 to about 0.16 molar %, 0.04 to about 0.16 molar %, about 0.005 to about 0.3 molar %, about 0.01 to about 0.3 molar %, about 0.05 to about 0.3 molar %, about 0.1 to 0.3 molar %, about 0.3 to about 1.0 molar %, about 1.0 to about 5.0 molar %, about 2 to about 5.0 molar %, about 5.0 to about 10.0 molar %, about 7 to about 10.0 molar %, about 10.0 to about 20.0 molar %, about 12 to about 20.0 molar %, about 16 to about 20.0 molar %, about 18 to about 20.0 molar %, about 18 to 23.2 molar %, about 18 to 23.6 molar %, about 18 to about 23.8 molar %, about 18 to about 24.0 molar %, about 18 to about 25.0 molar %, about 20 to about 30.0 molar %, about 30 to about 40.0 molar %, about 30 to about 50.0 molar %, about 30 to about 60.0 molar %, about 30 to about 70.0 molar %, about 30 to about 80.0 molar %, or about 30 to about 90.0 molar %

The percent conversion of carbon into isoprene (also referred to as “% carbon yield”) can be measured by dividing the moles carbon in the isoprene produced by the moles carbon in the carbon source (such as the moles of carbon in batched and fed glucose and yeast extract). This number is multiplied by 100% to give a percentage value (as indicated in Equation 1).

% Carbon Yield=(moles carbon in isoprene produced)/(moles carbon in carbon source)*100 Equation 1

The percent conversion of carbon into isoprene can be calculated as shown in Equation 2.

% Carbon Yield=(39.1 g isoprene*1/68.1 mol/g*5 C/mol)/[(181221 g glucose*1/180 mol/g*6 C/mol)+(17780 g yeast extract*0.5*1/12 mol/g)]*100=0.042% Equation 2

One skilled in the art can readily convert the rates of isoprene production or amount of isoprene produced into any other units. Exemplary equations are listed below for interconverting between units.

### Units for Rate of Isoprene Production (Total and Specific)

1 g isoprene/L_{broth}/hr=14.7 mmol isoprene/L_{broth}/hr(total volumetric rate) Equation 3

1 nmol isoprene/g_{wcm}/hr=1 nmol isoprene/L_{broth}/hr/OD₆₀₀(This conversion assumes that one liter of broth with an OD₆₀₀ value of 1 has a wet cell weight of 1 gram.) Equation 4

1 nmol isoprene/g_{wcm}/hr=68.1 ng isoprene/g_{wcm}/hr(given the molecular weight of isoprene) Equation 5

1 nmol isoprene/L_{gas} O₂/hr=90 nmol isoprene/L_{broth}/hr(at an O₂ flow rate of 90 L/hr per L of culture broth) Equation 6

1 ug isoprene/L_{gas} isoprene in off-gas=60 ug isoprene/L_{broth}/hr at a flow rate of 60 L_{gas} per L_{broth} (1 vvm) Equation 7

### Units for Titer (Total and Specific)

1 nmol isoprene/mg cell protein=150 nmol isoprene/L_{broth}/OD₆₀₀(This conversion assumes that one liter of broth with an OD₆₀₀ value of 1 has a total cell protein of approximately 150 mg)(specific productivity) Equation 8

1 g isoprene/L_{broth}=14.7 mmol isoprene/L_{broth}(total titer) Equation 9

If desired, Equation 10 can be used to convert any of the units that include the wet weight of the cells into the corresponding units that include the dry weight of the cells.

Dry weight of cells=(wet weight of cells)/3.3 Equation 10

In some embodiments encompassed by the invention, a cell comprising one or more heterologous nucleic acid encoding an phosphomevalonate decarboxylase and one or more heterologous nucleic acid encoding isopentenyl phosphate kinase produces an amount of isoprene that is at least or about 2-fold, 3-fold, 5-fold, 10-fold, 25-fold, 50-fold, 100-fold, 150-fold, 200-fold, 400-fold, or greater than the amount of isoprene produced from a corresponding cell grown under essentially the same conditions without the heterologous nucleic acid encoding the phosphomevalonate decarboxylase and/or isopentenyl phosphate kinase.

In some aspects, the isoprene produced by the recombinant cells in culture comprises at least about 1, 2, 5, 10, 15, 20, or 25% by volume of the fermentation offgas. In some aspects, the isoprene comprises between about 1 to about 25% by volume of the offgas, such as between about 5 to about 15%, about 15 to about 25%, about 10 to about 20%, or about 1 to about 10%.

In certain embodiments, the methods of producing isoprene can comprise the steps of: (a) culturing recombinant cells (including, but not limited to, E. coli cells) that do not endogenously express a phosphomevalonate polypeptide, wherein the cells heterologously express one or more copies of a gene encoding a phosphomevalonate decarboxylase polypeptide along with (i) one or more nucleic acids expressing an isopentenyl kinase (ii) one or more MVA pathway peptides and (iii) an isoprene synthase and (b) producing isoprene, wherein the recombinant cells display decreased oxygen uptake rate (OUR) as compared to that of the same cells lacking one or more heterologous copies of a gene encoding an phosphomevalonate polypeptide. In certain embodiments, the recombinant cells expressing one or more heterologous copies of a gene encoding an phosphomevalonate polypeptide display up to 1-fold, 2-fold, 3-fold, 4-fold, 5-fold, 6-fold or 7-fold decrease in OUR as compared to recombinant cells that do not express a phosphomevalonate decarboxylase polypeptide. In another embodiment, the methods of producing isoprene can comprise the steps of: (a) culturing recombinant cells (including, but not limited to, E. coli cells) that do not endogenously express a phosphomevalonate polypeptide and an isopentenyl kinase, wherein the cells heterologously express one or more copies of a gene encoding a phosphomevalonase decarboxylase polypeptide and isopentenyl kinase polypeptide along with (i) one or more nucleic acids expressing one or more MVA pathway peptides and (ii) an isoprene synthase and (b) producing isoprene, wherein the recombinant cells display decreased oxygen uptake rate (OUR) as compared to that of the same cells lacking one or more heterologous copies of a gene encoding an phosphomevalonatedecarboxylase polypeptide and isopentenyl kinase polypeptide. In certain embodiments, the recombinant cells expressing one or more heterologous copies of a gene encoding an phosphomevalonase decarboxylase polypeptide and isopentenyl kinase polypeptide display up to 1-fold, 2-fold, 3-fold, 4-fold, 5-fold, 6-fold or 7-fold decrease in OUR as compared to recombinant cells that do not express a phosphomevalonase decarboxylase polypeptide and isopentenyl kinase polypeptide.

In one aspect, described herein are compositions that comprise isoprene. In some embodiments, the composition comprising isoprene is produced by any one of the recombinant cells described herein. For example, a composition comprising isoprene can be produced by a recombinant cell comprising (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide, wherein culturing of said recombinant cell provides for the production of isoprene. In some embodiments, the nucleic acid encoding a polypeptide having phosphomevalonate decarboxylase activity is from an archaea. In further embodiments, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In some embodiments, the nucleic acid encoding a polypeptide having phosphomevalonate decarboxylase activity is from a microorganism selected from the group consisting of: Herpetosiphon aurantiacus, S378Pa3-2, and Anaerolinea thermophila. In some embodiments, the nucleic acid encoding a polypeptide having phosphomevalonate decarboxylase activity encodes a polypeptide having an amino acid sequence with at least 85% sequence identity to an amino acid sequence selected from the group consisting of SEQ ID NOs:16-18. In some embodiments, the nucleic acid encoding a polypeptide having isopentenyl kinase activity is from an archaea. In further embodiments, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In some embodiments, the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila. In some embodiments, the nucleic acid encoding a polypeptide having isopentenyl kinase activity encodes a polypeptide having an amino acid sequence with at least 85% sequence identity to an amino acid sequence selected from the group consisting of SEQ ID NOs:19-23. In some embodiments, the isoprene synthase polypeptide is a plant isoprene synthase polypeptide. In further embodiments, the plant isoprene synthase polypeptide is a polypeptide or variant thereof from Pueraria or Populus. In other further embodiments, the plant isoprene synthase polypeptide is a polypeptide or variant thereof from Pueraria montana or Pueraria lobata, Populus tremuloides, Populus alba, Populus nigra, Populus trichocarpa, or a hybrid Populus alba×Populus tremula. In some embodiments, the one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate. In some embodiments, the one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate. In some embodiments, a composition comprising isoprene is produced by a recombinant cell that further comprises one or more nucleic acids encoding an isopentenyl-diphosphate delta-isomerase (IDI) polypeptide. In some embodiments, a composition comprising isoprene is produced by a recombinant cell that comprises an attenuated enzyme that converts mevalonate 5-pyrophosphate to isopentenyl pyrophosphate. In some embodiments, a composition comprising isoprene is produced by a recombinant cell that comprises an attenuated enzyme that converts mevalonate 5-phosphate to mevalonate 5-pyrophosphate. In some embodiments, a composition comprising isoprene is produced by a recombinant cell that further comprises one or more nucleic acids encoding one or more 1-deoxy-D-xylulose 5-phosphate (DXP) pathway polypeptides. In some embodiments, a composition comprising isoprene is produced by a recombinant cell comprising one or more attenuated enzymes of the 1-deoxy-D-xylulose 5-phosphate (DXP) pathway. In some embodiments, a composition comprising isoprene is produced by a recombinant cell that further comprises a heterologous nucleic acid encoding a polypeptide having phosphoketolase activity. In any of the embodiments herein, a nucleic acid encoding a polypeptide of interest (e.g., a polypeptide having phosphomevalonate decarboxylase activity, a polypeptide having isopentenyl kinase activity, etc) can be a heterologous nucleic acid or an endogenous nucleic acid.

Recombinant Cells Capable of Production of Isoprenoid Precursors and/or Isoprenoids

Isoprenoids can be produced in many organisms from the synthesis of the isoprenoid precursor molecules which are the end products of the MVA pathway. As stated above, isoprenoids represent an important class of compounds and include, for example, food and feed supplements, flavor and odor compounds, and anticancer, antimalarial, antifungal, and antibacterial compounds.

As a class of molecules, isoprenoids are classified based on the number of isoprene units comprised in the compound. Monoterpenes comprise ten carbons or two isoprene units, sesquiterpenes comprise 15 carbons or three isoprene units, diterpenes comprise 20 carbons or four isoprene units, sesterterpenes comprise 25 carbons or five isoprene units, and so forth. Steroids (generally comprising about 27 carbons) are the products of cleaved or rearranged isoprenoids.

Isoprenoids can be produced from the isoprenoid precursor molecules IPP and DMAPP. These diverse compounds are derived from these rather simple universal precursors and are synthesized by groups of conserved polyprenyl pyrophosphate synthases (Hsieh et al., Plant Physiol. 2011 March; 155(3):1079-90). The various chain lengths of these linear prenyl pyrophosphates, reflecting their distinctive physiological functions, in general are determined by the highly developed active sites of polyprenyl pyrophosphate synthases via condensation reactions of allylic substrates (dimethylallyl diphosphate (C₅-DMAPP), geranyl pyrophosphate (C₁₀-GPP), farnesyl pyrophosphate (C₁₅-FPP), geranylgeranyl pyrophosphate (C₂₀-GGPP)) with corresponding number of isopentenyl pyrophosphates (C₅-IPP) (Hsieh et al., Plant Physiol. 2011 March; 155(3):1079-90).

Production of isoprenoid precursors and/or isoprenoids can be made by using any of the recombinant host cells that comprise a nucleic acid encoding a polypeptide having phosphomevalonate decarboxylase activity and a nucleic acid encoding a polypeptide having isopentenyl kinase activity for production of isoprenoid precursors and/or isoprenoids. In some aspects, these cells further comprise one or more heterologous nucleic acids encoding polypeptides of the MVA pathway, IDI, and/or the DXP pathway, as described above, and a heterologous nucleic acid encoding a polyprenyl pyrophosphate synthase polypeptide. Without being bound to theory, it is thought that increasing the cellular production of isopentenyl pyrophosphate from mevalonate via the alternative lower MVA pathway in recombinant cells by any of the compositions and methods described above will similarly result in the production of higher amounts of isoprenoid precursor molecules and/or isoprenoids. Increasing the molar yield of mevalonate production from glucose translates into higher molar yields of isoprenoid precursor molecules and/or isoprenoids, including isoprene, produced from glucose when combined with appropriate enzymatic activity levels of mevalonate kinase, phosphomevalonate decarboxylase, isopentenyl kinase, isopentenyl diphosphate isomerase and other appropriate enzymes for isoprene and isoprenoid production. The recombinant cells described herein that have various enzymatic pathways manipulated for increased carbon flow to mevalonate production can be used to produce isoprenoid precursors and/or isoprenoids. In some aspects, the recombinant cells can be further engineered to increase the activity of one or more of the following genes selected from the group consisting of rpiA, rpe, tktA, tal B, pta and/or eutD. In another aspect, these strains can be further engineered to decrease the activity of one or more genes of the following genes including zwf, pfkA, fba, gapA, ackA, gltA and/or pts.

### Types of Isoprenoids

The recombinant cells of the present invention are capable of production of isoprenoids and the isoprenoid precursor molecules DMAPP and IPP. Examples of isoprenoids include, without limitation, hemiterpenoids, monoterpenoids, sesquiterpenoids, diterpenoids, sesterterpenoids, triterpenoids, tetraterpenoids, and higher polyterpenoids. In some aspects, the hemiterpenoid is prenol (i.e., 3-methyl-2-buten-1-ol), isoprenol (i.e., 3-methyl-3-buten-1-ol), 2-methyl-3-buten-2-ol, or isovaleric acid. In some aspects, the monoterpenoid can be, without limitation, geranyl pyrophosphate, eucalyptol, limonene, or pinene. In some aspects, the sesquiterpenoid is farnesyl pyrophosphate, artemisinin, or bisabolol. In some aspects, the diterpenoid can be, without limitation, geranylgeranyl pyrophosphate, retinol, retinal, phytol, taxol, forskolin, or aphidicolin. In some aspects, the triterpenoid can be, without limitation, squalene or lanosterol. The isoprenoid can also be selected from the group consisting of abietadiene, amorphadiene, carene, α-famesene, β-farnesene, farnesol, geraniol, geranylgeraniol, linalool, limonene, myrcene, nerolidol, ocimene, patchoulol, β-pinene, sabinene, γ-terpinene, terpindene and valencene.

In some aspects, the tetraterpenoid is lycopene or carotene (a carotenoid). As used herein, the term “carotenoid” refers to a group of naturally-occurring organic pigments produced in the chloroplasts and chromoplasts of plants, of some other photosynthetic organisms, such as algae, in some types of fungus, and in some bacteria. Carotenoids include the oxygen-containing xanthophylls and the non-oxygen-containing carotenes. In some aspects, the carotenoids are selected from the group consisting of xanthophylls and carotenes. In some aspects, the xanthophyll is lutein or zeaxanthin. In some aspects, the carotenoid is α-carotene, β-carotene, γ-carotene, β-cryptoxanthin or lycopene.

### Heterologous Nucleic Acids Encoding Polyprenyl Pyrophosphate Synthases Polypeptides

In some aspects of the invention, the cells described in any of the compositions or methods herein further comprise one or more nucleic acids encoding a mevalonate (MVA) pathway polypeptide(s), as described above, as well as one or more nucleic acids encoding a polyprenyl pyrophosphate synthase polypeptides(s). The polyprenyl pyrophosphate synthase polypeptide can be an endogenous polypeptide. The endogenous nucleic acid encoding a polyprenyl pyrophosphate synthase polypeptide can be operably linked to a constitutive promoter or can similarly be operably linked to an inducible promoter. The endogenous nucleic acid encoding a polyprenyl pyrophosphate synthase polypeptide can additionally be operably linked to a strong promoter. Alternatively, the endogenous nucleic acid encoding a polyprenyl pyrophosphate synthase polypeptide can be operably linked to a weak promoter. In particular, the cells can be engineered to overexpress the endogenous polyprenyl pyrophosphate synthase polypeptide relative to wild-type cells.

In some aspects, the polyprenyl pyrophosphate synthase polypeptide is a heterologous polypeptide. The cells of the present invention can comprise more than one copy of a heterologous nucleic acid encoding a polyprenyl pyrophosphate synthase polypeptide. In some aspects, the heterologous nucleic acid encoding a polyprenyl pyrophosphate synthase polypeptide is operably linked to a constitutive promoter. In some aspects, the heterologous nucleic acid encoding a polyprenyl pyrophosphate synthase polypeptide is operably linked to an inducible promoter. In some aspects, the heterologous nucleic acid encoding a polyprenyl pyrophosphate synthase polypeptide is operably linked to a strong promoter. In some aspects, the heterologous nucleic acid encoding a polyprenyl pyrophosphate synthase polypeptide is operably linked to a weak promoter.

The nucleic acids encoding a polyprenyl pyrophosphate synthase polypeptide(s) can be integrated into a genome of the host cells or can be stably expressed in the cells. The nucleic acids encoding a polyprenyl pyrophosphate synthase polypeptide(s) can additionally be on a vector.

Exemplary polyprenyl pyrophosphate synthase nucleic acids include nucleic acids that encode a polypeptide, fragment of a polypeptide, peptide, or fusion polypeptide that has at least one activity of a polyprenyl pyrophosphate synthase. Polyprenyl pyrophosphate synthase polypeptides convert isoprenoid precursor molecules into more complex isoprenoid compounds. Exemplary polyprenyl pyrophosphate synthase polypeptides include polypeptides, fragments of polypeptides, peptides, and fusions polypeptides that have at least one activity of an isoprene synthase polypeptide. Exemplary polyprenyl pyrophosphate synthase polypeptides and nucleic acids include naturally-occurring polypeptides and nucleic acids from any of the source organisms described herein. In addition, variants of polyprenyl pyrophosphate synthase can possess improved activity such as improved enzymatic activity. In some aspects, a polyprenyl pyrophosphate synthase variant has other improved properties, such as improved stability (e.g., thermo-stability), and/or improved solubility. Exemplary polyprenyl pyrophosphate synthase nucleic acids can include nucleic acids which encode polyprenyl pyrophosphate synthase polypeptides such as, without limitation, geranyl diphosposphate (GPP) synthase, farnesyl pyrophosphate (FPP) synthase, and geranylgeranyl pyrophosphate (GGPP) synthase, or any other known polyprenyl pyrophosphate synthase polypeptide.

In some aspects of the invention, the cells described in any of the compositions or methods herein further comprise one or more nucleic acids encoding a farnesyl pyrophosphate (FPP) synthase. The FPP synthase polypeptide can be an endogenous polypeptide encoded by an endogenous gene. In some aspects, the FPP synthase polypeptide is encoded by an endogenous ispA gene in E. coli. The endogenous nucleic acid encoding an FPP synthase polypeptide can be operably linked to a constitutive promoter or can similarly be operably linked to an inducible promoter. The endogenous nucleic acid encoding an FPP synthase polypeptide can additionally be operably linked to a strong promoter. In particular, the cells can be engineered to overexpress the endogenous FPP synthase polypeptide relative to wild-type cells.

In some aspects, the FPP synthase polypeptide is a heterologous polypeptide. The cells of the present invention can comprise more than one copy of a heterologous nucleic acid encoding a FPP synthase polypeptide. In some aspects, the heterologous nucleic acid encoding a FPP synthase polypeptide is operably linked to a constitutive promoter. In some aspects, the heterologous nucleic acid encoding a FPP synthase polypeptide is operably linked to an inducible promoter. In some aspects, the heterologous nucleic acid encoding a polyprenyl pyrophosphate synthase polypeptide is operably linked to a strong promoter.

The nucleic acids encoding an FPP synthase polypeptide can be integrated into a genome of the host cells or can be stably expressed in the cells. The nucleic acids encoding an FPP synthase can additionally be on a vector.

Standard methods can be used to determine whether a polypeptide has polyprenyl pyrophosphate synthase polypeptide activity by measuring the ability of the polypeptide to convert IPP into higher order isoprenoids in vitro, in a cell extract, or in vivo. These methods are well known in the art and are described, for example, in U.S. Pat. No. 7,915,026; Hsieh et al., Plant Physiol. 2011 March; 155(3):1079-90; Danner et al., Phytochemistry. 2011 Apr. 12 [Epub ahead of print]; Jones et al., J Biol Chem. 2011 Mar. 24 [Epub ahead of print]; Keeling et al., BMC Plant Biol. 2011 Mar. 7; 11:43; Martin et al., BMC Plant Biol. 2010 Oct. 21; 10:226; Kumeta & Ito, Plant Physiol. 2010 December; 154(4):1998-2007; and Köllner & Boland, J Org Chem. 2010 Aug. 20; 75(16):5590-600.

Recombinant Cells Capable of Production of Isoprenoid Precursors and/or Isoprenoids Via the Alternative Lower MVA Pathway

The recombinant cells (e.g., recombinant bacterial cells) described herein have the ability to produce isoprenoid precursors and/or isoprenoids at a amount and/or concentration greater than that of the same cells lacking one or more copies of a nucleic acid encoding a phosphomevalonate decarboxylase polypeptide, one or more copies of a nucleic acid encoding an isopentenyl kinase polypeptide, one or more copies of a heterologous nucleic acid encoding a MVA pathway polypeptide, and one or more heterologous nucleic acids encoding a polyprenyl pyrophosphate synthase polypeptide when cultured under the same conditions. In certain aspects, the recombinant cells described herein comprise one or more copies of an endogenous nucleic acid encoding a phosphomevalonate decarboxylase from Herpetosiphon aurantiacus, Anaerolinea thermophila, or S378Pa3-2. In certain aspects, the recombinant cells described herein comprise a nucleic acid encoding an isopentenyl kinase from Herpetosiphon aurantiacus, Methanocaldococcus jannaschii, or Methanobrevibacter ruminantium.

In some of the embodiments, provided herein are recombinant cells capable of producing isoprenoid precursors, wherein the cells comprise (i) a nucleic acid encoding a polypeptide having phosphomevalonate decarboxylase activity, (ii) a nucleic acid encoding a polypeptide having isopentenyl kinase activity, and (iii) one or more nucleic acids encoding one or more polypeptides of the MVA pathway, wherein the total amount of ATP utilized by the cells during production of isoprenoid precursors is reduced as compared to isoprenoid precursor-producing cells that do not comprise a nucleic acid encoding a polypeptide having phosphomevalonate decarboxylase activity and/or a nucleic acid encoding a polypeptide having isopentenyl kinase activity. In some embodiments, the total amount of ATP utilized by the cells during production of isoprenoid precursors is reduced by at least 1 ATP net, 2 ATP net, 3ATP net, 4 ATP net or 5 ATP net. In some embodiments, the total amount of ATP utilized by the cells during production of isoprenoid precursors is reduced by 1 ATP net. In some of the embodiments, provided herein are recombinant cells capable of producing isoprenoids, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an polyprenyl pyrophosphate synthase polypeptide, wherein the total amount of ATP utilized by the cells during production of isoprenoids is reduced as compared to isoprenoid-producing cells that do not comprise a nucleic acid encoding a polypeptide having phosphomevalonate decarboxylase activity and/or a nucleic acid encoding a polypeptide having isopentenyl kinase activity. In some embodiments, the total amount of ATP utilized by the cells during production of isoprenoids is reduced by at least 1 ATP net, 2 ATP net, 3ATP net, 4 ATP net or 5 ATP net. In some embodiments, the total amount of ATP utilized by the cells during production of isoprenoids is reduced by 1 ATP net.

In some aspects, the one or more copies of a nucleic acid encoding a phosphomevalonate decarboxylase polypeptide, one or more copies of a nucleic acid encoding an isopentenyl kinase polypeptide, one or more copies of a heterologous nucleic acid encoding a MVA pathway polypeptide, and one or more heterologous nucleic acid encoding a polyprenyl pyrophosphate synthase polypeptide are heterologous nucleic acids that are integrated into the host cell's chromosome. The recombinant cells can produce at least 5% greater amounts of isoprenoid precursors and/or isoprenoids when compared to isoprenoids and/or isoprenoid precursor-producing recombinant cells that do not comprise phosphoketolase polypeptide. Alternatively, the recombinant cells can produce greater than about 1%, 2%, 3%, 4%, 5%, 6%, 7%, 8%, 9%, 10%, 11%, 12%, 13%, 14%, or 15% of isoprenoid precursors and/or isoprenoids, inclusive, as well as any numerical value in between these numbers compared to the production of isoprenoids and/or isoprenoid-precursors by isoprenoids and/or isoprenoid-precursors-producing cells which do not express of one or more copies of a nucleic acid encoding a phosphomevalonate decarboxylase polypeptide and/or an isopentenyl kinase polypeptide. In certain embodiments described herein, the methods herein comprise host cells have been further modified and/or engineered to increase carbon flux to MVA production thereby providing enhanced production of isoprenoids and/or isoprenoid-precursors as compared to the production of isoprenoids and/or isoprenoid-precursors by isoprenoids and/or isoprenoid-precursors-producing cells that do not express one or more heterologous nucleic acids encoding phosphomevalonate decarboxylase polypeptide and/or an isopentenyl kinase polypeptide and which have not been modified and/or engineered for increased carbon flux to mevalonate production.

In one aspect of the invention, there are provided recombinant cells comprising a nucleic acid encoding a polypeptide having phosphomevalonate decarboxylase activity, a nucleic acid encoding a polypeptide having isopentenyl kinase activity, one or more heterologous nucleic acids encoding one or more MVA pathway polypeptide(s) (i.e., the upper MVA pathway and MVK), one or more heterologous nucleic acids encoding polyprenyl pyrophosphate synthase and/or one or more heterologous nucleic acids encoding a DXP pathway polypeptide(s). The cells can further comprise one or more heterologous nucleic acids encoding an IDI polypeptide. The cells can further comprise one or more heterologous nucleic acids encoding an phosphoketolase polypeptide. Additionally, the polyprenyl pyrophosphate synthase polypeptide can be an FPP synthase polypeptide. In certain embodiments, the nucleic acid encoding a phosphomevalonate decarboxylase is from Herpetosiphon aurantiacus, Anaerolinea thermophila, or S378Pa3-2. In certain embodiments, the nucleic acid encoding an isopentenyl kinase is from Herpetosiphon aurantiacus, Methanocaldococcus jannaschii, or Methanobrevibacter ruminantium. The one or more nucleic acids can be operably linked to constitutive promoters, can be operably linked to inducible promoters, or can be operably linked to a combination of inducible and constitutive promoters. The one or more nucleic acids can additionally be operably linked strong promoters, weak promoters, and/or medium promoters. One or more of the nucleic acids encoding a phosphomevalonate decarboxylase polypeptide, isopentenyl kinase polypeptide, one or more MVA pathway polypeptide(s) (i.e., the upper MVA pathway and MVK), a polyprenyl pyrophosphate synthase polypeptide and/or one or more heterologous nucleic acids encoding a DXP pathway polypeptide(s) can be integrated into a genome of the host cells or can be stably expressed in the cells. The one or more nucleic acids can additionally be on one or more vectors.

Provided herein are recombinant cells capable of isoprenoid precursor and/or isoprenoid production. Recombinant cells produce isoprenoid precursors and/or isoprenoids by the expression of one or more of the nucleic acids encoding a phosphomevalonate decarboxylase polypeptide, isopentenyl kinase polypeptide, one or more MVA pathway polypeptide(s) (i.e., the upper MVA pathway and MVK), a polyprenyl pyrophosphate synthase polypeptide. In certain embodiments, the nucleic acid encoding a phosphomevalonate decarboxylase is from Herpetosiphon aurantiacus, Anaerolinea thermophila, or S378Pa3-2. In certain embodiments, the nucleic acid encoding an isopentenyl kinase is from Herpetosiphon aurantiacus, Methanocaldococcus jannaschii, or Methanobrevibacter ruminantium. As used herein, “enhanced” isoprenoid precursor and/or isoprenoid production refers to an increased cell productivity index (CPI) for isoprenoid precursor and/or isoprenoid production, an increased titer of isoprenoid precursors and/or isoprenoids, an increased mass yield of isoprenoid precursors and/or isoprenoids, and/or an increased specific productivity of isoprenoid precursors and/or isoprenoids by the cells described by any of the compositions and methods described herein compared to cells which do not have one or more of the nucleic acids encoding a phosphomevalonate decarboxylase polypeptide, isopentenyl kinase polypeptide, one or more MVA pathway polypeptide(s) (i.e., the upper MVA pathway and MVK), a polyprenyl pyrophosphate synthase polypeptide. The production of isoprenoid precursors and/or isoprenoids can be enhanced by about 5% to about 1,000,000 folds. The production of isoprenoid precursors and/or isoprenoids can be enhanced by about 10% to about 1,000,000 folds (e.g., about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of isoprenoid and/or isoprenoid precursors by cells without the expression of one or more heterologous nucleic acids encoding a phosphoketolase. In certain embodiments described herein, the recombinant host cells have been further modified and/or engineered to increase carbon flux to MVA production thereby providing enhanced production of isoprenoids and/or isoprenoid-precursors as compared to the production of isoprenoids and/or isoprenoid-precursors by isoprenoids and/or isoprenoid-precursors-producing cells that do not express one or more heterologous nucleic acids encoding phosphomevalonate decarboxylase polypeptide and/or isopentenyl kinase polypeptide, and which have not been modified and/or engineered for increased carbon flux to mevalonate production.

In other embodiments, the recombinant cells described herein can provide for the production of isoprenoid precursors and/or isoprenoids can also enhanced by at least about any of 5%, 10%, 20%, 30%, 40%, 50%, 60%, 70%, 80%, 90%, 1 fold, 2 folds, 5 folds, 10 folds, 20 folds, 50 folds, 100 folds, 200 folds, 500 folds, 1000 folds, 2000 folds, 5000 folds, 10,000 folds, 20,000 folds, 50,000 folds, 100,000 folds, 200,000 folds, 500,000 folds, or 1,000,000 folds compared to the production of isoprenoid precursors and/or isoprenoids by isoprenoid precursors and/or isoprenoids producing recombinant cells which do not express of one or more heterologous nucleic acids encoding a phosphomevalonate decarboxylase polypeptide and/or isopentenyl kinase polypeptide.

Methods of Using the Recombinant Cells to Produce Isoprenoids and/or Isoprenoid Precursor Molecules Via the Alternative Lower MVA Pathway

Also provided herein are methods of producing isoprenoid precursor molecules and/or isoprenoids comprising culturing recombinant cells (e.g., recombinant bacterial cells) that comprise one or more nucleic acids encoding a phosphomevalonate decarboxylase polypeptide, isopentenyl kinase polypeptide and an polyprenyl pyrophosphate synthase polypeptide. In certain embodiments, the recombinant cells further comprise one or more one or more heterologous nucleic acids encoding an upper MVA pathway polypeptide and an MVK polypeptide. The isoprenoid precursor molecules and/or isoprenoids can be produced from any of the cells described herein and according to any of the methods described herein. Any of the cells can be used for the purpose of producing isoprenoid precursor molecules and/or isoprenoids from a carbon source, including six carbon sugars such as glucose (e.g., a carbohydrate).

In certain aspects, provided herein are methods of making isoprenoid precursor molecules and/or isoprenoids comprising culturing recombinant cells comprising one or more nucleic acids encoding a phosphomevalonate decarboxylase is from Herpetosiphon aurantiacus, Anaerolinea thermophila, or S378Pa3-2, an isopentenyl kinase is from Herpetosiphon aurantiacus, Methanocaldococcus jannaschii, or Methanobrevibacter ruminantium, an mvaE and an mvaS polypeptide from L. grayi, E. faecium, E. gallinarum, E. casseliflavus, and/or E. faecalis, in a suitable condition for producing isoprenoid precursor molecules and/or isoprenoids, and (b) producing isoprenoid precursor molecules and/or isoprenoids. The cells can further comprise one or more nucleic acid molecules encoding the alternative lower MVA pathway polypeptide(s) described above (e.g., MVK and/or IDI) and any of the polyprenyl pyrophosphate synthase polypeptide(s) described above. In some aspects, the recombinant cells can be any of the cells described herein. Any of the polyprenyl pyrophosphate synthase or variants thereof described herein, any of the host cell strains described herein, any of the promoters described herein, and/or any of the vectors described herein can also be used to produce isoprenoid precursor molecules and/or isoprenoids using any of the energy sources (e.g. glucose or any other six carbon sugar) described herein. In some aspects, the method of producing isoprenoid precursor molecules and/or isoprenoids further comprises a step of recovering the isoprenoid precursor molecules and/or isoprenoids.

The method of producing isoprenoid precursor molecules and/or isoprenoids can similarly comprise the steps of: (a) culturing recombinant cells (including, but not limited to, E. coli cells) that do not endogenously express a phosphomevalonate decarboxylase polypeptide, wherein the cells heterologously express one or more copies of a gene encoding a phosphomevalonate decarboxylase polypeptide along with one or more nucleic acids expressing an isopentenyl kinase; and (b) producing isoprenoid precursor molecules and/or isoprenoids, wherein the recombinant cells produce greater amounts of isoprenoid precursors and/or isoprenoids when compared to isoprenoids and/or isoprenoid precursor-producing cells that do not comprise the phosphomevalonate decarboxylase polypeptide and/or isopentenyl kinase polypeptide.

The instant methods for the production of isoprenoid precursor molecules and/or isoprenoids can produce at least 5% greater amounts of isoprenoid precursors and/or isoprenoids when compared to isoprenoids and/or isoprenoid precursor-producing recombinant cells that do not comprise a phosphoketolase polypeptide. Alternatively, the recombinant cells can produce greater than about 1%, 2%, 3%, 4%, 5%, 6%, 7%, 8%, 9%, 10%, 11%, 12%, 13%, 14%, or 15% of isoprenoid precursors and/or isoprenoids, inclusive. In some aspects, the method of producing isoprenoid precursor molecules and/or isoprenoids further comprises a step of recovering the isoprenoid precursor molecules and/or isoprenoids.

Provided herein are methods of using any of the cells described above for enhanced isoprenoid and/or isoprenoid precursor molecule production. The production of isoprenoid precursor molecules and/or isoprenoids by the cells can be enhanced by the expression of one or more of the nucleic acids encoding a phosphomevalonate decarboxylase polypeptide, isopentenyl kinase polypeptide, one or more MVA pathway polypeptide(s) (i.e., the upper MVA pathway and MVK), and one or more heterologous nucleic acids encoding a polyprenyl pyrophosphate synthase polypeptide. As used herein, “enhanced” isoprenoid precursor and/or isoprenoid production refers to an increased cell productivity index (CPI) for isoprenoid precursor and/or isoprenoid production, an increased titer of isoprenoid precursors and/or isoprenoids, an increased mass yield of isoprenoid precursors and/or isoprenoids, and/or an increased specific productivity of isoprenoid precursors and/or isoprenoids by the cells described by any of the compositions and methods described herein compared to cells which do not have one or more of the nucleic acids encoding a phosphomevalonate decarboxylase polypeptide, isopentenyl kinase polypeptide, one or more MVA pathway polypeptide(s) (i.e., the upper MVA pathway and MVK), a polyprenyl pyrophosphate synthase polypeptide. The production of isoprenoid precursor molecules and/or isoprenoids can be enhanced by about 5% to about 1,000,000 folds. The production of isoprenoid precursor molecules and/or isoprenoids can be enhanced by about 10% to about 1,000,000 folds (e.g., about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of isoprenoid precursor molecules and/or isoprenoids by cells without the expression of one or more heterologous nucleic acids encoding a phosphoketolase polypeptide. In certain embodiments described herein, the methods comprise recombinant host cells that have been further modified and/or engineered to increased carbon flux to MVA production thereby providing enhanced production of isoprenoids and/or isoprenoid-precursors as compared to the production of isoprenoids and/or isoprenoid-precursors by isoprenoids and/or isoprenoid-precursors-producing cells that do not express one or more nucleic acids encoding phosphomevalonate decarboxylase polypeptide and/or isopentenyl kinase polypeptide and which have not been modified and/or engineered for increased carbon flux to mevalonate production.

The production of isoprenoid precursor molecules and/or isoprenoids can also enhanced by the methods described herein by at least about any of 10%, 20%, 30%, 40%, 50%, 60%, 70%, 80%, 90%, 1 fold, 2 folds, 5 folds, 10 folds, 20 folds, 50 folds, 100 folds, 200 folds, 500 folds, 1000 folds, 2000 folds, 5000 folds, 10,000 folds, 20,000 folds, 50,000 folds, 100,000 folds, 200,000 folds, 500,000 folds, or 1,000,000 folds compared to the production of isoprenoid precursor molecules and/or isoprenoids by isoprenoid precursors and/or isoprenoid-producing cells without the expression of one or more nucleic acids encoding a phosphomevalonate decarboxylase polypeptide and/or isopentenyl kinase polypeptide. In certain embodiments described herein, the methods comprise recombinant host cells that have been further modified and/or engineered to increase carbon flux to MVA production thereby providing enhanced production of isoprenoids and/or isoprenoid-precursors as compared to the production of isoprenoids and/or isoprenoid-precursors by isoprenoids and/or isoprenoid-precursors-producing cells that do not express one or more nucleic acids encoding phosphomevalonate decarboxylase polypeptide and/or isopentenyl kinase polypeptide and which have not been modified and/or engineered for increased carbon flux to mevalonate production.

In one aspect, described herein are compositions that comprise an isoprenoid precursor. In some embodiments, the composition comprising an isoprenoid precursor is produced by any one of the recombinant cells described herein. For example, a composition comprising an isoprenoid precursor can be produced by a recombinant cell comprising (i) a nucleic acid encoding a polypeptide having phosphomevalonate decarboxylase activity, (ii) a nucleic acid encoding a polypeptide having isopentenyl kinase activity, and (iii) one or more nucleic acids encoding one or more polypeptides of the MVA pathway, wherein culturing of said recombinant cell provides for the production of isoprenoid precursors. In some embodiments, the nucleic acid encoding a polypeptide having phosphomevalonate decarboxylase activity is from an archaea. In further embodiments, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In some embodiments, the nucleic acid encoding a polypeptide having phosphomevalonate decarboxylase activity is from a microorganism selected from the group consisting of: Herpetosiphon aurantiacus, S378Pa3-2, and Anaerolinea thermophila. In some embodiments, the nucleic acid encoding a polypeptide having phosphomevalonate decarboxylase activity encodes a polypeptide having an amino acid sequence with at least 85% sequence identity to an amino acid sequence selected from the group consisting of SEQ ID NOs:16-18. In some embodiments, the nucleic acid encoding a polypeptide having isopentenyl kinase activity is from an archaea. In further embodiments, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In some embodiments, the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila. In some embodiments, the nucleic acid encoding a polypeptide having isopentenyl kinase activity encodes a polypeptide having an amino acid sequence with at least 85% sequence identity to an amino acid sequence selected from the group consisting of SEQ ID NOs:19-23. In some embodiments, the one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate. In some embodiments, the one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate. In some embodiments, a composition comprising an isoprenoid precursor is produced by a recombinant cell that comprises an attenuated enzyme that converts mevalonate 5-pyrophosphate to isopentenyl pyrophosphate. In some embodiments, a composition comprising an isoprenoid precursor is produced by a recombinant cell that comprises an attenuated enzyme that converts mevalonate 5-phosphate to mevalonate 5-pyrophosphate. In some embodiments, a composition comprising an isoprenoid precursor is produced by a recombinant cell that further comprises one or more nucleic acids encoding one or more 1-deoxy-D-xylulose 5-phosphate (DXP) pathway polypeptides. In some embodiments, a composition comprising an isoprenoid precursor is produced by a recombinant cell comprising one or more attenuated enzymes of the 1-deoxy-D-xylulose 5-phosphate (DXP) pathway. In some embodiments, a composition comprising an isoprenoid precursor is produced by a recombinant cell that further comprises a heterologous nucleic acid encoding a polypeptide having phosphoketolase activity. In any of the embodiments herein, a nucleic acid encoding a polypeptide of interest (e.g., a polypeptide having phosphomevalonate decarboxylase activity, a polypeptide having isopentenyl kinase activity, etc) can be a heterologous nucleic acid or an endogenous nucleic acid.

In one aspect, described herein are compositions that comprise an isoprenoid. In some embodiments, the composition comprising an isoprenoid is produced by any one of the recombinant cells described herein. For example, a composition comprising an isoprenoid can be produced by a recombinant cell comprising (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an polyprenyl pyrophosphate synthase polypeptide, wherein culturing of said recombinant cell provides for the production of an isoprenoid. In some embodiments, the nucleic acid encoding a polypeptide having phosphomevalonate decarboxylase activity is from an archaea. In further embodiments, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In some embodiments, the nucleic acid encoding a polypeptide having phosphomevalonate decarboxylase activity is from a microorganism selected from the group consisting of: Herpetosiphon aurantiacus, S378Pa3-2, and Anaerolinea thermophila. In some embodiments, the nucleic acid encoding a polypeptide having phosphomevalonate decarboxylase activity encodes a polypeptide having an amino acid sequence with at least 85% sequence identity to an amino acid sequence selected from the group consisting of SEQ ID NOs:16-18. In some embodiments, the nucleic acid encoding a polypeptide having isopentenyl kinase activity is from an archaea. In further embodiments, the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota. In some embodiments, the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila. In some embodiments, the nucleic acid encoding a polypeptide having isopentenyl kinase activity encodes a polypeptide having an amino acid sequence with at least 85% sequence identity to an amino acid sequence selected from the group consisting of SEQ ID NOs:19-23. In some embodiments, the one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate. In some embodiments, the one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate. In some embodiments, a composition comprising an isoprenoid is produced by a recombinant cell that comprises an attenuated enzyme that converts mevalonate 5-pyrophosphate to isopentenyl pyrophosphate. In some embodiments, a composition comprising an isoprenoid is produced by a recombinant cell that comprises an attenuated enzyme that converts mevalonate 5-phosphate to mevalonate 5-pyrophosphate. In some embodiments, a composition comprising an isoprenoid is produced by a recombinant cell that further comprises one or more nucleic acids encoding one or more 1-deoxy-D-xylulose 5-phosphate (DXP) pathway polypeptides. In some embodiments, a composition comprising an isoprenoid is produced by a recombinant cell comprising one or more attenuated enzymes of the 1-deoxy-D-xylulose 5-phosphate (DXP) pathway. In some embodiments, a composition comprising an isoprenoid is produced by a recombinant cell that further comprises a heterologous nucleic acid encoding a polypeptide having phosphoketolase activity. In any of the embodiments herein, a nucleic acid encoding a polypeptide of interest (e.g., a polypeptide having phosphomevalonate decarboxylase activity, a polypeptide having isopentenyl kinase activity, etc) can be a heterologous nucleic acid or an endogenous nucleic acid. In any of the embodiments herein, the composition can comprise an isoprenoid selected from the group consisting of monoterpenes, diterpenes, triterpenes, tetraterpenes, sesquiterpene, and polyterpene. In any of the embodiments herein, the composition can comprise an isoprenoid selected from the group consisting of abietadiene, amorphadiene, carene, α-famesene, β-farnesene, farnesol, geraniol, geranylgeraniol, linalool, limonene, myrcene, nerolidol, ocimene, patchoulol, β-pinene, sabinene, γ-terpinene, terpindene and valencene.

### Vectors

Suitable vectors can be used for any of the compositions and methods described herein. For example, suitable vectors can be used to optimize the expression of one or more copies of a gene encoding a phosphomevalonate decarboxylase, an isopentenyl kinase, an upper MVA pathway polypeptide including, but not limited to, mvaE and an mvaS polypeptide, a lower MVA pathway polypeptide (e.g., MVK and IDI), an isoprene synthase, or a polyprenyl pyrophosphate synthase in a particular host cell (e.g., E. coli). In some aspects, the vector contains a selective marker. Examples of selectable markers include, but are not limited to, antibiotic resistance nucleic acids (e.g., kanamycin, ampicillin, carbenicillin, gentamicin, hygromycin, phleomycin, bleomycin, neomycin, or chloramphenicol) and/or nucleic acids that confer a metabolic advantage, such as a nutritional advantage on the host cell. In some aspects, one or more copies of a phosphomevalonate decarboxylase, an isopentenyl kinase, an upper MVA pathway polypeptide including, but not limited to, mvaE and an mvaS polypeptide, a lower MVA pathway polypeptide (e.g., MVK and IDI), an mvaE and an mvaS nucleic acid from L. grayi, E. faecium, E. gallinarum, E. casseliflavus, and/or E. faecalis, an isoprene synthase, or a polyprenyl pyrophosphate synthase nucleic acid(s) integrate into the genome of host cells without a selective marker.

Any one of the vectors characterized herein or used in the Examples of the present disclosure can be used in the present invention.

### Transformation Methods

Nucleic acids encoding one or more copies of a monophosphate decarboxylase, an isopentenyl kinase, an upper MVA pathway polypeptide including, but not limited to, mvaE and an mvaS polypeptide, a lower MVA pathway polypeptide, and/or lower MVA pathway polypeptides can be inserted into a cell using suitable techniques. Additionally, isoprene synthase, IDI, DXP pathway, and/or polyprenyl pyrophosphate synthase nucleic acids or vectors containing them can be inserted into a host cell (e.g., a plant cell, a fungal cell, a yeast cell, or a bacterial cell described herein) using standard techniques for introduction of a DNA construct or vector into a host cell, such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are known in the art (See, e.g., Current Protocols in Molecular Biology (F. M. Ausubel et al. (eds.) Chapter 9, 1987; Sambrook et al., Molecular Cloning: A Laboratory Manual, 2^{nd} ed., Cold Spring Harbor, 1989; and Campbell et al., Curr. Genet. 16:53-56, 1989). The introduced nucleic acids can be integrated into chromosomal DNA or maintained as extrachromosomal replicating sequences. Transformants can be selected by any method known in the art. Suitable methods for selecting transformants are described in International Publication No. WO 2009/076676, U.S. Patent Publ. No. 2009/0203102, WO 2010/003007, US Publ. No. 2010/0048964, WO 2009/132220, and US Publ. No. 2010/0003716.

### Exemplary Host Cells

One of skill in the art will recognize that expression vectors are designed to contain certain components which optimize gene expression for certain host strains. Such optimization components include, but are not limited to origin of replication, promoters, and enhancers. The vectors and components referenced herein are described for exemplary purposes and are not meant to narrow the scope of the invention.

Any cell or progeny thereof that can be used to heterologously express genes can be used to express one or more a monophosphate decarboxylase isolated from Herpetosiphon aurantiacus, Anaerolinea thermophila, and/or S378Pa3-2 along with one or more heterologous nucleic acids expressing isopentenyl kinase, one or more MVA pathway peptides, isoprene synthase, IDI, DXP pathway polypeptide(s), and/or polyprenyl pyrophosphate synthase polypeptides. Exemplary host cells include, for example, yeasts, such as species of Saccharomyces (e.g., S. cerevisiae), bacteria, such as species of Escherichia (e.g., E. coli), archaea, such as species of Methanosarcina (e.g., Methanosarcina mazei), plants, such as kudzu or poplar (e.g., Populus alba or Populus alba×tremula CAC35696) or aspen (e.g., Populus tremuloides).

Bacteria cells, including gram positive or gram negative bacteria can be used to express any of the heterologous genes described above. In some embodiments, the host cell is a gram-positive bacterium. Non-limiting examples include strains of Streptomyces (e.g., S. lividans, S. coelicolor, S. rubiginosus, or S. griseus), Streptococcus, Bacillus (e.g., B. lichenformis or B. subtilis), Listeria (e.g., L. monocytogenes), Corynebacteria (e.g., C. glutamicum), or Lactobacillus (e.g., L. spp). In some embodiments, the source organism is a gram-negative bacterium. Non-limiting examples include strains of Escherichia (e.g., E. coli), Pseudomonas (e.g., P. alcaligenes), Pantoea (e.g., P. citrea), Enterobacter, or Helicobacter (H. pylori). In particular, the nucleic acids described herein can be expressed in any one of P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, C. glutamicum, C. acetoacidophilum, C. efficiens, C. diphtheria, C. bovis, S. albus, S. lividans, S. coelicolor, S. griseus, Pseudomonas sp., and P. alcaligenes cells.

There are numerous types of anaerobic cells that can be used as host cells in the compositions and methods of the present invention. In one aspect of the invention, the cells described in any of the compositions or methods described herein are obligate anaerobic cells and progeny thereof. Obligate anaerobes typically do not grow well, if at all, in conditions where oxygen is present. It is to be understood that a small amount of oxygen may be present, that is, there is some tolerance level that obligate anaerobes have for a low level of oxygen. In one aspect, obligate anaerobes engineered to produce isoprenoid precursors, isoprene, and isoprenoids can serve as host cells for any of the methods and/or compositions described herein and are grown under substantially oxygen-free conditions, wherein the amount of oxygen present is not harmful to the growth, maintenance, and/or fermentation of the anaerobes.

In another aspect of the invention, the host cells described and/or used in any of the compositions or methods described herein are facultative anaerobic cells and progeny thereof. Facultative anaerobes can generate cellular ATP by aerobic respiration (e.g., utilization of the TCA cycle) if oxygen is present. However, facultative anaerobes can also grow in the absence of oxygen. This is in contrast to obligate anaerobes which die or grow poorly in the presence of greater amounts of oxygen. In one aspect, therefore, facultative anaerobes can serve as host cells for any of the compositions and/or methods provided herein and can be engineered to produce isoprenoid precursors, isoprene, and isoprenoids. Facultative anaerobic host cells can be grown under substantially oxygen-free conditions, wherein the amount of oxygen present is not harmful to the growth, maintenance, and/or fermentation of the anaerobes, or can be alternatively grown in the presence of greater amounts of oxygen.

The host cell can additionally be a filamentous fungal cell and progeny thereof. (See, e.g., Berka & Barnett, Biotechnology Advances, (1989), 7(2):127-154). In some aspects, the filamentous fungal cell can be any of Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp., such as A. oryzae, A. niger, A sojae, A. japonicus, A. nidulans, or A. awamori, Fusarium sp., such as F. roseum, F. graminum F. cerealis, F. oxysporuim, or F. venenatum, Neurospora sp., such as N. crassa, Hypocrea sp., Mucor sp., such as M. miehei, Rhizopus sp. or Emericella sp. In some aspects, the fungus is A. nidulans, A. awamori, A. oryzae, A. aculeatus, A. niger, A. japonicus, T. reesei, T. viride, F. oxysporum, or F. solani. In certain embodiments, plasmids or plasmid components for use herein include those described in U.S. patent pub. No. US 2011/0045563.

The host cell can also be a yeast, such as Saccharomyces sp., Schizosaccharomyces sp., Pichia sp., or Candida sp. In some aspects, the Saccharomyces sp. is Saccharomyces cerevisiae (See, e.g., Romanos et al., Yeast, (1992), 8(6):423-488). In certain embodiments, plasmids or plasmid components for use herein include those described in U.S. Pat. No. 7,659,097 and U.S. patent pub. No. US 2011/0045563.

The host cell can also be a species of plant, such as a plant from the family Fabaceae, such as the Faboideae subfamily. In some aspects, the host cell is kudzu, poplar (such as Populus alba×tremula CAC35696), aspen (such as Populus tremuloides), or Quercus robur.

The host cell can additionally be a species of algae, such as a green algae, red algae, glaucophytes, chlorarachniophytes, euglenids, chromista, or dinoflagellates. (See, e.g., Saunders & Warmbrodt, “Gene Expression in Algae and Fungi, Including Yeast,” (1993), National Agricultural Library, Beltsville, Md.). In certain embodiments, plasmids or plasmid components for use herein include those described in U.S. Patent Pub. No. US 2011/0045563. In some aspects, the host cell is a cyanobacterium, such as cyanobacterium classified into any of the following groups based on morphology: Chlorococcales, Pleurocapsales, Oscillatoriales, Nostocales, or Stigonematales (See, e.g., Lindberg et al., Metab. Eng., (2010) 12(1):70-79). In certain embodiments, plasmids or plasmid components for use herein include those described in U.S. patent pub. No. US 2010/0297749; US 2009/0282545 and Intl. Pat. Appl. No. WO 2011/034863.

E. coli host cells can be used to express one or more monophosphate decarboxylase enzymes from Herpetosiphon aurantiacus, Anaerolinea thermophila, or S378Pa3-2 along with one or more heterologous nucleic acids encoding isopentenyl kinase, one or more MVA pathway polypeptides, isoprene synthase, IDI, DXP pathway polypeptide(s), and/or polyprenyl pyrophosphate synthase polypeptides. In one aspect, the host cell is a recombinant cell of an Escherichia coli (E. coli) strain, or progeny thereof, capable of producing isoprene that expresses one or more nucleic acids encoding monophosphate decarboxylase from Herpetosiphon aurantiacus, Anaerolinea thermophila, or S378Pa3-2 along with one or more heterologous nucleic acids expressing isopentenyl kinase, one or more MVA pathway peptides, isoprene synthase, and IDI. The E. coli host cells can produce isoprene in amounts, peak titers, and cell productivities greater than that of the same cells lacking one or more heterologously expressed nucleic acids encoding monophosphate decarboxylase from Herpetosiphon aurantiacus, Anaerolinea thermophila, or S378Pa3-2 along with one or more heterologous nucleic acids expressing isopentenyl kinase, one or more MVA pathway peptides, isoprene synthase, and IDI. In addition, the one or more heterologously expressed nucleic acids encoding monophosphate decarboxylase from Herpetosiphon aurantiacus, Anaerolinea thermophila, or S378Pa3-2 along with one or more heterologous nucleic acids expressing one or more MVA pathway peptides in E. coli can be chromosomal copies (e.g., integrated into the E. coli chromosome). In other aspects, the E. coli cells are in culture. In some aspects the one or more monophosphate decarboxylase is from Herpetosiphon aurantiacus, Anaerolinea thermophila, or S378Pa3-2.

### Exemplary Host Cell Modifications

### Citrate Synthase Pathway

Citrate synthase catalyzes the condensation of oxaloacetate and acetyl-CoA to form citrate, a metabolite of the tricarboxylic acid (TCA) cycle (Ner, S. et al. 1983. Biochemistry, 22: 5243-5249; Bhayana, V. and Duckworth, H. 1984. Biochemistry 23: 2900-2905). In E. coli, this enzyme, encoded by gltA, behaves like a trimer of dimeric subunits. The hexameric form allows the enzyme to be allosterically regulated by NADH. This enzyme has been widely studied (Wiegand, G., and Remington, S. 1986. Annual Rev. Biophysics Biophys. Chem. 15: 97-117; Duckworth et al. 1987. Biochem Soc Symp. 54:83-92; Stockell, D. et al. 2003. J. Biol. Chem. 278: 35435-43; Maurus, R. et al. 2003. Biochemistry. 42:5555-5565). To avoid allosteric inhibition by NADH, replacement by or supplementation with the Bacillus subtilis NADH-insensitive citrate synthase has been considered (Underwood et al. 2002. Appl. Environ. Microbiol. 68:1071-1081; Sanchez et al. 2005. Met. Eng. 7:229-239).

The reaction catalyzed by citrate synthase is directly competing with the thiolase catalyzing the first step of the mevalonate pathway, as they both have acetyl-CoA as a substrate (Hedl et al. 2002. J. Bact. 184:2116-2122). Therefore, one of skill in the art can modulate citrate synthase expression (e.g., decrease enzyme activity) to allow more carbon to flux into the mevalonate pathway, thereby increasing the eventual production of mevalonate, isoprene, isoprenoid precursors, and isoprenoids. Decrease of citrate synthase activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, 99%, or 100%. In some aspects, the activity of citrate synthase is modulated by decreasing the activity of an endogenous citrate synthase gene. This can be accomplished by chromosomal replacement of an endogenous citrate synthase gene with a transgene encoding an NADH-insensitive citrate synthase or by using a transgene encoding an NADH-insensitive citrate synthase that is derived from Bacillus subtilis. The activity of citrate synthase can also be modulated (e.g., decreased) by replacing the endogenous citrate synthase gene promoter with a synthetic constitutively low expressing promoter. The gene encoding citrate synthase can also be deleted. The decrease of the activity of citrate synthase can result in more carbon flux into the mevalonate dependent biosynthetic pathway in comparison to cells that do not have decreased expression of citrate synthase. In any aspects of the invention, provided herein are recombinant cells comprising one or more expressed nucleic acids encoding monophosphate decarboxylase and/or isopentenyl kinase polypeptides as disclosed herein and further engineered to decrease the activity of citrate synthase (gltA). Activity modulation (e.g., decreased) of citrate synthase isozymes is also contemplated herein. In any aspects of the invention, provided herein are recombinant cells comprising one or more expressed nucleic acids encoding monophosphate decarboxylase and/or isopentenyl kinase polypeptides as disclosed herein and further engineered to decrease the activity of a citrate synthase isozyme.

Pathways Involving Phosphotransacetylase and/or Acetate Kinase

Phosphotransacetylase ((encoded in E. coli by (i) pta (Shimizu et al. 1969. Biochim. Biophys. Acta 191: 550-558 or (ii) eutD (Bologna et al. 2010. J of Microbiology. 48:629-636) catalyzes the reversible conversion between acetyl-CoA and acetyl phosphate (acetyl-P), while acetate kinase (encoded in E. coli by ackA) (Kakuda, H. et al. 1994. J. Biochem. 11:916-922) uses acetyl-P to form acetate. These genes can be transcribed as an operon in E. coli. Together, they catalyze the dissimulation of acetate, with the release of ATP. Thus, it is possible to increase the amount of acetyl-P going towards acetyl-CoA by enhancing the activity of phosphotransacetylase. In certain embodiments, enhancement is achieved by placing an upregulated promoter upstream of the gene in the chromosome, or to place a copy of the gene behind an adequate promoter on a plasmid. In order to decrease the amount of acetyl-coA going towards acetate, the activity of acetate kinase gene (e.g., the endogenous acetate kinase gene) can be decreased or attenuated. In certain embodiments, attenuation is achieved by deleting acetate kinase (ackA). This is done by replacing the gene with a chloramphenicol cassette followed by looping out of the cassette. In some aspects, the activity of acetate kinase is modulated by decreasing the activity of an endogenous acetate kinase. This can be accomplished by replacing the endogenous acetate kinase gene promoter with a synthetic constitutively low expressing promoter. In certain embodiments, it the attenuation of the acetated kinase gene should be done disrupting the expression of the phosphotransacetylase (pta) gene. Acetate is produced by E. coli for a variety of reasons (Wolfe, A. 2005. Microb. Mol. Biol. Rev. 69:12-50). Without being bound by theory, deletion of ackA could result in decreased carbon being diverted into acetate production (since ackA use acetyl-CoA) and thereby increase the yield of mevalonate, isoprenoid precursors, isoprene and/or isoprenoids.

In some aspects, the recombinant cells described herein produce decreased amounts of acetate in comparison to cells that do not have attenuated endogenous acetate kinase gene expression or enhanced phosphotransacetylase. Decrease in the amount of acetate produced can be measured by routine assays known to one of skill in the art. The amount of acetate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done to the endogenous acetate kinase gene expression or phosphotransacetylase gene expression.

The activity of phosphotransacetylase (pta and/or eutD) can be increased by other molecular manipulations of the enzymes. The increase of enzyme activity can be and increase in any amount of specific activity or total activity as compared to when no manipulation has been effectuated. In some instances, the increase of enzyme activity is increased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%. In one embodiment the activity of pta is increased by altering the promoter and/or rbs on the chromosome, or by expressing it from a plasmid. In any aspects of the invention, provided herein are recombinant cells comprising one or more heterologously expressed nucleic acids encoding monophosphate decarboxylase and/or isopentenyl kinase polypeptides as disclosed herein and further engineered to increase the activity of phosphotransacetylase (pta and/or eutD). Activity modulation (e.g., increased) of phosphotransacetylase isozymes is also contemplated herein. In any aspects of the invention, provided herein are recombinant cells comprising one or more expressed nucleic acids encoding monophosphate decarboxylase and/or isopentenyl kinase polypeptides as disclosed herein and further engineered to increase the activity of a phosphotransacetylase (pta and/or eutD) isozyme.

The activity of acetate kinase (ackA) can also be decreased by other molecular manipulations of the enzymes. The decrease of enzyme activity can be any amount of reduction of specific activity or total activity as compared to when no manipulation has been effectuated. In some instances, the enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%. In any aspects of the invention, provided herein are recombinant cells comprising one or more heterologously expressed nucleic acids encoding phosphoketolase polypeptides as disclosed herein and further engineered to decrease the activity of acetate kinase (ackA). Activity modulation (e.g., decreased) of acetate kinase isozymes is also contemplated herein. In any aspects of the invention, provided herein are recombinant cells comprising one or more expressed nucleic acids encoding monophosphate decarboxylase and/or isopentenyl kinase polypeptides as disclosed herein and further engineered to decrease the activity of a acetate kinase isozyme.

In some cases, attenuating the activity of the endogenous acetate kinase gene results in more carbon flux into the mevalonate dependent biosynthetic pathway in comparison to cells that do not have attenuated endogenous acetate gene expression.

### Pathways Involving Lactate Dehydrogenase

In E. coli, D-Lactate is produced from pyruvate through the enzyme lactate dehydrogenase (encoded by ldhA) (Bunch, P. et al. 1997. Microbiol. 143:187-195). Production of lactate is accompanied with oxidation of NADH, hence lactate is produced when oxygen is limited and cannot accommodate all the reducing equivalents. Thus, production of lactate could be a source for carbon consumption. As such, to improve carbon flow through to mevalonate production (and isoprene, isoprenoid precursor and isoprenoids production, if desired), one of skill in the art can modulate the activity of lactate dehydrogenase, such as by decreasing the activity of the enzyme.

Accordingly, in one aspect, the activity of lactate dehydrogenase can be modulated by attenuating the activity of an endogenous lactate dehydrogenase gene. Such attenuation can be achieved by deletion of the endogenous lactate dehydrogenase gene. Other ways of attenuating the activity of lactate dehydrogenase gene known to one of skill in the art may also be used. By manipulating the pathway that involves lactate dehydrogenase, the recombinant cell produces decreased amounts of lactate in comparison to cells that do not have attenuated endogenous lactate dehydrogenase gene expression. Decrease in the amount of lactate produced can be measured by routine assays known to one of skill in the art. The amount of lactate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of lactate dehydrogenase can also be decreased by other molecular manipulations of the enzyme. The decrease of enzyme activity can be any amount of reduction of specific activity or total activity as compared to when no manipulation has been effectuated. In some instances, the enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

Accordingly, in some cases, attenuation of the activity of the endogenous lactate dehydrogenase gene results in more carbon flux into the mevalonate dependent biosynthetic pathway in comparison to cells that do not have attenuated endogenous lactate dehydrogenase gene expression.

### Pathways Involving Glyceraldehyde 3-Phosphate

Glyceraldehyde 3-phosphate dehydrogenase (gapA and/or gapB) is a crucial enzyme of glycolysis catalyzes the conversion of glyceraldehyde 3-phosphate into 1,3-bisphospho-D-glycerate (Branlant G. and Branlant C. 1985. Eur. J. Biochem. 150:61-66).

In certain aspects, recombinant cells comprising one or more expressed nucleic acids encoding monophosphate decarboxylase and/or isopentenyl kinase polypeptides as disclosed herein further comprise one more nucleic acids encoding a phosphoketolase polypeptide. In order to direct carbon towards the phosphoketolase enzyme, glyceraldehyde 3-phosphate dehydrogenase expression can be modulated (e.g., decrease enzyme activity) to allow more carbon to flux towards fructose 6-phosphate and xylulose 5-phosphate, thereby increasing the eventual production of mevalonate, isoprenoid precursors, isoprene and/or isoprenoids. Decrease of glyceraldehyde 3-phosphate dehydrogenase activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, 99% or 100%. In some aspects, the activity of glyceraldehyde 3-phosphate dehydrogenase is modulated by decreasing the activity of an endogenous glyceraldehyde 3-phosphate dehydrogenase. This can be accomplished by replacing the endogenous glyceraldehyde 3-phosphate dehydrogenase gene promoter with a synthetic constitutively low expressing promoter. The gene encoding glyceraldehyde 3-phosphate dehydrogenase can also be deleted. The gene encoding glyceraldehyde 3-phosphate dehydrogenase can also be replaced by a Bacillus enzyme catalyzing the same reaction but producing NADPH rather than NADH. The decrease of the activity of glyceraldehyde 3-phosphate dehydrogenase can result in more carbon flux into the mevalonate-dependent biosynthetic pathway in comparison to cells that do not have decreased expression of glyceraldehyde 3-phosphate dehydrogenase. In any aspects of the invention, provided herein are recombinant cells comprising one or more expressed nucleic acids encoding monophosphate decarboxylase and/or isopentenyl kinase polypeptides as disclosed herein and further engineered to decrease the activity of glyceraldehyde 3-phosphate dehydrogenase (gapA and/or gapB). Activity modulation (e.g., decreased) of glyceraldehyde 3-phosphate dehydrogenase isozymes is also contemplated herein. In any aspects of the invention, provided herein are recombinant cells comprising one or more heterologously expressed nucleic acids encoding monophosphate decarboxylase and/or isopentenyl kinase polypeptides as disclosed herein and further engineered to decrease the activity of a glyceraldehyde 3-phosphate dehydrogenase (gapA and/or gapB) isozyme.

### Pathways Involving the Entner-Doudoroff Pathway

The Entner-Doudoroff (ED) pathway is an alternative to the Emden-Meyerhoff-Parnass (EMP-glycolysis) pathway. Some organisms, like E. coli, harbor both the ED and EMP pathways, while others have only one or the other. Bacillus subtilis has only the EMP pathway, while Zymomonas mobilis has only the ED pathway (Peekhaus and Conway. 1998. J. Bact. 180:3495-3502; Stulke and Hillen. 2000. Annu. Rev. Microbiol. 54, 849-880; Dawes et al. 1966. Biochem. J. 98:795-803). Fructose bisphosphate aldolase (fba, fbaA, fbaB, and/or fbaC) interacts with the Entner-Doudoroff pathway and reversibly catalyzes the conversion of fructose 1,6-bisphosphate into dihydroxyacetone phosphate (DHAP) and glyceraldehyde 3-phosphate (GAP) (Baldwin S. A., et. al., Biochem J. (1978) 169(3):633-41).

Phosphogluconate dehydratase (edd) removes one molecule of H₂O from 6-phospho-D-gluconate to form 2-dehydro-3-deoxy-D-gluconate 6-phosphate, while 2-keto-3-deoxygluconate 6-phosphate aldolase (eda) catalyzes an aldol cleavage (Egan et al. 1992. J. Bact. 174:4638-4646). The two genes are in an operon.

In certain aspects, recombinant cells comprising one or more expressed nucleic acids encoding monophosphate decarboxylase and/or isopentenyl kinase polypeptides as disclosed herein further comprise one more nucleic acids encoding a phosphoketolase polypeptide. Metabolites that can be directed into the phosphoketolase pathway can also be diverted into the ED pathway. To avoid metabolite loss to the ED-pathway, phosphogluconate dehydratase gene (e.g., the endogenous phosphogluconate dehydratase gene) and/or an 2-keto-3-deoxygluconate 6-phosphate aldolase gene (e.g., the endogenous 2-keto-3-deoxygluconate 6-phosphate aldolase gene) activity is attenuated. One way of achieving attenuation is by deleting phosphogluconate dehydratase (edd) and/or 2-keto-3-deoxygluconate 6-phosphate aldolase (eda). This can be accomplished by replacing one or both genes with a chloramphenicol or kanamycin cassette followed by looping out of the cassette. Without these enzymatic activities, more carbon can flux through the phosphoketolase enzyme, thus increasing the yield of mevalonate, isoprenoid precursors, isoprene and/or isoprenoids.

The activity of phosphogluconate dehydratase (edd) and/or 2-keto-3-deoxygluconate 6-phosphate aldolase (eda) can also be decreased by other molecular manipulations of the enzymes. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

In some cases, attenuating the activity of the endogenous phosphogluconate dehydratase gene and/or the endogenous 2-keto-3-deoxygluconate 6-phosphate aldolase gene results in more carbon flux into the mevalonate dependent biosynthetic pathway in comparison to cells that do not have attenuated endogenous phosphogluconate dehydratase gene and/or endogenous acetate kinase2-keto-3-deoxygluconate 6-phosphate aldolase gene expression.

Metabolites that can be directed into the phosphoketolase pathway can also be diverted into the ED pathway or EMP pathway. To avoid metabolite loss and to increase fructose-6-phosphate (F6P) concentration, fructose bisphosphate aldolase (e.g., the endogenous fructose bisphosphate aldolase) activity is attenuated. In some cases, attenuating the activity of the endogenous fructose bisphosphate aldolase (fba, fbaA, fbaB, and/or fbaC) gene results in more carbon flux into the mevalonate dependent biosynthetic pathway in comparison to cells that do not have attenuated endogenous fructose bisphosphate aldolase (fba, fbaA, fbaB, and/or fbaC) gene expression. In some aspects, attenuation is achieved by deleting fructose bisphosphate aldolase (fba, fbaA, fbaB, and/or fbaC). Deletion can be accomplished by replacing the gene with a chloramphenicol or kanamycin cassette followed by looping out of the cassette. In some aspects, the activity of fructose bisphosphate aldolase is modulated by decreasing the activity of an endogenous fructose bisphosphate aldolase. This can be accomplished by replacing the endogenous fructose bisphosphate aldolase gene promoter with a synthetic constitutively low expressing promoter. Without these enzymatic activities, more carbon can flux through the phosphoketolase enzyme, thus increasing the yield of isoprene, isoprenoid precursors, and isoprenoids via the alternative lower MVA pathway (e.g., MVK, PMevDC, IPK, and/or IDI). The activity of fructose bisphosphate aldolase can also be decreased by other molecular manipulations of the enzyme. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%. In any aspects of the invention, provided herein are recombinant cells comprising one or more expressed nucleic acids encoding monophosphate decarboxylase and/or isopentenyl kinase polypeptides as disclosed herein and further engineered to decrease the activity of fructose bisphosphate aldolase (fba, fbaA, fbaB, and/or fbaC). Activity modulation (e.g., decreased) of fructose bisphosphate aldolase isozymes is also contemplated herein. In any aspects of the invention, provided herein are recombinant cells comprising one or more expressed nucleic acids encoding monophosphate decarboxylase and/or isopentenyl kinase polypeptides as disclosed herein and further engineered to decrease the activity of a fructose bisphosphate aldolase isozyme.

### Pathways Involving the Oxidative Branch of the Pentose Phosphate Pathway

E. coli uses the pentose phosphate pathway to break down hexoses and pentoses and to provide cells with intermediates for various anabolic pathways. It is also a major producer of NADPH. The pentose phosphate pathway is composed from an oxidative branch (with enzymes like glucose 6-phosphate 1-dehydrogenase (zwf), 6-phosphogluconolactonase (pgl) or 6-phosphogluconate dehydrogenase (gnd)) and a non-oxidative branch (with enzymes such as transketolase (tktA and/or tktB), transaldolase (talA or talB), ribulose-5-phosphate-epimerase and (or) ribose-5-phosphate epimerase, ribose-5-phosphate isomerase (rpiA and/or rpiB) and/or ribulose-5-phosphate 3-epimerase (rpe)) (Sprenger. 1995. Arch. Microbiol. 164:324-330).

In certain aspects, recombinant cells comprising one or more expressed nucleic acids encoding monophosphate decarboxylase and/or isopentenyl kinase polypeptides as disclosed herein further comprise one more nucleic acids encoding a phosphoketolase polypeptide. In order to direct carbon towards the phosphoketolase enzyme, the non-oxidative branch of the pentose phosphate pathway (transketolase, transaldolase, ribulose-5-phosphate-epimerase and (or) ribose-5-phosphate epimerase, ribose-5-phosphate isomerase A, ribose-5-phosphate isomerase B, and/or ribulose-5-phosphate 3-epimerase) expression can be modulated (e.g., increase enzyme activity) to allow more carbon to flux towards fructose 6-phosphate and xylulose 5-phosphate, thereby increasing the eventual production of isoprene, isoprenoid precursors, and isoprenoids via the alternative lower MVA pathway (e.g., MVK, PMevDC, IPK, and/or IDI). Increase of transketolase, transaldolase, ribulose-5-phosphate-epimerase and (or) ribose-5-phosphate epimerase activity can be any amount of increase of specific activity or total activity as compared to when no manipulation has been effectuated. In some instances, the enzyme activity is increased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, 99%, or 100%. In some aspects, the activity of transketolase, transaldolase, ribulose-5-phosphate-epimerase and (or) ribose-5-phosphate epimerase is modulated by increasing the activity of an endogenous transketolase, transaldolase, ribulose-5-phosphate-epimerase and (or) ribose-5-phosphate epimerase. This can be accomplished by replacing the endogenous transketolase, transaldolase, ribulose-5-phosphate-epimerase and (or) ribose-5-phosphate epimerase gene promoter with a synthetic constitutively high expressing promoter. The genes encoding transketolase, transaldolase, ribulose-5-phosphate-epimerase and (or) ribose-5-phosphate epimerase can also be cloned on a plasmid behind an appropriate promoter. The increase of the activity of transketolase, transaldolase, ribulose-5-phosphate-epimerase and (or) ribose-5-phosphate epimerase can result in more carbon flux into the monophosphate mevalonate dependent biosynthetic pathway in comparison to cells that do not have increased expression of transketolase, transaldolase, ribulose-5-phosphate-epimerase and (or) ribose-5-phosphate epimerase.

In any aspects of the invention, provided herein are recombinant cells comprising one or more heterologously expressed nucleic acids encoding monophosphate decarboxylase and/or isopentenyl kinase polypeptides as disclosed herein and further engineered to increase the activity of transketolase (tktA and/or tktB). In any aspects of the invention, provided herein are recombinant cells comprising one or more expressed nucleic acids encoding monophosphate decarboxylase and/or isopentenyl kinase polypeptides as disclosed herein and further engineered to decrease the activity of transketolase (tktA and/or tktB). In any aspects of the invention, provided herein are recombinant cells comprising one or more expressed nucleic acids encoding monophosphate decarboxylase and/or isopentenyl kinase polypeptides as disclosed herein and further engineered to increase the activity of transaldolase (talA or talB). In any aspects of the invention, provided herein are recombinant cells comprising one or more expressed nucleic acids encoding monophosphate decarboxylase and/or isopentenyl kinase polypeptides as disclosed herein and further engineered to increase the activity of ribose-5-phosphate isomerase (rpiA and/or rpiB). In any aspects of the invention, provided herein are recombinant cells comprising one or more expressed nucleic acids encoding monophosphate decarboxylase and/or isopentenyl kinase polypeptides as disclosed herein and further engineered to increase the activity of ribulose-5-phosphate 3-epimerase (rpe). Activity modulation (e.g., decreased or increased) of glucose 6-phosphate 1-dehydrogenase (zwf), 6-phosphogluconolactonase (pgl), 6-phosphogluconate dehydrogenase (gnd), transketolase (tktA and/or tktB), transaldolase (talA or talB), ribulose-5-phosphate-epimerase, ribose-5-phosphate epimerase, ribose-5-phosphate isomerase (rpiA and/or rpiB) and/or ribulose-5-phosphate 3-epimerase (rpe) isozymes is also contemplated herein. In any aspects of the invention, provided herein are recombinant cells comprising one or more expressed nucleic acids encoding monophosphate decarboxylase and/or isopentenyl kinase polypeptides as disclosed herein and further engineered to increase the activity of a glucose 6-phosphate 1-dehydrogenase (zwf) isozyme. In any aspects of the invention, provided herein are recombinant cells comprising one or more expressed nucleic acids encoding monophosphate decarboxylase and/or isopentenyl kinase polypeptides as disclosed herein and further engineered to increase the activity of a transketolase (tktA and/or tktB) isozyme. In any aspects of the invention, provided herein are recombinant cells comprising one or more expressed nucleic acids encoding monophosphate decarboxylase and/or isopentenyl kinase polypeptides as disclosed herein and further engineered to decrease the activity of a transketolase (tktA and/or tktB) isozyme. In any aspects of the invention, provided herein are recombinant cells comprising one or more expressed nucleic acids encoding monophosphate decarboxylase and/or isopentenyl kinase polypeptides as disclosed herein and further engineered to increase the activity of a transaldolase (talA or talB) isozyme. In any aspects of the invention, provided herein are recombinant cells comprising one or more expressed nucleic acids encoding monophosphate decarboxylase and/or isopentenyl kinase polypeptides as disclosed herein and further engineered to increase the activity of a ribose-5-phosphate isomerase (rpiA and/or rpiB) isozyme. In any aspects of the invention, provided herein are recombinant cells comprising one or more expressed nucleic acids encoding monophosphate decarboxylase and/or isopentenyl kinase polypeptides as disclosed herein and further engineered to increase the activity of a ribulose-5-phosphate 3-epimerase (rpe) isozyme.

In certain aspects, recombinant cells comprising one or more expressed nucleic acids encoding monophosphate decarboxylase and/or isopentenyl kinase polypeptides as disclosed herein further comprise one more nucleic acids encoding a phosphoketolase polypeptide. In order to direct carbon towards the phosphoketolase enzyme, glucose 6-phosphate 1-dehydrogenase can be modulated (e.g., decrease enzyme activity). In some aspects, the activity of glucose 6-phosphate 1-dehydrogenase (zwf) (e.g., the endogenous glucose 6-phosphate 1-dehydrogenase gene) can be decreased or attenuated. In certain embodiments, attenuation is achieved by deleting glucose 6-phosphate 1-dehydrogenase. In some aspects, the activity of glucose 6-phosphate 1-dehydrogenase is modulated by decreasing the activity of an endogenous glucose 6-phosphate 1-dehydrogenase. This can be accomplished by replacing the endogenous glucose 6-phosphate 1-dehydrogenase gene promoter with a synthetic constitutively low expressing promoter. In any aspects of the invention, provided herein are recombinant cells comprising one or more heterologously expressed nucleic acids encoding monophosphate decarboxylase and/or isopentenyl kinase polypeptides as disclosed herein and further engineered to decrease the activity of glucose 6-phosphate 1-dehydrogenase (zwf). Activity modulation (e.g., decreased) of glucose 6-phosphate 1-dehydrogenase isozymes is also contemplated herein. In any aspects of the invention, provided herein are recombinant cells comprising one or more heterologously expressed nucleic acids encoding monophosphate decarboxylase and/or isopentenyl kinase polypeptides as disclosed herein and further engineered to decrease the activity of a glucose 6-phosphate 1-dehydrogenase isozyme.

### Pathways Involving Phosphofructokinase

Phosphofructokinase is a crucial enzyme of glycolysis which catalyzes the phosphorylation of fructose 6-phosphate. E. coli has two isozymes encoded by pfkA and pfkB. Most of the phosphofructokinase activity in the cell is due to pfkA (Kotlarz et al. 1975 Biochim. Biophys. Acta 381:257-268).

In certain aspects, recombinant cells comprising one or more expressed nucleic acids encoding monophosphate decarboxylase and/or isopentenyl kinase polypeptides as disclosed herein further comprise one more nucleic acids encoding a phosphoketolase polypeptide. In order to direct carbon towards the phosphoketolase enzyme, phosphofructokinase expression can be modulated (e.g., decrease enzyme activity) to allow more carbon to flux towards fructose 6-phosphate and xylulose 5-phosphate, thereby increasing the eventual production of mevalonate, isoprene, isoprenoid precursors, and isoprenoids via the alternative lower MVA pathway (e.g., MVK, PMevDC, IPK, and/or IDI). Decrease of phosphofructokinase activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, 99%. Or 100%. In some aspects, the activity of phosphofructokinase is modulated by decreasing the activity of an endogenous phosphofructokinase. This can be accomplished by replacing the endogenous phosphofructokinase gene promoter with a synthetic constitutively low expressing promoter. The gene encoding phosphofructokinase can also be deleted. The decrease of the activity of phosphofructokinase can result in more carbon flux into the mevalonate dependent biosynthetic pathway in comparison to cells that do not have decreased expression of phosphofructokinase.

In any aspects of the invention, provided herein are recombinant cells comprising one or more expressed nucleic acids encoding monophosphate decarboxylase and/or isopentenyl kinase polypeptides as disclosed herein and further engineered to decrease the activity of fructose 6-phosphate (pfkA and/or pfkB). Activity modulation (e.g., decreased) of fructose 6-phosphate isozymes is also contemplated herein. In any aspects of the invention, provided herein are recombinant cells comprising one or more expressed nucleic acids encoding monophosphate decarboxylase and/or isopentenyl kinase polypeptides as disclosed herein and further engineered to decrease the activity of a fructose 6-phosphate isozyme.

### Pathways Involving Pyruvate Dehydrogenase Complex

The pyruvate dehydrogenase complex, which catalyzes the decarboxylation of pyruvate into acetyl-CoA, is composed of the proteins encoded by the genes aceE, aceF and lpdA. Transcription of those genes is regulated by several regulators. Thus, one of skill in the art can increase acetyl-CoA by modulating the activity of the pyruvate dehydrogenase complex. Modulation can be to increase the activity and/or expression (e.g., constant expression) of the pyruvate dehydrogenase complex. This can be accomplished by different ways, for example, by placing a strong constitutive promoter, like PL.6 (aattcatataaaaaacatacagataaccatctgcggtgataaattatctctggcggtgttgacataaataccactggcggtgatactgagcaca tcagcaggacgcactgaccaccatgaaggtg—lambda promoter, GenBank NC_{—}001416), in front of the operon or using one or more synthetic constitutively expressing promoters.

Accordingly, in one aspect, the activity of pyruvate dehydrogenase is modulated by increasing the activity of one or more enzymes of the pyruvate dehydrogenase complex consisting of (a) pyruvate dehydrogenase (E1), (b) dihydrolipoyl transacetylase, and (c) dihydrolipoyl dehydrogenase. It is understood that any one, two or three of the genes encoding these enzymes can be manipulated for increasing activity of pyruvate dehydrogenase. In another aspect, the activity of the pyruvate dehydrogenase complex can be modulated by attenuating the activity of an endogenous pyruvate dehydrogenase complex repressor, further detailed below. The activity of an endogenous pyruvate dehydrogenase complex repressor can be attenuated by deletion of the endogenous pyruvate dehydrogenase complex repressor gene.

In some cases, one or more genes encoding the pyruvate dehydrogenase complex are endogenous genes. Another way to increase the activity of the pyruvate dehydrogenase complex is by introducing into the cell one or more heterologous nucleic acids encoding one or more polypeptides from the group consisting of (a) pyruvate dehydrogenase (E1), (b) dihydrolipoyl transacetylase, and (c) dihydrolipoyl dehydrogenase.

By using any of these methods, the recombinant cells can produce increased amounts of acetyl Co-A in comparison to cells wherein the activity of pyruvate dehydrogenase is not modulated. Modulating the activity of pyruvate dehydrogenase can result in more carbon flux into the mevalonate dependent biosynthetic pathway in comparison to cells that do not have modulated pyruvate dehydrogenase expression.

### Pathways Involving the Phosphotransferase System

The phosphoenolpyruvate dependent phosphotransferase system (PTS) is a multicomponent system that simultaneously transports and phosphorylates its carbohydrate substrates across a membrane in a process that is dependent on energy provided by the glycolytic intermediate phosphoenolpyruvate (PEP). The genes that regulate the PTS are mostly clustered in operons. For example, the pts operon (ptsHIcrr) of Escherichia coli is composed of the ptsH, ptsI and crr genes coding for three proteins central to the phosphoenolpyruvate dependent phosphotransferase system (PTS), the HPr (ptsH), enzyme I (ptsI) and EIIIGlc (crr) proteins. These three genes are organized in a complex operon in which the major part of expression of the distal gene, crr, is initiated from a promoter region within ptsI. In addition to the genes of the pts operon, ptsG encodes the glucose-specific transporter of the phosphotransferase system, ptsG Transcription from this promoter region is under the positive control of catabolite activator protein (CAP)-cyclic AMP (cAMP) and is enhanced during growth in the presence of glucose (a PTS substrate). Furthermore, the ppsA gene encodes for phosphoenolpyruvate synthetase for the production of phosphoenolpyruvate (PEP) which is required for activity of the phosphotransferase system (PTS). Carbon flux is directed by the phosphoenolpyruvate synthetase through the pyruvate dehydrogenase pathway or the PTS pathway. See Postma, P. W., et al., Microbiol Rev. (1993), 57(3):543-94) which is incorporated herein by reference in its entirety.

In certain embodiments described herein, the down regulation (e.g. attenuation) of the pts operon can enhance acetate utilization by the host cells. The down regulation of PTS operon activity can be any amount of reduction of specific activity or total activity as compared to when no manipulation has been effectuated. In some instances, the decrease of activity of the complex is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%. In certain embodiments, attenuation is achieved by deleting the pts operon. In some aspects, the activity of the PTS system is modulated by decreasing the activity of an endogenous pts operon. This can be accomplished by replacing the endogenous promoter(s) within the pts operon with synthetic constitutively low expressing promoter(s). In any aspects of the invention, provided herein are recombinant cells comprising one or more expressed nucleic acids encoding monophosphate decarboxylase and/or isopentenyl kinase polypeptides as disclosed herein and further engineered to decrease the activity of the pts operon. In any aspects of the invention, provided herein are recombinant cells comprising one or more expressed nucleic acids encoding monophosphate decarboxylase and/or isopentenyl kinase polypeptides as disclosed herein and further engineered to decrease the activity of EI (ptsI). In any aspects of the invention, provided herein are recombinant cells comprising one or more expressed nucleic acids encoding monophosphate decarboxylase and/or isopentenyl kinase polypeptides as disclosed herein and further engineered to decrease the activity of EIICB^{Glc} (ptsG). In any aspects of the invention, provided herein are recombinant cells comprising one or more expressed nucleic acids encoding monophosphate decarboxylase and/or isopentenyl kinase polypeptides as disclosed herein and further engineered to decrease the activity of EIIA^{Glc} (crr). In any aspects of the invention, provided herein are recombinant cells comprising one or more expressed nucleic acids encoding monophosphate decarboxylase and/or isopentenyl kinase polypeptides as disclosed herein and further engineered to decrease the activity of HPr (ptsH). To decrease carbon loss through pyruvate dehydrogenase while increasing the PEP pool for glucose uptake, the activity of phosphoenolpyruvate synthetase (ppsA) can be increased. In any aspects of the invention, provided herein are recombinant cells comprising one or more expressed nucleic acids encoding monophosphate decarboxylase and/or isopentenyl kinase polypeptides as disclosed herein and further engineered to increase the activity of phosphoenolpyruvate synthetase (ppsA). In any further aspect of the invention, the PTS is downregulated and a glucose transport pathway is upregulated. A glucose transport pathway includes, but is not limited to, galactose (galP) and glucokinase (glk). In some embodiments, the pts operon is downregulated, the galactose (galP) gene is upregulated, and the glucokinase (glk) gene is upregulated. Activity modulation (e.g., decreased) of isozymes of the PTS is also contemplated herein. In any aspects of the invention, provided herein are recombinant cells comprising one or more expressed nucleic acids encoding monophosphate decarboxylase and/or isopentenyl kinase polypeptides as disclosed herein and further engineered to decrease the activity of PTS isozymes.

### Pathways Involving Xylose Utilization

In certain aspects, recombinant cells comprising one or more expressed nucleic acids encoding monophosphate decarboxylase and/or isopentenyl kinase polypeptides as disclosed herein further comprise one more nucleic acids encoding a phosphoketolase polypeptide. In certain embodiments described herein, the utilization of xylose is desirable to convert sugar derived from plant biomass into desired products, such as mevalonate, such as isoprenoid precursors, isoprene and/or isoprenoids. In some organisms, xylose utilization requires use of the pentose phosphate pathway for conversion to fructose-6-phosphate for metabolism. Organisms can be engineered for enhanced xylose utilization, either by deactivating the catabolite repression by glucose, or by heterologous expression of genes from the xylose operon found in other organisms. The xylulose pathway can be engineered as described below to enhance production of mevalonate, isoprenoid precursors, isoprene and/or isoprenoids via the phosphoketolase pathway.

Enhancement of xylose uptake and conversion to xylulose-5-phosphate followed by direct entry into the phosphoketolase pathway would be a benefit. Without being bound by theory, this allows the carbon flux to bypass the pentose phosphate pathway (although some glyceraldehyde-3-phosphate may be cycled into PPP as needed). Enhanced expression of xyulokinase can be used to increase the overall production of xylulose-5-phosphate. Optimization of xyulokinase expression and activity can be used to enhance xylose utilization in a strain with a phosphoketolase pathway. The desired xyulokinase may be either the endogeneous host's enzyme, or any heterologous xyulokinase compatible with the host. In one embodiment, other components of the xylose operon can be overexpressed for increased benefit (e.g., xylose isomerase). In another embodiment, other xylose pathway enzymes (e.g. xylose reductase) may need to be attenuated (e.g., reduced or deleted activity).

Accordingly, the host cells engineered to have phosphoketolase enzymes as described herein can be further engineered to overexpress xylulose isomerase and/or xyulokinase, either the endogenous forms or heterologous forms, to improve overall yield and productivity of mevalonate, isoprenoid precursors, isoprene and/or isoprenoids via the alternative lower MVA pathway (e.g., MVK, PMevDC, IPK, and/or IDI).

### Pathways Involving Transaldolase and Transketolase Enzymes of Pentose Phosphate Pathway

In certain aspects, recombinant cells comprising one or more expressed nucleic acids encoding monophosphate decarboxylase and/or isopentenyl kinase polypeptides as disclosed herein further comprise one more nucleic acids encoding a phosphoketolase polypeptide. Some microorganisms capable of anaerobic or heterofermentative growth incorporate a phosphoketolase pathway instead of or in addition to a glycolytic pathway. This pathway depends on the activity of the pentose phosphate pathway enzymes transaldolase and transketolase. Accordingly, the host cells engineered to have phosphoketolase enzymes as described herein can be further engineered to overexpress a transketolase and transaldolase, either the endogeneous forms or heterologous forms, to improve pathway flux, decrease the levels of potentially toxic intermediates, reduce the diversion of intermediates to non-productive pathways, and improve the overall yield and productivity of mevalonate, isoprenoid precursors, isoprene and/or isoprenoids via the alternative lower MVA pathway (e.g., MVK, PMevDC, IPK, and/or IDI).

### Combinations of Mutations

It is understood that for any of the enzymes and/or enzyme pathways described herein, molecular manipulations that modulate any combination (two, three, four, five, six, seven, eight, nine, ten, eleven, twelve, thirteen, or fourteen) of the enzymes and/or enzyme pathways described herein is expressly contemplated. For ease of the recitation of the combinations, citrate synthase (gltA) is designated as A, phosphotransacetylase (pta) is designated as B, acetate kinase (ackA) is designated as C, lactate dehydrogenase (ldhA) is designated as D, glyceraldehyde 3-phosphate dehydrogenase (gap) is designated as E, and pyruvate decarboxylase (aceE, aceF, and/or lpdA) is designated as F, phosphogluconate dehydratase (edd) is designated as G, 2-keto-3-deoxygluconate 6-phosphate aldolase (eda) is designated as H phosphofructokinase is designated as I, transaldolase is designated as J, transketolase is designated as K, ribulose-5-phosphate-epimerase is designated as L, ribose-5-phosphate epimerase is designated as M, xylukinase is designated as N, xylose isomerase is designated as O, and xylitol reductase is designated as P, ribose-5-phosphate isomerase (rpi) is designated as Q, D-ribulose-5-phosphate 3-epimerase (rpe) is designated as R, phosphoenolpyruvate synthetase (pps) is designated as S, fructose bisphosphate aldolase (fba) is designated as T, EI (ptsI) is designated as U, EIICB^{Glc} (ptsG) is designated as V, EIIA^{Glc} (crr) is designated as W, HPr (ptsH) is designated as X, galactose (galP) is designated as Y, glucokinase (glk) is designated as Z, glucose-6-phosphate dehydrogenase (zwf) is designated as AA. As discussed above, aceE, aceF, and/or lpdA enzymes of the pyruvate decarboxylase complex can be used singly, or two of three enzymes, or three of three enzymes for increasing pyruvate decarboxylase activity. Thus, any and all combination of enzymes designated as A-M herein is expressly contemplated as well as any and all combination of enzymes designated as A-AA. Furthermore, any combination described above can be used in combination with any of the enzymes and/or enzyme pathways described herein (e.g., phosphomevalonate decarboxylase, isopentenyl kinase, phosphoketolase, MVA pathway polypeptides, IDI, isoprene synthase, DXP pathway polypeptides).

### Other Regulators and Factors for Increased Production

Other molecular manipulations can be used to increase the flow of carbon towards mevalonate production. One method is to reduce, decrease or eliminate the effects of negative regulators for pathways that feed into the mevalonate pathway. For example, in some cases, the genes aceEF-lpdA are in an operon, with a fourth gene upstream pdhR. The gene pdhR is a negative regulator of the transcription of its operon. In the absence of pyruvate, it binds its target promoter and represses transcription. It also regulates ndh and cyoABCD in the same way (Ogasawara, H. et al. 2007. J. Bact. 189:5534-5541). In one aspect, deletion of pdhR regulator can improve the supply of pyruvate, and hence the production of mevalonate, isoprenoid precursors, isoprene, and isoprenoids via the alternative lower MVA pathway (e.g., MVK, PMevDC, IPK, and/or IDI).

In other embodiments, any of the resultant strains described above can be further engineered to modulate the activity of the Entner-Doudoroff pathway. The gene coding for phosphogluconate dehydratase or aldolase can be attenuated or deleted. In other embodiments, any of the resultant strains described above may also be engineered to decrease or remove the activity of acetate kinase or citrate synthase. In other embodiments, any of the strains the resultant strain may also be engineered to decrease or remove the activity of phosphofructokinase. In other embodiments, any of the resultant strains described above may also be engineered to modulate the activity of glyceraldehyde-3-phosphate dehydrogenase. The activity of glyceraldehyde-3-phosphate dehydrogenase can be modulated by decreasing its activity. In other embodiments, the enzymes from the non-oxidative branch of the pentose phosphate pathway, such as transketolase, transaldolase, ribulose-5-phosphate-epimerase and (or) ribose-5-phosphate epimerase can be overexpressed.

In other aspects, the host cells can be further engineered to increase intracellular acetyl-phosphate concentrations by introducing heterologous nucleic acids encoding sedoheptulose-1,7-bisphosphatase/fructose-1,6-bisphosphate aldolase and sedoheptulose-1,7-bisphosphatase/fructose-1,6-bisphosphate phosphatase. In certain embodiments, the host cells having these molecular manipulations can be combined with attenuated or deleted transaldolase (talB) and phosphofructokinase (pfkA and/or pfkB) genes, thereby allowing faster conversion of erythrose 4-phosphate, dihydroxyacetone phosphate, and glyceraldehyde 3-phosphate into sedoheptulose 7-phosphate and fructose 1-phosphate.

In other aspects, the introduction of 6-phosphogluconolactonase (PGL) into cells (such as various E. coli strains) which lack PGL can be used to improve production of mevalonate, isoprenoid precursors, isoprene, and isoprenoids via the alternative lower MVA pathway (e.g., MVK, PMevDC, IPK, and/or IDI). PGL may be introduced by introduction of the encoding gene using chromosomal integration or extra-chromosomal vehicles, such as plasmids.

In addition to the host cell (e.g., bacterial host cell) mutations for modulating various enzymatic pathways described herein that increases carbon flux towards mevalonate production, the host cells described herein comprise genes encoding phosphomevalonate decarboxylase, isopentenyl kinase as well as other enzymes from the MVA pathway, including but not limited to, the mvaE and mvaS gene products. Non-limiting examples of MVA pathway polypeptides include acetyl-CoA acetyltransferase (AA-CoA thiolase) polypeptides, 3-hydroxy-3-methylglutaryl-CoA synthase (HMG-CoA synthase) polypeptides, 3-hydroxy-3-methylglutaryl-CoA reductase (HMG-CoA reductase) polypeptides, mevalonate kinase (MVK) polypeptides, phosphomevalonate kinase (PMK) polypeptides, diphosphomevalonte decarboxylase (MVD) polypeptides, phosphomevalonate decarboxylase (PMDC) polypeptides, isopentenyl phosphate kinase (IPK) polypeptides, IDI polypeptides, and polypeptides (e.g., fusion polypeptides) having an activity of two or more MVA pathway polypeptides. MVA pathway polypeptides can include polypeptides, fragments of polypeptides, peptides, and fusions polypeptides that have at least one activity of an MVA pathway polypeptide. Exemplary MVA pathway nucleic acids include nucleic acids that encode a polypeptide, fragment of a polypeptide, peptide, or fusion polypeptide that has at least one activity of an MVA pathway polypeptide. Exemplary MVA pathway polypeptides and nucleic acids include naturally-occurring polypeptides and nucleic acids from any of the source organisms described herein. In some aspects, the host cell further comprises genes encoding a phosphoketolase.

Non-limiting examples of MVA pathway polypeptides which can be used are described in International Patent Application Publication No. WO2009/076676; WO2010/003007 and WO2010/148150

### Exemplary Cell Culture Media

As used herein, the terms “minimal medium” or “minimal media” refer to growth media containing the minimum nutrients possible for cell growth, generally, but not always, without the presence of one or more amino acids (e.g., 1, 2, 3, 4, 5, 6, 7, 8, 9, 10, or more amino acids). Minimal medium typically contains: (1) a carbon source for host cell (e.g., bacterial cell) growth; (2) various salts, which can vary among host cell species and growing conditions; and (3) water. The carbon source can vary significantly, from simple sugars like glucose to more complex hydrolysates of other biomass, such as yeast extract, as discussed in more detail below. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

Any minimal medium formulation can be used to cultivate the host cells. Exemplary minimal medium formulations include, for example, M9 minimal medium and TM3 minimal medium. Each liter of M9 minimal medium contains (1) 200 ml sterile M9 salts (64 g Na₂HPO₄-7H₂O, 15 g KH₂PO₄, 2.5 g NaCl, and 5.0 g NH₄Cl per liter); (2) 2 ml of 1 M MgSO₄ (sterile); (3) 20 ml of 20% (w/v) glucose (or other carbon source); and (4) 100 μl of 1 M CaCl₂ (sterile). Each liter of TM3 minimal medium contains (1) 13.6 g K₂HPO₄; (2) 13.6 g KH₂PO₄; (3) 2 g MgSO₄*7H₂O; (4) 2 g Citric Acid Monohydrate; (5) 0.3 g Ferric Ammonium Citrate; (6) 3.2 g (NH₄)₂SO₄; (7) 0.2 g yeast extract; and (8) 1 ml of 1000X Trace Elements solution; pH is adjusted to ˜6.8 and the solution is filter sterilized. Each liter of 1000X Trace Elements contains: (1) 40 g Citric Acid Monohydrate; (2) 30 g MnSO₄*H₂O; (3) 10 g NaCl; (4) 1 g FeSO₄*7H₂O; (4) 1 g CoCl₂*6H₂O; (5) 1 g ZnSO₄*7H₂O; (6) 100 mg CuSO₄*5H₂O; (7) 100 mg H₃BO₃; and (8) 100 mg NaMoO₄*2H₂O; pH is adjusted to ˜3.0.

An additional exemplary minimal media includes (1) potassium phosphate K₂HPO₄, (2) Magnesium Sulfate MgSO₄*7H₂O, (3) citric acid monohydrate C₆H₈O₇*H₂O, (4) ferric ammonium citrate NH₄FeC₆H₅O₇, (5) yeast extract (from biospringer), (6) 1000X Modified Trace Metal Solution, (7) sulfuric acid 50% w/v, (8) foamblast 882 (Emerald Performance Materials), and (9) Macro Salts Solution 3.36 ml. All of the components are added together and dissolved in deionized H₂O and then heat sterilized. Following cooling to room temperature, the pH is adjusted to 7.0 with ammonium hydroxide (28%) and q.s. to volume. Vitamin Solution and spectinomycin are added after sterilization and pH adjustment.

Any carbon source can be used to cultivate the host cells. The term “carbon source” refers to one or more carbon-containing compounds capable of being metabolized by a host cell or organism. For example, the cell medium used to cultivate the host cells can include any carbon source suitable for maintaining the viability or growing the host cells. In some aspects, the carbon source is a carbohydrate (such as monosaccharide, disaccharide, oligosaccharide, or polysaccharides), or invert sugar (e.g., enzymatically treated sucrose syrup).

In some aspects, the carbon source includes yeast extract or one or more components of yeast extract. In some aspects, the concentration of yeast extract is 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. In some aspects, the carbon source includes both yeast extract (or one or more components thereof) and another carbon source, such as glucose.

Exemplary monosaccharides include glucose and fructose; exemplary oligosaccharides include lactose and sucrose, and exemplary polysaccharides include starch and cellulose. Exemplary carbohydrates include C6 sugars (e.g., fructose, mannose, galactose, or glucose) and C5 sugars (e.g., xylose or arabinose).

In some aspects, the cells described herein are capable of using syngas as a source of energy and/or carbon. In some embodiments, the syngas includes at least carbon monoxide and hydrogen. In some embodiments, the syngas further additionally includes one or more of carbon dioxide, water, or nitrogen. In some embodiments, the molar ratio of hydrogen to carbon monoxide in the syngas is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, or 10.0. In some embodiments, the syngas comprises 10, 20, 30, 40, 50, 60, 70, 80, or 90% by volume carbon monoxide. In some embodiments, the syngas comprises 10, 20, 30, 40, 50, 60, 70, 80, or 90% by volume hydrogen. In some embodiments, the syngas comprises 10, 20, 30, 40, 50, 60, 70, 80, or 90% by volume carbon dioxide. In some embodiments, the syngas comprises 10, 20, 30, 40, 50, 60, 70, 80, or 90% by volume water. In some embodiments, the syngas comprises 10, 20, 30, 40, 50, 60, 70, 80, or 90% by volume nitrogen.

Synthesis gas may be derived from natural or synthetic sources. The source from which the syngas is derived is referred to as a “feedstock.” In some embodiments, the syngas is derived from biomass (e.g., wood, switch grass, agriculture waste, municipal waste) or carbohydrates (e.g., sugars). In other embodiments, the syngas is derived from coal, petroleum, kerogen, tar sands, oil shale, or natural gas. In other embodiments, the syngas is derived from rubber, such as from rubber tires.

Syngas can be derived from a feedstock by a variety of processes, including methane reforming, coal liquefaction, co-firing, fermentative reactions, enzymatic reactions, and biomass gasification. Biomass gasification is accomplished by subjecting biomass to partial oxidation in a reactor at temperatures above about 700° C. in the presence of less than a stoichiometric amount of oxygen. The oxygen is introduced into the bioreactor in the form of air, pure oxygen, or steam. Gasification can occur in three main steps: 1) initial heating to dry out any moisture embedded in the biomass; 2) pyrolysis, in which the biomass is heated to 300-500° C. in the absence of oxidizing agents to yield gas, tars, oils and solid char residue; and 3) gasification of solid char, tars and gas to yield the primary components of syngas. Co-firing is accomplished by gasification of a coal/biomass mixture. The composition of the syngas, such as the identity and molar ratios of the components of the syngas, can vary depending on the feedstock from which it is derived and the method by which the feedstock is converted to syngas.

Synthesis gas can contain impurities, the nature and amount of which vary according to both the feedstock and the process used in production. Fermentations may be tolerant to some impurities, but there remains the need to remove from the syngas materials such as tars and particulates that might foul the fermentor and associated equipment. It is also advisable to remove compounds that might contaminate the isoprene product such as volatile organic compounds, acid gases, methane, benzene, toluene, ethylbenzene, xylenes, H₂S, COS, CS₂, HCl, O₃, organosulfur compounds, ammonia, nitrogen oxides, nitrogen-containing organic compounds, and heavy metal vapors. Removal of impurities from syngas can be achieved by one of several means, including gas scrubbing, treatment with solid-phase adsorbents, and purification using gas-permeable membranes.

### Exemplary Cell Culture Conditions

Materials and methods suitable for the maintenance and growth of the recombinant cells of the invention are described infra, e.g., in the Examples section. Other materials and methods suitable for the maintenance and growth of cell cultures are well known in the art. Exemplary techniques can be found in International Publication No. WO 2009/076676, U.S. Patent Publ. No. 2009/0203102, WO 2010/003007, US Publ. No. 2010/0048964, WO 2009/132220, US Publ. No. 2010/0003716, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. In some aspects, the cells are cultured in a culture medium under conditions permitting the expression of phosphomevalonate decarboxylase polypeptide, isopentenyl kinase polypeptide, as well as other enzymes from the upper and lower MVA pathway, including but not limited to, the mvaE and mvaS gene products, isoprene synthase, DXP pathway (e.g., DXS), IDI, or PGL polypeptides encoded by a nucleic acid inserted into the host cells.

Standard cell culture conditions can be used to culture the cells (see, for example, WO 2004/033646 and references cited therein). In some aspects, cells are grown and maintained at an appropriate temperature, gas mixture, and pH (such as at about 20° C. to about 37° C., at about 6% to about 84% CO₂, and at a pH between about 5 to about 9). In some aspects, cells are grown at 35° C. in an appropriate cell medium. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the host cells. In addition, more specific cell culture conditions can be used to culture the cells. For example, in some embodiments, the recombinant cells (such as E. coli cells) comprise one or more heterologous nucleic acids encoding a phosphomevalonate decarboxylase polypeptide, isopentenyl kinase polypeptide as well as enzymes from the upper, including but not limited to, the mvaE and mvaS gene products mvaE and mvaS polypeptides from L. grayi, E. faecium, E. gallinarum, E. casseliflavus and/or E. faecalis under the control of a strong promoter in a low to medium copy plasmid and are cultured at 34° C.

Standard culture conditions and modes of fermentation, such as batch, fed-batch, or continuous fermentation that can be used are described in International Publication No. WO 2009/076676, U.S. Patent Publ. No. 2009/0203102, WO 2010/003007, US Publ. No. 2010/0048964, WO 2009/132220, US Publ. No. 2010/0003716. Batch and Fed-Batch fermentations are common and well known in the art and examples can be found in Brock, Biotechnology: A Textbook of Industrial Microbiology, Second Edition (1989) Sinauer Associates, Inc.

In some aspects, the cells are cultured under limited glucose conditions. By “limited glucose conditions” is meant that the amount of glucose that is added is less than or about 105% (such as about 100%, 90%, 80%, 70%, 60%, 50%, 40%, 30%, 20%, or 10%) of the amount of glucose that is consumed by the cells. In particular aspects, the amount of glucose that is added to the culture medium is approximately the same as the amount of glucose that is consumed by the cells during a specific period of time. In some aspects, the rate of cell growth is controlled by limiting the amount of added glucose such that the cells grow at the rate that can be supported by the amount of glucose in the cell medium. In some aspects, glucose does not accumulate during the time the cells are cultured. In various aspects, the cells are cultured under limited glucose conditions for greater than or about 1, 2, 3, 5, 10, 15, 20, 25, 30, 35, 40, 50, 60, or 70 hours. In various aspects, the cells are cultured under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions can allow more favorable regulation of the cells.

In some aspects, the recombinant cells are grown in batch culture. The recombinant cells can also be grown in fed-batch culture or in continuous culture. Additionally, the recombinant cells can be cultured in minimal medium, including, but not limited to, any of the minimal media described above. The minimal medium can be further supplemented with 1.0% (w/v) glucose, or any other six carbon sugar, or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

### Exemplary Purification Methods

In some aspects, any of the methods described herein further include a step of recovering the compounds produced (e.g., isoprene, isoprenoid precursors, or isoprenoids). In some aspects, any of the methods described herein further include a step of recovering the isoprene. For example, the isoprene produced using the compositions and methods of the invention can be recovered using standard techniques, such as gas stripping, membrane enhanced separation, fractionation, adsorption/desorption, pervaporation, thermal or vacuum desorption of isoprene from a solid phase, or extraction of isoprene immobilized or absorbed to a solid phase with a solvent (see, for example, U.S. Pat. Nos. 4,703,007 and 4,570,029, which are each hereby incorporated by reference in their entireties, particularly with respect to isoprene recovery and purification methods). In one aspect, the isoprene is recovered by absorption stripping (see, e.g., US Pub. No. 2011/0178261). In particular aspects, extractive distillation with an alcohol (such as ethanol, methanol, propanol, or a combination thereof) is used to recover the isoprene. In some aspects, the recovery of isoprene involves the isolation of isoprene in a liquid form (such as a neat solution of isoprene or a solution of isoprene in a solvent). Gas stripping involves the removal of isoprene vapor from the fermentation off-gas stream in a continuous manner. Such removal can be achieved in several different ways including, but not limited to, adsorption to a solid phase, partition into a liquid phase, or direct condensation (such as condensation due to exposure to a condensation coil or do to an increase in pressure). In some aspects, membrane enrichment of a dilute isoprene vapor stream above the dew point of the vapor resulting in the condensation of liquid isoprene. In some aspects, the isoprene is compressed and condensed.

The recovery of isoprene may involve one step or multiple steps. In some aspects, the removal of isoprene vapor from the fermentation off-gas and the conversion of isoprene to a liquid phase are performed simultaneously. For example, isoprene can be directly condensed from the off-gas stream to form a liquid. In some aspects, the removal of isoprene vapor from the fermentation off-gas and the conversion of isoprene to a liquid phase are performed sequentially. For example, isoprene may be adsorbed to a solid phase and then extracted from the solid phase with a solvent. In one aspect, the isoprene is recovered by using absorption stripping as described in U.S. application Ser. No. 12/969,440 (US Publ. No. 2011/0178261).

In some aspects, any of the methods described herein further include purifying the isoprene. For example, the isoprene produced using the compositions and methods of the invention can be purified using standard techniques. Purification refers to a process through which isoprene is separated from one or more components that are present when the isoprene is produced. In some aspects, the isoprene is obtained as a substantially pure liquid. Examples of purification methods include (i) distillation from a solution in a liquid extractant and (ii) chromatography. As used herein, “purified isoprene” means isoprene that has been separated from one or more components that are present when the isoprene is produced. In some aspects, the isoprene is at least about 20%, by weight, free from other components that are present when the isoprene is produced. In various aspects, the isoprene is at least or about 25%, 30%, 40%, 50%, 60%, 70%, 75%, 80%, 90%, 95%, or 99%, by weight, pure. Purity can be assayed by any appropriate method, e.g., by column chromatography, HPLC analysis, or GC-MS analysis. Suitable purification methods are described in more detail in U.S. Patent Application Publication US2010/0196977 A1.

In some aspects, at least a portion of the gas phase remaining after one or more recovery steps for the removal of isoprene is recycled by introducing the gas phase into a cell culture system (such as a fermentor) for the production of isoprene.

In some aspects, any of the methods described herein further include a step of recovering the isoprenoid precursor or isoprenoid.

In some aspects, any of the methods described herein further include a step of recovering the heterologous nucleic acid. In some aspects, any of the methods described herein further include a step of recovering the heterologous polypeptide.

The invention will be more fully understood by reference to the following examples. They should not, however, be construed as limiting the scope of the invention. All citations throughout the disclosure are hereby expressly incorporated by reference.

### EXAMPLES

### Example 1

### In Vitro and In Vivo Testing of Candidate Archaeal Isopentenyl Kinases (IPK) and Phosphomevalonate Decarboxylases (PMevDC)

Isopentenyl kinases (IPKs) are readily found in archaeal genome but only very limited information and no direct biochemical evidence is available for archaeal candidate genes encoding polypeptides with phosphomevalonate decarboxylase (PMevDC) activity. Based on a comparative genome analysis focusing on MVA-pathway clusters and in combination with archaeal genome analyses, IPK and PMevDC candidate genes from Methanocaldococcus jannaschii and Methanobrevibacter ruminantium were each tested for the ability to establish a functional archaeal lower MVA pathway in E. coli. See Grochowski et al., J. Bacteriol., 188(9):3192-8, (2006) and Matsumi et al., Res Microbiol., 162(1)39-52, (2011).

The two IPKs from M. jannaschii and Mbb. ruminantium were amplified from chromosomal DNA and cloned into pET-expression vectors. The pET-expression vectors encoding the IPKs were transformed into a T7-expression system established in E. coli BL21. SDS-PAGE analyses of cellular lysates isolated from the transformed bacteria demonstrated strong expression of the proteins encoded by the cloned genes. Furthermore, solubility of the proteins was at least 50% or higher. When crude extracts of induced cells were tested for in vitro IPK activity by GC/MS or LC/MS analyses, only trace amounts of isopentenyl pyrophosphate (IPP) were formed from isopentenyl phosphate (IP). The IPP signal was minimally above background, and significant substrate consumption could not be demonstrated, even when ¹³C-labelling experiments were conducted.

For in vivo IPK activity analysis, IP conversion was tested in E. coli strain MCM724 DispG DispH which expressed the classical lower MVA-pathway and was transformed with a plasmid expressing the IPK-gene from either M. jannaschii or Mbb. ruminantium. In the absence of exogenous MVA, the strain could not grow, while addition of 500 μM of MVA fully restored growth. Addition of 600 μM of IP to the medium also allowed for unrestricted growth (“IP-rescue”) clearly demonstrating in vivo activity of the cloned IPK genes from both methanogens. The finding that recombinant E. coli cells expressing a methanogen IPK could grow when supplemented with IP allowed for the construction of a host for in vivo activity based screening.

Similar to cloning of the IPK genes, the candidate PMevDCs from M. jannaschii and Mbb. ruminantium were amplified from chromosomal DNA and cloned into pET-expression vectors. The pET-expression vectors encoding the candidate PMevDCs were transformed into a T7-expression system established in E. coli BL21. Preliminary analysis of cellular lysates from the bacteria demonstrated that the solubility of the candidate PMevDCs was 50% or less. Therefore, solubility enhancing factors were fused to proteins and subsequent solubility analysis demonstrated that at least 50% of the synthesized protein was found in the soluble fraction after expression in the E. coli T7-system. Corresponding cell-free extracts containing native and fusion proteins of the candidate PMevDC were generated and tested for in vitro PMevDC activity. Generation of extracts was done in different set-ups, including addition of low molecular weight fractions (<3 kDa filtrate) obtained from Methanothermobacter thermoautotrophicus cell extracts to complement for small molecules present in Archaea but absent in E. coli. An additional set-up comprised anoxic cultivation and expression of E. coli cells in combination with down-stream processing under strictly anoxic conditions including enzyme assays. In the systems tested, no in vitro PMevDC activity was demonstrated for the native or fusion proteins. Cell free extracts of M. thermoautotrophicus treated identically under strictly anoxic conditions yielded extracts showed activity on the substrates MVA and mevalonate phosphate, but not on IP.

Similar IP-rescue experiments conducted for in vivo testing of IPK activity were conducted for in vivo testing of PMevDC activity. Dual constructs encoding PMevDC together with IPK of the same donor organism, M. jannaschii or Mbb. ruminantium, were generated. When expressed in E. coli strain MCM724 DispG DispH, the constructs were shown to express proteins with IPK-activity, since addition of IP resulted in cell growth. However, when mevalonate phosphate instead of IP was supplemented to the medium, growth was not sustained. Supplementation of MVA, however, restored growth. Thus, it was concluded that the PMevDC candidate genes from these methanogens could not be expressed in active form in this strain of E. coli, that another protein was missing, or that the cloned candidate genes did not encode the PMevDC-activity under investigation.

### Example 2

### Activity-Based Screening of Candidate Phosphomevalonate Decarboxylases (PMevDC)

When experiments with IPKs from methanogens demonstrated in vivo activity of the corresponding genes in E. coli, this finding was used to design a host that was eventually utilized for activity based screening. A fundamental requirement of activity based screening for a novel lower MVA pathway was the absence of alternative pathways or activities that synthesize DMAPP. Fosmidomycin-induced silencing of the endogenous DXP-pathway of standard E. coli strains was shown to be inappropriate for screening purposes. Hence, a stable genetic inactivation of the DXP pathway was pursued. To inactivate the DXP pathway, an E. coli strain MCM724 was produced with inactivated for genes encoding HMBPP synthase (ispG) and HMPPP reductase (ispH). This double mutant E. coli strain could not be complemented by small insert metagenomic libraries, an important finding that allowed its use as the basis for a host that was utilized for screening from metagenomic resources. To generate the screening host, strain MCM724 was used to express the chromosomally encoded synthetic classical lower MVA pathway under control of a strong constitutive promoter. The synthetic classical lower MVA pathway comprised mevalonate-kinase (MVK), phosphomevlonate kinase (PMK), diphosphomevalonate decarboxylase (MVD) and ispopentenyldiphosphate isomerase (IDI). In order to enable screening for enzymes constituting a novel alternative lower MVA pathway, specific enzymes in the synthetic lower MVA pathway had to be inactivated while sustaining growth at the same time. Inactivation of the classical lower MVA pathway was done by the replacement of PMK and MVD with a gene-cassette encoding IPK from M. jannaschii and a chloramphenicol resistance marker. Introduction of the new genes did not affect the expression of MVK and IDI. Furthermore, since MCM724 was previously inactivated for genes ispG and ispH, this screening host was an MVA auxotrophic mutant. The resulting strain did not grow in presence or absence of MVA and could grow only in the presence of 600 μM IP supplemented to the LB-medium (“IP rescue”). This screening host was termed V. 05. When V. 05 additionally harbored a plasmid encoding the PMevDC candidate gene from M. jannaschii, it was termed V.06.

For activity based screening, biomass or chromosomal DNA from different archaeal species comprising methanogens, halobacteria, Sulfolobales and environmental samples enriched in archaeal prokaryotes were tested. Chromosomal libraries were constructed in vector pUR2 that employs an antitermination design to ensure transcription of long inserts. Libraries were constructed with average insert sizes between 6-8 kbp from genomic DNA of M. jannaschii and Mbb. ruminantium. An additional library of pooled genomic DNA from three different Sulfolobus strains was generated. Together, the libraries encoded all types of candidate PMevDC genes such as COG 1355, 1586 and 3407. See Matsumi et al., Res Microbiol., 162(1)39-52, (2011). The quality of each library guaranteed at least 2× coverage of the respective genome at p=0.99. Screening was done with screening hosts V.05 and V.06. Preliminary screening of the genomic archaeal libraries did not yield any clones, therefore metagenomic libraries were constructed from soil samples and used for further screening. Utilization of host V.05 for screening of the metagenomic libraries resulted in the isolation of three colonies that grew in the presence of 500 μM MVA. Insert analysis of the three clones demonstrated redundancy, and one clone was chosen for further experiments. This respective clone was termed S378Pa3-2.

Plasmid DNA from clone S378Pa3-2 was isolated and retransformed into screening host V. 05 in three independent trials, each time with success (i.e. sustained growth in the presence of MVA). Retransformed clones grew unrestricted in presence of otherwise inhibitory concentrations of Fosmidomycin (32 μg/ml) when 500 μM MVA was supplied, demonstrating that complementation was done via the MVA pathway and not via the DXP-pathway. The isolated plasmid encoded metagenomic DNA of 4.6 kbp. Four coding sequences (cds) were identified in the metagenomic DNA insert, and each cds was subcloned into expression vector pTrcHis2b. The individual vectors were transformed back into screening host V.05. Only the vector encoding cds#2 was able to complement the lower MVA pathway in the screening host V. 05.

Bioinformatic analyses of the protein encoded by cds#2 revealed limited similarities to a gene found in a bacterium belonging to the Chloroflexus group within the bacterial kingdom. The protein encoded by the newly isolated gene showed only a 46% amino acid sequence identity to a coding sequence of Herpetosiphon aurantiacus in the National Center for Biotechnology Non-Redundant (NR) database available at the worldwide web blast.ncbi.nlm.nih.gov/Blast.cgi. This H. aurantiacus was annotated as a putative DPMevDC. No homolog genes of the S378Pa3-2 sequence were detected in the genomes of methanogens or M. jannaschii. Sequence comparisons also revealed distinct positioning from clusters of decarboxylases belonging to candidate PMevDC genes COG 1355, 1586 and 3407. Extensive pairwise comparisons of the S378Pa3-2 sequence with all sequences of the NR-database (CLANS analysis) demonstrated a rather isolated positioning of S378Pa3-2 at the edge of the known sequence space. Hence, a PMevDC biochemical activity was demonstrated for the first time with a protein that shows only very limited relationship to any protein available in public databases, clearly demonstrating novelty of the discovery. Moreover, the enzymatic activity encoded by the novel genes was identical to the activity found in Mtb. thermoautotrophicus.

### Example 3

### Construction of Isoprene Producing Strains Expressing Candidate Archaeal Isopentenyl Kinases (IPK) and Phosphomevalonate Decarboxylases (PMevDC)

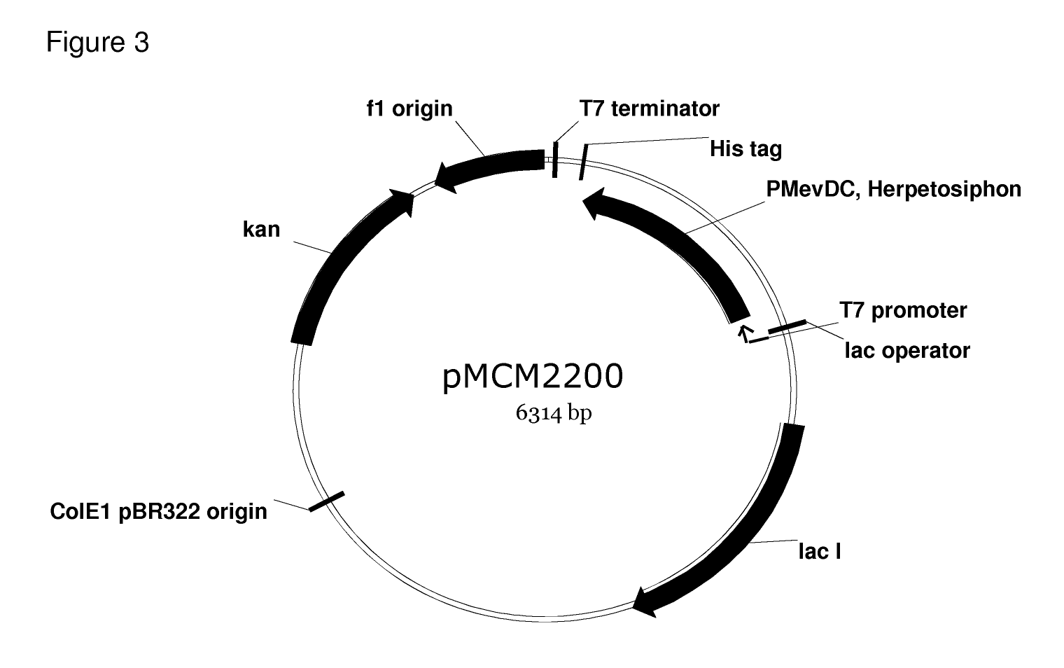

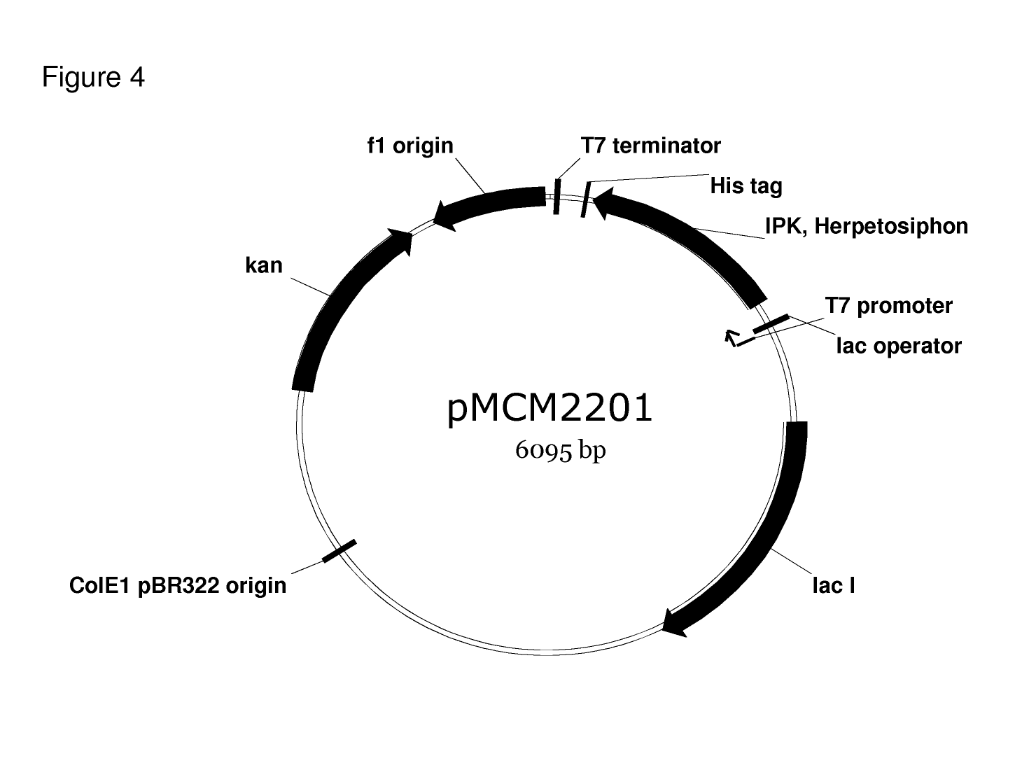

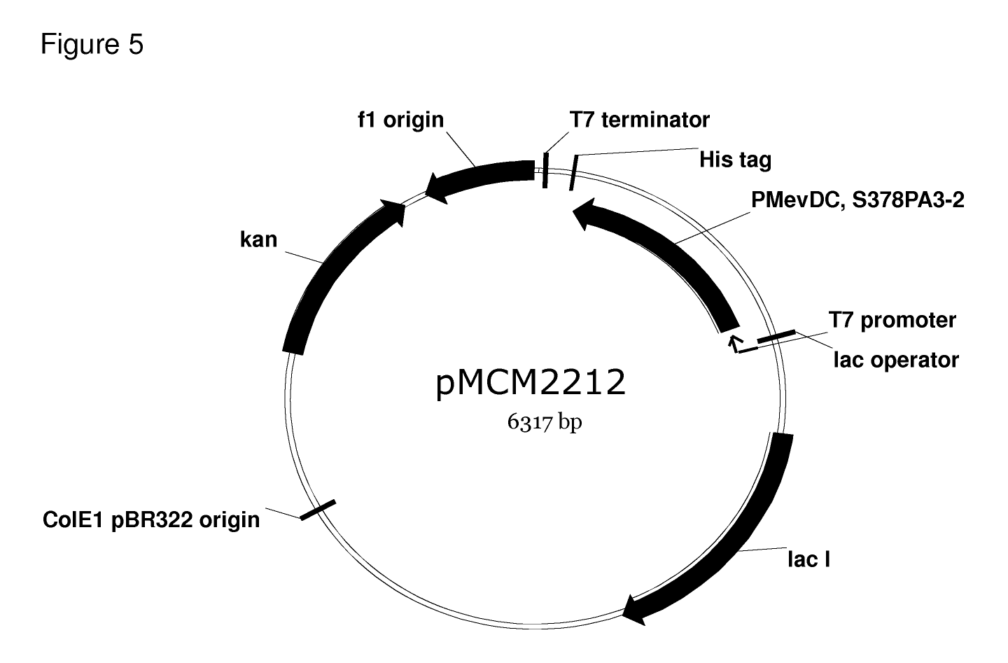

Plasmids encoding His-tagged versions of candidate IPK (FIG. 4) and PMevDC (FIGS. 3 and 5) genes were synthesized (Table 3). Genes were codon-optimized for expression in E. coli and included an N-terminal 6×His-tag followed by a TEV protease cleavage site. Plasmids were purified and transformed into chemically competent BL21(DE3) pLysS cells (Invitrogen #44-0307) following the manufacturer's protocol. Transformants were selected on LB plates supplemented with 50 μg/ml kanamycin and 25 μg/ml chloramphenicol after incubation at 37° C. overnight. The cultures were subsequently used for protein expression analysis.

TABLE 3
Plasmids pMCM2200, pMCM2201 and pMCM2212

| | | | | Expected |
|---|---|---|---|---|
| Plasmid | Protein Identifier | Source | Annotated Function | Function |
| pMCM2200 | Genbank | Herpetosiphon | Diphosphomevalonate | HIS-TEV- |
| | YP_001544383 | aurantiacus DSM | decarbox- | PMevDC |
| | | 785 | ylase | |
| pMCM2201 | Genbank | Herpetosiphon | aspartate/glutamate/uridylate | HIS-TEV-IPK |
| | YP_001545053 | aurantiacus dsm | kinase | |
| | | 785 | | |
| pMCM2212 | S378Pa3-2 | Metagenomic | n/a | HIS-TEV- |
| | | library | | PMevDC |

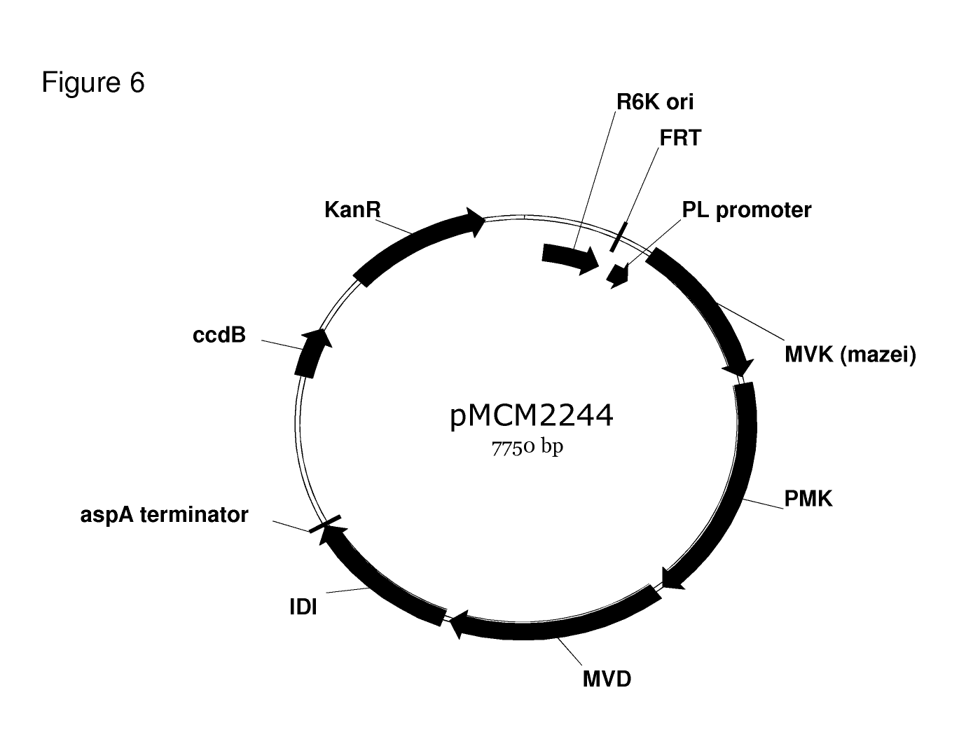

For generation of a plasmid that encodes the classical lower MVA pathway (pMCM2244, FIG. 6), Herculase II Fusion Enzyme with dNTPs Combo (Catalog #600679) was used according to the manufacturer's protocol. For amplification of the vector, about 50 ng/μL of plasmid pMCM881 was subjected to PCR using primers MCM851 and MCM852 in a reaction consisting of 35 μL ddH2O, 0.5 μL ddNTPs, 1.25 μL of each 10 μM primer, 1 μL pMCM881 and 1 μL enzyme. The PCR reaction was cycled as follows: 95° C. for 2 minutes; (95° C., 20 seconds; 55° C., 20 seconds; 72° C., 2 minutes) for 30 cycles; and 72° C. for 3 minutes before being held at 4° C. This reaction was treated with 2 μL of DpnI (Roche) at 37° C. overnight and then purified using a Qiagen QIAquick PCR Purification Kit (Cat. #28106). Likewise, the lower MVA pathway insert was amplified from 50 ng/μL chromosomal DNA of strain HMB, also known as MD314 or MD09-314 (see U.S. patent application Ser. No. 13/283,564), using primers MCM849 and MCM850 (Table 4). Four reactions consisting of 35 μL ddH2O, 0.5 μL ddNTPs, 1.25 μL of each 10 μM primer, 1 μL HMB DNA and 1 μL enzyme were cycled as follows: 95° C. for 2 minutes; (95° C., 20 seconds; 55° C., 20 seconds; 72° C., 4 minutes) for 30 cycles and 72° C. for 3 minutes before being held at 4° C. Vector and insert fragments were assembled using the GENEART® Seamless Cloning and Assembly Kit (Invitrogen Catalog no. A13288). About 1 μL ddH2O, 2 μL vector amplicon (pMCM881), 4 μL insert amplicon (lower MVA pathway insert), 2 μL buffer and 1 μL of enzyme were mixed and incubated at room temperature for 30 minutes. A 6 μL aliquot was used to transform chemically-competent Pir2 cells (Invitrogen C1111-10) and transformation reactions were recovered in LB media for 30 minutes before selection on LB plates supplemented with 50 μg/ml kanamycin at 30° C. with overnight incubation. Transformants were screened by PCR and the insert was verified by DNA sequencing. Strain MCM2244 carries pMCM2244, which has the expected sequence for the R6K-lower pathway fusion that encodes PMK and MVD from S. cerevisiae and MVK from M. mazei.

TABLE 4
Primers

| Name | Sequence |
|---|---|
| MCM849 | TCGGTTACGGTTGAGTAATAAATGGA |
| | (SEQ ID NO: 25) |
| MCM850 | AAAGTAGCCGAAGATGACGGTTTGTCACAT |
| | (SEQ ID NO: 26) |
| MCM851 | TGGCCGTCGTTTTACAACGT |
| | (SEQ ID NO: 27) |
| MCM852 | TTCAGGCTGTCAGCCGTTAAGT |
| | (SEQ ID NO: 28) |
| MCM855 | AAATGACTCTGAATTGCTGCCGGCTGAAAA |
| | GCAGGCTCTCGGAGGAGGAAATATGACTGC |
| | CGACAACAATAGT (SEQ ID NO: 29) |
| MCM856 | GTTCCGATCAAAGAGCTATCCTGGTTAATC |
| | TACTTTCAGACCTTGCTCGGTC |
| | (SEQ ID NO: 30) |
| MCM857 | CCAGGATAGCTCTTTGATCGGAACAAACGA |
| | AAATCAAAGGAGGAACCAACAATGTATGTC |
| | CGGAACGGA (SEQ ID NO: 31) |
| MCM858 | GCTATGGTCCGTGGCATCTACAAATCAGCC |
| | AACAAGACGAGC (SEQ ID NO: 32) |
| MCM859 | TTTGTAGATGCCACGGACCATAGCAATATA |
| | CTGCGAGAAGGGAGGGTTAACTTATGAACA |
| | AGCCGATTTTT (SEQ ID NO: 33) |
| MCM860 | GCCGGCAGCAATTCAGAGTCATTTTCAATC |
| | CAATTTTATAATGGTTCCCGGCC |
| | (SEQ ID NO: 34) |
| MCM889 | CCAGGATAGCTCTTTGATCGGAACTGAACT |
| | TCAGTTTAGCAAAGGAGAGTATCGATGGAT |
| | TACTATTACCGCGT (SEQ ID NO: 35) |
| MCM890 | GCTATGGTCCGTGGCATCTACAAATCAAAT |
| | CAGCTGAGCACCCTGC |
| | (SEQ ID NO: 36) |

For generation of strains expressing H. aurantiacus IPK together with H. aurantiacus PMevDC or with S378Pa3-2 PMevDC, DNA fragments were amplified by PCR using the Herculase II Fusion Enzyme with dNTPs Combo (Catalog #600679) kit according to the manufacturer's protocol (Table 5). Reactions consisting of 35 μL ddH₂O, 0.5 μL dNTPs, 1.25 μL, 10 μM of forward and reverse primer each, 1 μL template (˜50 ng/uL) and 1 μL enzyme were cycled as follows: 95° C. for 2 minutes; (95° C., 20 seconds; 55° C., 20 seconds; 72° C., as noted in Table 5) for 30 cycles and 72° C. for 3 minutes before being held at 4° C. overnight.

TABLE 5
PCR amplification of PMevDC and IPK

| | | | | Exten- |
|---|---|---|---|---|
| | | | | sion |
| Target | Template | Primer1 | Primer2 | (min) |
| Linearized pMCM2244 | pMCM2244 | MCM855 | MCM856 | 3:00 |
| lacking PMK and MVD | | | | |
| PMevDC, Herpetosiphon | pMCM2200 | MCM857 | MCM858 | 0:30 |
| IPK, Herpetosiphon | pMCM2201 | MCM859 | MCM860 | 0:30 |
| PMevDC, S378Pa3-2 | pMCM2212 | MCM889 | MCM890 | 0:30 |

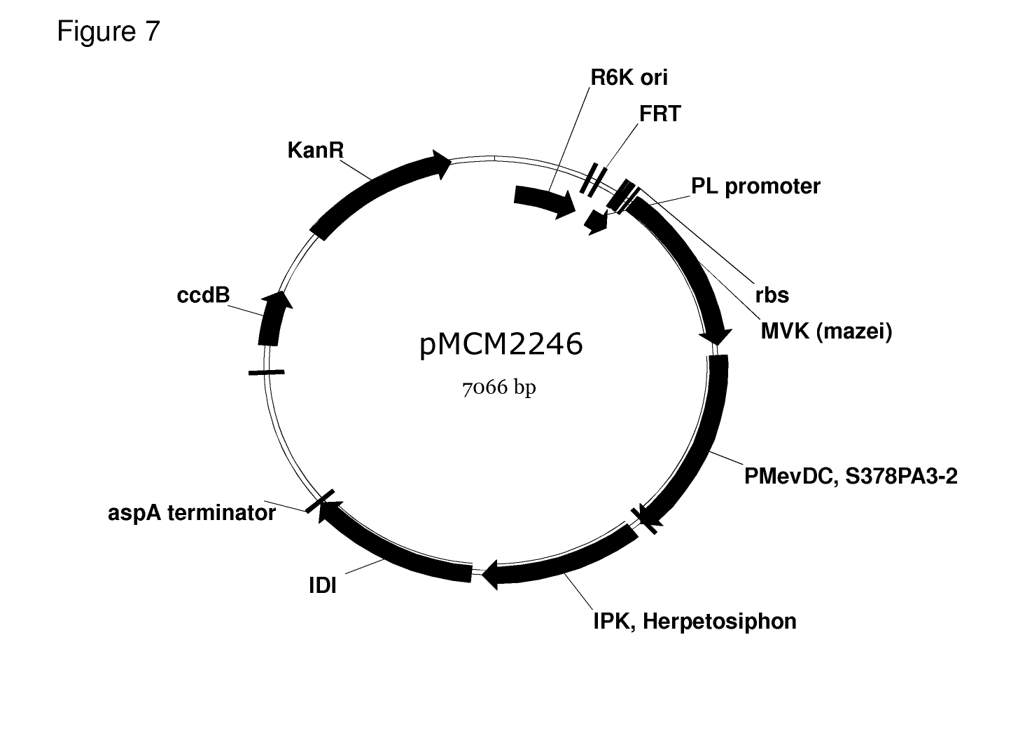

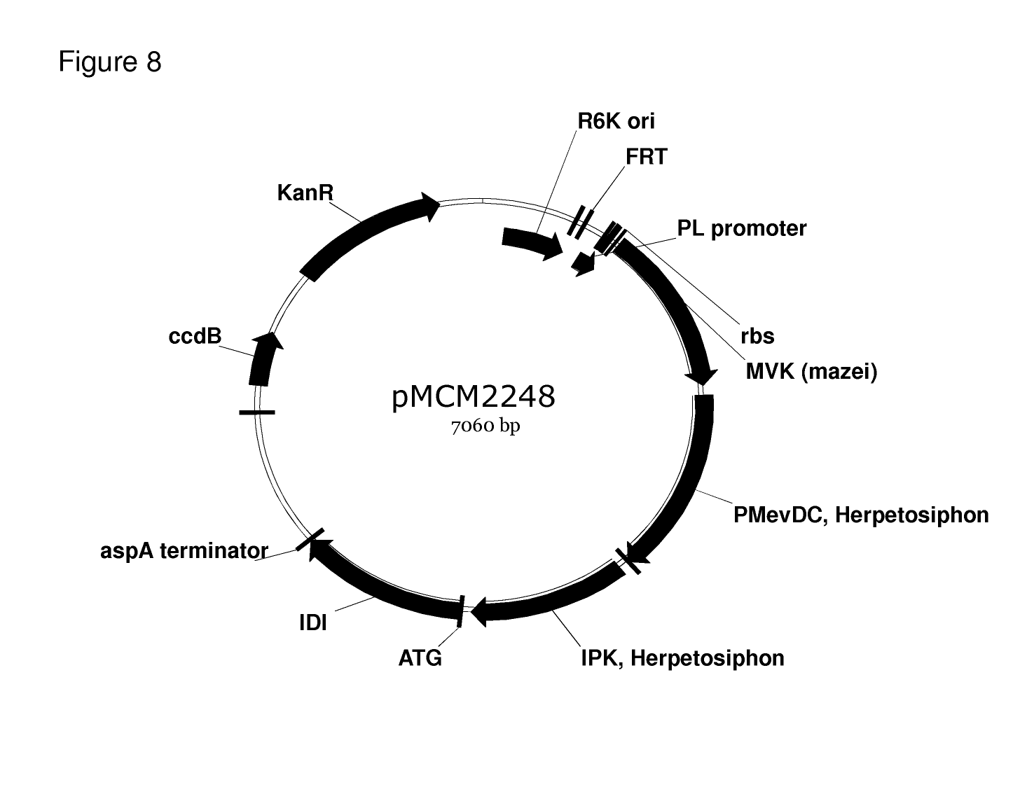

Reactions were treated with 2 μL DpnI (Roche) for 2 hours at 37° C. and then purified using the Qiagen QIAquick PCR Purification Kit (Cat. #28106). The linearized pMCM2244 plasmid was fused to H. aurantiacus IPK and to H. aurantiacus PMevDC or to S378Pa3-2 PMevDC using the GENEART® Seamless Cloning and Assembly Kit (Invitrogen Catalog no. A13288). A mixture of 1 μL ddH₂O, 2 μL of each of three amplicons, 2 μL buffer and 1 μL of enzyme were mixed and incubated at room temperature for 30 minutes. A 5 μL sample of the mixture was used to transform chemically-competent Pir2 cells (Invitrogen C1111-10) and transformation reactions were recovered in SOC media for 30 minutes at 30° C. and selection of transformants on LB plates supplemented with 50 μg/ml kanamycin at 30° C. with overnight incubation. Transformants were screened by PCR and the insert sequence was verified by DNA sequencing (Table 6, FIGS. 7 and 8).

TABLE 6
Strains expressing archaeal enzymes

| Strain | Plasmid | Vector | IPK | PMevDC |
|---|---|---|---|---|
| MCM2246 | pMCM2246 | Linearized pMCM2244 lacking | IPK, Herpetosiphon | PMevDC, S378Pa3-2 |
| | | PMK and MVD | | |
| MCM2248 | pMCM2248 | Linearized pMCM2244 lacking | IPK, Herpetosiphon | PMevDC, |
| | | PMK and MVD | | Herpetosiphon |

Plasmids pMCM82 (see U.S. Patent Appl. Pub. No. US 2011/0159557) and pCHL243, also known as pDW72 (see U.S. patent application Ser. No. 13/283,564), were both electroporated into strains MCM2244, MCM2246 and MCM2248. For electroporation, cells were grown in LB plates supplemented with 50 μg/ml kanamycin, washed three times in iced ddH₂O and electroporated with 1 μL each plasmid in a 2 mm electroporation cuvette at 25 uFD, 200 ohms, and 2.5 kV. Reactions were immediately quenched with 500 μL LB media and recovered at 37° C. with shaking for 1 hour before plating on LB plates supplemented with 50 μg/ml kanamycin and 50 μg/ml carbenicillin or on LB plates supplemented with 50 μg/ml kanamycin, 50 μg/ml carbenicillin, and 50 μg/ml spectinomycin and incubated overnight at 37° C. After incubation the selection plates were moved to room temperature for 8 hours before the transformants were patched and incubated at room temperature for 3 days for production of the strains (Table 7). Strain MCM2257 expressed the classical lower MVA pathway and isoprene synthase but did not express the upper MVA pathway. Strains MCM2258 and MCM2259 expressed the alternative lower MVA pathway and isoprene synthase but did not express the upper MVA pathway. Strain MCM2260 expressed the upper MVA pathway, the classical lower MVA pathway, and isoprene synthase. Strains MCM2261 and MCM2262 expressed the upper MVA pathway, the alternative lower MVA pathway, and isoprene synthase.

TABLE 7
Strains expressing the MVA pathway and archaeal enzymes

| Resulting | | Parent | | |
|---|---|---|---|---|
| Strain | Genotype | Strain | Plasmids | Antibiotics |
| MCM2257 | pir2 pR6K-pw518 + pTrcAlba(IspS | MCM2244 | pMCM2244 | kan50 carb50 |
| | MEA variant)-mMVK | | pCHL243 | |
| MCM2258 | pir2 pR6K-pw PMevDC S378Pa3-2 + | MCM2246 | pMCM2246 | kan50 carb50 |
| | pTrcAlba(IspS MEA variant)-mMVK | | pCHL243 | |
| MCM2259 | pir2 pR6K-cI857-pw PMevDC | MCM2248 | pMCM2248 | kan50 carb50 |
| | Herpetosiphon + pTrcAlba(IspS MEA | | pCHL243 | |
| | variant)-mMVK | | | |
| MCM2260 | pir2 pR6K-cI857-pw518 + | MCM2244 | pMCM2244 | kan50 carb50 |
| | pTrcAlba(IspS MEA variant)-mMVK + | | pCHL243 | spec50 |
| | pCL-Ptrc-Upper_faecalis | | pMCM82 | |
| MCM2261 | pir2 pR6K-cI857-pw PMevDC, | MCM2246 | pMCM2246 | kan50 carb50 |
| | S378Pa3-2 + pTrcAlba(IspS MEA | | pCHL243 | spec50 |
| | variant)-mMVK + pCL-Ptrc- | | pMCM82 | |
| | Upper_faecalis | | | |
| MCM2262 | pir2 pR6K-cI857-pw PMevDC | MCM2248 | pMCM2248 | kan50 carb50 |
| | Herpetosiphon + pTrcAlba(IspS MEA | | pCHL243 | spec50 |
| | variant)-mMVK + pCL-Ptrc- | | pMCM82 | |
| | Upper_faecalis | | | |

Note:
kan50 is 50 μg/ml kanamycin;
carb50 is 50 μg/ml carbenicillin;
and spec50 is 50 μg/ml spectinomycin

### Example 4

### Characterization of Candidate Phosphomevalonate Decarboxylases

Substrate specificity, solubility, and kinetic properties of PMevDC isolated from S378Pa3-2, and Herpetosiphon aurantiacus ATCC 23779 were studied and characterized.

### (i) Materials and Methods

### Growth, Expression and Purification of Proteins

Strains MCM2257, MCM2258, MCM2259, MCM2260, MCM2261, and MCM2262 were inoculated in 1 liter of LB medium containing the appropriate antibiotic and incubated at 34° C. for 7 hours from overnight cultures grown at 34° C. in LB broth containing the appropriate antibiotic (Table 7). Cultures at an OD 0.5-0.7 were induced with 200 μM IPTG. After induction, cells were harvested by centrifugation at 10000×g for 10 minutes. After removal of the supernatant, the cell pellets were resuspended in 40 mL lysis buffer containing 50 mM KPO4, pH 8.0, 0.3 M NaCl, 0.02 mM imidizole, 1 mg/mL lysozyme, and 1 mg/mL DNAase. The cells were lysed using a french pressure cell at 14,000 psi and the cell lysate was centrifuged at 50,000×g for 1 hour. The supernatant was collected, passed over a Ni-affinity resin before the resin was washed with 10 column volumes of lysis buffer containing 50 mM imidazole. The protein was eluted with 5 column volumes of lysis buffer containing 250 mM imidizole. Collected fractions were concentrated and passed over PD-10 columns for buffer exchange and the final collected protein samples were >95% pure according to SDS-PAGE analysis. The purified samples were incubated with TEV protease overnight at 4° C. to remove histidine tags from the purified proteins. The digested samples were subsequently passed over Ni-affinity resin and the flow-through was collected and analyzed by SDS-PAGE.

### Kinetic Characterization of Decarboxylases

PMevDCs were incubated in the presence of mevalonate, phosphomevalonate, diphosphomevalonate, ATP, MgCl₂ and the products of the reactions were confirmed by LC-MS. Mevalonate decarboxylase (MVD) from Saccharomyces cerevisiae was used as a reference. The catalytic activities of the decarboxylases were measured using a modified spectrophotometric assay that coupled ADP formation to pyruvate synthesis and reduction to lactate. The initial rate of disappearance of NADH was monitored at 340 nm on a SpectraMax M5 (Molecular Devices) to measure the reaction rate catalyzed by the PMevDCs. Samples for reaction rate studies contained 0.8 mM phosphoenolpyruvate, 0.05 mM DTT, 0.32 mM NADH, 10 mM MgCl₂, 4 U lactate dehydrogenase, 4 U pyruvate kinase, 5 mM ATP and 10-250 μM (R)-phosphomevalonate or 10-250 μM (R)-diphosphomevalonate. All reactions were performed at 34° C. Reaction rate data was processed using Microsoft Excel and kinetic parameters were determined using Kaleidagraph.

### (ii) Results

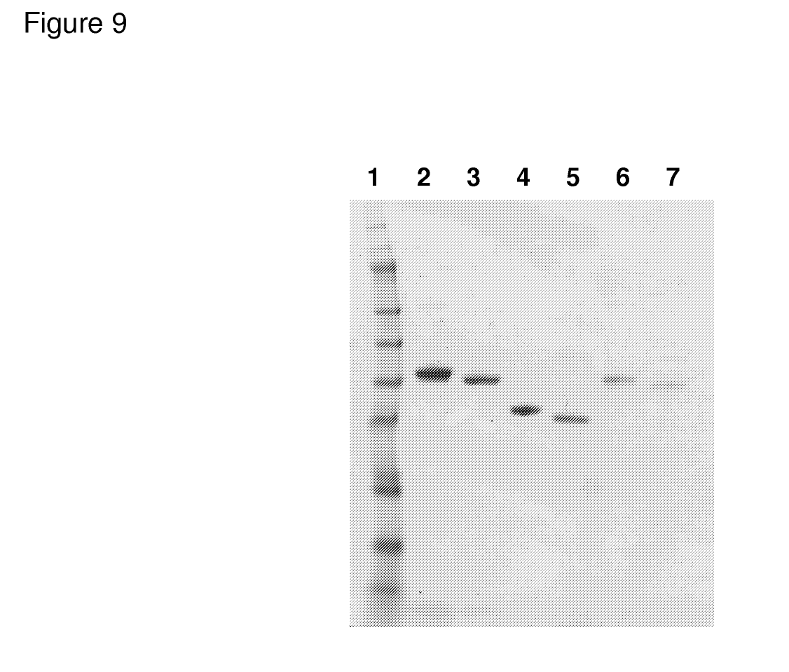

Analysis of His-tagged and TEV protease cleaved PMevDCs and IPKs by SDS-PAGE revealed that the strains expressed soluble enzymes. H. aurantiacus PMevDC (lanes 2 and 3), H. aurantiacus IPK (lanes 4 and 5), and S378Pa3-2 PMevDC (lanes 6 and 7) were all soluble whether they were expressed with an attached His-tag or without a His-Tag (FIG. 9).

The K_{M} and k_{cat} catalytic constants for yeast MVD, S378Pa3-2 PMevDC, and H. aurantiacus PMevDC were determined (Table 8). The results indicate that the decarboxylases can be distinguished based on their substrate specificity. S. cerevisiae MVD catalyzes the conversion of diphosphomevalonate with a k_{cat} of 11.6 s⁻¹ with a K_{M} of 44 μM, however, no reaction rate was detected for the S. cerevisiae MVD catalyzed decarboxylation of phosphomevalonate. Based on the limit of detection of the assay the catalytic rate for the decarboxylation of phosphomevalonate catalyzed by S. cerevisiae decarboxylase was less than 0.02 s⁻¹ using 1 mM phosphomevalonate. The S378Pa3-2 PMevDC catalyzed the decarboxylation of phosphomevalonate with a k_{cat} of 2.9 s⁻¹ with a K_{M} of 26 μM and catalyzed the decarboxylation of diphosphomevalonate with a k_{cat} of 1.09 s⁻¹ with a K_{M} of 22 μM. The Herpetosiphon aurantiacus ATCC 23779 PMevDC catalyzed the decarboxylation of phosphomevalonate with a k_{cat} of 3.3 s⁻¹ with a K_{M} of 57 μM, however decarboxylation of diphosphomevalonate was undetectable using the assay conditions as described. Based on the limit of detection of the assay the catalytic rate for the decarboxylation of diphosphomevalonate catalyzed by Herpetosiphon aurantiacus ATCC 23779 decarboxylase was less than 0.02 s⁻¹ using 1 mM diphosphomevalonate.

TABLE 8
Kinetic Characterization of Decarboxylases

| | Phosphomevalonate | | Diphosphomevalonate | |
|---|---|---|---|---|
| | k_{cat} ± | k_{M} ± | k_{cat} ± | K_{M} ± |
| Decarboxylase | SD (s⁻¹) | SD (μM) | SD (s⁻¹) | SD (μM) |
| Herpetosiphon | 3.3 ± 0.2 | 57 ± 13 | <0.02* | ND |
| PMevDC | | | | |
| S378Pa3-2 | 2.9 ± 0.5 | 26 ± 8 | 1.09 ± 0.09 | 22 ± 8 |
| PMevDC | | | | |
| S. cerevisiae MVD | <0.02* | ND | 11.6 ± 0.6 | 44 ± 7 |

Errors reported are the standard error for each curve fit.
*Using 1 mM substrate

### Example 5

### Metabolite Production in Recombinant Cells Expressing Archaeal PMevDC and IPK

Substrate conversion and product formation by PMevDC isolated from S378Pa3-2 and Herpetosiphon aurantiacus ATCC 23779 were studied and analyzed.

### (i) Materials

### TM3 Media Recipe (Per Liter Fermentation Medium):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 0.2 g, 1000X Trace Metals Solution 1 ml. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### TM3+1% Glu+0.02% YE Media Recipe (Per Liter):

K₂HPO₄ 13.6 g, KH₂PO₄ 13.6 g, citric acid monohydrate 2 g, ferric ammonium citrate 0.3 g, (NH₄)₂SO₄ 3.2 g, 1000X Trace Metals Solution 1 ml. Supplemented with 0.02% yeast extract. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### Supplemented TM3+1% Glu+0.1% YE Media Recipe (Per Liter):

K₂HPO₄ 13.6 g, KH₂PO₄13.6 g, citric acid monohydrate 2 g, ferric ammonium citrate 0.3 g, (NH₄)₂SO₄ 3.2 g, 1000X Trace Metals Solution 1 ml. Supplemented with 0.1% yeast extract. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### Supplemented TM3+1% Glu+0.02% YE+1% Cas-Amino Acid Media Recipe (Per Liter):

K₂HPO₄ 13.6 g, KH₂PO₄13.6 g, citric acid monohydrate 2 g, ferric ammonium citrate 0.3 g, (NH₄)₂SO₄ 3.2 g, 1000X Trace Metals Solution 1 ml. Supplemented with 0.02% yeast extract and 0.1% cas-amino acids. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### LB Media Recipe+1% Glucose (Per Liter):

Luria Broth (LB) media was supplemented with 10.0 g glucose and antibiotic after sterilization.

### 1000X Modified Trace Metal Solution (Per Liter):

Citric Acids*H2O 40 g, MnSO4*H2O 30 0 g, NaCl 10 g, FeSO4*7H2O 1 g, CoCl2*6H2O 1 g, ZnSO*7H2O 1 g, CuSO4*5H2O 100 mg, H3BO3 100 mg, NaMoO4*2H2O 100 mg. Each component was dissolved one at a time in Di H2O, pH was adjusted to 3.0 with HCl/NaOH, and then the solution was q.s. to volume and filter sterilized with a 0.22 micron filter.

### (ii) Experimental Procedure

### In Vitro Metabolite Measurement

In vitro assays were done with crude extracts from E. coli DH10b overexpressing pMCM2212. This strain of E. coli did not encode any known MVA genes. Negative control assays were done with extracts of E. coli harboring pTrcHis2b without insert. Substrates for in vitro conversions were mevalonate (MVA), mevalonate phosphate (MVP) also referred to as mevalonate 5-phosphate, mevalonate diphosphate (MVPP) also referred to as mevalonate 5-pyrophosphate, and isopentenyl phosphate (IP). Substrate conversion and product formation was analyzed by LC/MS.

### In Vivo Metabolite Measurement

Shake tubes containing 5 ml LB media and appropriate antibiotics were inoculated with glycerol culture stocks (Table 7). Cultures were incubated for approximately 15 hours at 30° C., 220 rpm. A 2 mL sample of day culture was diluted to a final OD₆₀₀ of 0.2 and placed in each well of a 48-well sterile block containing one of four types of media 1) TM3 with 1% glucose and 0.02% YE, 2) TM3 with 1% glucose and 0.1% YE, 3) TM3 with 1% glucose 0.02% YE, 0.1% cas-amino acids, and 4) LB with 1% glucose. Blocks were sealed with Breathe Easier membranes and incubated for 1.5 hours at 34° C., 600 rpm. After 1.5 hours of growth, the OD₆₀₀ was measured in the micro-titer plate and cells were induced with 200 μM final concentration of IPTG. An OD₆₀₀ reading and specific productivity sample collection was taken at 2 hours and four hours after IPTG induction. OD₆₀₀ was measured in the microtiter plate at the appropriate dilution in the TM3 media. Measurements were performed using a SpectraMax M5 (Molecular Devices). A 1000 μL cell culture sample was collected and centrifuged to collect the pellet. The cell pellet was subsequently quenched with 100 μL methanol as the first extraction step for isolating intracellular metabolites. The sample was further extracted with 100 μL of 75% methanol/10 mM NH₄Ac buffer (pH 7.0) before a final extraction with 70 μL of 75% methanol/10 mM NH₄Ac buffer (pH 7.0). The combined extraction volume was 270 μL and the obtained samples were analyzed by LC/MS.

Mass spectrometric analysis of metabolites was performed using a TSQ Quantim triple quadrupole instrument (Thermo Scientific). System control, data acquisition, and data analysis were done with XCalibur and LCQuan software (Thermo Scientific). About 10 μL samples were applied to a C18 Synergi MAX-RP HPLC column (150×2 mm, 4 uM, 80 A, Phenomenex) equipped with the manufacturer-recommended guard cartridge. The column was eluted with a gradient of 15 mM acetic acid+10 mM tributylamine in MilliQ-grade water (solvent A) and LCMS-grade methanol from Honeywell, Burdick & Jackson (solvent B). The 14 min gradient was as follows: t=0 min, 20% B; t=1 min, 30% B; t=9 min, 55% B; t=10 min, 90% B; t=12 min, 90% B; t=13 min, 20% B; t=14 min, 20% B; flow rate 0.4 mL/min, column temperature 35° C. Mass detection was carried out using electrospray ionization in the negative mode at ESI spray voltage of 3.0-3.5 kV and ion transfer tube temperature of 350° C. The following SRM transitions were selected for metabolites of interest: 227→79 at 40 eV for mevalonate phosphate (MVP), 307→209 at 17 eV for mevalonate diphosphate (MVPP), 165→79 at 40 eV for isopentenyl (IP), and 245→79 at 40 eV for isopentenyl pyrophosphate (IPP). Argon was used as the collision gas at 1.7 mTorr, scan time for each SRM transition was 0.1 s with a scan width set at 0.7 m/z. Concentrations of metabolites in cell extracts were determined based on calibration curves obtained by injection of commercial standards dissolved in 20% methanol/50 mM NH₄Ac buffer (pH 7.0) to 0.5 ppm to 50 ppm final concentration. Metabolite standards used were MVP*Li (Sigma), MVPP*4Li (Sigma), IP*2NH4 (Sigma), and IPP*4NH4 (Echelon Biosciences Inc.).

(iii) Results

Crude extract isolated from E. coli DH10b overexpressing S378Pa3-2 demonstrated activity on MVP and some activity on MVPP, whereas MVA and IP were not used as substrates for this enzyme (Table 9). MVP was quantitatively converted to IP, MVPP was converted to IPP, indicating a somewhat relaxed substrate spectrum for S378Pa3-2. Somewhat elevated levels of IP in the sample supplemented with MVPP was explained by the presence of a small amount of MVP in the commercial MVPP and/or IPP phosphatase activity in the E. coli extracts. Control assays revealed presence of endogenous phosphatase activities within E. coli acting on IPP and MVPP suggesting targets for improvement of the MVA pathway.

TABLE 9
Substrate conversion by crude E. coli lysate

| | | Product or substrate detected | | | |
|---|---|---|---|---|---|
| | | (% of control with no cell | | | |
| | Substrate for | lysate added) | | | |
| Cell culture | conversion | MVP | MVPP | IP | IPP |
| DH10b, pCR-ctrl, no insert | MVA | 0.2 | 0.1 | 0.5 | 0.0 |
| DH10b, pCR-ctrl, no insert | MVP | 64.9 | 0.0 | 0.1 | N/D |
| DH10b, pCR-ctrl, no insert | MVPP | 6.1 | 40.5 | 0.1 | 0.1 |
| DH10b, pCR-ctrl, no insert | IP | 0.2 | 0.3 | 57.2 | 0.0 |
| DH10b, pCR_S378Pa3-2 | MVA | 0.1 | 0.1 | 0.5 | N/D |
| DH10b, pCR_S378Pa3-2 | MVP | 56.7 | 0.1 | 33.9 | N/D |
| DH10b, pCR_S378Pa3-2 | MVPP | 7.3 | 42.4 | 2.5 | 6.0 |
| DH10b, pCR_S378Pa3-2 | IP | 0.3 | 0.5 | 77.0 | 0.1 |

Crude extracts isolated from strains MCM2257, MCM2258, MCM2259, MCM2260, MCM2261, and MCM2262 were analyzed for formation of MVP, MVPP, IP, and IPP (Table 10). Analysis of metabolite formation in strains grown for two hours in LB media demonstrated that strain MCM2260 which expresses the full upper MVA pathway and the classical lower MVA pathway predominantly produced IPP at 0.66 mM or 0.91 mM when grown in TM3 media for two hours or four hours, respectively. Strain MCM2260 also produced predominantly more IPP at 0.13 mM for four hours when grown in LB media, albeit lower than when grown in TM3 media (Table 10). Strain MCM2261 which expressed the full upper MVA pathway and the lower MVA pathway with S378Pa3-2 PMevDC predominantly produced MVP at 12.68 mM or 31.05 mM when grown in TM3 media for two hours or four hours, respectively. Strain MCM2261 also produced more MVP at 30.24 mM for four hours when grown in LB media. However, in comparison to strain MCM2260, strain MCM2261 produced more IP in all conditions and in certain conditions, such as when grown in LB media for four hours, surpassed strain MCM2260 in IPP production. In regards to IP and IPP production, similar results were seen in strain MCM2262 which expressed the full upper pathway and the lower MVA pathway with H. aurantiacus PMevDC. In comparison to strain MCM2260, strain MCM2262 produced more IP in all conditions and in certain conditions, such as when grown in LB media for four hours, surpassed strain MCM2260 in IPP production. In contrast to strain MCM2261, strain MCM2262 did not accumulate high levels of MVP.

TABLE 10
Metabolite production

| | | Metabolites, mM intracellular* | | | |
|---|---|---|---|---|---|
| Strain | Conditions (Time/Media) | MVP | MVPP | IP | IPP |
| MCM2257 | 4 hr/TM3 | 0.17 | 0.03 | 0.02 | 0.06 |
| MCM2258 | | 0.70 | 0.18 | 0.04 | 0.05 |
| MCM2259 | | 0.39 | 0.09 | 0.00 | 0.03 |
| MCM2260 | | 0.09 | 0.05 | 0.10 | 0.91 |
| MCM2261 | | 31.05 | 0.04 | 1.34 | 0.54 |
| MCM2262 | | 0.19 | 0.02 | 0.62 | 0.56 |
| MCM2257 | 4 hr/LB | 0.03 | 0.01 | 0.00 | 0.00 |
| MCM2258 | | 0.27 | 0.04 | 0.00 | 0.03 |
| MCM2259 | | 0.38 | 0.00 | 0.00 | 0.02 |
| MCM2260 | | 0.02 | 0.00 | 0.02 | 0.13 |
| MCM2261 | | 30.24 | 0.01 | 2.59 | 0.30 |
| MCM2262 | | 0.63 | 0.00 | 1.66 | 0.53 |
| MCM2257 | 2 hr/TM3 | 0.10 | 0.00 | 0.05 | 0.05 |
| MCM2258 | | 0.22 | 0.05 | 0.00 | 0.02 |
| MCM2259 | | 0.13 | 0.06 | 0.00 | 0.01 |
| MCM2260 | | 0.07 | 0.04 | 0.10 | 0.66 |
| MCM2261 | | 12.68 | 0.04 | 0.96 | 0.45 |
| MCM2262 | | 0.17 | 0.03 | 0.85 | 0.46 |

*Intracellular concentrations of metabolites were calculated from optical densities of the cultures measured at 600 nm (OD₆₀₀) assuming that total intracellular volume of 1 mL of E. coli cells grown to OD₆₀₀ = 4.0 is equal to 1 μL.

### Example 6

### Production of Isoprene by Recombinant Host Cells Expressing PMevD, IPK, and the Upper MVA Pathway at Small Scale

Isoprene production by strains expressing the upper MVA pathway and the alternative archaeal lower MVA pathway was compared to strains expressing the upper MVA pathway and classical lower pathway.

### (i) Materials

### TM3 Media Recipe (Per Liter Fermentation Medium):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 0.2 g, 1000X Trace Metals Solution 1 ml. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### TM3+1% Glu+0.02% YE Media Recipe (Per Liter):

K₂HPO₄ 13.6 g, KH₂PO₄ 13.6 g, citric acid monohydrate 2 g, ferric ammonium citrate 0.3 g, (NH₄)₂SO₄ 3.2 g, 1000X Trace Metals Solution 1 ml. Supplemented with 0.02% yeast extract. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### Supplemented TM3+1% Glu+0.1% YE Media Recipe (Per Liter):

K₂HPO₄ 13.6 g, KH₂PO₄ 13.6 g, citric acid monohydrate 2 g, ferric ammonium citrate 0.3 g, (NH₄)₂SO₄ 3.2 g, 1000X Trace Metals Solution 1 ml. Supplemented with 0.1% yeast extract. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### Supplemented TM3+1% Glu+0.02% YE+1% Cas-Amino Acid Media Recipe (Per Liter):

K₂HPO₄ 13.6 g, KH₂PO₄ 13.6 g, citric acid monohydrate 2 g, ferric ammonium citrate 0.3 g, (NH₄)₂SO₄ 3.2 g, 1000X Trace Metals Solution 1 ml. Supplemented with 0.02% yeast extract and 0.1% cas-amino acids. All of the components were added together and dissolved in diH₂O. The pH was adjusted to 6.8 with ammonium hydroxide (30%) and brought to volume. Media was filter-sterilized with a 0.22 micron filter. Glucose 10.0 g and antibiotic were added after pH adjustment and sterilization.

### LB Media Recipe+1% Glucose (Per Liter):

Luria Broth (LB) media was supplemented with 10.0 g glucose and antibiotic after sterilization.

### 1000X Modified Trace Metal Solution (Per Liter):

Citric Acids*H2O 40 g, MnSO4*H2O 30 0 g, NaCl 10 g, FeSO4*7H2O 1 g, CoCl2*6H2O 1 g, ZnSO*7H2O 1 g, CuSO4*5H2O 100 mg, H3BO3 100 mg, NaMoO4*2H2O 100 mg. Each component was dissolved one at a time in Di H2O, pH was adjusted to 3.0 with HCl/NaOH, and then the solution was q.s. to volume and filter sterilized with a 0.22 micron filter.

### (ii) Experimental Procedure

### Growth Rate Measurement

Shake tubes containing 5 ml LB media and appropriate antibiotics were inoculated with glycerol culture stocks (Table 7). Cultures were incubated for approximately 15 hours at 30° C., 220 rpm. A 2 mL sample of day culture was diluted to a final OD₆₀₀ of 0.2 and placed in each well of a 48-well sterile block containing one of four types of media 1) TM3 with 1% glucose and 0.02% YE, 2) TM3 with 1% glucose and 0.1% YE, 3) TM3 with 1% glucose 0.02% YE, 0.1% cas-amino acids, and 4) LB with 1% glucose. Blocks were sealed with Breathe Easier membranes and incubated for 1.5 hours at 34° C., 600 rpm. After 1.5 hours of growth, the OD₆₀₀ was measured in the micro-titer plate and cells were induced with 200 μM final concentration of IPTG. An OD₆₀₀ reading and specific productivity sample collection was taken every hour after the IPTG induction for 4 hours. OD₆₀₀ was measured in the microtiter plate at the appropriate dilution in the TM3 media. Measurements were performed using a SpectraMax M5 (Molecular Devices).

### Isoprene Specific Productivity Measurement

For the isoprene headspace assay, a 100 μl of culture sample was collected in a 96-well glass block every hour after IPTG induction for 4 hours. The glass block was sealed with aluminum foil and incubated at 34° C. while shaking at 450 rpm for 30 minutes using a Thermomixer. After 30 minutes, the block the cells were killed in a 70° C. water bath for 2 minutes and levels of isoprene in the headspace measurement were determined using gas chromatography-mass spectrometry. Measured isoprene from the 100 μl culture head space was converted to OD normalized isoprene specific productivity.

(iii) Results

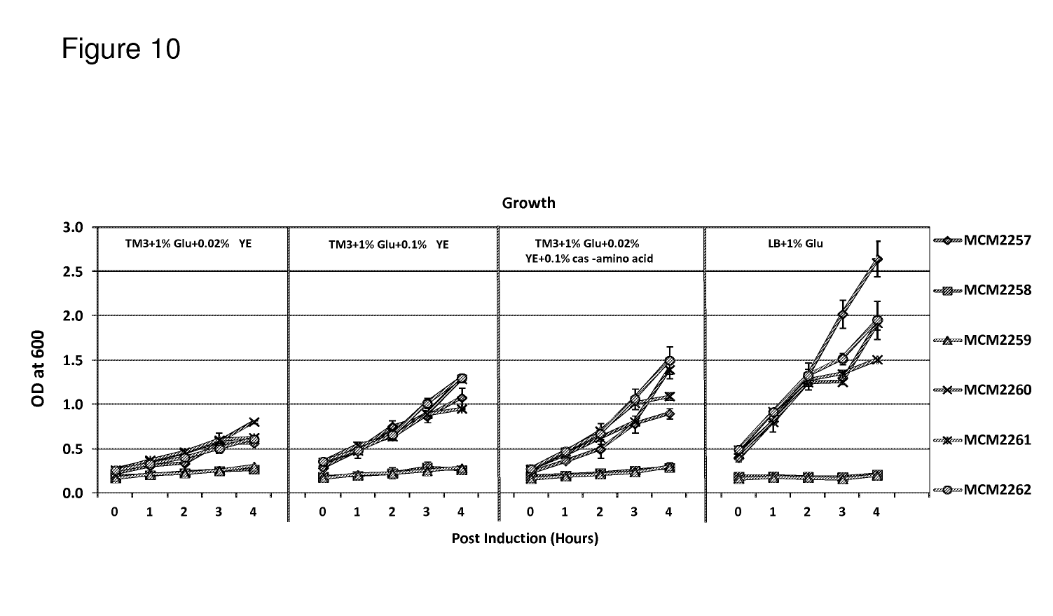

Analysis of growth by engineered E. coli strains expressing H. aurantiacus IPK and S378Pa3-2 PMevDc (strain MCM2261) or H. aurantiacus IPK and H. aurantiacus PMevDC (strain MCM2262) demonstrated comparable growth to a control E. coli strain expressing S. cerevisiae PMK and S. cerevisiae MVD (strain MCM2260) in the presence of IPTG induction across the four different media compositions that were tested (FIG. 10).

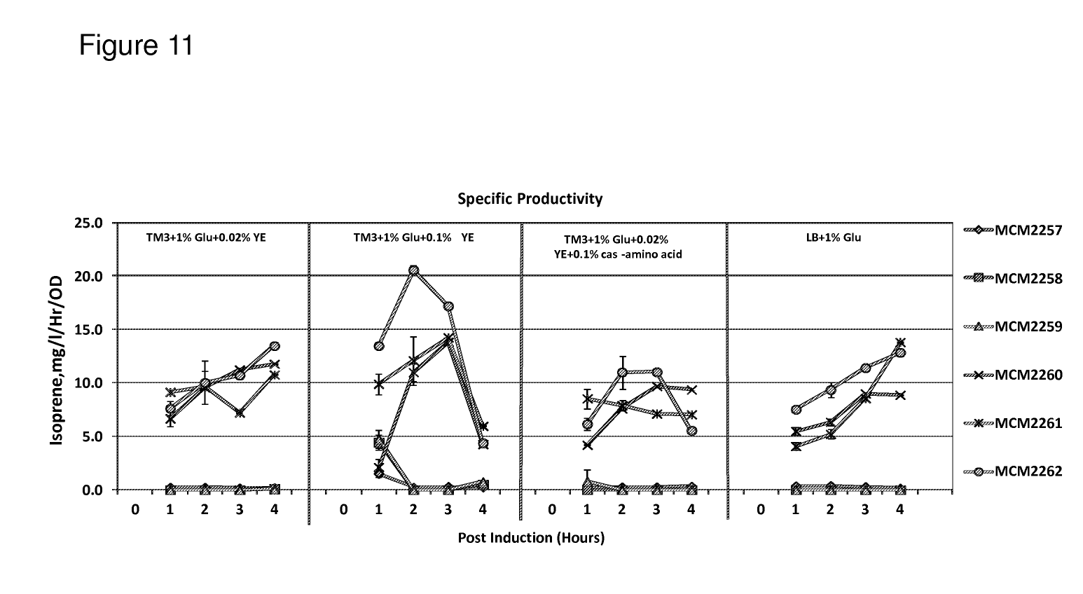

Analysis of isoprene produced from glucose by engineered E. coli strains expressing H. aurantiacus IPK and S378Pa3-2 PMevDc (strain MCM2261) or H. aurantiacus IPK and H. aurantiacus PMevDC (strain MCM2262), as compared to a control E. coli strain expressing S. cerevisiae PMK and S. cerevisiae MVD (strain MCM2260) demonstrated that both S378Pa3-2 PMevDc and H. aurantiacus PMevDC in the presence of an archaeal IPK, such as H. aurantiacus IPK, allowed for the production of isoprene at comparable levels to the control strain (FIG. 11). Furthermore, increasing isoprene yield correlated with increasing IPTG induction. The amount of isoprene produced by the tested strains varied with the growth media that was used (FIG. 11).

Overall, these results demonstrated that alternative lower MVA pathway enzymes, such as archaeal PMevDCs and archaeal IPKs, can be used in place of classical lower MVA pathway enzymes, such as PMK and MVD, in recombinant cells to produce isoprene.

### Example 7

### Production of Isoprene by Recombinant Host Cells Expressing PMevD, IPK, and the Upper MVA Pathway at 15-L Scale

Isoprene production by strains expressing the upper MVA pathway and the alternative archaeal lower MVA pathway are compared to strains expressing the upper MVA pathway and the classical lower pathway.

### (i) Materials

### TM3 Media Recipe (Per Liter Fermentation Media):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 0.2 g, 1000X Trace Metals Solution 1 ml. All of the components are added together and dissolved in diH₂O. The pH is adjusted to 6.8 with ammonium hydroxide (30%) and brought to volume. Media is filter-sterilized with a 0.22 micron filter. Glucose 10.0 g and antibiotics are added after pH adjustment and sterilization.

### 1000X Trace Metal Solution (Per Liter Fermentation Media):

Citric Acid*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component is dissolved one at a time in diH₂O. The pH is adjusted to 3.0 with HCl/NaOH, and then the solution is brought to volume and filter-sterilized with a 0.22 micron filter.

### (ii) Experimental Procedure

Cells are grown overnight in Luria-Bertani broth+antibiotics. The day after, they are diluted to an OD600 of 0.1 in 20 mL TM3 medium containing 50 ug/ml of spectinomycin, 25 ug/mL chloramphenicol and 50 ug/mL carbenicillin (in a 250-mL baffled Erlenmeyer flask), and incubated at 34° C. and 200 rpm. After 2 h of growth, OD600 is measured and 200 uM IPTG is added. Samples are taken regularly during the course of the fermentation. At each timepoint, OD600 is measured. Also, off-gas analysis of isoprene is performed using a gas chromatograph-mass spectrometer (GC-MS) (Agilent) headspace assay. One hundred microliters of whole broth are placed in a sealed GC vial and incubated at 34° C. and 200 rpm for a fixed time of 30 minutes. Following a heat kill step, consisting of incubation at 70° C. for 7 minutes, the sample is loaded on the GC. The reported specific productivity is the amount of isoprene in ug/L read by the GC divided by the incubation time (30 min) and the measured OD600.

### Example 8

### Isoprenoid Production by Recombinant Host Cells Expressing PMevD, IPK, and the Upper MVA Pathway

Isoprenoid production by strains expressing the upper MVA pathway and the alternative archaeal lower MVA pathway are compared to strains expressing the upper MVA pathway and the classical lower pathway.

(i) Materials

### TM3 Media Recipe (Per Liter Fermentation Media):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 0.2 g, 1000X Trace Metals Solution 1 ml. All of the components are added together and dissolved in diH₂O. The pH is adjusted to 6.8 with ammonium hydroxide (30%) and brought to volume. Media is then filter-sterilized with a 0.22 micron filter. Glucose 10.0 g and antibiotics are added after sterilization and pH adjustment.

### 1000X Trace Metal Solution (Per Liter Fermentation Media):

Citric Acid*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component is dissolved one at a time in diH₂O. The pH is adjusted to 3.0 with HCl/NaOH, and then the solution is brought to volume and filter-sterilized with a 0.22 micron filter.

(ii) Experimental Procedure

Cells are grown overnight in Luria-Bertani broth+antibiotics. The day after, they are diluted to an OD600 of 0.05 in 20 mL TM3 medium containing 50 ug/ml of spectinomycin and 50 ug/mL carbenicillin (in a 250-mL baffled Erlenmeyer flask), and incubated at 34° C. and 200 rpm. Prior to inoculation, an overlay of 20% (v/v) dodecane (Sigma-Aldrich) is added to the culture flask to trap the volatile sesquiterpene product as described previously (Newman et. al., Biotechnol. Bioeng. 95:684-691, 2006).

After 2 hours of growth, OD600 is measured and 0.05-0.40 mM isopropyl β-d-1-thiogalactopyranoside (IPTG) is added. Samples are taken regularly during the course of the fermentation. At each time point, OD600 is measured. Also, isoprenoid concentration in the organic layer is assayed by diluting the dodecane overlay into ethyl acetate. Dodecane/ethyl acetate extracts are analyzed by GC-MS methods as previously described (Martin et. al., Nat. Biotechnol. 2003, 21:96-802). Isoprenoid samples of known concentration are injected to produce standard curves for isoprenoid. The amount of isoprenoid per sample is calculated using the isoprenoid standard curves.

### Sequences

pMCM2200 nucleic acid sequence
(SEQ ID NO: 1)
atccggatatagttcctcctttcagcaaaaaacccctcaagacccgt
ttagaggccccaaggggttatgctagttattgctcagcggtggcagc
agccaactcagcttcctttcgggctttgttagcagccggatctcagt
ggtggtggtggtggtgctcgagtcatcagccaacaagacgagcttct
gggccggctccgttaactatggtccattgtactgcgtcaagttcgca
caagcgtgcttccacttctggcgcatcttttgcttcacagatcacgt
gcacattagggccggcgtctatcgtccagtaggactgcaaattatct
tgggctctccagcgttgaacggcttgcatgaccgctaaagtgcctgg
caaccagtacattgttgaaggctgtgcggtcatcgctattacgtgca
tagacatggcgtccgcctctgacgcccgtccgagccgttcaatatca
cgctcaaggataccctgtcttacatctgctaaccgctgttcaattcc
ttccagacgcacagaaaagtatggactagtggttgccacggagtggc
cgcttgtagatgcaacatgtttagcttccgtggagataacagcaaca
atatcgacgagattccaatgttccggtggagcgatctgtgccgcata
agagccagcatgggttccatcattgtaccactctacaaaaccagcag
ggatactgcgacaagcacttcccgaaccacttaagcgggtaaggcga
gagagttctgcctcatctaactccagtctaaatgcactggcagcagc
ccgagtaagggcggcaaacgccgcagcggagctcgcgatacctgcat
cagacgggaaattattacgactgcggacttccacgcgttcggttaca
ccagccagctggcgcaagcgctcaatctgctggataactctttcgaa
ctggcgtcccttagcctgcacttcctcacctccagaaagtgccaacc
acacggaatcgtcaactgcctctggaagacattgcacggttgtttca
gtgaggcaaccatccaagttcatggaaatcgagccattggtaggaag
ggtcaactgactgtcgtgctggccccaatatttgatgaacgcaatgt
tggcacaagcgacagccgtcgctgcgtgagacagctgtttcattccg
ttccggacatacataccctggaagtataagttctctccaccggcccc
atggtgatgatggtggtgcatatgtatatctccttcttaaagttaaa
caaaattatttctagaggggaattgttatccgctcacaattccccta
tagtgagtcgtattaatttcgcgggatcgagatctcgatcctctacg
ccggacgcatcgtggccggcatcaccggcgccacaggtgcggttgct
ggcgcctatatcgccgacatcaccgatggggaagatcgggctcgcca
cttcgggctcatgagcgcttgtttcggcgtgggtatggtggcaggcc
ccgtggccgggggactgttgggcgccatctccttgcatgcaccattc
cttgcggcggcggtgctcaacggcctcaacctactactgggctgctt
cctaatgcaggagtcgcataagggagagcgtcgagatcccggacacc
atcgaatggcgcaaaacctttcgcggtatggcatgatagcgcccgga
agagagtcaattcagggtggtgaatgtgaaaccagtaacgttatacg
atgtcgcagagtatgccggtgtctcttatcagaccgtttcccgcgtg
gtgaaccaggccagccacgtttctgcgaaaacgcgggaaaaagtgga
agcggcgatggcggagctgaattacattcccaaccgcgtggcacaac
aactggcgggcaaacagtcgttgctgattggcgttgccacctccagt
ctggccctgcacgcgccgtcgcaaattgtcgcggcgattaaatctcg
cgccgatcaactgggtgccagcgtggtggtgtcgatggtagaacgaa
gcggcgtcgaagcctgtaaagcggcggtgcacaatcttctcgcgcaa
cgcgtcagtgggctgatcattaactatccgctggatgaccaggatgc
cattgctgtggaagctgcctgcactaatgttccggcgttatttcttg
atgtctctgaccagacacccatcaacagtattattttctcccatgaa
gacggtacgcgactgggcgtggagcatctggtcgcattgggtcacca
gcaaatcgcgctgttagcgggcccattaagttctgtctcggcgcgtc
tgcgtctggctggctggcataaatatctcactcgcaatcaaattcag
ccgatagcggaacgggaaggcgactggagtgccatgtccggttttca
acaaaccatgcaaatgctgaatgagggcatcgttcccactgcgatgc
tggttgccaacgatcagatggcgctgggcgcaatgcgcgccattacc
gagtccgggctgcgcgttggtgcggatatctcggtagtgggatacga
cgataccgaagacagctcatgttatatcccgccgttaaccaccatca
aacaggattttcgcctgctggggcaaaccagcgtggaccgcttgctg
caactctctcagggccaggcggtgaagggcaatcagctgttgcccgt
ctcactggtgaaaagaaaaaccaccctggcgcccaatacgcaaaccg
cctctccccgcgcgttggccgattcattaatgcagctggcacgacag
gtttcccgactggaaagcgggcagtgagcgcaacgcaattaatgtaa
gttagctcactcattaggcaccgggatctcgaccgatgcccttgaga
gccttcaacccagtcagctccttccggtgggcgcggggcatgactat
cgtcgccgcacttatgactgtcttctttatcatgcaactcgtaggac
aggtgccggcagcgctctgggtcattttcggcgaggaccgctttcgc
tggagcgcgacgatgatcggcctgtcgcttgcggtattcggaatctt
gcacgccctcgctcaagccttcgtcactggtcccgccaccaaacgtt
tcggcgagaagcaggccattatcgccggcatggcggccccacgggtg
cgcatgatcgtgctcctgtcgttgaggacccggctaggctggcgggg
ttgccttactggttagcagaatgaatcaccgatacgcgagcgaacgt
gaagcgactgctgctgcaaaacgtctgcgacctgagcaacaacatga
atggtcttcggtttccgtgtttcgtaaagtctggaaacgcggaagtc
agcgccctgcaccattatgttccggatctgcatcgcaggatgctgct
ggctaccctgtggaacacctacatctgtattaacgaagcgctggcat
tgaccctgagtgatttttctctggtcccgccgcatccataccgccag
ttgtttaccctcacaacgttccagtaaccgggcatgttcatcatcag
taacccgtatcgtgagcatcctctctcgtttcatcggtatcattacc
cccatgaacagaaatcccccttacacggaggcatcagtgaccaaaca
ggaaaaaaccgcccttaacatggcccgctttatcagaagccagacat
taacgcttctggagaaactcaacgagctggacgcggatgaacaggca
gacatctgtgaatcgcttcacgaccacgctgatgagctttaccgcag
ctgcctcgcgcgtttcggtgatgacggtgaaaacctctgacacatgc
agctcccggagacggtcacagcttgtctgtaagcggatgccgggagc
agacaagcccgtcagggcgcgtcagcgggtgttggcgggtgtcgggg
cgcagccatgacccagtcacgtagcgatagcggagtgtatactggct
taactatgcggcatcagagcagattgtactgagagtgcaccatatat
gcggtgtgaaataccgcacagatgcgtaaggagaaaataccgcatca
ggcgctcttccgcttcctcgctcactgactcgctgcgctcggtcgtt
cggctgcggcgagcggtatcagctcactcaaaggcggtaatacggtt
atccacagaatcaggggataacgcaggaaagaacatgtgagcaaaag
gccagcaaaaggccaggaaccgtaaaaaggccgcgttgctggcgttt
ttccataggctccgcccccctgacgagcatcacaaaaatcgacgctc
aagtcagaggtggcgaaacccgacaggactataaagataccaggcgt
ttccccctggaagctccctcgtgcgctctcctgttccgaccctgccg
cttaccggatacctgtccgcctttctcccttcgggaagcgtggcgct
ttctcatagctcacgctgtaggtatctcagttcggtgtaggtcgttc
gctccaagctgggctgtgtgcacgaaccccccgttcagcccgaccgc
tgcgccttatccggtaactatcgtcttgagtccaacccggtaagaca
cgacttatcgccactggcagcagccactggtaacaggattagcagag
cgaggtatgtaggcggtgctacagagttcttgaagtggtggcctaac
tacggctacactagaaggacagtatttggtatctgcgctctgctgaa
gccagttaccttcggaaaaagagttggtagctcttgatccggcaaac
aaaccaccgctggtagcggtggtttttttgtttgcaagcagcagatt
acgcgcagaaaaaaaggatctcaagaagatcctttgatcttttctac
ggggtctgacgctcagtggaacgaaaactcacgttaagggattttgg
tcatgaacaataaaactgtctgcttacataaacagtaatacaagggg
tgttatgagccatattcaacgggaaacgtcttgctctaggccgcgat
taaattccaacatggatgctgatttatatgggtataaatgggctcgc
gataatgtcgggcaatcaggtgcgacaatctatcgattgtatgggaa
gcccgatgcgccagagttgtttctgaaacatggcaaaggtagcgttg
ccaatgatgttacagatgagatggtcagactaaactggctgacggaa
tttatgcctcttccgaccatcaagcattttatccgtactcctgatga
tgcatggttactcaccactgcgatccccgggaaaacagcattccagg
tattagaagaatatcctgattcaggtgaaaatattgttgatgcgctg
gcagtgttcctgcgccggttgcattcgattcctgtttgtaattgtcc
ttttaacagcgatcgcgtatttcgtctcgctcaggcgcaatcacgaa
tgaataacggtttggttgatgcgagtgattttgatgacgagcgtaat
ggctggcctgttgaacaagtctggaaagaaatgcataaacttttgcc
attctcaccggattcagtcgtcactcatggtgatttctcacttgata
accttatttttgacgaggggaaattaataggttgtattgatgttgga
cgagtcggaatcgcagaccgataccaggatcttgccatcctatggaa
ctgcctcggtgagttttctccttcattacagaaacggctttttcaaa
aatatggtattgataatcctgatatgaataaattgcagtttcatttg
atgctcgatgagtttttctaagaattaattcatgagcggatacatat
ttgaatgtatttagaaaaataaacaaataggggttccgcgcacattt
ccccgaaaagtgccacctgaaattgtaaacgttaatattttgttaaa
attcgcgttaaatttttgttaaatcagctcattttttaaccaatagg
ccgaaatcggcaaaatcccttataaatcaaaagaatagaccgagata
gggttgagtgttgttccagtttggaacaagagtccactattaaagaa
cgtggactccaacgtcaaagggcgaaaaaccgtctatcagggcgatg
gcccactacgtgaaccatcaccctaatcaagttttttggggtcgagg
tgccgtaaagcactaaatcggaaccctaaagggagcccccgatttag
agcttgacggggaaagccggcgaacgtggcgagaaaggaagggaaga
aagcgaaaggagcgggcgctagggcgctggcaagtgtagcggtcacg
ctgcgcgtaaccaccacacccgccgcgcttaatgcgccgctacaggg
cgcgtcccattcgcca
pMCM2201 nucleic acid sequence
(SEQ ID NO: 2)
atccggatatagttcctcctttcagcaaaaaacccctcaagacccgt
ttagaggccccaaggggttatgctagttattgctcagcggtggcagc
agccaactcagcttcctttcgggctttgttagcagccggatctcagt
ggtggtggtggtggtgctcgagtcatcaatccaattttataatggtt
cccggcccattcagttggccactcaacgcagattggagctgttgggg
accgcaaatccaaatttccaactgcggggcctgctggacaagctgcc
acatagcttctaccttattgcgcattcctcctgtgacgtccacgcca
tgagacccgccaagccgtgctataatggtagcgtagttggttctgtt
gatgagtggaataggctgggcatcggcatgttgccgagggtcggcat
catacacggcctgctctcccaacagaatgatctgcgtcggctgtaag
ggaccgaccagggcactaaaaatgcgctctgtactagcgatggtaca
accctgggccacatccagcagtacatcgccatatataactggaatcg
tcccggctgctaaaagcgtcgccaacggctgagagccaatctgctga
atttcccccgcgttcgcgagactactggccatcggttgaataccaat
tgctggtaagtctgcgtctaagcaagctccgacaaccgcacgattca
gccgggccatggcatccgccacacgagcaacgccccaccaactttgt
tcgttgataataccctgggcggtctggtaccgttctgcccagtaatg
gccgaatgagccacctccatgtcccaacagaattggctggttaggat
gggcctggcgccatgcactcagatccgtcacgacctgtttaagtgtt
tggtcaactaaccgttcggccgttgtcttatctgtgagcatagaacc
acccagcttgataaaaatcggcttgttcattccctggaagtacagat
tctctccgccagctccgtggtggtgatgatggtgcatatgtatatct
ccttcttaaagttaaacaaaattatttctagaggggaattgttatcc
gctcacaattcccctatagtgagtcgtattaatttcgcgggatcgag
atctcgatcctctacgccggacgcatcgtggccggcatcaccggcgc
cacaggtgcggttgctggcgcctatatcgccgacatcaccgatgggg
aagatcgggctcgccacttcgggctcatgagcgcttgtttcggcgtg
ggtatggtggcaggccccgtggccgggggactgttgggcgccatctc
cttgcatgcaccattccttgcggcggcggtgctcaacggcctcaacc
tactactgggctgcttcctaatgcaggagtcgcataagggagagcgt
cgagatcccggacaccatcgaatggcgcaaaacctttcgcggtatgg
catgatagcgcccggaagagagtcaattcagggtggtgaatgtgaaa
ccagtaacgttatacgatgtcgcagagtatgccggtgtctcttatca
gaccgtttcccgcgtggtgaaccaggccagccacgtttctgcgaaaa
cgcgggaaaaagtggaagcggcgatggcggagctgaattacattccc
aaccgcgtggcacaacaactggcgggcaaacagtcgttgctgattgg
cgttgccacctccagtctggccctgcacgcgccgtcgcaaattgtcg
cggcgattaaatctcgcgccgatcaactgggtgccagcgtggtggtg
tcgatggtagaacgaagcggcgtcgaagcctgtaaagcggcggtgca
caatcttctcgcgcaacgcgtcagtgggctgatcattaactatccgc
tggatgaccaggatgccattgctgtggaagctgcctgcactaatgtt
ccggcgttatttcttgatgtctctgaccagacacccatcaacagtat
tattttctcccatgaagacggtacgcgactgggcgtggagcatctgg
tcgcattgggtcaccagcaaatcgcgctgttagcgggcccattaagt
tctgtctcggcgcgtctgcgtctggctggctggcataaatatctcac
tcgcaatcaaattcagccgatagcggaacgggaaggcgactggagtg
ccatgtccggttttcaacaaaccatgcaaatgctgaatgagggcatc
gttcccactgcgatgctggttgccaacgatcagatggcgctgggcgc
aatgcgcgccattaccgagtccgggctgcgcgttggtgcggatatct
cggtagtgggatacgacgataccgaagacagctcatgttatatcccg
ccgttaaccaccatcaaacaggattttcgcctgctggggcaaaccag
cgtggaccgcttgctgcaactctctcagggccaggcggtgaagggca
atcagctgttgcccgtctcactggtgaaaagaaaaaccaccctggcg
cccaatacgcaaaccgcctctccccgcgcgttggccgattcattaat
gcagctggcacgacaggtttcccgactggaaagcgggcagtgagcgc
aacgcaattaatgtaagttagctcactcattaggcaccgggatctcg
accgatgcccttgagagccttcaacccagtcagctccttccggtggg
cgcggggcatgactatcgtcgccgcacttatgactgtcttctttatc
atgcaactcgtaggacaggtgccggcagcgctctgggtcattttcgg
cgaggaccgctttcgctggagcgcgacgatgatcggcctgtcgcttg
cggtattcggaatcttgcacgccctcgctcaagccttcgtcactggt
cccgccaccaaacgtttcggcgagaagcaggccattatcgccggcat
ggcggccccacgggtgcgcatgatcgtgctcctgtcgttgaggaccc
ggctaggctggcggggttgccttactggttagcagaatgaatcaccg
atacgcgagcgaacgtgaagcgactgctgctgcaaaacgtctgcgac
ctgagcaacaacatgaatggtcttcggtttccgtgtttcgtaaagtc
tggaaacgcggaagtcagcgccctgcaccattatgttccggatctgc
atcgcaggatgctgctggctaccctgtggaacacctacatctgtatt
aacgaagcgctggcattgaccctgagtgatttttctctggtcccgcc
gcatccataccgccagttgtttaccctcacaacgttccagtaaccgg
gcatgttcatcatcagtaacccgtatcgtgagcatcctctctcgttt
catcggtatcattacccccatgaacagaaatcccccttacacggagg
catcagtgaccaaacaggaaaaaaccgcccttaacatggcccgcttt
atcagaagccagacattaacgcttctggagaaactcaacgagctgga
cgcggatgaacaggcagacatctgtgaatcgcttcacgaccacgctg
atgagctttaccgcagctgcctcgcgcgtttcggtgatgacggtgaa
aacctctgacacatgcagctcccggagacggtcacagcttgtctgta
agcggatgccgggagcagacaagcccgtcagggcgcgtcagcgggtg
ttggcgggtgtcggggcgcagccatgacccagtcacgtagcgatagc
ggagtgtatactggcttaactatgcggcatcagagcagattgtactg
agagtgcaccatatatgcggtgtgaaataccgcacagatgcgtaagg
agaaaataccgcatcaggcgctcttccgcttcctcgctcactgactc
gctgcgctcggtcgttcggctgcggcgagcggtatcagctcactcaa
aggcggtaatacggttatccacagaatcaggggataacgcaggaaag
aacatgtgagcaaaaggccagcaaaaggccaggaaccgtaaaaaggc
cgcgttgctggcgtttttccataggctccgcccccctgacgagcatc
acaaaaatcgacgctcaagtcagaggtggcgaaacccgacaggacta
taaagataccaggcgtttccccctggaagctccctcgtgcgctctcc
tgttccgaccctgccgcttaccggatacctgtccgcctttctccctt
cgggaagcgtggcgctttctcatagctcacgctgtaggtatctcagt
tcggtgtaggtcgttcgctccaagctgggctgtgtgcacgaaccccc
cgttcagcccgaccgctgcgccttatccggtaactatcgtcttgagt
ccaacccggtaagacacgacttatcgccactggcagcagccactggt
aacaggattagcagagcgaggtatgtaggcggtgctacagagttctt
gaagtggtggcctaactacggctacactagaaggacagtatttggta
tctgcgctctgctgaagccagttaccttcggaaaaagagttggtagc
tcttgatccggcaaacaaaccaccgctggtagcggtggtttttttgt
ttgcaagcagcagattacgcgcagaaaaaaaggatctcaagaagatc
ctttgatcttttctacggggtctgacgctcagtggaacgaaaactca
cgttaagggattttggtcatgaacaataaaactgtctgcttacataa
acagtaatacaaggggtgttatgagccatattcaacgggaaacgtct
tgctctaggccgcgattaaattccaacatggatgctgatttatatgg
gtataaatgggctcgcgataatgtcgggcaatcaggtgcgacaatct
atcgattgtatgggaagcccgatgcgccagagttgtttctgaaacat
ggcaaaggtagcgttgccaatgatgttacagatgagatggtcagact
aaactggctgacggaatttatgcctcttccgaccatcaagcatttta
tccgtactcctgatgatgcatggttactcaccactgcgatccccggg
aaaacagcattccaggtattagaagaatatcctgattcaggtgaaaa
tattgttgatgcgctggcagtgttcctgcgccggttgcattcgattc
ctgtttgtaattgtccttttaacagcgatcgcgtatttcgtctcgct
caggcgcaatcacgaatgaataacggtttggttgatgcgagtgattt
tgatgacgagcgtaatggctggcctgttgaacaagtctggaaagaaa
tgcataaacttttgccattctcaccggattcagtcgtcactcatggt
gatttctcacttgataaccttatttttgacgaggggaaattaatagg
ttgtattgatgttggacgagtcggaatcgcagaccgataccaggatc
ttgccatcctatggaactgcctcggtgagttttctccttcattacag
aaacggctttttcaaaaatatggtattgataatcctgatatgaataa
attgcagtttcatttgatgctcgatgagtttttctaagaattaattc
atgagcggatacatatttgaatgtatttagaaaaataaacaaatagg
ggttccgcgcacatttccccgaaaagtgccacctgaaattgtaaacg
ttaatattttgttaaaattcgcgttaaatttttgttaaatcagctca
ttttttaaccaataggccgaaatcggcaaaatcccttataaatcaaa
agaatagaccgagatagggttgagtgttgttccagtttggaacaaga
gtccactattaaagaacgtggactccaacgtcaaagggcgaaaaacc
gtctatcagggcgatggcccactacgtgaaccatcaccctaatcaag
ttttttggggtcgaggtgccgtaaagcactaaatcggaaccctaaag
ggagcccccgatttagagcttgacggggaaagccggcgaacgtggcg
agaaaggaagggaagaaagcgaaaggagcgggcgctagggcgctggc
aagtgtagcggtcacgctgcgcgtaaccaccacacccgccgcgctta
atgcgccgctacagggcgcgtcccattcgcca
pMCM2212 nucleic acid sequence
(SEQ ID NO: 3)
atccggatatagttcctcctttcagcaaaaaacccctcaagacccgt
ttagaggccccaaggggttatgctagttattgctcagcggtggcagc
agccaactcagcttcctttcgggctttgttagcagccggatctcagt
ggtggtggtggtggtgctcgagtcatcaaatcagctgagcaccctgc
cccgcgcgagctttgaatattgattgtactcccggacattctttaag
aagcgcctcgactttctcagcctcgctcgaaagggttaacacgtgca
catttggaccggcatccaccgttgaacagactggtatacccttcttg
cgccaatgaattactttccataagatcacttcagtttccggtaacca
ataattgagtggtggtttacttgttctcatgaccgcgtgcatgagat
tactatcctcctccacaacgctcgcgaagtgttcaaaatcacggtca
aggatcgctttccgacagatttctatgcgttcttctacacgttcctg
ccgtaaaaggtgaagatcggaagtgctcgccagagcatgaccgcctg
tggagcctacagttttgtgttcggagttaaggacgcaaatcagatct
acaagatcccaatgatccgccggtgctatactccatgcaaatgaatc
ctggtctgtcgagcccgcttgccattccacaaagccatccggaatgc
tacgacaggcagacccactacctctccgcgcgagacggctcagtgct
tcttcatccagagaaaggccagcggctttagacgcggccaaagcaag
ggccgcaaacgcggaagctgaactagctatcccagctccagatggga
agctgttctccgactcgacttttgcgaagaaggaaatgcccgccaga
tcgcgaacgatttccaggaaatcgctaacgcgacgcagcgcgtccca
ctcaataggcttgccggacaacttaaactggtctgccgaaagggatg
gatcaaactgtaccgatgtttttgtttcgagccctgaaaggttcatt
gacaaggatccgttgcatggcagacgcaaatcattgtcgcgattacc
ccagtacttaatgaatgcgatattcgggtgagccagggccgacactt
ccagaaattctggcgatttcatagggatttcattattgttgatcacg
cggtaatagtaatccatgccttgaaaatacagattctcgccgcctgc
accgtgatggtgatggtggtgcatatgtatatctccttcttaaagtt
aaacaaaattatttctagaggggaattgttatccgctcacaattccc
ctatagtgagtcgtattaatttcgcgggatcgagatctcgatcctct
acgccggacgcatcgtggccggcatcaccggcgccacaggtgcggtt
gctggcgcctatatcgccgacatcaccgatggggaagatcgggctcg
ccacttcgggctcatgagcgcttgtttcggcgtgggtatggtggcag
gccccgtggccgggggactgttgggcgccatctccttgcatgcacca
ttccttgcggcggcggtgctcaacggcctcaacctactactgggctg
cttcctaatgcaggagtcgcataagggagagcgtcgagatcccggac
accatcgaatggcgcaaaacctttcgcggtatggcatgatagcgccc
ggaagagagtcaattcagggtggtgaatgtgaaaccagtaacgttat
acgatgtcgcagagtatgccggtgtctcttatcagaccgtttcccgc
gtggtgaaccaggccagccacgtttctgcgaaaacgcgggaaaaagt
ggaagcggcgatggcggagctgaattacattcccaaccgcgtggcac
aacaactggcgggcaaacagtcgttgctgattggcgttgccacctcc
agtctggccctgcacgcgccgtcgcaaattgtcgcggcgattaaatc
tcgcgccgatcaactgggtgccagcgtggtggtgtcgatggtagaac
gaagcggcgtcgaagcctgtaaagcggcggtgcacaatcttctcgcg
caacgcgtcagtgggctgatcattaactatccgctggatgaccagga
tgccattgctgtggaagctgcctgcactaatgttccggcgttatttc
ttgatgtctctgaccagacacccatcaacagtattattttctcccat
gaagacggtacgcgactgggcgtggagcatctggtcgcattgggtca
ccagcaaatcgcgctgttagcgggcccattaagttctgtctcggcgc
gtctgcgtctggctggctggcataaatatctcactcgcaatcaaatt
cagccgatagcggaacgggaaggcgactggagtgccatgtccggttt
tcaacaaaccatgcaaatgctgaatgagggcatcgttcccactgcga
tgctggttgccaacgatcagatggcgctgggcgcaatgcgcgccatt
accgagtccgggctgcgcgttggtgcggatatctcggtagtgggata
cgacgataccgaagacagctcatgttatatcccgccgttaaccacca
tcaaacaggattttcgcctgctggggcaaaccagcgtggaccgcttg
ctgcaactctctcagggccaggcggtgaagggcaatcagctgttgcc
cgtctcactggtgaaaagaaaaaccaccctggcgcccaatacgcaaa
ccgcctctccccgcgcgttggccgattcattaatgcagctggcacga
caggtttcccgactggaaagcgggcagtgagcgcaacgcaattaatg
taagttagctcactcattaggcaccgggatctcgaccgatgcccttg
agagccttcaacccagtcagctccttccggtgggcgcggggcatgac
tatcgtcgccgcacttatgactgtcttctttatcatgcaactcgtag
gacaggtgccggcagcgctctgggtcattttcggcgaggaccgcttt
cgctggagcgcgacgatgatcggcctgtcgcttgcggtattcggaat
cttgcacgccctcgctcaagccttcgtcactggtcccgccaccaaac
gtttcggcgagaagcaggccattatcgccggcatggcggccccacgg
gtgcgcatgatcgtgctcctgtcgttgaggacccggctaggctggcg
gggttgccttactggttagcagaatgaatcaccgatacgcgagcgaa
cgtgaagcgactgctgctgcaaaacgtctgcgacctgagcaacaaca
tgaatggtcttcggtttccgtgtttcgtaaagtctggaaacgcggaa
gtcagcgccctgcaccattatgttccggatctgcatcgcaggatgct
gctggctaccctgtggaacacctacatctgtattaacgaagcgctgg
cattgaccctgagtgatttttctctggtcccgccgcatccataccgc
cagttgtttaccctcacaacgttccagtaaccgggcatgttcatcat
cagtaacccgtatcgtgagcatcctctctcgtttcatcggtatcatt
acccccatgaacagaaatcccccttacacggaggcatcagtgaccaa
acaggaaaaaaccgcccttaacatggcccgctttatcagaagccaga
cattaacgcttctggagaaactcaacgagctggacgcggatgaacag
gcagacatctgtgaatcgcttcacgaccacgctgatgagctttaccg
cagctgcctcgcgcgtttcggtgatgacggtgaaaacctctgacaca
tgcagctcccggagacggtcacagcttgtctgtaagcggatgccggg
agcagacaagcccgtcagggcgcgtcagcgggtgttggcgggtgtcg
gggcgcagccatgacccagtcacgtagcgatagcggagtgtatactg
gcttaactatgcggcatcagagcagattgtactgagagtgcaccata
tatgcggtgtgaaataccgcacagatgcgtaaggagaaaataccgca
tcaggcgctcttccgcttcctcgctcactgactcgctgcgctcggtc
gttcggctgcggcgagcggtatcagctcactcaaaggcggtaatacg
gttatccacagaatcaggggataacgcaggaaagaacatgtgagcaa
aaggccagcaaaaggccaggaaccgtaaaaaggccgcgttgctggcg
tttttccataggctccgcccccctgacgagcatcacaaaaatcgacg
ctcaagtcagaggtggcgaaacccgacaggactataaagataccagg
cgtttccccctggaagctccctcgtgcgctctcctgttccgaccctg
ccgcttaccggatacctgtccgcctttctcccttcgggaagcgtggc
gctttctcatagctcacgctgtaggtatctcagttcggtgtaggtcg
ttcgctccaagctgggctgtgtgcacgaaccccccgttcagcccgac
cgctgcgccttatccggtaactatcgtcttgagtccaacccggtaag
acacgacttatcgccactggcagcagccactggtaacaggattagca
gagcgaggtatgtaggcggtgctacagagttcttgaagtggtggcct
aactacggctacactagaaggacagtatttggtatctgcgctctgct
gaagccagttaccttcggaaaaagagttggtagctcttgatccggca
aacaaaccaccgctggtagcggtggtttttttgtttgcaagcagcag
attacgcgcagaaaaaaaggatctcaagaagatcctttgatcttttc
tacggggtctgacgctcagtggaacgaaaactcacgttaagggattt
tggtcatgaacaataaaactgtctgcttacataaacagtaatacaag
gggtgttatgagccatattcaacgggaaacgtcttgctctaggccgc
gattaaattccaacatggatgctgatttatatgggtataaatgggct
cgcgataatgtcgggcaatcaggtgcgacaatctatcgattgtatgg
gaagcccgatgcgccagagttgtttctgaaacatggcaaaggtagcg
ttgccaatgatgttacagatgagatggtcagactaaactggctgacg
gaatttatgcctcttccgaccatcaagcattttatccgtactcctga
tgatgcatggttactcaccactgcgatccccgggaaaacagcattcc
aggtattagaagaatatcctgattcaggtgaaaatattgttgatgcg
ctggcagtgttcctgcgccggttgcattcgattcctgtttgtaattg
tccttttaacagcgatcgcgtatttcgtctcgctcaggcgcaatcac
gaatgaataacggtttggttgatgcgagtgattttgatgacgagcgt
aatggctggcctgttgaacaagtctggaaagaaatgcataaactttt
gccattctcaccggattcagtcgtcactcatggtgatttctcacttg
ataaccttatttttgacgaggggaaattaataggttgtattgatgtt
ggacgagtcggaatcgcagaccgataccaggatcttgccatcctatg
gaactgcctcggtgagttttctccttcattacagaaacggctttttc
aaaaatatggtattgataatcctgatatgaataaattgcagtttcat
ttgatgctcgatgagtttttctaagaattaattcatgagcggataca
tatttgaatgtatttagaaaaataaacaaataggggttccgcgcaca
tttccccgaaaagtgccacctgaaattgtaaacgttaatattttgtt
aaaattcgcgttaaatttttgttaaatcagctcattttttaaccaat
aggccgaaatcggcaaaatcccttataaatcaaaagaatagaccgag
atagggttgagtgttgttccagtttggaacaagagtccactattaaa
gaacgtggactccaacgtcaaagggcgaaaaaccgtctatcagggcg
atggcccactacgtgaaccatcaccctaatcaagttttttggggtcg
aggtgccgtaaagcactaaatcggaaccctaaagggagcccccgatt
tagagcttgacggggaaagccggcgaacgtggcgagaaaggaaggga
agaaagcgaaaggagcgggcgctagggcgctggcaagtgtagcggtc
acgctgcgcgtaaccaccacacccgccgcgcttaatgcgccgctaca
gggcgcgtcccattcgcca
pMCM2244 nucleic acid sequence
(SEQ ID NO: 4)
atggccaagttgaccagtgccgttccggtgctcaccgcgcgcgacgt
cgccggagcggtcgagttctggaccgaccggctcgggttctccccta
gtaacggccgccagtgtgctggaattcaggcagttcaacctgttgat
agtacgtactaagctctcatgtttcacgtactaagctctcatgttta
acgtactaagctctcatgtttaacgaactaaaccctcatggctaacg
tactaagctctcatggctaacgtactaagctctcatgtttcacgtac
taagctctcatgtttgaacaataaaattaatataaatcagcaactta
aatagcctctaaggttttaagttttataagaaaaaaaagaatatata
aggcttttaaagcttttaaggtttaacggttgtggacaacaagccag
ggatgtaacgcactgagaagcccttagagcctctcaaagcaattttc
agtgacacaggaacacttaacggctgacagcctgaaaattaaccctc
actaaagggcggccgcgaagttcctattctctagaaagtataggaac
ttcctcgagccctatagtgagtcgtattaaattcatataaaaaacat
acagataaccatctgcggtgataaattatctctggcggtgttgacgt
aaataccactggcggtgatactgagcacatcagcaggacgcactgac
caccatgaaggtgcaaaggaggtaaaaaaacatggtatcctgttctg
cgccgggtaagatttacctgttcggtgaacacgccgtagtttatggc
gaaactgcaattgcgtgtgcggtggaactgcgtacccgtgttcgcgc
ggaactcaatgactctatcactattcagagccagatcggccgcaccg
gtctggatttcgaaaagcacccttatgtgtctgcggtaattgagaaa
atgcgcaaatctattcctattaacggtgttttcttgaccgtcgattc
cgacatcccggtgggctccggtctgggtagcagcgcagccgttacta
tcgcgtctattggtgcgctgaacgagctgttcggctttggcctcagc
ctgcaagaaatcgctaaactgggccacgaaatcgaaattaaagtaca
gggtgccgcgtccccaaccgatacgtatgtttctaccttcggcggcg
tggttaccatcccggaacgtcgcaaactgaaaactccggactgcggc
attgtgattggcgataccggcgttttctcctccaccaaagagttagt
agctaacgtacgtcagctgcgcgaaagctacccggatttgatcgaac
cgctgatgacctctattggcaaaatctctcgtatcggcgaacaactg
gttctgtctggcgactacgcatccatcggccgcctgatgaacgtcaa
ccagggtctcctggacgccctgggcgttaacatcttagaactgagcc
agctgatctattccgctcgtgcggcaggtgcgtttggcgctaaaatc
acgggcgctggcggcggtggctgtatggttgcgctgaccgctccgga
aaaatgcaaccaagtggcagaagcggtagcaggcgctggcggtaaag
tgactatcactaaaccgaccgagcaaggtctgaaagtagattaagct
aatttgcgataggcctgcacccttaaggaggaaaaaaacatgtcaga
gttgagagccttcagtgccccagggaaagcgttactagctggtggat
atttagttttagatacaaaatatgaagcatttgtagtcggattatcg
gcaagaatgcatgctgtagcccatccttacggttcattgcaagggtc
tgataagtttgaagtgcgtgtgaaaagtaaacaatttaaagatgggg
agtggctgtaccatataagtcctaaaagtggcttcattcctgtttcg
ataggcggatctaagaaccctttcattgaaaaagttatcgctaacgt
atttagctactttaaacctaacatggacgactactgcaatagaaact
tgttcgttattgatattttctctgatgatgcctaccattctcaggag
gatagcgttaccgaacatcgtggcaacagaagattgagttttcattc
gcacagaattgaagaagttcccaaaacagggctgggctcctcggcag
gtttagtcacagttttaactacagctttggcctccttttttgtatcg
gacctggaaaataatgtagacaaatatagagaagttattcataattt
agcacaagttgctcattgtcaagctcagggtaaaattggaagcgggt
ttgatgtagcggcggcagcatatggatctatcagatatagaagattc
ccacccgcattaatctctaatttgccagatattggaagtgctactta
cggcagtaaactggcgcatttggttgatgaagaagactggaatatta
cgattaaaagtaaccatttaccttcgggattaactttatggatgggc
gatattaagaatggttcagaaacagtaaaactggtccagaaggtaaa
aaattggtatgattcgcatatgccagaaagcttgaaaatatatacag
aactcgatcatgcaaattctagatttatggatggactatctaaacta
gatcgcttacacgagactcatgacgattacagcgatcagatatttga
gtctcttgagaggaatgactgtacctgtcaaaagtatcctgaaatca
cagaagttagagatgcagttgccacaattagacgttcctttagaaaa
ataactaaagaatctggtgccgatatcgaacctcccgtacaaactag
cttattggatgattgccagaccttaaaaggagttcttacttgcttaa
tacctggtgctggtggttatgacgccattgcagtgattactaagcaa
gatgttgatcttagggctcaaaccgctaatgacaaaagattttctaa
ggttcaatggctggatgtaactcaggctgactggggtgttaggaaag
aaaaagatccggaaacttatcttgataaataacttaaggtagctgca
tgcagaattcgcccttaaggaggaaaaaaaaatgaccgtttacacag
catccgttaccgcacccgtcaacatcgcaacccttaagtattggggg
aaaagggacacgaagttgaatctgcccaccaattcgtccatatcagt
gactttatcgcaagatgacctcagaacgttgacctctgcggctactg
cacctgagtttgaacgcgacactttgtggttaaatggagaaccacac
agcatcgacaatgaaagaactcaaaattgtctgcgcgacctacgcca
attaagaaaggaaatggaatcgaaggacgcctcattgcccacattat
ctcaatggaaactccacattgtctccgaaaataactttcctacagca
gctggtttagcttcctccgctgctggctttgctgcattggtctctgc
aattgctaagttataccaattaccacagtcaacttcagaaatatcta
gaatagcaagaaaggggtctggttcagcttgtagatcgttgtttggc
ggatacgtggcctgggaaatgggaaaagctgaagatggtcatgattc
catggcagtacaaatcgcagacagctctgactggcctcagatgaaag
cttgtgtcctagttgtcagcgatattaaaaaggatgtgagttccact
cagggtatgcaattgaccgtggcaacctccgaactatttaaagaaag
aattgaacatgtcgtaccaaagagatttgaagtcatgcgtaaagcca
ttgttgaaaaagatttcgccacctttgcaaaggaaacaatgatggat
tccaactctttccatgccacatgtttggactctttccctccaatatt
ctacatgaatgacacttccaagcgtatcatcagttggtgccacacca
ttaatcagttttacggagaaacaatcgttgcatacacgtttgatgca
ggtccaaatgctgtgttgtactacttagctgaaaatgagtcgaaact
ctttgcatttatctataaattgtttggctctgttcctggatgggaca
agaaatttactactgagcagcttgaggctttcaaccatcaatttgaa
tcatctaactttactgcacgtgaattggatcttgagttgcaaaagga
tgttgccagagtgattttaactcaagtcggttcaggcccacaagaaa
caaacgaatctttgattgacgcaaagactggtctaccaaaggaataa
gatcaattcgctgcatcgcccttaggaggtaaaaaaaaatgactgcc
gacaacaatagtatgccccatggtgcagtatctagttacgccaaatt
agtgcaaaaccaaacacctgaagacattttggaagagtttcctgaaa
ttattccattacaacaaagacctaatacccgatctagtgagacgtca
aatgacgaaagcggagaaacatgtttttctggtcatgatgaggagca
aattaagttaatgaatgaaaattgtattgttttggattgggacgata
atgctattggtgccggtaccaagaaagtttgtcatttaatggaaaat
attgaaaagggtttactacatcgtgcattctccgtctttattttcaa
tgaacaaggtgaattacttttacaacaaagagccactgaaaaaataa
ctttccctgatctttggactaacacatgctgctctcatccactatgt
attgatgacgaattaggtttgaagggtaagctagacgataagattaa
gggcgctattactgcggcggtgagaaaactagatcatgaattaggta
ttccagaagatgaaactaagacaaggggtaagtttcactttttaaac
agaatccattacatggcaccaagcaatgaaccatggggtgaacatga
aattgattacatcctattttataagatcaacgctaaagaaaacttga
ctgtcaacccaaacgtcaatgaagttagagacttcaaatgggtttca
ccaaatgatttgaaaactatgtttgctgacccaagttacaagtttac
gccttggtttaagattatttgcgagaattacttattcaactggtggg
agcaattagatgacctttctgaagtggaaaatgacaggcaaattcat
agaatgctataacaacgcgtctacaaataaaaaaggcacgtcagatg
acgtgccttttttcttggggcccaagaaaaatgccccgcttacgcag
ggcatccatttattactcaaccgtaaccgattttgccaggttacgcg
gctggtcaacgtcggtgcctttgatcagcgcgacatggtaagccagc
agctgcagcggaacggtgtagaagatcggtgcaatcacctcttccac
atgcggcatctcgatgatgtgcatgttatcgctacttacaaaacccg
catcctgatcggcgaagacatacaactgaccgccacgcgcgcgaact
tcttcaatgttggattttagtttttccagcaattcgttgttcggtgc
aacgacgataaccggcatatcggcatcaatcagcgccagcggaccgt
gtttcagttcacctgcaccgtaggcttcagcgtgaatgtaagagatc
tctttcagcttcaatgcgccttccagcgcgattgggtactgatcgcc
acggcccaggaacagcgcgtgatgtttgtcagagaaatcttctgcca
gagcttcaatgcgtttgtcctgagacagcatctgctcaatacggctc
ggcaacgcctgcagaccatgcacaatgtcatgttcaatggaggcatc
cagacctttcaggcgagacagcttcgccaccagcatcaacagcacag
ttaactgagtggtgaatgctttagtggatgccacgccgatttctgta
cccgcgttggtcattagcgccagatggccgtcgttttacaacgtcgt
gactgggaaaaccctggcgttacccaacttaatcgccttgcagcaca
tccccctttcgccagctggcgtaatagcgaagaggcccgcaccgatc
gcccttcccaacagttgcgcagcctatacgtacggcagtttaaggtt
tacacctataaaagagagagccgttatcgtctgtttgtggatgtaca
gagtgatattattgacacgccggggcgacggatggtgatccccctgg
ccagtgcacgtctgctgtcagataaagtctcccgtgaactttacccg
gtggtgcatatcggggatgaaagctggcgcatgatgaccaccgatat
ggccagtgtgccggtctccgttatcggggaagaagtggctgatctca
gccaccgcgaaaatgacatcaaaaacgccattaacctgatgttctgg
ggaatataaatgtcaggcatgagattatcaaaaaggatcttcaccta
gatccttttcacgtagaaagccagtccgcagaaacggtgctgacccc
ggatgaatgtcagctactgggctatctggacaagggaaaacgcaagc
gcaaagagaaagcaggtagcttgcagtgggcttacatggcgatagct
agactgggcggttttatggacagcaagcgaaccggaattgccagctg
gggcgccctctggtaaggttgggaagccctgcaaagtaaactggatg
gctttctcgccgccaaggatctgatggcgcaggggatcaagctctga
tcaagagacaggatgaggatcgtttcgcatgattgaacaagatggat
tgcacgcaggttctccggccgcttgggtggagaggctattcggctat
gactgggcacaacagacaatcggctgctctgatgccgccgtgttccg
gctgtcagcgcaggggcgcccggttctttttgtcaagaccgacctgt
ccggtgccctgaatgaactgcaagacgaggcagcgcggctatcgtgg
ctggccacgacgggcgttccttgcgcagctgtgctcgacgttgtcac
tgaagcgggaagggactggctgctattgggcgaagtgccggggcagg
atctcctgtcatctcaccttgctcctgccgagaaagtatccatcatg
gctgatgcaatgcggcggctgcatacgcttgatccggctacctgccc
attcgaccaccaagcgaaacatcgcatcgagcgagcacgtactcgga
tggaagccggtcttgtcgatcaggatgatctggacgaagagcatcag
gggctcgcgccagccgaactgttcgccaggctcaaggcgagcatgcc
cgacggcgaggatctcgtcgtgacccatggcgatgcctgcttgccga
atatcatggtggaaaatggccgcttttctggattcatcgactgtggc
cggctgggtgtggcggaccgctatcaggacatagcgttggctacccg
tgatattgctgaagagcttggcggcgaatgggctgaccgcttcctcg
tgctttacggtatcgccgctcccgattcgcagcgcatcgccttctat
cgccttcttgacgagttcttctgaattattaacgcttacaatttcct
gatgcggtattttctccttacgcatctgtgcggtatttcacaccgca
tacaggtggcacttttcggggaaatgtgcgcggaacccctatttgtt
tatttttctaaatacattcaaatatgtatccgctcatgagacaataa
ccctgataaatgcttcaataatagcacgtgaggagggccacc
pMCM2246 nucleic acid sequence
(SEQ ID NO: 5)
atggccaagttgaccagtgccgttccggtgctcaccgcgcgcgacgt
cgccggagcggtcgagttctggaccgaccggctcgggttctccccta
gtaacggccgccagtgtgctggaattcaggcagttcaacctgttgat
agtacgtactaagctctcatgtttcacgtactaagctctcatgttta
acgtactaagctctcatgtttaacgaactaaaccctcatggctaacg
tactaagctctcatggctaacgtactaagctctcatgtttcacgtac
taagctctcatgtttgaacaataaaattaatataaatcagcaactta
aatagcctctaaggttttaagttttataagaaaaaaaagaatatata
aggcttttaaagcttttaaggtttaacggttgtggacaacaagccag
ggatgtaacgcactgagaagcccttagagcctctcaaagcaattttc
agtgacacaggaacacttaacggctgacagcctgaaaattaaccctc
actaaagggcggccgcgaagttcctattctctagaaagtataggaac
ttcctcgagccctatagtgagtcgtattaaattcatataaaaaacat
acagataaccatctgcggtgataaattatctctggcggtgttgacgt
aaataccactggcggtgatactgagcacatcagcaggacgcactgac
caccatgaaggtgcaaaggaggtaaaaaaacatggtatcctgttctg
cgccgggtaagatttacctgttcggtgaacacgccgtagtttatggc
gaaactgcaattgcgtgtgcggtggaactgcgtacccgtgttcgcgc
ggaactcaatgactctatcactattcagagccagatcggccgcaccg
gtctggatttcgaaaagcacccttatgtgtctgcggtaattgagaaa
atgcgcaaatctattcctattaacggtgttttcttgaccgtcgattc
cgacatcccggtgggctccggtctgggtagcagcgcagccgttacta
tcgcgtctattggtgcgctgaacgagctgttcggctttggcctcagc
ctgcaagaaatcgctaaactgggccacgaaatcgaaattaaagtaca
gggtgccgcgtccccaaccgatacgtatgtttctaccttcggcggcg
tggttaccatcccggaacgtcgcaaactgaaaactccggactgcggc
attgtgattggcgataccggcgttttctcctccaccaaagagttagt
agctaacgtacgtcagctgcgcgaaagctacccggatttgatcgaac
cgctgatgacctctattggcaaaatctctcgtatcggcgaacaactg
gttctgtctggcgactacgcatccatcggccgcctgatgaacgtcaa
ccagggtctcctggacgccctgggcgttaacatcttagaactgagcc
agctgatctattccgctcgtgcggcaggtgcgtttggcgctaaaatc
acgggcgctggcggcggtggctgtatggttgcgctgaccgctccgga
aaaatgcaaccaagtggcagaagcggtagcaggcgctggcggtaaag
tgactatcactaaaccgaccgagcaaggtctgaaagtagattaacca
ggatagctctttgatcggaactgaacttcagtttagcaaaggagagt
atcgatggattactattaccgcgtgatcaacaataatgaaatcccta
tgaaatcgccagaatttctggaagtgtcggccctggctcacccgaat
atcgcattcattaagtactggggtaatcgcgacaatgatttgcgtct
gccatgcaacggatccttgtcaatgaacctttcagggctcgaaacaa
aaacatcggtacagtttgatccatccctttcggcagaccagtttaag
ttgtccggcaagcctattgagtgggacgcgctgcgtcgcgttagcga
tttcctggaaatcgttcgcgatctggcgggcatttccttcttcgcaa
aagtcgagtcggagaacagcttcccatctggagctgggatagctagt
tcagcttccgcgtttgcggcccttgctttggccgcgtctaaagccgc
tggcctttctctggatgaagaagcactgagccgtctcgcgcggagag
gtagtgggtctgcctgtcgtagcattccggatggctttgtggaatgg
caagcgggctcgacagaccaggattcatttgcatggagtatagcacc
ggcggatcattgggatcttgtagatctgatttgcgtccttaactccg
aacacaaaactgtaggctccacaggcggtcatgctctggcgagcact
tccgatcttcaccttttacggcaggaacgtgtagaagaacgcataga
aatctgtcggaaagcgatccttgaccgtgattttgaacacttcgcga
gcgttgtggaggaggatagtaatctcatgcacgcggtcatgagaaca
agtaaaccaccactcaattattggttaccggaaactgaagtgatctt
atggaaagtaattcattggcgcaagaagggtataccagtctgttcaa
cggtggatgccggtccaaatgtgcacgtgttaaccctttcgagcgag
gctgagaaagtcgaggcgcttcttaaagaatgtccgggagtacaatc
aatattcaaagctcgcgcggggcagggtgctcagctgatttgatttg
tagatgccacggaccatagcaatatactgcgagaagggagggttaac
ttatgaacaagccgatttttatcaagctgggtggttctatgctcaca
gataagacaacggccgaacggttagttgaccaaacacttaaacaggt
cgtgacggatctgagtgcatggcgccaggcccatcctaaccagccaa
ttctgttgggacatggaggtggctcattcggccattactgggcagaa
cggtaccagaccgcccagggtattatcaacgaacaaagttggtgggg
cgttgctcgtgtggcggatgccatggcccggctgaatcgtgcggttg
tcggagcttgcttagacgcagacttaccagcaattggtattcaaccg
atggccagtagtctcgcgaacgcgggggaaattcagcagattggctc
tcagccgttggcgacgcttttagcagccgggacgattccagttatat
atggcgatgtactgctggatgtggcccagggttgtaccatcgctagt
acagagcgcatttttagtgccctggtcggtcccttacagccgacgca
gatcattctgttgggagagcaggccgtgtatgatgccgaccctcggc
aacatgccgatgcccagcctattccactcatcaacagaaccaactac
gctaccattatagcacggcttggcgggtctcatggcgtggacgtcac
aggaggaatgcgcaataaggtagaagctatgtggcagcttgtccagc
aggccccgcagttggaaatttggatttgcggtccccaacagctccaa
tctgcgttgagtggccaactgaatgggccgggaaccattataaaatt
ggattgaaaatgactctgaattgctgccggctgaaaagcaggctctc
ggaggaggaaatatgactgccgacaacaatagtatgccccatggtgc
agtatctagttacgccaaattagtgcaaaaccaaacacctgaagaca
ttttggaagagtttcctgaaattattccattacaacaaagacctaat
acccgatctagtgagacgtcaaatgacgaaagcggagaaacatgttt
ttctggtcatgatgaggagcaaattaagttaatgaatgaaaattgta
ttgttttggattgggacgataatgctattggtgccggtaccaagaaa
gtttgtcatttaatggaaaatattgaaaagggtttactacatcgtgc
attctccgtctttattttcaatgaacaaggtgaattacttttacaac
aaagagccactgaaaaaataactttccctgatctttggactaacaca
tgctgctctcatccactatgtattgatgacgaattaggtttgaaggg
taagctagacgataagattaagggcgctattactgcggcggtgagaa
aactagatcatgaattaggtattccagaagatgaaactaagacaagg
ggtaagtttcactttttaaacagaatccattacatggcaccaagcaa
tgaaccatggggtgaacatgaaattgattacatcctattttataaga
tcaacgctaaagaaaacttgactgtcaacccaaacgtcaatgaagtt
agagacttcaaatgggtttcaccaaatgatttgaaaactatgtttgc
tgacccaagttacaagtttacgccttggtttaagattatttgcgaga
attacttattcaactggtgggagcaattagatgacctttctgaagtg
gaaaatgacaggcaaattcatagaatgctataacaacgcgtctacaa
ataaaaaaggcacgtcagatgacgtgccttttttcttggggcccaag
aaaaatgccccgcttacgcagggcatccatttattactcaaccgtaa
ccgattttgccaggttacgcggctggtcaacgtcggtgcctttgatc
agcgcgacatggtaagccagcagctgcagcggaacggtgtagaagat
cggtgcaatcacctcttccacatgcggcatctcgatgatgtgcatgt
tatcgctacttacaaaacccgcatcctgatcggcgaagacatacaac
tgaccgccacgcgcgcgaacttcttcaatgttggattttagtttttc
cagcaattcgttgttcggtgcaacgacgataaccggcatatcggcat
caatcagcgccagcggaccgtgtttcagttcacctgcagcgtaggct
tcagcgtgaatgtaagagatctctttcagcttcaatgcgccttccag
cgcgattgggtactgatcgccacggcccaggaacagcgcgtgatgtt
tgtcagagaaatcttctgccagagcttcaatgcgtttgtcctgagac
agcatctgctcaatacggctcggcaacgcctgcagaccatgcacaat
gtcatgttcaatggaggcatccagacctttcaggcgagacagcttcg
ccaccagcatcaacagcacagttaactgagtggtgaatgctttagtg
gatgccacgccgatttctgtacccgcgttggtcattagcgccagatg
gccgtcgttttacaacgtcgtgactgggaaaaccctggcgttaccca
acttaatcgccttgcagcacatccccctttcgccagctggcgtaata
gcgaagaggcccgcaccgatcgcccttcccaacagttgcgcagccta
tacgtacggcagtttaaggtttacacctataaaagagagagccgtta
tcgtctgtttgtggatgtacagagtgatattattgacacgccggggc
gacggatggtgatccccctggccagtgcacgtctgctgtcagataaa
gtctcccgtgaactttacccggtggtgcatatcggggatgaaagctg
gcgcatgatgaccaccgatatggccagtgtgccggtctccgttatcg
gggaagaagtggctgatctcagccaccgcgaaaatgacatcaaaaac
gccattaacctgatgttctggggaatataaatgtcaggcatgagatt
atcaaaaaggatcttcacctagatccttttcacgtagaaagccagtc
cgcagaaacggtgctgaccccggatgaatgtcagctactgggctatc
tggacaagggaaaacgcaagcgcaaagagaaagcaggtagcttgcag
tgggcttacatggcgatagctagactgggcggttttatggacagcaa
gcgaaccggaattgccagctggggcgccctctggtaaggttgggaag
ccctgcaaagtaaactggatggctttctcgccgccaaggatctgatg
gcgcaggggatcaagctctgatcaagagacaggatgaggatcgtttc
gcatgattgaacaagatggattgcacgcaggttctccggccgcttgg
gtggagaggctattcggctatgactgggcacaacagacaatcggctg
ctctgatgccgccgtgttccggctgtcagcgcaggggcgcccggttc
tttttgtcaagaccgacctgtccggtgccctgaatgaactgcaagac
gaggcagcgcggctatcgtggctggccacgacgggcgttccttgcgc
agctgtgctcgacgttgtcactgaagcgggaagggactggctgctat
tgggcgaagtgccggggcaggatctcctgtcatctcaccttgctcct
gccgagaaagtatccatcatggctgatgcaatgcggcggctgcatac
gcttgatccggctacctgcccattcgaccaccaagcgaaacatcgca
tcgagcgagcacgtactcggatggaagccggtcttgtcgatcaggat
gatctggacgaagagcatcaggggctcgcgccagccgaactgttcgc
caggctcaaggcgagcatgcccgacggcgaggatctcgtcgtgaccc
atggcgatgcctgcttgccgaatatcatggtggaaaatggccgcttt
tctggattcatcgactgtggccggctgggtgtggcggaccgctatca
ggacatagcgttggctacccgtgatattgctgaagagcttggcggcg
aatgggctgaccgcttcctcgtgctttacggtatcgccgctcccgat
tcgcagcgcatcgccttctatcgccttcttgacgagttcttctgaat
tattaacgcttacaatttcctgatgcggtattttctccttacgcatc
tgtgcggtatttcacaccgcatacaggtggcacttttcggggaaatg
tgcgcggaacccctatttgtttatttttctaaatacattcaaatatg
tatccgctcatgagacaataaccctgataaatgcttcaataatagca
cgtgaggagggccacc
pMCM2248 nucleic acid sequence
(SEQ ID NO: 6)
atggccaagttgaccagtgccgttccggtgctcaccgcgcgcgacgt
cgccggagcggtcgagttctggaccgaccggctcgggttctccccta
gtaacggccgccagtgtgctggaattcaggcagttcaacctgttgat
agtacgtactaagctctcatgtttcacgtactaagctctcatgttta
acgtactaagctctcatgtttaacgaactaaaccctcatggctaacg
tactaagctctcatggctaacgtactaagctctcatgtttcacgtac
taagctctcatgtttgaacaataaaattaatataaatcagcaactta
aatagcctctaaggttttaagttttataagaaaaaaaagaatatata
aggcttttaaagcttttaaggtttaacggttgtggacaacaagccag
ggatgtaacgcactgagaagcccttagagcctctcaaagcaattttc
agtgacacaggaacacttaacggctgacagcctgaaaattaaccctc
actaaagggcggccgcgaagttcctattctctagaaagtataggaac
ttcctcgagccctatagtgagtcgtattaaattcatataaaaaacat
acagataaccatctgcggtgataaattatctctggcggtgttgacgt
aaataccactggcggtgatactgagcacatcagcaggacgcactgac
caccatgaaggtgcaaaggaggtaaaaaaacatggtatcctgttctg
cgccgggtaagatttacctgttcggtgaacacgccgtagtttatggc
gaaactgcaattgcgtgtgcggtggaactgcgtacccgtgttcgcgc
ggaactcaatgactctatcactattcagagccagatcggccgcaccg
gtctggatttcgaaaagcacccttatgtgtctgcggtaattgagaaa
atgcgcaaatctattcctattaacggtgttttcttgaccgtcgattc
cgacatcccggtgggctccggtctgggtagcagcgcagccgttacta
tcgcgtctattggtgcgctgaacgagctgttcggctttggcctcagc
ctgcaagaaatcgctaaactgggccacgaaatcgaaattaaagtaca
gggtgccgcgtccccaaccgatacgtatgtttctaccttcggcggcg
tggttaccatcccggaacgtcgcaaactgaaaactccggactgcggc
attgtgattggcgataccggcgttttctcctccaccaaagagttagt
agctaacgtacgtcagctgcgcgaaagctacccggatttgatcgaac
cgctgatgacctctattggcaaaatctctcgtatcggcgaacaactg
gttctgtctggcgactacgcatccatcggccgcctgatgaacgtcaa
ccagggtctcctggacgccctgggcgttaacatcttagaactgagcc
agctgatctattccgctcgtgcggcaggtgcgtttggcgctaaaatc
acgggcgctggcggcggtggctgtatggttgcgctgaccgctccgga
aaaatgcaaccaagtggcagaagcggtagcaggcgctggcggtaaag
tgactatcactaaaccgaccgagcaaggtctgaaagtagattaacca
ggatagctctttgatcggaacaaacgaaaatcaaaggaggaaccaac
aatgtatgtccggaacggaatgaaacagctgtctcacgcagcgacgg
ctgtcgcttgtgccaacattgcgttcatcaaatattggggccagcac
gacagtcagttgacccttcctaccaatggctcgatttccatgaactt
ggatggttgcctcactgaaacaaccgtgcaatgtcttccagaggcag
ttgacgattccgtgtggttggcactttctggaggtgaggaagtgcag
gctaagggacgccagttcgaaagagttatccagcagattgagcgctt
gcgccagctggctggtgtaaccgaacgcgtggaagtccgcagtcgta
ataatttcccgtctgatgcaggtatcgcgagctccgctgcggcgttt
gccgcccttactcgggctgctgccagtgcatttagactggagttaga
tgaggcagaactctctcgccttacccgcttaagtggttcgggaagtg
cttgtcgcagtatccctgctggttttgtagagtggtacaatgatgga
acccatgctggctcttatgcggcacagatcgctccaccggaacattg
gaatctcgtcgatattgttgctgttatctccacggaagctaaacatg
ttgcatctacaagcggccactccgtggcaaccactagtccatacttt
tctgtgcgtctggaaggaattgaacagcggttagcagatgtaagaca
gggtatccttgagcgtgatattgaacggctcggacgggcgtcagagg
cggacgccatgtctatgcacgtaatagcgatgaccgcacagccttca
acaatgtactggttgccaggcactttagcggtcatgcaagccgttca
acgctggagagcccaagataatttgcagtcctactggacgatagacg
ccggccctaatgtgcacgtgatctgtgaagcaaaagatgcgccagaa
gtggaagcacgcttgtgcgaacttgacgcagtacaatggaccatagt
taacggagccggcccagaagctcgtcttgttggctgatttgtagatg
ccacggaccatagcaatatactgcgagaagggagggttaacttatga
acaagccgatttttatcaagctgggtggttctatgctcacagataag
acaacggccgaacggttagttgaccaaacacttaaacaggtcgtgac
ggatctgagtgcatggcgccaggcccatcctaaccagccaattctgt
tgggacatggaggtggctcattcggccattactgggcagaacggtac
cagaccgcccagggtattatcaacgaacaaagttggtggggcgttgc
tcgtgtggcggatgccatggcccggctgaatcgtgcggttgtcggag
cttgcttagacgcagacttaccagcaattggtattcaaccgatggcc
agtagtctcgcgaacgcgggggaaattcagcagattggctctcagcc
gttggcgacgcttttagcagccgggacgattccagttatatatggcg
atgtactgctggatgtggcccagggttgtaccatcgctagtacagag
cgcatttttagtgccctggtcggtcccttacagccgacgcagatcat
tctgttgggagagcaggccgtgtatgatgccgaccctcggcaacatg
ccgatgcccagcctattccactcatcaacagaaccaactacgctacc
attatagcacggcttggcgggtctcatggcgtggacgtcacaggagg
aatgcgcaataaggtagaagctatgtggcagcttgtccagcaggccc
cgcagttggaaatttggatttgcggtccccaacagctccaatctgcg
ttgagtggccaactgaatgggccgggaaccattataaaattggattg
aaaatgactctgaattgctgccggctgaaaagcaggctctcggagga
ggaaatatgactgccgacaacaatagtatgccccatggtgcagtatc
tagttacgccaaattagtgcaaaaccaaacacctgaagacattttgg
aagagtttcctgaaattattccattacaacaaagacctaatacccga
tctagtgagacgtcaaatgacgaaagcggagaaacatgtttttctgg
tcatgatgaggagcaaattaagttaatgaatgaaaattgtattgttt
tggattgggacgataatgctattggtgccggtaccaagaaagtttgt
catttaatggaaaatattgaaaagggtttactacatcgtgcattctc
cgtctttattttcaatgaacaaggtgaattacttttacaacaaagag
ccactgaaaaaataactttccctgatctttggactaacacatgctgc
tctcatccactatgtattgatgacgaattaggtttgaagggtaagct
agacgataagattaagggcgctattactgcggcggtgagaaaactag
atcatgaattaggtattccagaagatgaaactaagacaaggggtaag
tttcactttttaaacagaatccattacatggcaccaagcaatgaacc
atggggtgaacatgaaattgattacatcctattttataagatcaacg
ctaaagaaaacttgactgtcaacccaaacgtcaatgaagttagagac
ttcaaatgggtttcaccaaatgatttgaaaactatgtttgctgaccc
aagttacaagtttacgccttggtttaagattatttgcgagaattact
tattcaactggtgggagcaattagatgacctttctgaagtggaaaat
gacaggcaaattcatagaatgctataacaacgcgtctacaaataaaa
aaggcacgtcagatgacgtgccttttttcttggggcccaagaaaaat
gccccgcttacgcagggcatccatttattactcaaccgtaaccgatt
ttgccaggttacgcggctggtcaacgtcggtgcctttgatcagcgcg
acatggtaagccagcagctgcagcggaacggtgtagaagatcggtgc
aatcacctcttccacatgcggcatctcgatgatgtgcatgttatcgc
tacttacaaaacccgcatcctgatcggcgaagacatacaactgaccg
ccacgcgcgcgaacttcttcaatgttggattttagtttttccagcaa
ttcgttgttcggtgcaacgacgataaccggcatatcggcatcaatca
gcgccagcggaccgtgtttcagttcacctgcagcgtaggcttcagcg
tgaatgtaagagatctctttcagcttcaatgcgccttccagcgcgat
tgggtactgatcgccacggcccaggaacagcgcgtgatgtttgtcag
agaaatcttctgccagagcttcaatgcgtttgtcctgagacagcatc
tgctcaatacggctcggcaacgcctgcagaccatgcacaatgtcatg
ttcaatggaggcatccagacctttcaggcgagacagcttcgccacca
gcatcaacagcacagttaactgagtggtgaatgctttagtggatgcc
acgccgatttctgtacccgcgttggtcattagcgccagatggccgtc
gttttacaacgtcgtgactgggaaaaccctggcgttacccaacttaa
tcgccttgcagcacatccccctttcgccagctggcgtaatagcgaag
aggcccgcaccgatcgcccttcccaacagttgcgcagcctatacgta
cggcagtttaaggtttacacctataaaagagagagccgttatcgtct
gtttgtggatgtacagagtgatattattgacacgccggggcgacgga
tggtgatccccctggccagtgcacgtctgctgtcagataaagtctcc
cgtgaactttacccggtggtgcatatcggggatgaaagctggcgcat
gatgaccaccgatatggccagtgtgccggtctccgttatcggggaag
aagtggctgatctcagccaccgcgaaaatgacatcaaaaacgccatt
aacctgatgttctggggaatataaatgtcaggcatgagattatcaaa
aaggatcttcacctagatccttttcacgtagaaagccagtccgcaga
aacggtgctgaccccggatgaatgtcagctactgggctatctggaca
agggaaaacgcaagcgcaaagagaaagcaggtagcttgcagtgggct
tacatggcgatagctagactgggcggttttatggacagcaagcgaac
cggaattgccagctggggcgccctctggtaaggttgggaagccctgc
aaagtaaactggatggctttctcgccgccaaggatctgatggcgcag
gggatcaagctctgatcaagagacaggatgaggatcgtttcgcatga
ttgaacaagatggattgcacgcaggttctccggccgcttgggtggag
aggctattcggctatgactgggcacaacagacaatcggctgctctga
tgccgccgtgttccggctgtcagcgcaggggcgcccggttctttttg
tcaagaccgacctgtccggtgccctgaatgaactgcaagacgaggca
gcgcggctatcgtggctggccacgacgggcgttccttgcgcagctgt
gctcgacgttgtcactgaagcgggaagggactggctgctattgggcg
aagtgccggggcaggatctcctgtcatctcaccttgctcctgccgag
aaagtatccatcatggctgatgcaatgcggcggctgcatacgcttga
tccggctacctgcccattcgaccaccaagcgaaacatcgcatcgagc
gagcacgtactcggatggaagccggtcttgtcgatcaggatgatctg
gacgaagagcatcaggggctcgcgccagccgaactgttcgccaggct
caaggcgagcatgcccgacggcgaggatctcgtcgtgacccatggcg
atgcctgcttgccgaatatcatggtggaaaatggccgcttttctgga
ttcatcgactgtggccggctgggtgtggcggaccgctatcaggacat
agcgttggctacccgtgatattgctgaagagcttggcggcgaatggg
ctgaccgcttcctcgtgctttacggtatcgccgctcccgattcgcag
cgcatcgccttctatcgccttcttgacgagttcttctgaattattaa
cgcttacaatttcctgatgcggtattttctccttacgcatctgtgcg
gtatttcacaccgcatacaggtggcacttttcggggaaatgtgcgcg
gaacccctatttgtttatttttctaaatacattcaaatatgtatccg
ctcatgagacaataaccctgataaatgcttcaataatagcacgtgag
gagggccacc
Amino acid sequence of Herpetosiphon aurantiacus
phosphomevalonate decarboxylase
(SEQ ID NO: 16)
MKQLSHAATAVACANIAFIKYWGQHDSQLTLPTNGSISMNLDGCLTE
TTVQCLPEAVDDSVWLALSGGEEVQAKGRQFERVIQQIERLRQLAGV
TERVEVRSRNNFPSDAGIASSAAAFAALTRAAASAFRLELDEAELSR
LTRLSGSGSACRSIPAGFVEWYNDGTHAGSYAAQIAPPEHWNLVDIV
AVISTEAKHVASTSGHSVATTSPYFSVRLEGIEQRLADVRQGILERD
IERLGRASEADAMSMHVIAMTAQPSTMYWLPGTLAVMQAVQRWRAQD
NLQSYWTIDAGPNVHVICEAKDAPEVEARLCELDAVQWTIVNGAGPE
ARLVG
Amino acid sequence of Anaerolinea thennophila
phosphomevalonate decarboxylase
(SEQ ID NO: 17)
MGQATAIAHPNIAFIKYWGNRDAVLRIPENGSISMNLAELTVKTTVI
FEKHSREDTLILNGALADEPALKRVSHFLDRVREFAGISWHAHVISE
NNFPTGAGIASSAAAFAALALAATSAIGLHLSERDLSRLARKGSGSA
CRSIPGGFVEWIPGETDEDSYAVSIAPPEHWALTDCIAILSTQHKPI
GSTQGHALASTSPLQPARVADTPRRLEIVRRAILERDFLSLAEMIEH
DSNLMHAVMMTSTPPLFYWEPVSLVIMKSVREWRESGLPCAYTLDAG
PNVHVICPSEYAEEVIFRLTSIPGVQTVLKASAGDSAKLIEQSL
Amino acid sequence of S378Pa3-2
phosphomevalonate decarboxylase
(SEQ ID NO: 18)
MDYYYRVINNNEIPMKSPEFLEVSALAHPNIAFIKYWGNRDNDLRLP
CNGSLSMNLSGLETKTSVQFDPSLSADQFKLSGKPIEWDALRRVSDF
LEIVRDLAGISFFAKVESENSFPSGAGIASSASAFAALALAASKAAG
LSLDEEALSRLARRGSGSACRSIPDGFVEWQAGSTDQDSFAWSIAPA
DHWDLVDLICVLNSEHKTVGSTGGHALASTSDLHLLRQERVEERIEI
CRKAILDRDFEHFASVVEEDSNLMHAVMRTSKPPLNYWLPETEVILW
KVIHWRKKGIPVCSTVDAGPNVHVLTLSSEAEKVEALLKECPGVQSI
FKARAGQGAQLI
Amino acid sequence of Herpetosiphon aurantiacus
isopentenyl kinase
(SEQ ID NO: 19)
MNKPIFIKLGGSMLTDKTTAERLVDQTLKQVVTDLSAWRQAHPNQPI
LLGHGGGSFGHYWAERYQTAQGIINEQSWWGVARVADAMARLNRAVV
GACLDADLPAIGIQPMASSLANAGEIQQIGSQPLATLLAAGTIPVIY
GDVLLDVAQGCTIASTERIFSALVGPLQPTQIILLGEQAVYDADPRQ
HADAQPIPLINRTNYATIIARLGGSHGVDVTGGMRNKVEAMWQLVQQ
APQLEIWICGPQQLQSALSGQLNGPGTIIKLD
Amino acid sequence of Methanocaldococcus
jannaschii DSM 2661 isopentenyl kinase
(SEQ ID NO: 20)
MLTILKLGGSILSDKNVPYSIKWDNLERIAMEIKNALDYYKNQNKEI
KLILVHGGGAFGHPVAKKYLKIEDGKKIFINMEKGFWEIQRAMRRFN
NIIIDTLQSYDIPAVSIQPSSFVVFGDKLIFDTSAIKEMLKRNLVPV
IHGDIVIDDKNGYRIISGDDIVPYLANELKADLILYATDVDGVLIDN
KPIKRIDKNNIYKILNYLSGSNSIDVTGGMKYKIDMIRKNKCRGFVF
NGNKANNIYKALLGEVEGTEIDFSE
Amino acid sequence of Methanobrevibacter
ruminantium isopentenyl kinase
(SEQ ID NO: 21)
MIILKIGGSILTEKDSAEPKVDYANLNRIAEEIRQSLYSDEMSNDLI
DGLVIVHGAGSFGHPPAKKYRIGEPFDMEDYLSKKIGFSEVQNEVKK
LNSIICQSLIEHGIPAVAIPPSAFITSHNKRIYDCNLELIKTYIGEG
FVPVLFGDVVLDDEVKIAVISGDQILQYIAKFLKSDRIVLGTDVDGV
YTKNPKTHDDAVHIDKVSSIEDIKFLESTTNVDVTGGMVGKVKELLD
LAEYGISSEIIDANEKGAISKALQGMEVRGTKISKE
Amino acid sequence of Methanobacterium
thermoautotrophicum isopentenyl kinase
(SEQ ID NO: 22)
MIILKLGGSVITRKDSEEPAIDRDNLERIASEIGNASPSSLMIVHGA
GSFGHPFAGEYRIGSEIENEEDLRRRRFGFALTQNWVKKLNSHVCDA
LLAEGIPAVSMQPSAFIRAHAGRISHADISLIRSYLEEGMVPVVYGD
VVLDSDRRLKFSVISGDQLINHFSLRLMPERVILGTDVDGVYTRNPK
KHPDARLLDVIGSLDDLESLDGTLNTDVTGGMVGKIRELLLLAEKGV
ESEIINAAVPGNIERALLGEEVRGTRITGKH
Amino acid sequence of Anaerolinea
thermophila isopentenyl kinase
(SEQ ID NO: 23)
MSMDSNLTFLKLGGSLITEKDKPRTPRAKIIQQIAWEIREALREIPN
LRLIIGHGSGSFGHATAKKYRTREGVYTLEDWYGFVHVWYDARALNQ
LVIDALFSAGLPVIAFPPSAITFREGKKVQIATQLIQIAIEKGLIPV
VQGDVIFDLDQGGTILSTEEVFAELSFHLRPQRILLAGVEEGVWADF
PLRHSLVTEISEDTIKSENIQISGSIATDVTGGMAEKVKSMLDLCQR
VPGLEVWIFNGLKKGNVLNALRGFPMGTKILSRNS

What is claimed is:

1. Recombinant cells capable of producing isoprene, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an isoprene synthase polypeptide, wherein culturing of said recombinant cells provides for the production of isoprene.

2. The recombinant cells of claim 1, wherein the nucleic acid encoding a polypeptide having phosphomevalonate decarboxylase activity catalyzes the conversion of mevalonate 5-phosphate to isopentenyl phosphate.

3. The recombinant cells of claim 1 or 2, wherein the nucleic acid encoding a polypeptide having phosphomevalonate decarboxylase activity catalyzes the conversion of mevalonate 5-pyrophosphate to isopentenyl phosphate and/or isopentenyl pyrophosphate.

4. The recombinant cells of any one of claims 1-3, wherein the nucleic acid encoding a polypeptide having phosphomevalonate decarboxylase activity is from an archaea.

5. The recombinant cells of claim 4, wherein the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota.

6. The recombinant cells of any one of claims 1-3, wherein the nucleic acid encoding a polypeptide having phosphomevalonate decarboxylase activity is from a microorganism selected from the group consisting of: Herpetosiphon aurantiacus, S378Pa3-2, and Anaerolinea thermophila.

7. The recombinant cells of any one of claims 1-3, wherein the nucleic acid encoding a polypeptide having phosphomevalonate decarboxylase activity encodes a polypeptide having an amino acid sequence with at least 85% sequence identity to an amino acid sequence selected from the group consisting of SEQ ID NOs:16-18.

8. The recombinant cells of any one of claims 1-7, wherein the nucleic acid encoding a polypeptide having isopentenyl kinase activity is from an archaea.

9. The recombinant cells of claim 8, wherein the archaea is selected from the group consisting of desulforococcales, sulfolobales, thermoproteales, cenarchaeales, nitro sopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota.

10. The recombinant cells of any one of claims 1-7, wherein the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila.

11. The recombinant cells of claim 10, wherein the microorganism is Herpetosiphon aurantiacus or Methanococcus jannaschii.

12. The recombinant cells of any one of claims 1-7, wherein the nucleic acid encoding a polypeptide having isopentenyl kinase activity encodes a polypeptide having an amino acid sequence with at least 85% sequence identity to an amino acid sequence selected from the group consisting of SEQ ID NOs:19-23.

13. The recombinant cells of any one of claims 1-12, wherein the isoprene synthase polypeptide is a plant isoprene synthase polypeptide.

14. The recombinant cells of claim 13, wherein the plant isoprene synthase polypeptide is a polypeptide or variant thereof from Pueraria or Populus.

15. The recombinant cells of claim 13, wherein the plant isoprene synthase polypeptide is a polypeptide or variant thereof from Pueraria montana or Pueraria lobata, Populus tremuloides, Populus alba, Populus nigra, Populus trichocarpa, or a hybrid Populus alba×Populus tremula.

16. The recombinant cells of any one of claims 1-15, wherein one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate.

17. The recombinant cells of any one of claims 1-16, wherein one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate.

18. The recombinant cells of any one of claims 1-17, further comprising one or more nucleic acids encoding an isopentenyl-diphosphate delta-isomerase (IDI) polypeptide.

19. The recombinant cells of any one of claims 1-18, wherein the recombinant cells comprise an attenuated enzyme that converts mevalonate 5-pyrophosphate to isopentenyl pyrophosphate.

20. The recombinant cells of any of claims 1-19, wherein the recombinant cells comprise an attenuated enzyme that converts mevalonate 5-phosphate to mevalonate 5-pyrophosphate.

21. The recombinant cells of any one of claims 1-20, wherein the recombinant cells further comprise one or more nucleic acids encoding one or more 1-deoxy-D-xylulose 5-phosphate (DXP) pathway polypeptides.

22. The recombinant cells of any one of claims 1-21, wherein the recombinant cells comprise one or more attenuated enzymes of the 1-deoxy-D-xylulose 5-phosphate (DXP) pathway.

23. The recombinant cells of any one of claims 1-22, further comprising a heterologous nucleic acid encoding a polypeptide having phosphoketolase activity.

24. The recombinant cells of any one of claims 1-23, wherein the nucleic acid encoding a polypeptide having phosphomevalonate decarboxylase activity is a heterologous nucleic acid.

25. The recombinant cells of any one of claims 1-23, wherein the nucleic acid encoding a polypeptide having phosphomevalonate decarboxylase activity is an endogenous nucleic acid.

26. The recombinant cells of any one of claims 1-25, wherein the nucleic acid encoding a polypeptide having isopentenyl kinase activity is a heterologous nucleic acid.

27. The recombinant cells of any one of claims 1-25, wherein the nucleic acid encoding a polypeptide having isopentenyl kinase activity is an endogenous nucleic acid.

28. The recombinant cells of any one of claims 1-27, wherein at least one of the nucleic acids encoding a polypeptide of (i)-(iv) is placed under an inducible promoter or a constitutive promoter.

29. The recombinant cells of any one of claims 1-28, wherein at least one of the nucleic acids encoding a polypeptide of (i)-(iv) is cloned into one or more multicopy plasmids.

30. The recombinant cells of any one of claims 1-29, wherein at least one of the nucleic acids encoding a polypeptide of (i)-(iv) is integrated into a chromosome of the cells.

31. The recombinant cells of any one of claims 1-30, wherein the recombinant cells are gram-positive bacterial cells, gram-negative bacterial cells, fungal cells, filamentous fungal cells, plant cells, algal cells or yeast cells.

32. The recombinant cells of claim 31, wherein the bacterial cells are selected from the group consisting of E. coli, L. acidophilus, Corynebacterium sp., P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells.

33. The recombinant cells of claim 31, wherein the yeast cells are selected from the group consisting of Saccharomyces sp., Schizosaccharomyces sp., Pichia sp., or Candida sp.

34. The recombinant cells of any one of claims 1-30, wherein the recombinant cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisieae and Yarrowia lipolytica.

35. Recombinant cells capable of producing isoprenoid precursors, wherein the cells comprise (i) a nucleic acid encoding a polypeptide having phosphomevalonate decarboxylase activity, (ii) a nucleic acid encoding a polypeptide having isopentenyl kinase activity, and (iii) one or more nucleic acids encoding one or more polypeptides of the MVA pathway, wherein culturing of said recombinant cells provides for the production of isoprenoid precursors.

36. The recombinant cells of claim 35, wherein the nucleic acid encoding a polypeptide having phosphomevalonate decarboxylase activity catalyzes the conversion of mevalonate 5-phosphate to isopentenyl phosphate.

37. The recombinant cells of claim 35 or 36, wherein the nucleic acid encoding a polypeptide having phosphomevalonate decarboxylase activity catalyzes the conversion of mevalonate 5-pyrophosphate to isopentenyl phosphate and/or isopentenyl pyrophosphate.

38. The recombinant cells of any one of claims 35-37, wherein the nucleic acid encoding a polypeptide having phosphomevalonate decarboxylase activity is from an archaea.

39. The recombinant cells of claim 38, wherein the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota.

40. The recombinant cells of any one of claims 35-37, wherein the nucleic acid encoding a polypeptide having phosphomevalonate decarboxylase activity is from a microorganism selected from the group consisting of: Herpetosiphon aurantiacus, S378Pa3-2, and Anaerolinea thermophila.

41. The recombinant cells of any one of claims 35-37, wherein the nucleic acid encoding a polypeptide having phosphomevalonate decarboxylase activity encodes a polypeptide having an amino acid sequence with at least 85% sequence identity to an amino acid sequence selected from the group consisting of SEQ ID NOs:16-18.

42. The recombinant cells of any one of claims 35-41, wherein the nucleic acid encoding a polypeptide having isopentenyl kinase activity is from an archaea.

43. The recombinant cells of claim 42, wherein the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota.

44. The recombinant cells of any one of claims 35-41, wherein the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila.

45. The recombinant cells of claim 44, wherein the microorganism is Herpetosiphon aurantiacus or Methanococcus jannaschii.

46. The recombinant cells of any one of claims 35-41, wherein the nucleic acid encoding a polypeptide having isopentenyl kinase activity encodes a polypeptide having an amino acid sequence with at least 85% sequence identity to an amino acid sequence selected from the group consisting of SEQ ID NOs:19-23.

47. The recombinant cells of any one of claims 35-46, wherein one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate.

48. The recombinant cells of any one of claims 35-47, wherein one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate.

49. The recombinant cells of any one of claims 35-48, wherein the recombinant cells comprise an attenuated enzyme that converts mevalonate 5-pyrophosphate to isopentenyl pyrophosphate.

50. The recombinant cells of any one of claims 35-49, wherein the recombinant cells comprise an attenuated enzyme that converts mevalonate 5-phosphate to mevalonate 5-pyrophosphate.

51. The recombinant cells of any one of claims 35-50, wherein the recombinant cells further comprise one or more nucleic acids encoding one or more 1-deoxy-D-xylulose 5-phosphate (DXP) pathway polypeptides.

52. The recombinant cells of any one of claims 35-51, wherein the recombinant cells comprise one or more attenuated enzymes of the 1-deoxy-D-xylulose 5-phosphate (DXP) pathway.

53. The recombinant cells of any one of claims 35-52, further comprising a heterologous nucleic acid encoding a polypeptide having phosphoketolase activity.

54. The recombinant cells of any one of claims 35-53, wherein the nucleic acid encoding a polypeptide having phosphomevalonate decarboxylase activity is a heterologous nucleic acid.

55. The recombinant cells of any one of claims 35-53, wherein the nucleic acid encoding a polypeptide having phosphomevalonate decarboxylase activity is an endogenous nucleic acid.

56. The recombinant cells of any one of claims 35-55, wherein the nucleic acid encoding a polypeptide having isopentenyl kinase activity is a heterologous nucleic acid.

57. The recombinant cells of any one of claims 35-55, wherein the nucleic acid encoding a polypeptide having isopentenyl kinase activity is an endogenous nucleic acid.

58. The recombinant cells of any one of claims 35-57, wherein at least one of the nucleic acids encoding a polypeptide of (i)-(iv) is placed under an inducible promoter or a constitutive promoter.

59. The recombinant cells of any one of claims 35-58, wherein at least one of the nucleic acids encoding a polypeptide of (i)-(iv) is cloned into one or more multicopy plasmids.

60. The recombinant cells of any one of claims 35-59, wherein at least one of the nucleic acids encoding a polypeptide of (i)-(iv) is integrated into a chromosome of the cells.

61. The recombinant cells of any one of claims 35-60, wherein the recombinant cells are gram-positive bacterial cells, gram-negative bacterial cells, fungal cells, filamentous fungal cells, plant cells, algal cells or yeast cells.

62. The recombinant cells of claim 61, wherein the bacterial cells are selected from the group consisting of E. coli, L. acidophilus, Corynebacterium sp., P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells.

63. The recombinant cells of claim 61, wherein the yeast cells are selected from the group consisting of Saccharomyces sp., Schizosaccharomyces sp., Pichia sp., or Candida sp.

64. The recombinant cells of any one of claims 35-60, wherein the recombinant cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisieae and Yarrowia lipolytica.

65. Recombinant cells capable of producing of isoprenoids, wherein the cells comprise (i) a nucleic acid encoding a polypeptide having phosphomevalonate decarboxylase activity, (ii) a nucleic acid encoding a polypeptide having isopentenyl kinase activity, (iii) one or more nucleic acids encoding one or more polypeptides of the MVA pathway, and (iv) a heterologous nucleic acid encoding an polyprenyl pyrophosphate synthase polypeptide, wherein culturing of said recombinant cells in a suitable media provides for the production of isoprenoids.

66. The recombinant cells of claim 65, wherein the nucleic acid encoding a polypeptide having phosphomevalonate decarboxylase activity catalyzes the conversion of mevalonate 5-phosphate to isopentenyl phosphate.

67. The recombinant cells of claim 65 or 66, wherein the nucleic acid encoding a polypeptide having phosphomevalonate decarboxylase activity catalyzes the conversion of mevalonate 5-pyrophosphate to isopentenyl phosphate and/or isopentenyl pyrophosphate.

68. The recombinant cells of any one of claims 65-67, wherein the nucleic acid encoding a polypeptide having phosphomevalonate decarboxylase activity is from an archaea.

69. The recombinant cells of claim 68, wherein the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota.

70. The recombinant cells of any one of claims 65-67, wherein the nucleic acid encoding a polypeptide having phosphomevalonate decarboxylase activity is from a microorganism selected from the group consisting of: Herpetosiphon aurantiacus, S378Pa3-2, and Anaerolinea thermophila.

71. The recombinant cells of any one of claims 65-67, wherein the nucleic acid encoding a polypeptide having phosphomevalonate decarboxylase activity encodes a polypeptide having an amino acid sequence with at least 85% sequence identity to an amino acid sequence selected from the group consisting of SEQ ID NOs:19-23.

72. The recombinant cells of any one of claims 65-71, wherein the nucleic acid encoding a polypeptide having isopentenyl kinase activity is from an archaea.

73. The recombinant cells of claim 72, wherein the archaea is selected from the group consisting of desulforococcales, sulfolobales, thermoproteales, cenarchaeales, nitrosopumilales, archeaoglobales, halobacteriales, methanococcales, methanocellales, methanosarcinales, methanobacteriales, mathanomicrobiales, methanopyrales, thermococcales, thermoplasmatales, korarchaeota, and nanoarchaeota.

74. The recombinant cells of any one of claims 65-71, wherein the nucleic acid encoding a polypeptide having isopentenyl kinase activity is from a microorganism selected from the group consisting of: Herpetosiphon aurantiacus, Methanococcus jannaschii, Methanobacterium thermoautotrophicum, Methanobrevibacter ruminantium, and Anaerolinea thermophila.

75. The recombinant cells of claim 74, wherein the microorganism is Herpetosiphon aurantiacus or Methanococcus jannaschii.

76. The recombinant cells of any one of claims 65-71, wherein the nucleic acid encoding a polypeptide having isopentenyl kinase activity encodes a polypeptide having an amino acid sequence with at least 85% sequence identity to an amino acid sequence selected from the group consisting of SEQ ID NOs:19-23.

77. The recombinant cells of any one of claims 65-76, wherein the isoprenoid is selected from group consisting of monoterpenes, diterpenes, triterpenes, tetraterpenes, sesquiterpene, and polyterpene.

78. The recombinant cells of any one of claims 65-76, wherein the isoprenoid is a sesquiterpene.

79. The recombinant cells of any one of claims 65-76, wherein the isoprenoid is selected from the group consisting of abietadiene, amorphadiene, carene, α-famesene, β-farnesene, farnesol, geraniol, geranylgeraniol, linalool, limonene, myrcene, nerolidol, ocimene, patchoulol, β-pinene, sabinene, γ-terpinene, terpindene and valencene.

80. The recombinant cells of any one of claims 65-79, wherein one or more polypeptides of the MVA pathway is selected from (a) an enzyme that condenses two molecules of acetyl-CoA to form acetoacetyl-CoA; (b) an enzyme that condenses malonyl-CoA with acetyl-CoA to form acetoacetyl-CoA; (c) an enzyme that condenses acetoacetyl-CoA with acetyl-CoA to form HMG-CoA; (d) an enzyme that converts HMG-CoA to mevalonate; and (e) an enzyme that phosphorylates mevalonate to mevalonate 5-phosphate.

81. The recombinant cells of any one of claims 65-80, wherein one or more polypeptides of the MVA pathway is selected from (a) an enzyme that phosphorylates mevalonate to form mevalonate 5-phosphate; (b) an enzyme that phosphorylates mevalonate 5-phosphate to form mevalonate 5-pyrophosphate; and (c) an enzyme that decarboxylates mevalonate 5-pyrophosphate to form isopentenyl pyrophosphate.

82. The recombinant cells of any one of claims 65-81, wherein the recombinant cells comprise an attenuated enzyme that converts mevalonate 5-pyrophosphate to isopentenyl pyrophosphate.

83. The recombinant cells of any one of claims 65-82, wherein the recombinant cells comprise an attenuated enzyme that converts mevalonate 5-phosphate to mevalonate 5-pyrophosphate.

84. The recombinant cells of any one of claims 65-83, wherein the recombinant cells further comprise one or more nucleic acids encoding one or more 1-deoxy-D-xylulose 5-phosphate (DXP) pathway polypeptides.

85. The recombinant cells of any one of claims 65-84, wherein the recombinant cells comprise one or more attenuated enzymes of the 1-deoxy-D-xylulose 5-phosphate (DXP) pathway.

86. The recombinant cells of any one of claims 65-85, further comprising a heterologous nucleic acid encoding a polypeptide having phosphoketolase activity.

87. The recombinant cells of any one of claims 65-86, wherein the nucleic acid encoding a polypeptide having phosphomevalonate decarboxylase activity is a heterologous nucleic acid.

88. The recombinant cells of any one of claims 65-87, wherein the nucleic acid encoding a polypeptide having phosphomevalonate decarboxylase activity is an endogenous nucleic acid.

89. The recombinant cells of any one of claims 65-88, wherein the nucleic acid encoding a polypeptide having isopentenyl kinase activity is a heterologous nucleic acid.

90. The recombinant cells of any one of claims 65-89, wherein the nucleic acid encoding a polypeptide having isopentenyl kinase activity is an endogenous nucleic acid.

91. The recombinant cells of any one of claims 65-90, wherein at least one of the nucleic acids encoding a polypeptide of (i)-(iv) is placed under an inducible promoter or a constitutive promoter.

92. The recombinant cells of any one of claims 65-91, wherein at least one of the nucleic acids encoding a polypeptide of (i)-(iv) is cloned into one or more multicopy plasmids.

93. The recombinant cells of any one of claims 65-92, wherein at least one of the nucleic acids encoding a polypeptide of (i)-(iv) is integrated into a chromosome of the cells.

94. The recombinant cells of any one of claims 65-93, wherein the recombinant cells are gram-positive bacterial cells, gram-negative bacterial cells, fungal cells, filamentous fungal cells, plant cells, algal cells or yeast cells.

95. The recombinant cells of claim 94, wherein the bacterial cells are selected from the group consisting of E. coli, L. acidophilus, Corynebacterium sp., P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells.

96. The recombinant cells of claim 94, wherein the yeast cells are selected from the group consisting of Saccharomyces sp., Schizosaccharomyces sp., Pichia sp., or Candida sp.

97. The recombinant cells of any one of claims 65-93, wherein the recombinant cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisieae and Yarrowia lipolytica.

98. A method of producing isoprene comprising: (a) culturing the recombinant cell of any one of claims 1-34 under conditions suitable for producing isoprene and (b) producing the isoprene.

99. The method of claim 98, further comprising (c) recovering the isoprene.

100. A method of producing an isoprenoid precursor comprising: (a) culturing the recombinant cell of any one of claims 35-64 under conditions suitable for producing an isoprenoid precursor and (b) producing an isoprenoid precursor.

101. The method of claim 100, further comprising (c) recovering the isoprenoid precursor.

102. A method of producing an isoprenoid comprising: (a) culturing the recombinant cell of any one of claims 65-97 under conditions suitable for producing an isoprenoid and (b) producing an isoprenoid.

103. The method of claim 102, further comprising (c) recovering the isoprenoid.

104. A composition comprising isoprene produced by the recombinant cells of any one of claims 1-34.

105. A composition comprising an isoprenoid precursor produced by the recombinant cells of any one of claims 35-64.

106. A composition comprising an isoprenoid produced by the recombinant cells of any one of claims 65-97.

107. An isolated nucleic acid encoding a polypeptide having phosphomevalonate decarboxylase activity, wherein said polypeptide comprises at least 85% sequence identity to the amino acid sequence of SEQ ID NO:18.

108. An isolated polypeptide having phosphomevalonate decarboxylase activity, wherein said polypeptide comprises at least 85% sequence identity to the amino acid sequence of SEQ ID NO:18.

109. An isolated cell comprising a nucleic acid encoding a polypeptide having phosphomevalonate decarboxylase activity, wherein said polypeptide comprises at least 85% sequence identity to the amino acid sequence of SEQ ID NO:18.

110. A recombinant cell comprising a nucleic acid encoding a polypeptide having phosphomevalonate decarboxylase activity, wherein said polypeptide comprises at least 85% sequence identity to the amino acid sequence of SEQ ID NO:18.

111. A cell extract comprising a polypeptide having phosphomevalonate decarboxylase activity, wherein said polypeptide comprises at least 85% sequence identity to the amino acid sequence of SEQ ID NO:18.
